(19) United States
(12) Patent Application Publication
(10) Pub. No.: US 2026/0055096 A1
(43) Pub. Date: Feb. 26, 2026

(54) AZOLE PESTICIDAL COMPOUNDS

(71) Applicant: BASF SE, Ludwigshafen (DE)

(72) Inventors: Rupsha Chaudhuri, Navi Mumbai (IN); Pulakesh Maity, Navi Mumbai (IN); Christian Defieber, Ludwigshafen (DE); Kishor Handore, Navi Mumbai (IN); Ashokkumar Adisechan, Navi Mumbai (IN); Christian Harald Winter, Ludwigshafen (DE); Karsten Koerber, Ludwigshafen (DE); Matthew Charles Linford Wakeham, Ludwigshafen (DE)

(21) Appl. No.: 19/112,314

(22) PCT Filed: Sep. 15, 2023
(86) PCT No.: PCT/EP2023/075459

§ 371 Date: Mar. 17, 2025

(30) Foreign Application Priority Data
Sep. 19, 2022 (EP) 22196425.7

(51) Int. Cl. C07D 417/12; A01N 47/36; A01P 5/00; A01P 7/04

(52) U.S. Cl. CPC C07D 417/12; A01N 47/36; A01P 5/00; A01P 7/04

(57) ABSTRACT

The invention relates to the compounds of formula (I), and the N-oxides, stereoisomers, tautomers and agriculturally or veterinarily acceptable salts thereof, wherein the variables are defined according to the description. The compounds of formula (I), the N-oxides, stereoisomers, tautomers, and agriculturally or veterinarily acceptable salts thereof, are useful for combating or controlling invertebrate pests, in particular arthropod pests and nematodes. The invention also to plant propagation material and to an agricultural and a veterinary composition comprising said compounds.

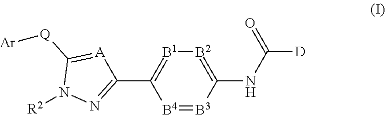

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

Carbamoylated and thiocarbamoylated oxime derivatives are known for pesticidal use, for example, in patent publications WO2016/156076, semi-carbazones and thiosemicarbazones derivatives are known for pesticidal use in patent publication WO2016/116445 and pyrazolo pesticidal compounds are known for pesticidal use in patent publication WO2021/013561.

Due to the ability of target pests to develop resistance to pesticidally-active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes.

It is therefore an object of the invention to identify and provide compounds, which exhibit a high pesticidal activity and have a broad activity spectrum against invertebrate pests.

It has been found that these objects can be achieved by substituted bicyclic compounds of formula I, as depicted and defined below, including their stereoisomers, their salts, in particular their agriculturally or veterinarily acceptable salts, their tautomers and their N-oxides.

In a first aspect, the invention relates to compounds of formula I

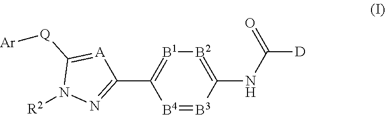

wherein
Q is —C(═O)—N(R⁵)—, or —N(R⁵)—C(═O)—;
R⁵ is H, C₁-C₆-alkyl, C₃-C₆-cycloalkyl, C₁-C₆-alkyl-C₁-C₆-alkoxy, C₁-C₆-alkyl-C₃-C₆-cycloalkyl, phenyl, 5- or 6-membered hetaryl, —CH₂-phenyl, —CH₂-5- or 6-membered hetaryl, 1,3-dioxolan-2-ylmethyl, or halogen, wherein the alkyl, cycloalkyl, phenyl and hetaryl moieties are unsubstituted or substituted with halogen or CN;
A is N or CR^{A};
R^{A} is H, C₁-C₆-alkyl, C₃-C₆-cycloalkyl, C₁-C₆-alkoxy, halogen, CN, or NR⁶R⁷ wherein the alkyl, alkoxy and cycloalkyl moieties are unsubstituted or substituted with halogen or CN;
R² is H or C₁-C₆-alkyl, or C₃-C₆-cycloalkyl, wherein the alkyl and cycloalkyl moieties are unsubstituted or substituted with halogen, CN, or C₁-C₆-alkoxy;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA or DB,

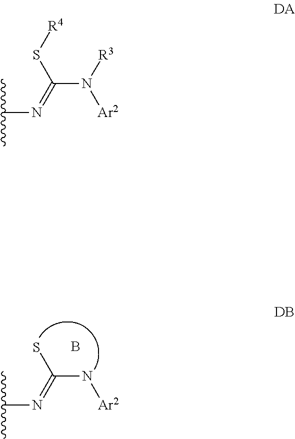

R³ is H, C₁-C₆-alkyl, or C₃-C₆-cycloalkyl, wherein the alkyl and cycloalkyl moieties are unsubstituted or substituted with halogen or CN;
R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkoxy, —O—(C═O)—C₁-C₆-alkyl or CN; or
B is a 5- or 6-membered carbocyclic group, wherein 1 or 2 CH₂ moieties of the carbocyclic group may be replaced by a carbonyl group, O, or S, wherein the carbocyclic group is unsubstituted or substituted with R^{h};
Ar¹ is phenyl or 5- or 6-membered heteroaryl, which are unsubstituted or substituted with R^{Ar1}, wherein
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f},
   C(═O)—OR^{a}, NR^{b}R^{c}, C₁-C₆-alkylene-CN, C(═O)—NR^{b}R^{c}, C(═O)—R^{d}, NHS(═O)ₘR^{e}, S(═O)ₘR^{e}, —N═S(═O)—(C₁-C₆-alkyl)₂, or SO₂NR^{b}R^{c};
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl and heteroaryl moieties are unsubstituted or substituted with halogen, CN, C₁-C₆-alkyl or C₁-C₆-alkoxy;
Ar² is phenyl or 5- or 6-membered heteroaryl, which are unsubstituted or substituted with R^{Ar2}, wherein
R^{Ar2} is halogen, CN, —SCN, —SF₅, C₁-C₆-alkyl, C₁-C₆-alkoxy, C₂-C₆-alkenyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, cycloalkyl and cycloalkoxy moieties are unsubstituted or substituted with halogen or CN; C(═O)—OR^{a}, NR^{b}R^{c}, C₁-C₆-alkylene-CN, C(═O)—NR^{b}R^{c};
R^{a}, R^{b} and R^{c} are, identical or different, H, C₁-C₆-alkyl, C₂-C₆-alkenyl, C₃-C₆-cycloalkyl, C₃-C₆-cycloalkyl-C₁-C₄-alkyl, —C(═O)—C₁-C₆-alkyl wherein the alkyl, alkenyl and cycloalkyl moieties are unsubstituted or substituted with halogen;
R^{d} is H or C₁-C₆-alkyl;
R^{e} is C₁-C₆-alkyl or C₃-C₆-cycloalkyl, wherein the alkyl, cycloalkyl moieties are unsubstituted or substituted with halogen;
m is 0, 1, or 2;
R^{f} is halogen, OH, CN, SCN, SFS, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, or C₃-C₆-cycloalkoxy-C₁-C₄-alkyl, wherein the alkyl, alkoxy, alkenyl, alkynyl, cycloalkyl and cycloalkoxy moieties are unsubstituted or substituted with halogen;
R^{h} is halogen, C₁-C₆-alkyl, or C₁-C₆-alkoxy;
and the N-oxides, stereoisomers, tautomers and agriculturally or veterinarily acceptable salts thereof.

Moreover, the invention also relates to processes and intermediates for preparing compounds of formula I and to active compound combinations comprising them. Moreover, the invention relates to agricultural or veterinary compositions comprising the compounds of formula I, and to the use of the compounds of formula I or compositions comprising them for combating or controlling invertebrate pests and/or for protecting crops, plants, plant propagation material and/or growing plants from attack and/or infestation by invertebrate pests. The invention also relates to methods of applying the compounds of formula I. The invention also relates to method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);

Furthermore, the invention relates to seed comprising compounds of formula I. Wherein the compounds of formula I includes N-oxides, stereoisomers, tautomers and agriculturally or veterinarily acceptable salts thereof.

With due modification of the starting compounds, the compounds of formula I can be prepared by procedures as given in below schemes.

The compounds of the formula (I) can be prepared by methods of organic chemistry, e.g., by the methods described herein after in schemes 1 to 24 in the synthesis description of the examples. In schemes 1 to 24 the radicals Ar¹, A, B¹, B², B³, B⁴, D, Q, X, R^{A}, R², R³, R⁴, R⁵, R⁶, R⁷, R^{h}, and Ar² are as defined above for formula (I), unless otherwise specified.

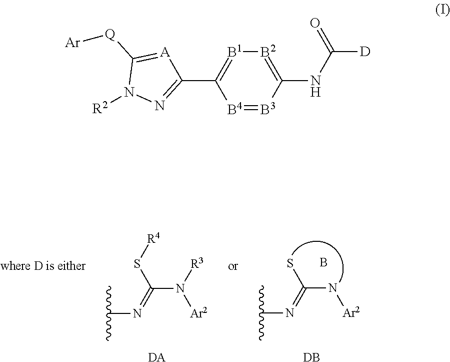

Compounds of formula (1), wherein R⁴ is not H are the compounds of formula (Ia) can be prepared analogously to the methods described in WO 2021/011722 or methods described in Scheme 1:

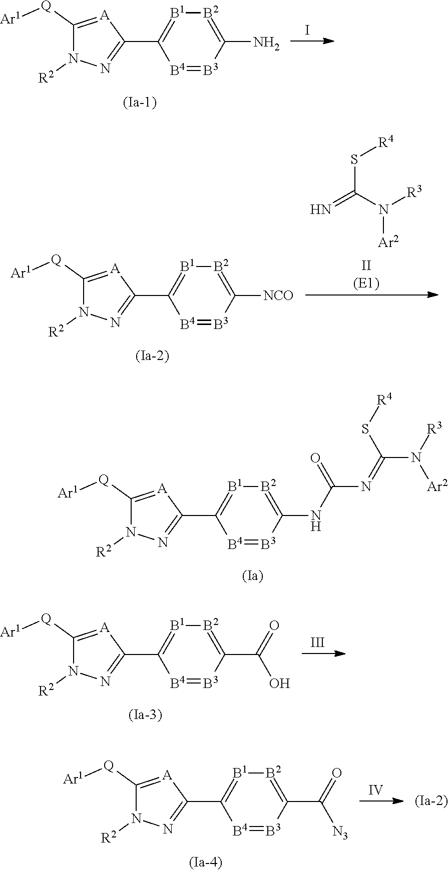

In one embodiment of Scheme 1, compounds of formula (Ia-2) are reacted directly with a compound of formula (E1) in the presence of an inorganic base to form a compound of formula (Ia). An isocyanate compound of formula (Ia-2) can be generated in situ from either an amine of the formula (Ia-1) by using one of the common reagents such as phosgene, diphosgene, triphosgene or carbonyldiimidazole (Step 1) in a mixed solvent system and in the presence of a base as described in March's Advanced Organic Chemistry 6^{th} edition, Michael B. Smith and Jerry March.

According to another embodiment of Scheme 1, an isocyanate compound of formula (Ia-2) is generated via a Curtius rearrangement of an acyl azide (Ia-4), e.g. analogously to the method described in WO2014/204622. The acyl azide of the formula (Ia-4) can be prepared from the corresponding carboxylic acid precursor of formula (Ia-3) by treatment with ethyl chloroformate and sodium azide in the presence of an amine base such as triethylamine, or with diphenyl-phosphoryl azide in the presence of an amine base such as triethylamine.

Compounds of formula (1), wherein the compounds of formula (Ia) can also be prepared analogously to the methods described in WO2021/011722 or methods described in Scheme 2:

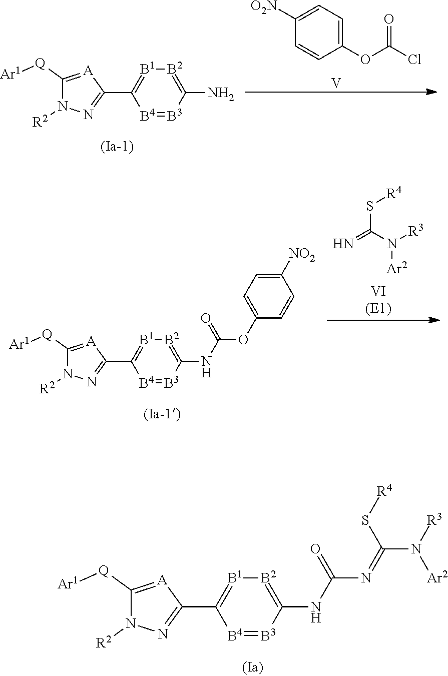

According to the method depicted in scheme 2, amine of formula (Ia-1) can be treated with an activating agent such as 4-nitrophenyl chloroformate in the presence of a polar aprotic solvent preferably tetrahydrofuran to generate an activated carbamate intermediate (Ia-1′), which in turn is reacted with the compound of formula (E1), in the presence of organic base such as DIPEA to form compound of formula (Ia). Compounds of formula (E1) can be prepared analogously to the methods described in WO2021/011722.

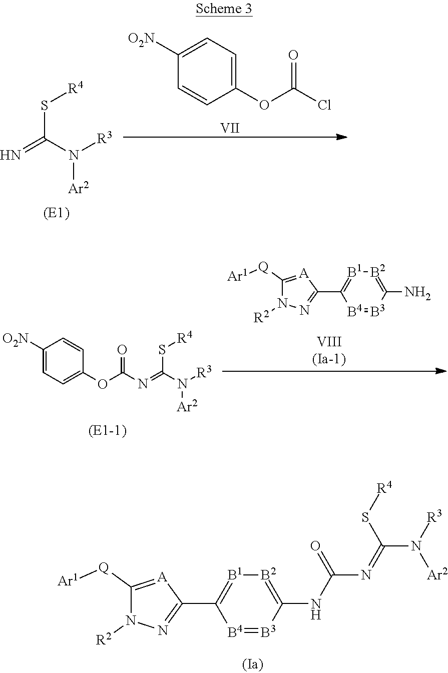

As depicted in scheme 3, compound of formula (E1) can be treated with an activating agent such as 4-nitrophenyl chloroformate in the presence of a polar aprotic solvent preferably tetrahydrofuran and an inorganic base such as cesium carbonate or potassium carbonate to generate an activated carbamate intermediate (E1-1), which in turn is reacted with amine of formula (Ia-1) in the presence of an inorganic base such as cesium carbonate or potassium carbonate to form compound of formula (Ia).

Compounds of formula (I), wherein R⁴ is H are the compounds of formula (Ia′) and can be prepared analogously to the methods described in WO2021/011722 or methods described in Scheme 4:

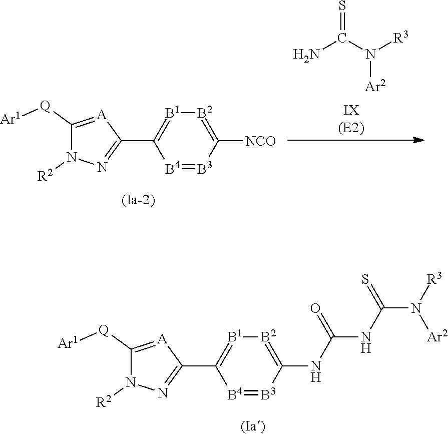

As depicted in scheme 4, compounds of the formula (Ia′) can be prepared by treating aryl thiourea of formula (E2) with the isocyanate of formula (Ia-2) in the presence of inorganic bases such as cesium carbonate in an aprotic solvent. Compounds of formula (E2) can be prepared analogously to the methods described in WO2021/011722.

Compounds of formula (E1-1), (E1-II), (E1-III) and (E1-IV) can be prepared analogously to the methods described in J. Med. Chem. 2010, 53(10), 4198-4211 or methods described in Scheme 5:

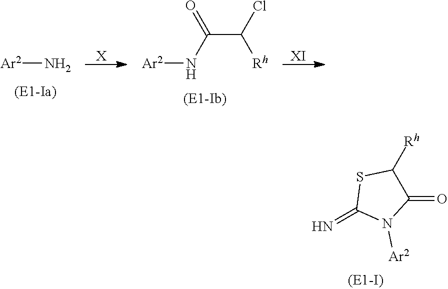

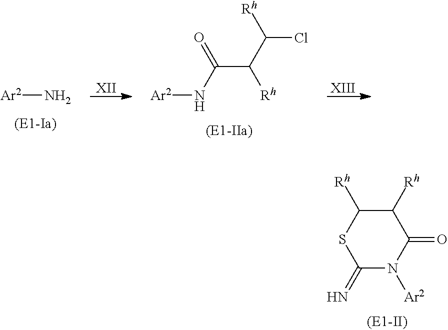

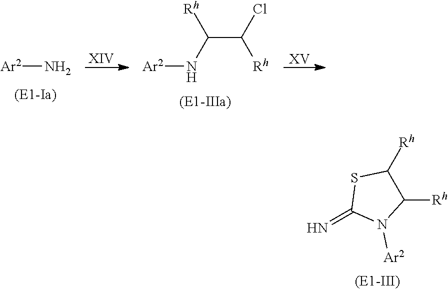

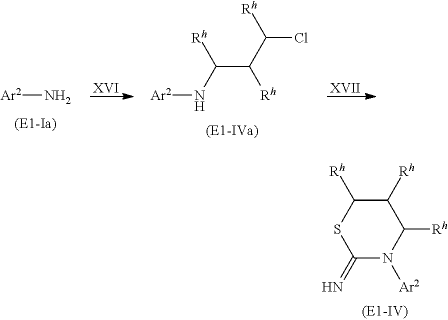

In the above reactions, compounds of formula (E1-Ia) can be converted into a variety of cyclized analogs of formula (E1-I), (E1-II), (E1-III) and (E1-IV). Compounds of formula (E1-I) and (E1-II) can be prepared by treatment of compounds of formula (E1-Ia) with unsubstituted or mono- or di-substituted 2-chloroacetylchloride and 3-chloropropanoylchloride in two steps as depicted in J. Med. Chem. 2010, 53(10), 4198-4211. Compounds of formula (E1-IIIa) and (E1-IVa) can be prepared by treatment of compounds of formula (E1-Ia) with unsubstituted or mono- or di-substituted 2-chloroactaldehyde and 3-chloro-propanal in two steps as mentioned in J. Het. Chem. 2006, 43(6), 1523-1531. Compounds of formula (E1-III) and (E1-IV) can be prepared by treatment of compounds of formula (E1-IIIa) and (E1-IVa) with potassium thiocyanate in presence of inorganic bases such as cesium carbonate in an aprotic solvent such as acetone.

Compounds of formula (Ia-1), (Ia-II), (Ia-III) and (Ia-IV) can be prepared from the compound of formula (Ia′) analogously to the methods described in WO2021/011722 or methods described in Scheme 6:

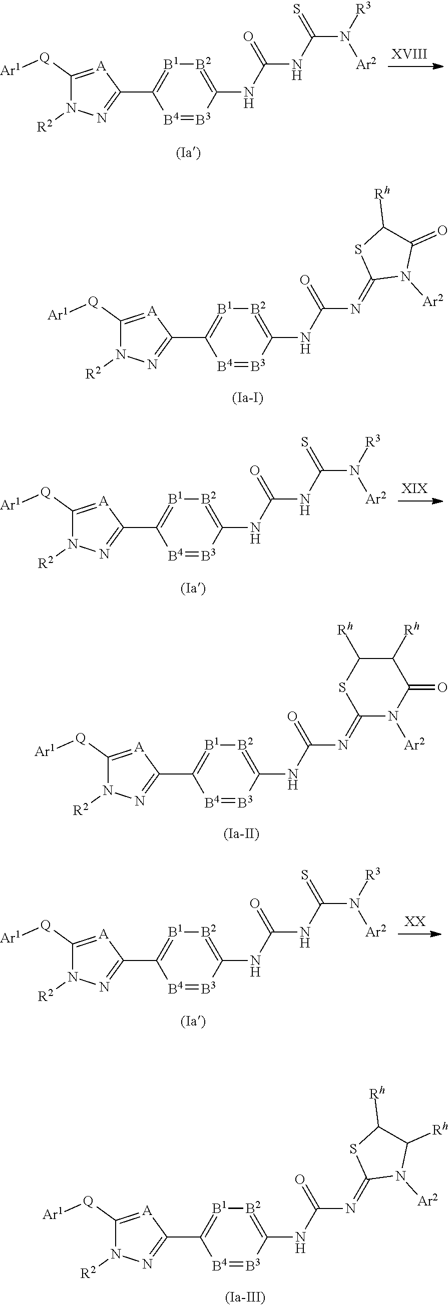

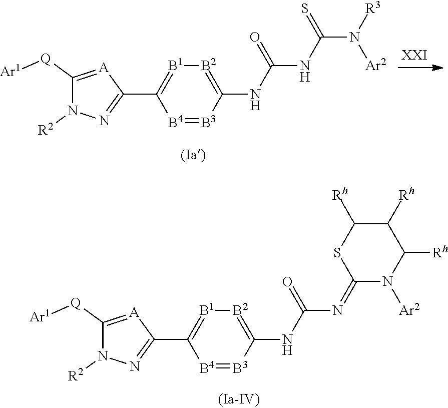

Compounds of formula (Ia′, Scheme 6) can be converted into a variety of cyclized analogs of formula (Ia-I), (Ia-II), (Ia-III) and (Ia-IV). Cyclization can be achieved by treatment of compounds of formula (Ia′) with a-halo esters such as methyl bromoacetate or methyl bromo propanoate to form compounds of formula (Ia-I) and (Ia-II) unsubstituted or mono- or di-substituted with Rh. Compounds of formula (Ia-III) and (Ia-IV) unsubstituted or mono- or di-substituted with R^{h} can be prepared by treatment of compounds of formula (Ia′) with vicinal dihalides. For steps XVIII and XIX, use of sodium acetate in a protic solvent such as ethanol, at temperatures ranging from about 20° C. to about 70° C. is preferred. For steps XX and XXI, use of an inorganic base such as potassium carbonate in a solvent such as ACN or 2-butanone, at a temperature between about 0° C. and about 80° C., is preferred. All the above reactions can be performed analogously to the methods described in WO2021/011722.

Compounds of formula (Ia-1) can be prepared analogously to the methods described in scheme 7:

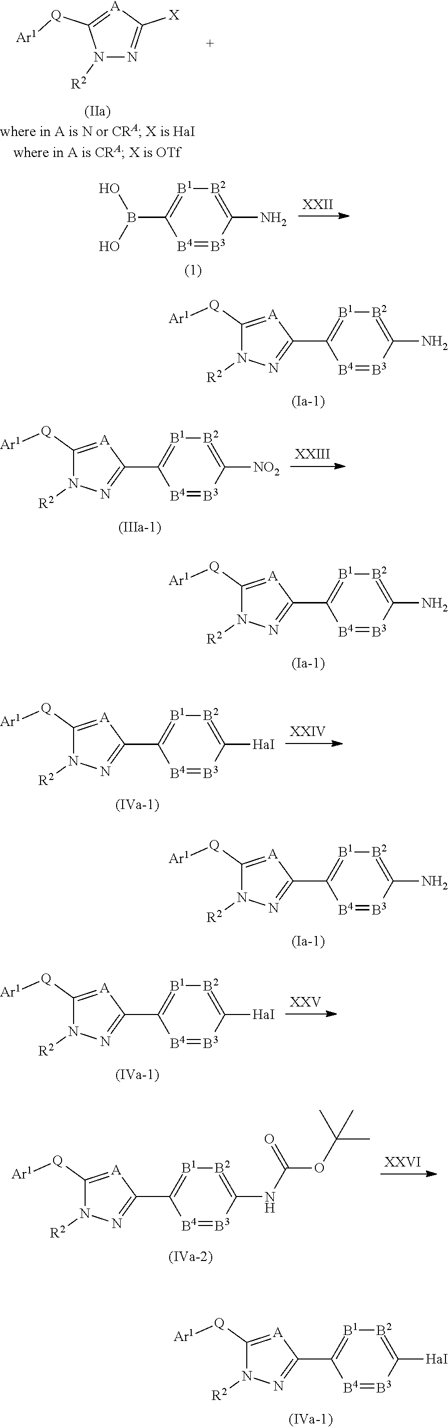

As depicted in scheme 7, compounds of formula (Ia-1) can be prepared with different synthetic routes. Step XXII can be performed via Suzuki cross coupling reaction starting from an appropriate aryl boronic acid precursor (1) as described in either Tetrahedron, 2009, 65(37), 7817-7824 or WO2018/075937.

Alternatively, compounds of formula (Ia-1) can be prepared by reduction of nitro compounds of formula (IIIa-1) using reducing agents such as SnCl₂ in acid medium as shown in step XXIII.

Alternatively, compounds of formula (Ia-1) can also be prepared by reacting compounds of the formula (IVa-1) with ammonia in the presence of a metal catalyst or its salts, preferably copper or its salts as described in Chem. Commun., 2009, 3035-3037.

Additionally, compounds of formula (Ia-1) can also be prepared in two steps from compounds of the formula (IVa-1). Treatment of compounds of formula (IVa-1) with tert-butyl carbamate in the presence of metal catalyst or its salts, preferably palladium or its salts to form compounds of formula (IVa-2) in step XXV, followed by Boc-deprotection using trifluoroacetic acid or diluted hydrochloric acid to form the desired compound in step XXVI. All these reactions are performed as described in March's Advanced Organic Chemistry 6^{th} edition, Michael B. Smith and Jerry March.

Compounds of formula (IIIa-1) can be prepared analogously to the methods described in scheme 8:

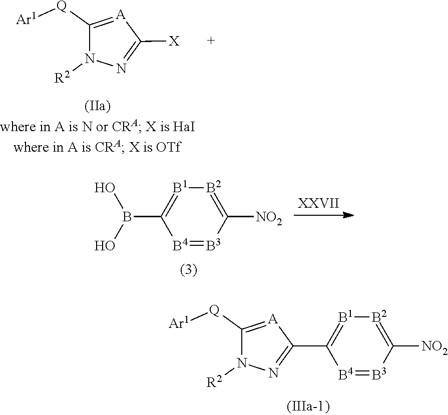

Step XXVII can be performed via Suzuki cross coupling reaction starting from an aryl boronic acid precursor (3) as described in Tetrahedron, 2009, 65(37), 7817-7824 or WO2018/075937.

Compounds of formula (IVa-1) can be prepared analogously to the methods described in scheme 9:

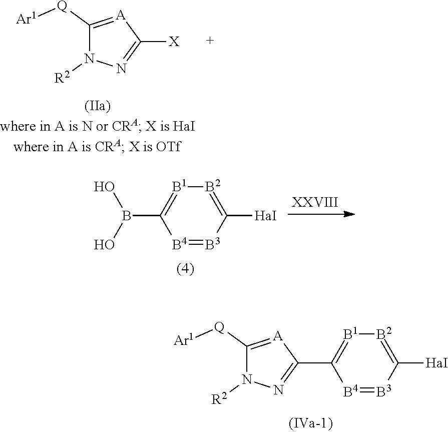

Step XXVIII can be performed via Suzuki cross coupling reaction starting from an aryl boronic acid precursor (4) as described in Tetrahedron, 2009, 65(37), 7817-7824 or WO2018/075937.

Compounds of formula (IVa-1) wherein Q is —C(═O)—N(R⁵)—, A is N or CR^{A} and R⁵ is H are the compounds of formula (IVa-1-1) can be prepared analogously to the methods described in scheme 10:

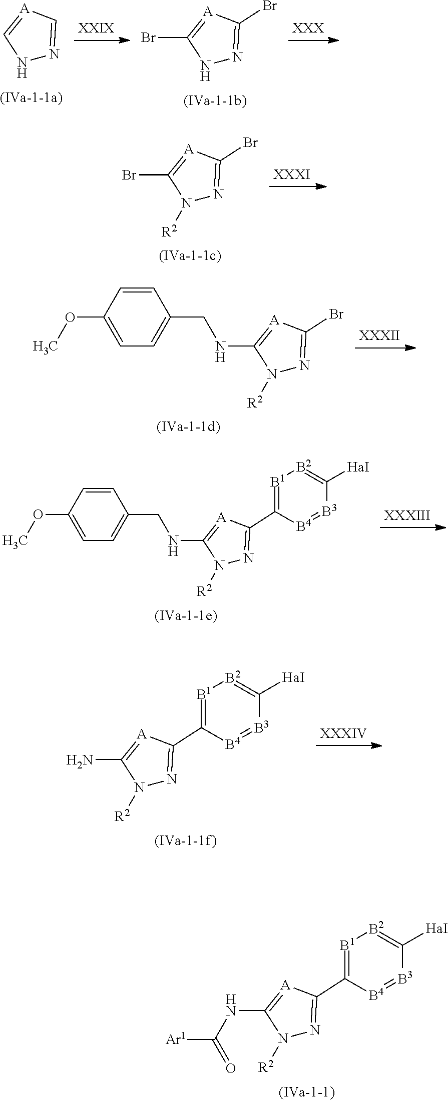

Compounds of formula (IVa-1-1) can be prepared from compounds of formula (IVa-1-1a) in six steps. Step XXIX includes bromination by reacting the compounds of formula (IVa-1-1a) with bromine in presence of a weak base like sodium acetate, a protic solvent like ethanol and water.

Step XXX includes alkylation by reacting with corresponding commercially available alkyl halides preferably iodides or bromides in presence of bases like cesium carbonate and polar aprotic solvent like DMF, analogous to as described in March's Advanced Organic Chemistry 6^{th} edition, Michael B. Smith and Jerry March. Step XXXI includes introduction of protecting group such as 2,4-dimethoxybenzylamine. Step XXXII can be performed via Suzuki cross coupling reaction starting from an aryl boronic acid precursor (4) as described in WO2018/075937. Step XXXIII deprotection of compounds of formula (IVa-1-1e) by reacting compound of formula (IVa-1-1e) with 5N Hydrochloric acid. Step XXXIV involves amide formation by reacting the compounds of formula (IVa-1-1f) with Ar¹—COOH in presence of suitable coupling reagent like HATU and base like DIPEA. All these steps can be performed as described in March's Advanced Organic Chemistry 6^{th} edition, Michael B. Smith and Jerry March. The compounds of formula (IVa-1-1) can also be synthesized by treating compounds of formula (IVa-1-1f) with commercially available benzoyl chlorides in presence of base, as described in March's Advanced Organic Chemistry 6^{th} edition, Michael B. Smith and Jerry March. Alternatively, the compounds of formula (IVa-1-1) can also be synthesized by in-situ generation of benzoyl chlorides from the corresponding benzoic acids with POCl₃ or SOCl₂ then followed by treatment with compounds of formula (IVa-1-1f) in presence of base as described in March's Advanced Organic Chemistry 6^{th} edition, Michael B. Smith and Jerry March.

Compounds of formula (IVa-1) wherein Q is —C(═O)—N(R⁵)—, A is CR^{A}, R^{A} is H or C₁-C₆-alkyl or C₃-C₆-cycloalkyl or CN or C₁-C₆-alkoxy and R⁵ is H are the compounds of formula (IVa-1-1A) can be prepared analogously to the methods described in scheme 11:

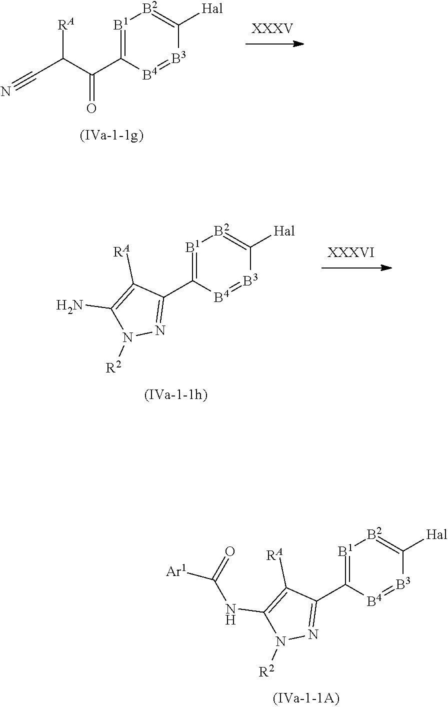

Compounds of formula (IVa-1-1A) can be prepared from commercially available benzoyl acetonitrile derivative (IVa-1-1g) by reacting with R²—NHNH₂ as described in WO2018/069222. Common intermediate of formula (IVa-1-1A) can be prepared via amide formation by reacting the compounds of formula (IVa-1-1h) with Ar¹—COOH in presence of suitable coupling reagent like HATU and base like DIPEA. The compounds of formula (IVa-1-1A) can also be synthesized by treating compounds of formula (IVa-1-1h) with commercially available benzoyl chlorides in presence of base, as described in March's Advanced Organic Chemistry 6^{th} edition, Michael B. Smith and Jerry March. Alternatively, compounds of formula (IVa-1-1A) can also be synthesized by in-situ generation of benzoyl chlorides from the corresponding benzoic acids with POCl₃ or SOCl₂ then followed by treatment with compounds of formula (IVa-1-1h) in presence of base as described in March's Advanced Organic Chemistry 6^{th} edition, Michael B. Smith and Jerry March.

Compounds of formula (IVa-1) wherein Q is —C(═O)—N(R⁵)—, A is N and R⁵ is H are the compounds of formula (IVa-1-1B) can also be prepared analogously to the methods described in scheme 12:

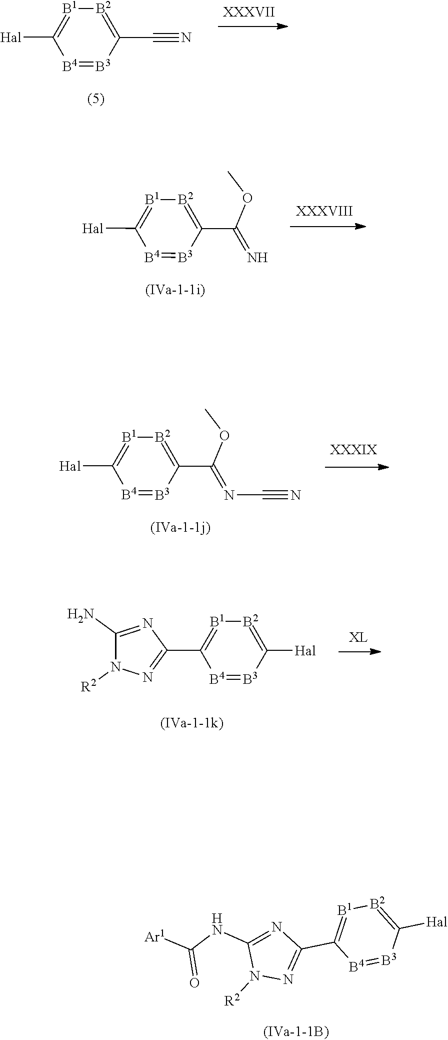

Compounds of formula (IVa-1-1B) can be prepared from commercially available benzonitriles (5) in four steps. Step XXXVII can be performed as mentioned in Green Chemistry, 2013, 15(8), 2252-2260. Step XXXVIII can be performed as mentioned in US2007/0078141. Step XXXIX involves the formation of triazole ring by heating the compounds of formula (IVa-1-1j) with R²NHNH₂ in a polar protic solvent like methanol. Common intermediate of formula (IVa-1-1 B) can be prepared via amide formation by reacting the compounds of formula (IVa-1-1k) with Ar¹—COOH in presence of suitable coupling reagent like HATU and base like DIPEA. The compounds of formula (IVa-1-1 B) can also be synthesized by treating compounds of formula (IVa-1-1k) with commercially available benzoyl chlorides in presence of base, as described in March's Advanced Organic Chemistry 6^{th} edition, Michael B. Smith and Jerry March. Alternatively, compounds of formula (IVa-1-1B) can also be synthesized by in-situ generation of benzoyl chlorides from the corresponding benzoic acids with POCl₃ or SOCl₂ then followed by treatment with the compounds of formula (IVa-1-1k) in presence of base as described in March's Advanced Organic Chemistry 6^{th} edition, Michael B. Smith and Jerry March.

Compounds of formula (IVa-1) wherein Q is —N(R⁵)—C(═O)—, A is CR^{A}, R^{A} is H or C₁-C₆-alkyl or C₃-C₆-cycloalkyl or CN or C₁-C₆-alkoxy and R⁵ is H are the compounds of formula (IVa-1-2) can be prepared analogously to the methods described in scheme 13:

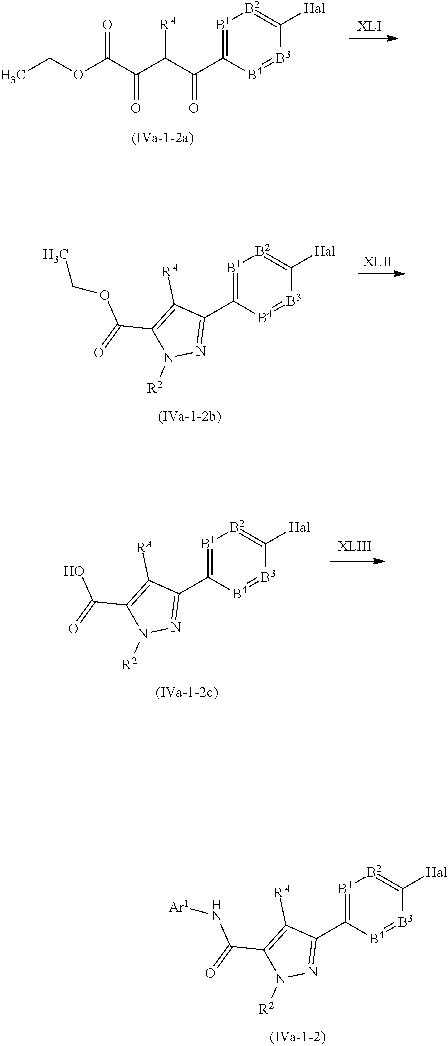

Compounds of formula (IVa-1-2c) can be prepared from a suitable starting point 2,4 dioxo-4-aryl-butyric acid ethyl ester derivative (IVa-1-2a, commercially available) in two steps as described in Chem. Central Journal, 2016, 10 (40), 1-6. Compounds of formula (IVa-1-2c) can be prepared from ester intermediate (IVa-1-2b) by hydrolysis with suitable base like LiOH, NaOH, as mentioned in WO2011/050245. Common intermediate of formula (IVa-1-2) can be prepared via amide formation by reacting the compounds of formula (IVa-1-2c) with Ar¹—NH₂ in presence of suitable coupling reagent like HATU and base like DIPEA.

Compounds of formula (IVa-1), wherein A is CR^{A} or N, Q is —N(R⁴)—C(═O)— and R⁵ is H, are compounds of formula (IVa-1-3) and can be prepared analogously to the methods described in scheme 14:

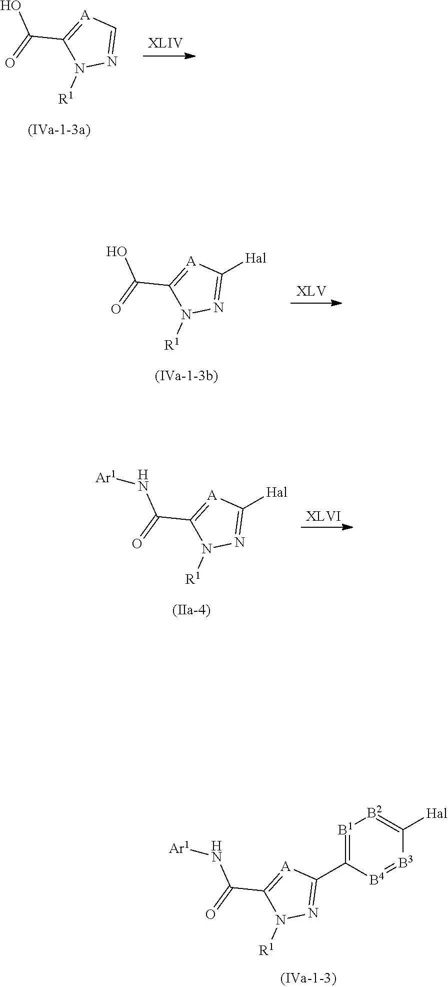

Compounds of formula (IVa-1-3) can be prepared from compounds of formula (IVa-1-3a) in three steps. Step XLIV includes halogenation and step XLV includes amide formation by reacting the compounds of formula (IVa-1-3b) with Ar¹—NH₂ in presence of a suitable coupling reagent like HATU and base like DIPEA and step XLVI includes Suzuki cross coupling of compounds of formula (IIa-4) with 4-halo-phenyl boronic acid derivatives. All these steps can be performed as described in March's Advanced Organic Chemistry 6^{th} edition, Michael B. Smith and Jerry March.

Compounds of formula (IVa-1) wherein Q is —C(═O)—N(R⁵)—, A is CR^{A}, R^{A} is Cl and R⁵ is H, are the compounds of formula (IVa-1-4) that can be prepared analogously to the methods described in scheme 15:

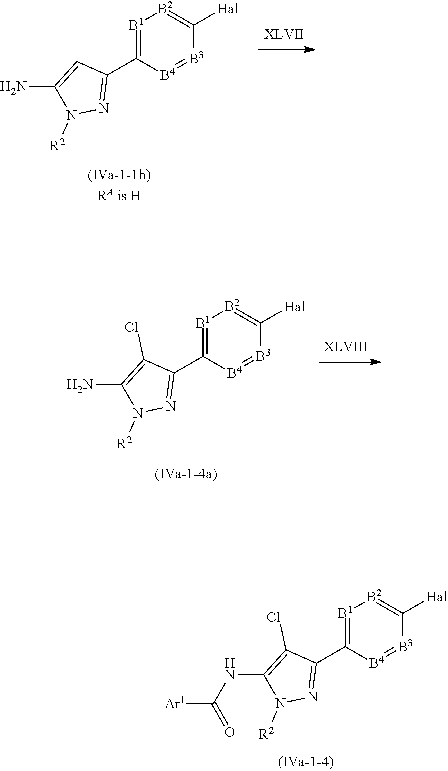

Compounds of formula (IVa-1-4) can be prepared from compounds of formula (IVa-1-1h) in two steps. Step XLVII involves chlorination using N-chlorosuccinimide in a polar aprotic solvent like ACN as described in WO2014/078325. Step XLVIII involves amide coupling reaction as described in March's Advanced Organic Chemistry 6^{th} edition, Michael B. Smith and Jerry March.

Compounds of formula (IVa-1) wherein Q is —C(═O)—N(R⁵)—, R^{A} is NR⁶R⁷ and R⁵ is H, are compounds of formula (IVa-1-5) that can be prepared analogously to the methods described in scheme 16:

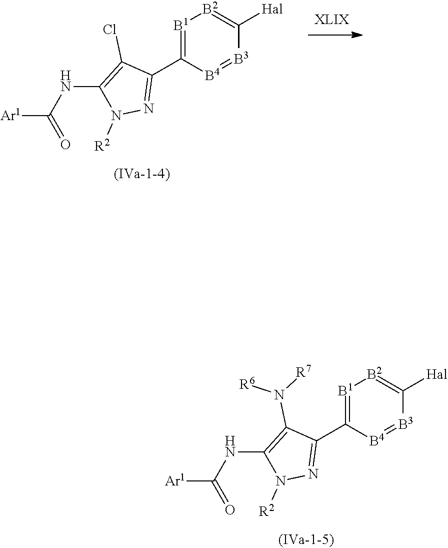

Compounds of formula (IVa-1-5) can be prepared from compounds of formula (IVa-1-4) in one step. Amination of compounds of formula (IVa-1-4) wherein R⁷ or R⁶ is C₁-C₆-alkyl or C₃-C₆-cycloalkyl —CH₂-phenyl or —CH₂-5- or 6-membered hetaryl or 1,3-dioxolan-2-ylmethyl or 2-(methylamino)-2-oxo-ethyl, can be performed by reacting with corresponding commercially available alkyl amines or benzyl amines in presence of bases like TEA and polar aprotic solvents like DMF to get compounds of formula (IVa-1-5). Compounds of formula (IVa-1-5), wherein R⁷ or R⁶ is a phenyl or 5- or 6-membered hetaryl ring, can be prepared from compounds of formula (IVa-1-4) by metal catalyzed reaction with corresponding aryl halides or 5- or 6-membered hetaryl halide preferably iodides or bromides as describe in Chinese J. Chem. 2012, 30(10), 2356-2362. Compounds of formula (IVa-1-5) wherein R⁷ and R⁶ are H can be prepared from compounds of formula (IVa-1-4) with ammonia in the presence of a metal catalyst or its salts, preferably copper or its salts as described in Chem. Commun., 2009, 3035-3037.

Compounds of formula (IVa-1) wherein Q is —N(R⁴)—C(═O)—, R^{A} is Cl and R⁵ is H, are compounds of formula (IVa-1-6) that can be prepared analogously to the methods described in scheme 17:

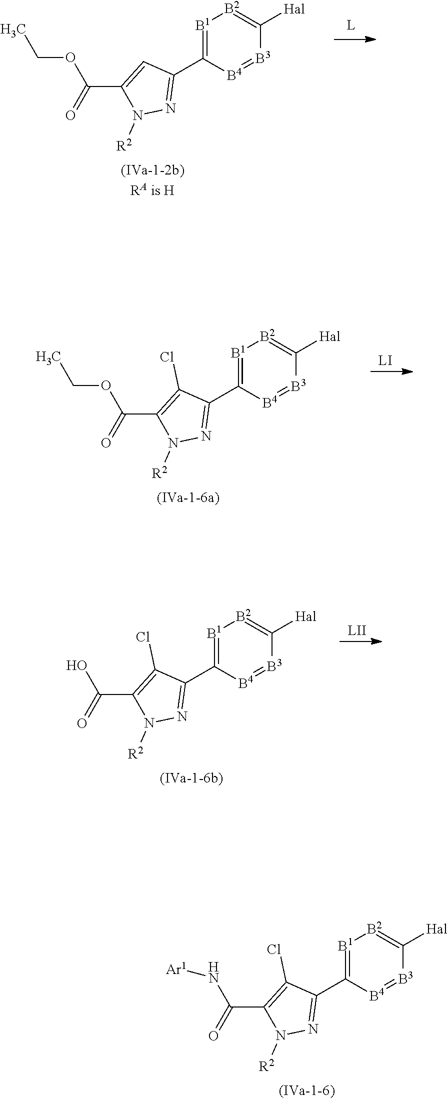

Compounds of formula (IVa-1-6) can be prepared from compounds of formula (IVa-1-2b) in three steps. Step L involves chlorination using SOCl₂ as described in Youji Huaxue, 2010, 30(11), 1726-1731. Step LI involves hydrolysis with suitable base like LiOH, NaOH, as mentioned in WO 2011/050245. Step LII involves amide formation by reacting the compounds of formula (IVa-1-6b) with Ar¹—NH₂ in presence of suitable coupling reagent like HATU and base like DIPEA.

Compounds of formula (IIa) wherein Q is —C(═O)—N(R⁵)—, A is CR^{A}, X is Br and R⁵ is H are the compounds of formula (IIa-1) can be prepared analogously to the methods described in scheme 18:

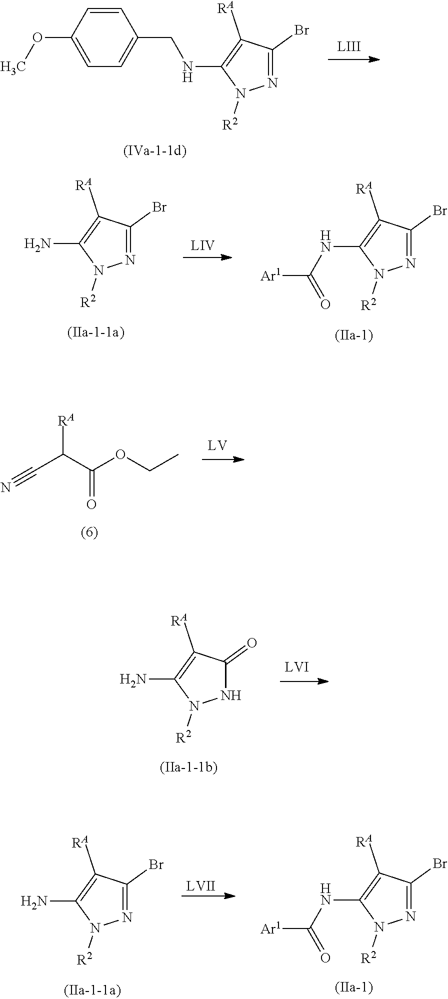

Compounds of formula (IIa-1) can be prepared from compounds of formula (IVa-1-1d) in two steps. Step LIII involves deprotection of compounds of formula (IVa-1-1d) by reacting compound of formula (IVa-1-1d) with 5N hydrochloric acid or TFA in 1,4-dioxane. Step LIV involves amide formation by reacting the compounds of formula (IIa-1-1a) with Ar¹—COOH in presence of suitable coupling reagent like HATU and base like DIPEA. The compounds of formula (IIa-1) can also be synthesized by treating compounds of formula (IIa-1-1a) with commercially available benzoyl chlorides in presence of base. Alternatively, the compounds of formula (IIa-1) can also be synthesized by in-situ generation of benzoyl chlorides from the corresponding benzoic acids with POCl₃ or SOCl₂ then followed by treatment with compounds of formula (IIa-1-1a) in presence of base. All these steps can be performed as described in March's Advanced Organic Chemistry 6^{th} edition, Michael B. Smith and Jerry March.

Alternatively, compounds of formula (IIa-1) can also be prepared from commercially available substituted ethyl cyanoacetates (6) via a common intermediate (IIa-1-1a) in three steps. Compounds of formula (IIa-1-1a) can be prepared from compounds of formula (6) analogously to the methods described in WO2014/78323.

Compounds of formula (IIa) wherein Q is —C(═O)—N(R⁵)—, A is CR^{A}, X is OTf and R⁵ is H are the compounds of formula (IIa-2) can be prepared analogously to the methods described in scheme 19:

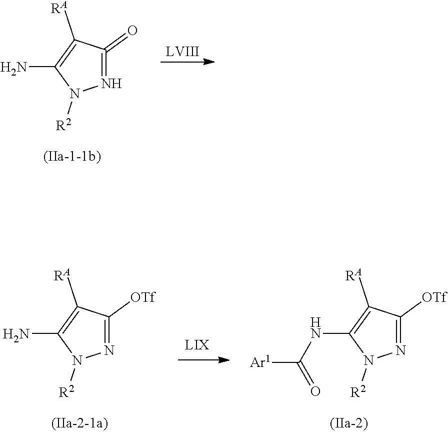

Compounds of formula (IIa-2) can be prepared from a common intermediate (IIa-1-1b) analogously to the methods described in WO2014/78323.

Compounds of formula (IIa) wherein Q is —C(═O)—N(R⁵)—, A is N and R⁵ is H are the compounds of formula (IIa-3) can be prepared analogously to the methods described in scheme 20:

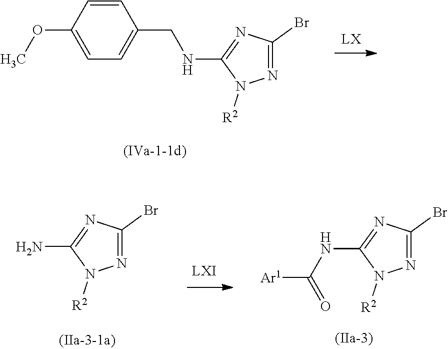

Compounds of formula (IIa-3) can be prepared from compounds of formula (IVa-1-1d) in two steps. Step LX involves deprotection of compounds of formula (IVa-1-1d) by reacting compound of formula (IVa-1-1d) with 5N hydrochloric acid or TFA in 1,4-dioxane. Step LXI involves amide formation by reacting the compounds of formula (IIa-3-1a) with Ar¹—COOH in presence of suitable coupling reagent like HATU and base like DIPEA. The compounds of formula (IIa-3) can also be synthesized by treating compounds of formula (IIa-3-1a) with commercially available benzoyl chlorides in presence of base. Alternatively, the compounds of formula (IIa-3) can also be synthesized by in-situ generation of benzoyl chlorides from the corresponding benzoic acids with POCl₃ or SOCl₂ then followed by treatment with compounds of formula (IIa-3-1a) in presence of a base. All these steps can be performed as described in March's Advanced Organic Chemistry 6^{th} edition, Michael B. Smith and Jerry March.

Compounds of formula (IIa) wherein Q is —N(R⁵)—C(═O)—, A is CR^{A}, X is OTf and R⁵ is H are the compounds of formula (IIa-5) analogously to the methods described in scheme 21:

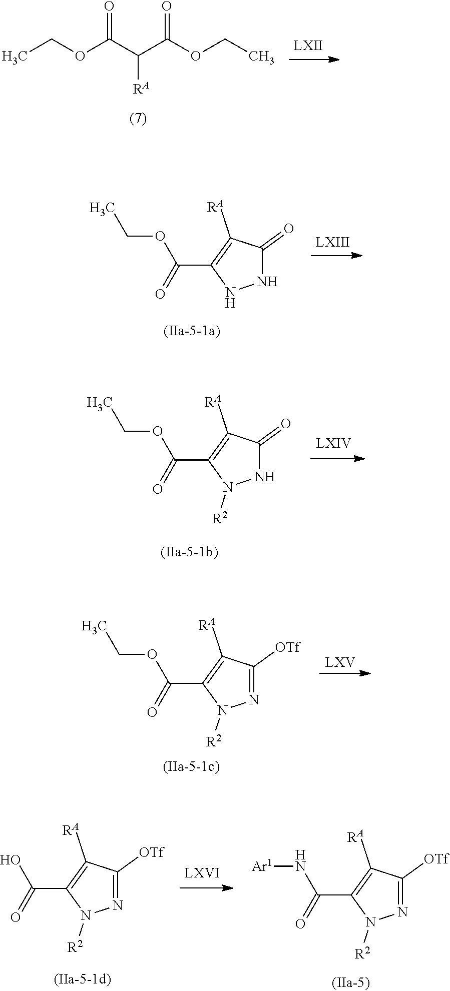

Compounds of formula (IIa-5) can be prepared from commercially available substituted diethyl propanedioate (7) in five steps. Step LXII can be performed as described in Org. Lett., 2014, 16(23), 6120-6123. Compounds of formula (IIa-5-1b) can be prepared from compounds of formula (IIa-5-1a) analogously to the methods described in US 2019/0127358. Step LXIV can be performed as described in WO 2018/125961. Last two steps involve hydrolysis with suitable base like LiOH, NaOH, as mentioned in WO 2011/050245 followed by amide formation by reacting the compounds of formula (IIa-5-1d) with Ar¹—NH₂ in presence of suitable coupling reagent like HATU and base like DIPEA.

Compounds of formula (Ia-1) wherein Q is —C(═O)—N(R⁵)—, R⁵ is C₁-C₆-alkyl or C₃-C₆-cycloalkyl or —CH₂-phenyl or —CH₂-5- or 6-membered hetaryl or 1,3-dioxolan-2-ylmethyl or phenyl or 5- or 6-membered hetaryl, are the compounds of formula (Ia-1-1) can be prepared analogously to the methods described in scheme 22:

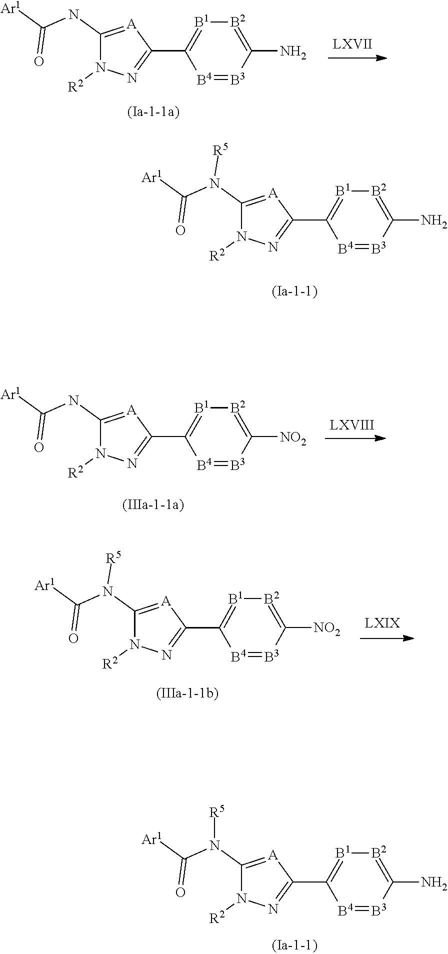

Compounds of formula (Ia-1-1) can be prepared from compounds of formula (Ia-1-1a) with R⁵ as C₁-C₆-alkyl or C₃-C₆-cycloalkyl or —CH₂-phenyl or —CH₂-5- or 6-membered hetaryl or 1,3-dioxolan-2-ylmethyl, by reacting with corresponding commercially available alkyl halides or benzyl halides preferably iodides or bromides in presence of bases like cesium carbonate and polar aprotic solvent like DMF, analogous to as described in March's Advanced Organic Chemistry 6^{th} edition, Michael B. Smith and Jerry March. Compounds of formula (Ia-1-1), wherein R⁵ is a phenyl or 5- or 6-membered hetaryl ring, can be prepared from compounds of formula (Ia-1-1a) by metal catalyzed reaction with corresponding aryl halides or 5- or 6-membered hetaryl halide preferably iodides or bromides as describe in Chinese J. Chem. 2012, 30(10), 2356-2362. Alternatively, compounds of formula (Ia-1-1) can be prepared from compounds of formula (IIIa-1-1a) in two steps. Step LXVIII can be performed similarly to step LXVII and step LXIX involves reduction using reducing agents such as SnCl₂ in ethanol or Fe with NH₄Cl in a mixture of ethanol, THF and water.

Compounds of formula (Ia-1) wherein Q is —N(R⁵)—C(═O)—, R⁵ is C₁-C₆-alkyl or C₃-C₆-cycloalkyl or —CH₂-phenyl or —CH₂-5- or 6-membered hetaryl or 1,3-dioxolan-2-ylmethyl or phenyl or 5- or 6-membered hetaryl, are the compounds of formula (Ia-1-2) can be prepared analogously to the methods described in scheme 23:

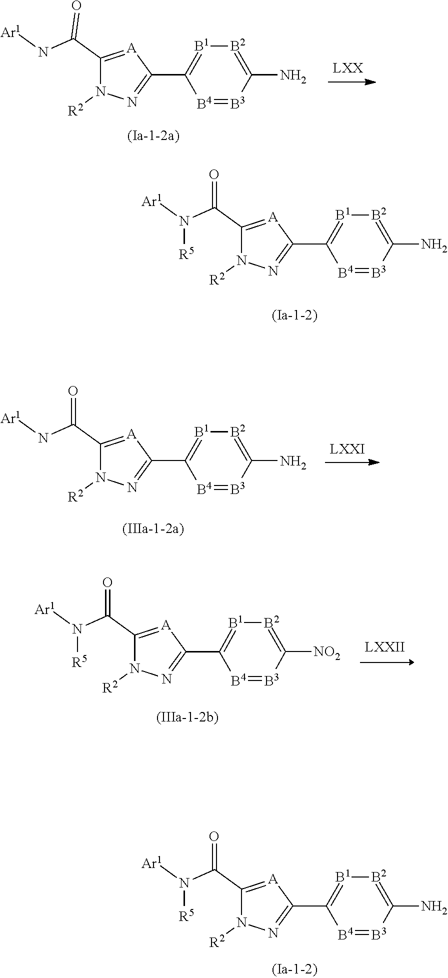

Compounds of formula (Ia-1-2) can be prepared from compounds of formula (Ia-1-2a) or from compounds of formula (IIIa-1-2a) using the reaction conditions disclosed under scheme 16.

Compounds of formula (Ia-1) wherein B² or B³ is Hal, are the compounds of formula (Ia-1-3) can be prepared analogously to the methods described in scheme 24:

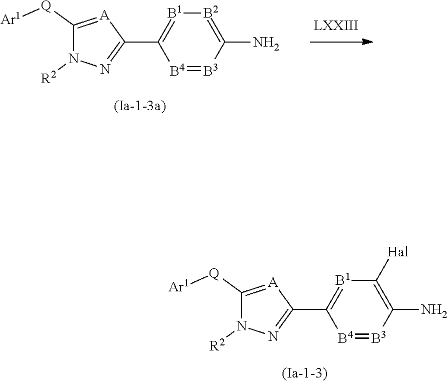

Compounds of formula (Ia-1-3) can be prepared by treating compounds of formula (Ia-1-3a) with electrophilic halogenating agent such as NXS (X═Cl, Br, I etc.) in a polar aprotic solvent like ACN analogous to as described in March's Advanced Organic Chemistry 6^{th} edition, Michael B. Smith and Jerry March.

Individual compounds of formula I can also be prepared by derivatisation of other compounds of formula I or the intermediates thereof.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

Unless otherwise indicated, the term “compound(s) according to the invention” or “compound(s) of the invention” or “compound(s) of formula (I)”, refers to
the compounds of formula I.

The term “compound(s) according to the invention”, or “compounds of formula I” comprises the compound(s) as defined herein as well as a stereoisomer, salt, tautomer or N-oxide thereof. The term “compound(s) of the invention” is to be understood as equivalent to the term “compound(s) according to the invention”, therefore also comprising a stereoisomer, salt, tautomer or N-oxide thereof.

The term “composition(s) according to the invention” or “composition(s) of the invention” encompasses composition(s) comprising at least one compound of formula I according to the invention as defined above. The compositions of the invention are preferably agricultural or veterinary compositions.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The invention relates to amorphous and crystalline compounds according to the invention, mixtures of different crystalline states of the respective compounds according to the invention, as well as amorphous or crystalline salts thereof.

The term “tautomers” encompasses isomers, which are derived from the compounds of formula I by the shift of an H-atom involving at least one H-atom located at a nitrogen, oxygen or sulphur atom. Examples of tautomeric forms are keto-enol forms, imine-enamine forms, urea-isourea forms, thiourea-isothiourea forms, (thio)amide-(thio)imidate forms etc.

The term “stereoisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO). To be more precise, it relates to any compound of the invention which has at least one tertiary nitrogen atom that is oxidized to an N-oxide moiety. N-oxides of compounds I can in particular be prepared by oxidizing e.g. the ring nitrogen atom of an N-heterocycle, e.g. a pyridine or pyrimidine ring present in Ar or R¹¹, or an imino-nitrogen present in central tricyclic core, with a suitable oxidizing agent, such as peroxo carboxylic acids or other peroxides. The person skilled in the art knows if and in which positions compounds of the invention may form N-oxides.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinarily acceptable salts are especially the salts of those cations or the acid addition salts of those acids whose cations and anions, which are known and accepted in the art for the formation of salts for agricultural or veterinary use respectively, and do not have any adverse effect on the action of the compounds according to the invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or —CH₂-phenyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium. Suitable acid addition veterinarily acceptable salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual members. The prefix Cₙ-Cₘ indicates in each case the possible number of carbon atoms in the group.

The term halogen denotes in each case F, Br, Cl or I, in particular F, Cl or Br.

The term “alkyl” as used herein and in the alkyl moieties of alkoxy, alkylthio, and the like refers to saturated straight-chain or branched hydrocarbon radicals having 1 to 2 (“C₁-C₂-alkyl”), 1 to 3 (“C₁-C₃-alkyl”),1 to 4 (“C₁-C₄-alkyl”) or 1 to 6 (“C₁-C₆-alkyl”) carbon atoms. C₁-C₂-Alkyl is CH₃ or C₂H₅. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“CG-C₄-haloalkyl”) or 1 to 6 (“C₁-C₆-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

The term “alkylene” (or alkanediyl) as used herein in each case denotes an alkyl radical as defined above, wherein one hydrogen atom at any position of the carbon backbone is replaced by one further binding site, thus forming a bivalent moiety. Alkylene has preferably 1 to 6 carbon atoms (C₁-C₆-alkylene), 2 to 6 carbon atoms (C₂—C-alkylene), in particular 1 to 4 carbon atoms (C₁-C₄-alkylene) or 2 to 4 carbon atoms (C₂-C₄-alkylene). Examples of alkylene are methylene (CH2), 1,1-ethandiyl, 1,2-ethandiyl, 1,3-propandiyl, 1,2-propandiyl, 2,2-propandiyl, 1,4-butandiyl, 1,2-butandiyl, 1,3-butandiyl, 2,3-butandiyl, 2,2-butandiyl, 1,5-pentandiyl, 2,2-dimethylpropan-1,3-diyl, 1,3-dimethyl-1,3-propandiyl, 1,6-hexandiyl etc.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”) or 2 to 6 (“C₂-C₆-alkenyl) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like.

The term “alkynyl” as used herein refers to straight-chain or branched hydrocarbon groups having 2 to 3 (“C₂-C₃-alkynyl”), 2 to 4 (“C₂-C₄-alkynyl”) or 2 to 6 (“C₂-C₆-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “cycloalkyl” as used herein refers to mono- or bi- or polycyclic saturated hydrocarbon radicals having in particular 3 to 6 (“C₃-C₆-cycloalkyl”) or 3 to 5 (“C₃-C₆-cycloalkyl”) or 3 to 4 (“C₃-C₄-cycloalkyl”) carbon atoms. Examples of monocyclic radicals having 3 to 4 carbon atoms comprise cyclopropyl and cyclobutyl. Examples of monocyclic radicals having 3 to 5 carbon atoms comprise cyclopropyl, cyclobutyl and cyclopentyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “cycloalkoxy” as used herein refers to a cycloalkyl radical, in particular a monocyclic cycloalkyl radical, as defined above having in particular 3 to 6 (“C₃-C₆-cycloalkoxy”) or 3 to 5 (“C₃-C₆-cycloalkoxy”) or 3 to 4 (“C₃-C₄-cycloalksoxy”) carbon atoms, which is bound via an oxygen atom to the remainder of the molecule.

The term “cycloalkyl-C₁-C₄-alkyl” refers to a C₃-C₈-cycloalkyl (“C₃-C₈-cycloalkyl-C₁-C₄-alkyl”), preferably a C₃-C₆-cycloalkyl (“C₃-C₆-cycloalkyl-C₁-C₄-alkyl”), more preferably a C₃-C₄-cycloalkyl (“C₃-C₄-cycloalkyl-C₁-C₄-alkyl”) as defined above (preferably a monocyclic cycloalkyl group) which is bound to the remainder of the molecule via a C₁-C₄-alkyl group, as defined above.

Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is OCH₃ or OC₂H₅. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₆-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₆-alkoxy-C₁-C₄-alkyl” as used herein, refers to a straight-chain or branched alkyl having 1 to 4 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “alkoxyalkoxy” as used herein refers to an alkoxyalkyl radical, in particular a C₁-C₆-alkoxy-C₁-C₄-alkyl radical, as defined above, which is bound via an oxygen atom to the remainder of the molecule. Examples thereof are OCH₂—OCH₃, OCH₂—OC₂H₅, n-propoxymethoxy, OCH₂—OCH(CH₃)₂, n-butoxymethoxy, (1-methylpropoxy)methoxy, (2-methylpropoxy)methoxy, OCH₂—OC(CH₃)₃, 2-(methoxy)ethoxy, 2-(ethoxy)ethoxy, 2-(n-propoxy)ethoxy, 2-(1-methylethoxy)ethoxy, 2-(n-butoxy)ethoxy, 2-(1-methylpropoxy)ethoxy, 2-(2-methylpropoxy)ethoxy, 2-(1,1-dimethylethoxy)ethoxy, etc.

The substituent “oxo” replaces a CH₂ by a C(═O) group.

The term “aryl” relates to phenyl and bi- or polycyclic carbocycles having at least one fused phenylene ring, which is bound to the remainder of the molecule. Examples of bi- or polycyclic carbocycles having at least one phenylene ring include naphthyl, tetrahydronaphthyl, indanyl, indenyl, anthracenyl, fluorenyl etc.

The term “aryl-C₁-C₄-alkyl” relates to C₁-C₄-alkyl, as defined above, wherein one hydrogen atom has been replaced by an aryl radical, in particular a phenyl radical. Particular examples of aryl-C₁-C₄-alkyl include —CH₂-phenyl, 1-phenethyl, 2-phenetyl, 1-phenylpropyl, 2-phenylpropyl, 3-phenyl-1-propyl and 2-phenyl-2-propyl.

The term “aryloxy-C₁-C₄-alkyl” relates to C₁-C₄-alkyl, as defined above, wherein one hydrogen atom has been replaced by an aryloxy radical, in particular a phenoxy radical. Particular examples of aryloxy-C₁-C₄-alkyl include phenoxymethyl, 1-phenoxyethyl, 2-phenoxyetyl, 1-phenoxypropyl, 2-phenoxypropyl, 3-phenoxy-1-propyl and 2-phenoxy-2-propyl.

The term “aryl-C₁-C₄-carbonyl” relates to aryl as defined above, in particular a phenyl radical, which is bound by a carbonyl to the remainder of the molecule. Particular examples of arylcarbonyl include benzoyl, 1-naphthoyl and 2-naphthoyl.

The term “hetaryl” relates to aromatic heterocyclyl or heterocycles having either 5 or 6 ring atoms (5- or 6-membered hetaryl) and being monocyclic or 8, 9 or 10 ring atoms and bing bicyclic. Hetaryl will generally have at least one ring atom selected from O, S and N, which in case of N may be an imino-nitrogen or an amino-nitrogen, which carries hydrogen or a radical different from hydrogen. Hetaryl may have 1, 2, 3 or 4 further nitrogen atoms as ring members, which are imino nitrogens. Examples of 5- or 6-membered hetaryl include 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 1,3,4-oxadiazolyl-2-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl and 1,3,5-triazin-2-yl. Examples of 8-, 9- or 10-membered hetaryl include, for example, quinolinyl, isoquinolinyl, cinnolinyl, indolyl, indolizynyl, isoindolyl, indazolyl, benzofuryl, benzothienyl, benzo[b]thiazolyl, benzoxazolyl, benzthiazolyl, benzimidazolyl, imidazo[1,2-a]pyridine-2-yl, thieno[3,2-b]pyridine-5-yl, imidazo-[2,1-b]-thiazol-6-yl and 1,2,4-triazolo[1,5-a]pyridine-2-yl. Examples of N-bound 5-, 6-, 7 or 8-membered saturated heterocyclyl or heterocycles include: pyrrolidin-1-yl, pyrazolidin-1-yl, imidazolidin-1-yl, oxazolidin-3-yl, isoxazolidin-2-yl, thiazolidin-3-yl, isothiazolidin-2-yl, piperidin-1-yl, piperazin-1-yl, morpholin-4-yl, thiomorpholin-4-yl, 1-oxothiomorpholin-4-yl, 1,1-dioxothiomorpholin-4-yl, azepan-1-yl and the like.

The term “hetaryl-C₁-C₄-alkyl” relates to C₁-C₄-alkyl, as defined above, wherein one hydrogen atom has been replaced by a hetaryl radical, in particular a pyridyl radical. Particular examples of hetaryl-C₁-C₄-alkyl include 2-pyridylmethyl, 3-pyridylmethyl, 4-pyridylmethyl, 1-(2-pyridyl)ethyl, 2-(2-pyridyl)ethyl, 1-(3-pyridyl)ethyl, 2-(3-pyridyl)ethyl, 1-(4-pyridyl)ethyl, 2-(4-pyridyl)ethyl etc.

The term “hetaryloxy-C₁-C₄-alkyl” relates to C₁-C₄-alkyl, as defined above, wherein one hydrogen atom has been replaced by an hetaryloxy radical, in particular a pyridyloxy radical. Particular examples of hetaryloxy-C₁-C₄-alkyl include 2-pyridyloxymethyl, 3-pyridyloxymethyl, 4-pyridyloxymethyl, 1-(2-pyridyloxy)ethyl, 2-(2-pyridyloxy)ethyl, 1-(3-pyridyloxy)ethyl, 2-(3-pyridyl-oxy)ethyl, 1-(4-pyridyloxy)ethyl, 2-(4-pyridyloxy)ethyl etc.

The term “hetaryl-C₁-C₄-carbonyl” relates to hetaryl as defined above, in particular a C-bound hetaryl radical, e.g. 2-, 3- or 4-pyridyl, 2- or 3-thienyl, 2- or 3-furyl, 1-, 2- or 3-pyrrolyl, 2- or 4-pyrimidinyl, pyridazinyl, 1-, 3- or 4-pyrazolyl, 1-, 2- or 4-imidazolyl radical, which is bound by a carbonyl to the remainder of the molecule.

The term “substituted” if not specified otherwise refers to substituted with 1, 2, or up to maximum possible number of substituents. If substituents as defined in compounds of formula I are more than one then they are independently from each other are same or different if not mentioned otherwise.

With respect to the variables, the embodiments of the compounds of the formula I are,
In one preferred embodiment, maximum two of B¹, B,² and B³ can be N;
In another embodiment, B¹ is CR^{B1}, B² is CR^{B2}, and B³ is CR^{B3};
In another embodiment, B¹ is N, B² is CR^{B2}, and B³ is CR^{B3};
In another embodiment, B¹ is CR^{B1}, B² is N, and B³ is CR^{B3};
In another embodiment, B¹ is CR^{B1}, B² is N, and B³ is N;
In another embodiment, B¹ is N, B² is N, and B³ is CR^{B3};
In another embodiment, B¹ is CR^{B1}, and B² is N or CR^{B2}, B³ is N or CR^{B3};
In another embodiment, B³ is CR^{B3}, and B¹ is N or CR^{B2}, B² is N or CR^{B3};
In one embodiment, A is N;
In another embodiment, A is CR^{A}.

In one embodiment, R^{A} is H, halogen, CN, C₁-C₆-alkyl, C₁-C₆-alkoxy, or C₃-C₆-cycloalkyl, wherein the alkyl, alkoxy and cycloalkyl moieties are unsubstituted or substituted with halogen or CN;
In another embodiment, R^{A} is H, halogen, CN, C₁-C₆-alkyl, or C₃-C₆-cycloalkyl;
In another embodiment, R^{A} is H, CN, or C₁-C₆-alkyl, wherein the alkyl moieties are unsubstituted or substituted with halogen or CN;
In another embodiment, R^{A} is H, CN, or C₁-C₆-alkyl, wherein the alkyl moieties are unsubstituted or substituted with halogen or CN;
In another embodiment, R^{A} is CN, or C₁-C₆-alkyl, which is unsubstituted or substituted with halogen or CN;
In another embodiment, R^{A} is CN;
In another embodiment, R^{A} is C₀-C₆-alkyl, which is unsubstituted;
In another embodiment, R^{A} is C₁-C₆-alkyl, which is substituted with halogen or CN;
In another embodiment, R^{A} is C₃-C₆-cycloalkyl, which is unsubstituted or substituted with halogen or CN;
In another embodiment, R^{A} is C₃-C₆-cycloalkyl, which is unsubstituted;
In another embodiment, R^{A} is C₃-C₆-cycloalkyl, which is substituted with halogen or CN;
In another embodiment, R^{A} is H or halogen;
In another embodiment, R^{A} is H;
In another embodiment, R^{A} is halogen;
In another embodiment, R^{A} is H, CH₃, or CN;
In another embodiment, R^{A} is CH₃ or CN;
In another embodiment, R^{A} is NR⁶R⁷;
In another embodiment, A is N, C(C₁-C₆-alkyl), or C(CN);
In another embodiment, A is N, C(CH₃), or C(CN);
In one embodiment, R⁶ and R⁷ are, identical or different, H, C₁-C₆-alkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, phenyl and heteroaryl moieties are unsubstituted or substituted with halogen, CN, or C₁-C₆-alkyl.

In one embodiment, R⁶ and R⁷ are, identical or different, H, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen or CN,
In another embodiment, R⁶ and R⁷ are, identical or different, H, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen, CN, or C₁-C₆-alkyl;
In another embodiment, R⁶ and R⁷ are, identical or different, H, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen, CN, or C₁-C₆-alkyl;
In another embodiment, R⁶ is H and R⁷ is —CH₂-5- or 6-membered heteroaryl, 1,3-dioxolan-2-ylmethyl, or 2-(methylamino)-2-oxo-ethyl, wherein the alkyl, phenyl and heteroaryl moieties are unsubstituted or substituted with halogen, CN, or C₁-C₆-alkyl;
In another embodiment, R⁶ and R⁷ independently of each other are selected from Rx-1 to Rx-7 as shown in table Rx.

TABLE Rx

| | |
|---|---|
| Rx-1 | 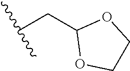 [SMILES: *CC1OCCO1] |
| Rx-2 | 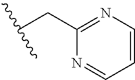 [SMILES: *Cc1ncccn1] |
| Rx-3 | 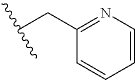 [SMILES: *Cc1ccccn1] |
| Rx-4 | 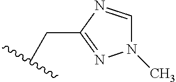 [SMILES: *Cc1ncn(C)n1] |
| Rx-5 | 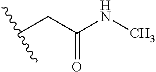 [SMILES: *CC(=O)NC] |
| Rx-6 | 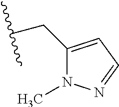 [SMILES: *Cc1ccnn1C] |
| Rx-7 | H |
| Rx-8 | 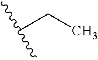 [SMILES: *CC] |

In another embodiment, R⁶ and R⁷ independently of each other are selected from Rx-1, Rx-2, Rx-7, and Rx-8. In one embodiment, R² is H, C₁-C₆-alkyl, or C₃-C₆-cycloalkyl, wherein the alkyl and cycloalkyl moieties are unsubstituted or substituted with halogen or CN;
In one embodiment, R² is H, C₁-C₆-alkyl, or C₃-C₆-cycloalkyl;
In another embodiment, R² is H or C₁-C₆-alkyl, wherein the alkyl moieties are unsubstituted or substituted with halogen or CN;
In another embodiment, R² is H or C₃-C₆-cycloalkyl, wherein the cycloalkyl moieties are unsubstituted or substituted with halogen or CN;
In another embodiment, R² is C₁-C₆-alkyl, which is unsubstituted or substituted with halogen or CN;
In another embodiment, R² is C₁-C₆-alkyl, which is unsubstituted;
In another embodiment, R² is C₁-C₆-alkyl, which is substituted with halogen or CN;
In another embodiment, R² is C₃-C₆-cycloalkyl, which is unsubstituted or substituted with halogen or CN;
In another embodiment, R² is C₃-C₆-cycloalkyl, which is unsubstituted;
In another embodiment, R² is C₃-C₆-cycloalkyl, which is substituted with halogen or CN;
In another embodiment, R² is H, CH₃, C₂H₅, n-C₃H₇, isopropyl, cyclopropyl, CH₂F, CHF₂, or CF₃.

In another embodiment, R² is H, CH₃, C₂H₅, isopropyl, or cyclopropyl;
In another embodiment, R² is H or CH₃;
In another embodiment, R² is H;
In another embodiment, R² is CH₃;
In another embodiment, R² is C₂H₅;
In another embodiment, R² is isopropyl;
In another embodiment, R² is cyclopropyl;
In one embodiment, R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
In another embodiment, R^{B1}, R^{B2}, R^{B3}, and R^{B4} independently of each other are H, halogen, CN, C₁-C₆-alkyl, or C₁-C₆-alkoxy, wherein the alkyl, and alkoxy moieties are unsubstituted or substituted with halogen;
In another embodiment, R^{B1}, R^{B2}, R^{B3}, and R^{B4} independently of each other are H, halogen, or C₁-C₆-alkyl, wherein the alkyl moieties are unsubstituted or substituted with halogen;
In another embodiment, R^{B1}, R^{B2}, R^{B3}, and R^{B4} independently of each other are H, C, Br, F, CN, CH₃, C₂H₅, n-C₃H₇, isopropyl, cyclopropyl, CF₃, CH₂F, OCH₃, or OCHF₂;
In another embodiment, R^{B1}, R^{B2}, R^{B3}, and R^{B4} independently of each other are H, C, Br, F, CN, CH₃, isopropyl, OCH₃, or OCHF₂;
In another embodiment, R^{B1}, R^{B2}, R^{B3}, and R^{B4} independently of each other are H, C, F, CN, CH₃, or OCH₃;
In another embodiment, R^{B1} and R^{B4} independently of each other are H, CI, or CH₃;
In another embodiment, R^{B2} and R^{B3} independently of each other are H, CI, Br, F, CN, CH₃, isopropyl, cyclopropyl, OCH₃, or OCHF₂;
In another embodiment, R^{B2} and R^{B3} independently of each other are H, CI, F, Br, CN, CH₃, or OCH₃;
In one embodiment, Q is —C(═O)—N(R⁵)—.

In another embodiment, Q is —N(R⁵)—C(═O)—.

In one embodiment, R⁵ is H, C₁-C₆-alkyl, C₃-C₆-cycloalkyl, or C₁-C₆-alkyl-C₃-C₆-cycloalkyl, wherein the alkyl and cycloalkyl moieties are unsubstituted or substituted with halogen or CN;
In another embodiment, R⁵ is H, C₁-C₆-alkyl, or C₁-C₆-alkyl-C₃-C₆-cycloalkyl, wherein the alkyl and cycloalkyl moieties are unsubstituted or substituted with halogen or CN;
In another embodiment, R⁵ is H, C₁-C₆-alkyl, or C₁-C₆-alkyl-C₃-C₆-cycloalkyl, wherein the alkyl and cycloalkyl moieties are unsubstituted;
In another embodiment, R⁵ is H, C₁-C₆-alkyl, or C₁-C₆-alkyl-C₃-C₆-cycloalkyl, wherein the alkyl and cycloalkyl moieties are substituted with halogen or CN;
In another embodiment, R⁵ is H, CH₃, C₂H₅, or —CH₂—CN;
In another embodiment, R⁵ is H, CH₃ or C₂H₅;
In another embodiment, R⁵ is H or CH₃;
In one embodiment, D is DA;
In one embodiment, R³ is H, C₁-C₆-alkyl, or C₃-C₆-cycloalkyl, wherein the alkyl and cycloalkyl moieties are unsubstituted or substituted with halogen or CN;
In another embodiment, R³ is H or C₁-C₆-alkyl, wherein the alkyl moieties are unsubstituted or substituted with halogen or CN;
In another embodiment, R³ is H or C₃-C₆-cycloalkyl, wherein the cycloalkyl moieties are unsubstituted or substituted with halogen or CN;
In another embodiment, R³ is H;
In another embodiment, R³ is H and DA exists in any of below tautomeric forms D3 and D4;

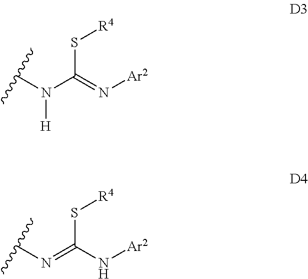

In one embodiment, R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkyl, —O—(C═O)—C₁-C₆-alkoxy, or CN;
In another embodiment, R⁴ is H or C₁-C₆-alkyl, wherein the alkyl moieties are unsubstituted or substituted with halogen, —O—(C═O)—C₁-C₆-alkyl, —O—(C═O)—C₁-C₆-alkoxy, or CN;
In another embodiment, R⁴ is H or C₁-C₆-alkyl which is unsubstituted or substituted with —O—(C═O)—C₁-C₆-alkoxy;
In another embodiment, R⁴ is H or C₃-C₆-cycloalkyl, wherein the cycloalkyl moieties are unsubstituted or substituted with halogen or CN;
In another embodiment, R⁴ is H or C₁-C₆-alkyl, wherein the alkyl moieties are unsubstituted;
In another embodiment, R⁴ is H;
In another embodiment, R⁴ is H and DA exists in any of below tautomeric forms D1 and D2;

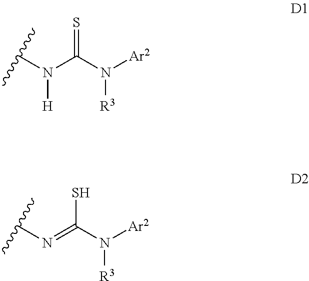

In another embodiment, D is DB wherein B is a 5- or 6-membered carbocyclic group, wherein 1 or 2 CH₂ moieties of the carbocyclic group may be replaced by a carbonyl group, wherein the carbocyclic group is unsubstituted or substituted with R^{h};
In another embodiment, D is DB wherein B is a 5- or 6-membered carbocyclic group, wherein one CH₂ moiety of the carbocyclic group may be replaced by a carbonyl group, wherein the carbocyclic group is unsubstituted or substituted with R^{h};
In another embodiment, D is DB wherein B is a 5- or 6-membered carbocyclic group, wherein one CH₂ moiety of the carbocyclic group may be replaced by a carbonyl group;
In another embodiment, D is DB wherein B is a 5- or 6-membered carbocyclic group, wherein one CH₂ moiety of the carbocyclic group is replaced by a carbonyl group;
In another embodiment, D is DB which is selected from D5 to D7, and wherein the carbocyclic group of D5 to D7 moeities are unsubstituted or substituted with 1 or 2 substituents R^{h},

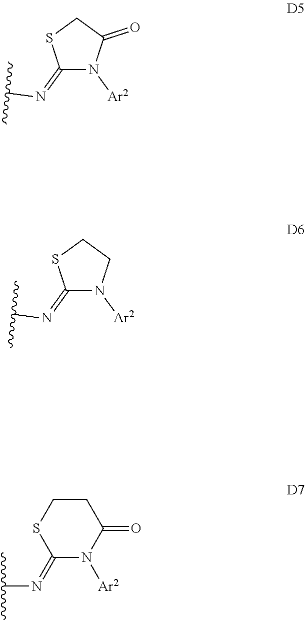

In another embodiment, D is a group selected from of D5 to D7 moieties, which are unsubstituted or substituted with 1 or 2 substituents R^{h},

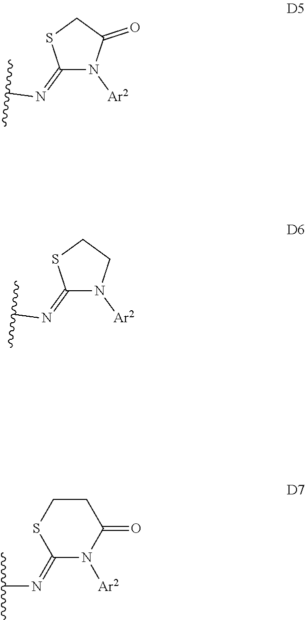

In another embodiment, D is DB which is selected from D5 to D7, and wherein the carbocyclic group of D5 to D7 moieties are unsubstituted;
In another embodiment, D is DB which is selected from D5 to D7, and wherein wherein the carbocyclic group of D5 to D7 moieties are substituted with 1 or 2 substituents R^{h};
In another embodiment, D is selected from DA, D5, D6, and D7 as defined herein;
In another embodiment, D is selected from DA, D5, D6, and D7, wherein the carbocyclic group of D5, D6, and D7 moeities are unsubstituted or substituted with 1 or 2 substituents R^{h}.

In another embodiment, D is DA or D5, wherein D5 is unsubstituted or substituted with 1 substituent R^{h};
In another embodiment, D is selected from DA or D5, wherein the carbocyclic group of D5 is substituted with 1 substituent R^{h};
In another embodiment, D is selected from DA or D5, wherein the carbocyclic group of D5 is unsubstituted;
In another embodiment, D is D5, wherein the carbocyclic group of D5 is unsubstituted or substituted with 1 substituent R^{h};
In one embodiment, Ar¹ is phenyl which is unsubstituted or substituted with R^{Ar1}.

In another embodiment, Ar¹ is 5- or 6-membered hetaryl, which is unsubstituted or substituted with R^{Ar1}.

In more embodiment, Ar¹ is phenyl, pyrimidinyl, pyridazinyl, thiophenyl, thiazolyl, or pyridyl, preferably phenyl, which moieties are unsubstituted or substituted with R^{Ar1}.

In one embodiment, R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
In another embodiment, R^{Ar1} is halogen, SF₅, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
In another embodiment, R^{Ar1} is halogen, SF₅, CN, C₁-C₆-alkyl, C₁-C₆-alkoxy, C₂-C₆-alkenyl, C₂-C₆-alkynyl wherein the alkyl, alkoxy, alkenyl, and alkynyl moieties are unsubstituted or substituted with R^{f},
or S(═O)ₘR^{e};
In another embodiment, R^{Ar1} is halogen, CN, C₁-C₆-alkyl, C₁-C₆-alkoxy, wherein the alkyl and alkoxy moieties are unsubstituted or substituted with R^{f},
In another embodiment of compound of formula I, wherein
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1,or 2.

In another embodiment of compound of formula I, wherein
Ar¹ is phenyl, which is unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂—C-alkynyl wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1,or 2.

In another embodiment of compound of formula I, wherein
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment, Ariis selected from Ar¹-1 to Ar¹-30 as shown in Table Ar¹,

TABLE Ar¹

| Ar¹ | Structure |
|---|---|
| Ar¹-1 | 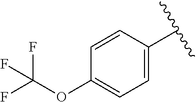 [SMILES: *c1ccc(OC(F)(F)F)cc1] |
| Ar¹-2 | 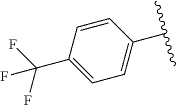 [SMILES: *c1ccc(C(F)(F)F)cc1] |
| Ar¹-3 | 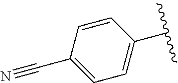 [SMILES: *c1ccc(C#N)cc1] |
| Ar¹-4 | 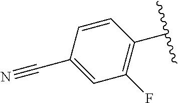 [SMILES: *c1ccc(C#N)cc1F] |
| Ar¹-5 | 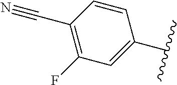 [SMILES: *c1ccc(C#N)c(F)c1] |
| Ar¹-6 | 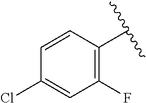 [SMILES: *c1ccc(Cl)cc1F] |
| Ar¹-7 | 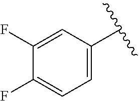 [SMILES: *c1ccc(F)c(F)c1] |
| Ar¹-8 | 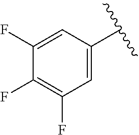 [SMILES: *c1cc(F)c(F)c(F)c1] |
| Ar¹-9 | 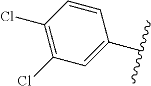 [SMILES: *c1ccc(Cl)c(Cl)c1] |
| Ar¹-10 | 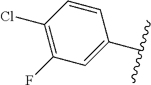 [SMILES: *c1ccc(Cl)c(F)c1] |
| Ar¹-11 | 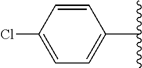 [SMILES: *c1ccc(Cl)cc1] |
| Ar¹-12 | 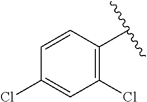 [SMILES: *c1ccc(Cl)cc1Cl] |
| Ar¹-13 | 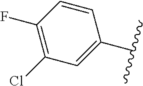 [SMILES: *c1ccc(F)c(Cl)c1] |
| Ar¹-14 | 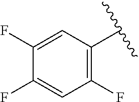 [SMILES: *c1cc(F)c(F)cc1F] |
| Ar¹-15 | 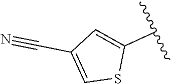 [SMILES: *c1cc(C#N)cs1] |
| Ar¹-16 | 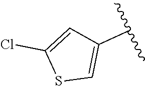 [SMILES: *c1csc(Cl)c1] |
| Ar¹-17 | 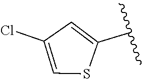 [SMILES: *c1cc(Cl)cs1] |
| Ar¹-18 | 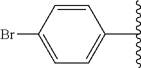 [SMILES: *c1ccc(Br)cc1] |
| Ar¹-19 | 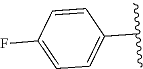 [SMILES: *c1ccc(F)cc1] |
| Ar¹-20 | 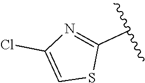 [SMILES: *c1nc(Cl)cs1] |
| Ar¹-21 | 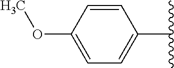 [SMILES: *c1ccc(OC)cc1] |
| Ar¹-22 | 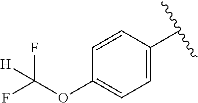 [SMILES: *c1ccc(OC(F)F)cc1] |
| Ar¹-23 | 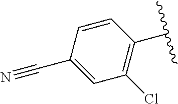 [SMILES: *c1ccc(C#N)cc1Cl] |
| Ar¹-24 | 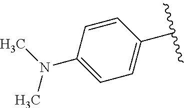 [SMILES: *c1ccc(N(C)C)cc1] |
| Ar¹-25 | 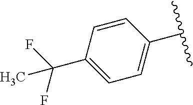 [SMILES: *c1ccc(C(C)(F)F)cc1] |
| Ar¹-26 | 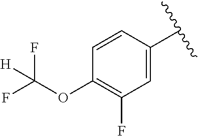 [SMILES: *c1ccc(OC(F)F)c(F)c1] |
| Ar¹-27 | 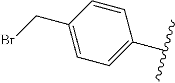 [SMILES: *c1ccc(CBr)cc1] |
| Ar¹-28 | 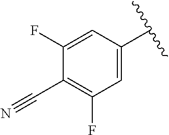 [SMILES: *c1cc(F)c(C#N)c(F)c1] |
| Ar¹-29 | 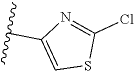 [SMILES: *c1csc(Cl)n1] |
| Ar¹-30 | 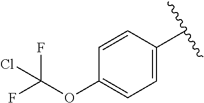 [SMILES: *c1ccc(OC(F)(F)Cl)cc1] |

In another embodiment, Ar¹ is selected from Ar¹-1 to Ar¹-5;
In another embodiment, Ar¹ is selected from Ar¹-1 to Ar¹-5;
In one embodiment, Ar² is phenyl which is unsubstituted or substituted with R^{Ar2};
In another embodiment, Ar² is 5- or 6-membered hetaryl, which is unsubstituted or substituted with R^{Ar2};
In another embodiment, Ar² is phenyl, pyrimidinyl, thiophenyl, thiazolyl, or pyridyl, which are unsubstituted or substituted with R^{Ar2}.

In one embodiment, R^{Ar2} is halogen, CN, SCN, SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl and cycloalkoxy moieties are unsubstituted or substituted with halogen,
C(═O)—OR^{a}, NR^{b}R^{c}, or C₁-C₆-alkylene-CN;
In another embodiment, R^{Ar2} is halogen, ON, C₁-C₆-alkyl, C₁-C₆-alkoxy, C₁-C₆-alkoxy-C₁-C₄-alkyl, C₃-C₆-cycloalkyl, wherein the alkyl, alkoxy, and cycloalkyl moieties are unsubstituted or substituted with halogen,
or
NR^{b}R^{c};
In another embodiment, R^{Ar2} is halogen, ON, C₁-C₆-alkyl, wherein the alkyl, moieties are unsubstituted or substituted with halogen;
In another embodiment, R^{Ar2} is halogen or C₁-C₆-alkyl, wherein the alkyl moieties are unsubstituted or substituted with halogen;
In another embodiment, R^{Ar2} is halogen or C₁-C₆-alkyl;
In another embodiment, R^{Ar2} is halogen;
In another embodiment, R^{Ar2} is C₁-C₆-alkyl;
In another embodiment, R^{Ar2} is halogen or CN,
In another embodiment, Ar² is selected from Ar²-1 to Ar²-20 as shown in Table Ar2,

TABLE Ar²

| Ar² | Structure |
|---|---|
| Ar²-1 | 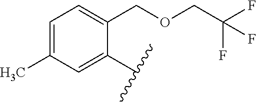 [SMILES: *c1cc(C)ccc1COCC(F)(F)F] |
| Ar²-2 | 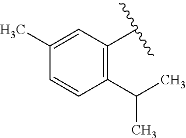 [SMILES: *c1cc(C)ccc1C(C)C] |
| Ar²-3 | 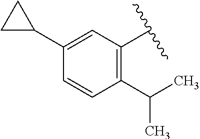 [SMILES: *c1cc(C2CC2)ccc1C(C)C] |
| Ar²-4 | 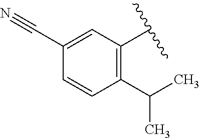 [SMILES: *c1cc(C#N)ccc1C(C)C] |
| Ar²-5 | 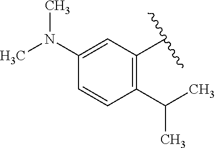 [SMILES: *c1cc(N(C)C)ccc1C(C)C] |
| Ar²-6 | 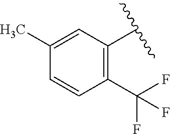 [SMILES: *c1cc(C)ccc1C(F)(F)F] |
| Ar²-7 | 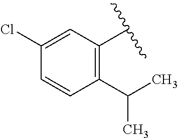 [SMILES: *c1cc(Cl)ccc1C(C)C] |
| Ar²-8 | 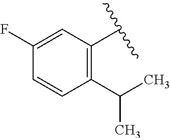 [SMILES: *c1cc(F)ccc1C(C)C] |
| Ar²-9 | 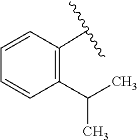 [SMILES: *c1ccccc1C(C)C] |
| Ar²-10 | 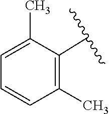 [SMILES: *c1c(C)cccc1C] |
| Ar²-11 | 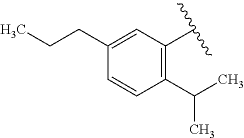 [SMILES: *c1cc(CCC)ccc1C(C)C] |
| Ar²-12 | 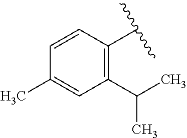 [SMILES: *c1ccc(C)cc1C(C)C] |
| Ar²-13 | 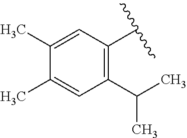 [SMILES: *c1cc(C)c(C)cc1C(C)C] |
| Ar²-14 | 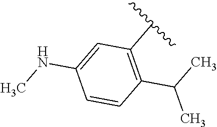 [SMILES: *c1cc(NC)ccc1C(C)C] |
| Ar²-15 | 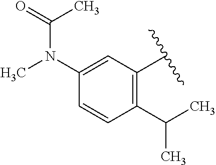 [SMILES: *c1cc(N(C)C(C)=O)ccc1C(C)C] |
| Ar²-16 | 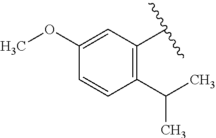 [SMILES: *c1cc(OC)ccc1C(C)C] |
| Ar²-17 | 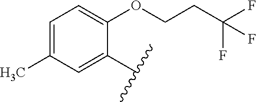 [SMILES: *c1cc(C)ccc1OCCC(F)(F)F] |
| Ar²-18 | 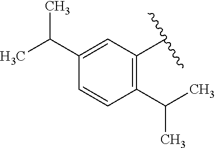 [SMILES: *c1cc(C(C)C)ccc1C(C)C] |
| Ar²-19 | 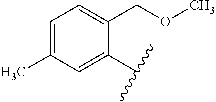 |
| Ar²-20 | 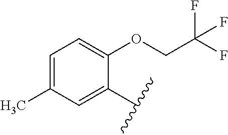 [SMILES: *c1cc(C)ccc1OCC(F)(F)F] |

In another embodiment, Ar² is selected from Ar²-1 to Ar²-20;
In another embodiment, Ar² is selected from Ar²-1 to Ar²-6;
In one embodiment, R^{a}, R^{b} and R^{c} identical or different, are H, C₁-C₆-alkyl, C₂-C₆-alkenyl, which are unsubstituted or substituted with halogen;
In another embodiment, R^{a}, R^{b} and R^{c} identical or different, are H, C₁-C₆-alkyl, which are unsubstituted or substituted with halogen;
In a embodiment, R^{d} is H;
In another embodiment, R^{d} is C₁-C₆-alkyl.

In one embodiment, R^{e} is C₁-C₆-alkyl, C₁-C₆-haloalkyl, C₃-C₆-cycloalkyl, or C₃-C₆-halo-cycloalkyl;
In another embodiment, R^{e} is C₁-C₆-alkyl or C₁-C₆-haloalkyl;
In one embodiment, R^{f} is halogen, OH, CN, C₁-C₆-alkyl, C₁-C₆-alkoxy, C₂-C₆-alkenyl, C₂-C₆-alkynyl, C₃-C₆-cycloalkyl, or C₃-C₆-cycloalkoxy, which are unsubstituted or substituted with halogen;
In another embodiment, R^{f} is halogen, OH, CN, or C₁-C₆-alkyl.

In a embodiment, R^{h} is halogen or C₁-C₆-alkyl;
In one embodiment, m is 0;
In another embodiment, m is 1;
In another embodiment, m is 2;
In another embodiment, m is 0 or 1;
In another embodiment, m is 1 or 2.

In another embodiment of compound of formula I, wherein
Q is —C(═O)—N(R⁵)—, or —N(R⁵)—C(═O)—;
D is DA, D5, D6, or D7, preferably D5;
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, and alkoxymoieties are unsubstituted or substituted with halogen
R⁵ is H, C₁-C₆-alkyl, or C₁-C₆-alkyl-C₃-C₆-cycloalkyl;
R^{A} is H, halogen, CN, C₁-C₆-alkyl, or C₃-C₆-cycloalkyl;
R² is H or C₁-C₆-alkyl;
R³ is H or C₁-C₆-alkyl;
R⁴ is H or C₁-C₆-alkyl which is unsubstituted or substituted with —O—(C═O)—C₁-C₆-alkoxy;
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment of compound of formula I, wherein
Q is —C(═O)—N(R⁵)—;
D is DB, preferably D5;
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-C₃-C₆-cycloalkyl;
R^{A} is H, halogen, CN, C₁-C₆-alkyl, or C₃-C₆-cycloalkyl;
R² is C₁-C₆-alkyl;
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen,
NR^{b}R^{c};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment, compounds of formula I are selected from compounds of formulae A.1 to A.20,

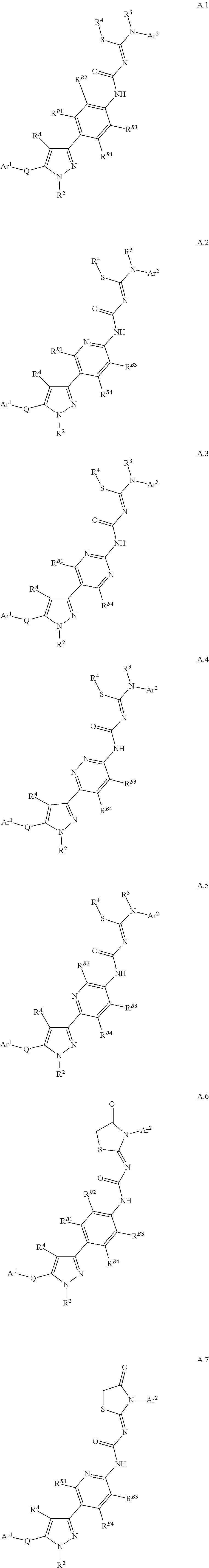

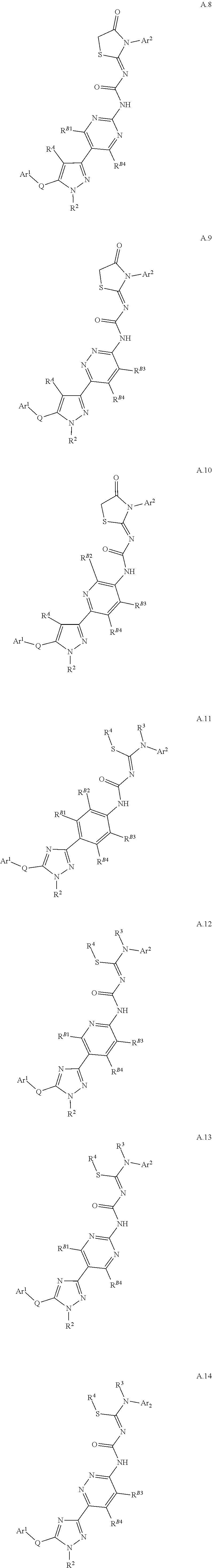

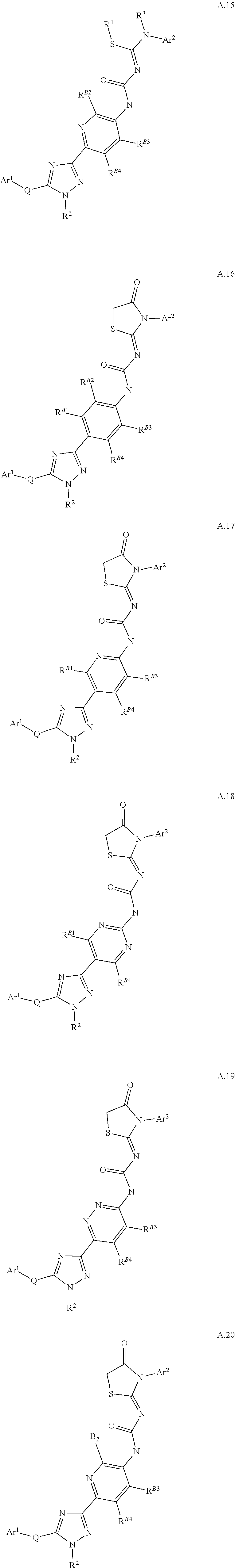

In another embodiment compounds of formula I are selected from compounds of formula A.1 to A.5
In another embodiment compounds of formula I are selected from compounds of formula A.6 to A.10;
In another embodiment compounds of formula I are selected from compounds of formula A.11 to A.15
In another embodiment compounds of formula I are selected from compounds of formula A.16 to A.20
In another embodiment compounds of formula I are selected from compounds of formula A.1 and A.6;
In another embodiment compounds of formula I are selected from compounds of formula A.11 and A.16;
In another embodiment compounds of formula I are selected from compounds of formula A.12 and A.17;
In another embodiment compounds of formula I are selected from compounds of formula A.15 and A.20;
In another embodiment compounds of formula I are selected from compounds of formula A.1, A.6, A.16, A.17, and A.20;
In another embodiment compounds of formula I are selected from compounds of formula A.1, A.6, A.16, and A.20;
In another embodiment compounds of formula I are selected from compounds of formula A.1, A.6, A.11, and A.16;
In another embodiment compounds of formula I are selected from compounds of formula A.2, A.7, A.12 and A.17;
In another embodiment compounds of formula I are selected from compounds of formula A.3, A.8, A.13, and A.18;
In another embodiment compounds of formula I are selected from compounds of formula A.4, A.9, A.14, and A.19;
In another embodiment compounds of formula I are selected from compounds of formula A.5, A.10, A.15, and A.20;
In another embodiment compounds of formula I is A.1;
In another embodiment compounds of formula I is A.2;
In another embodiment compounds of formula I is A.3;
In another embodiment compounds of formula I is A.4;
In another embodiment compounds of formula I is A.5;
In another embodiment compounds of formula I is A.6;
In another embodiment compounds of formula I is A.7;
In another embodiment compounds of formula I is A.8;
In another embodiment compounds of formula I is A.9;
In another embodiment compounds of formula I is A.10;
In another embodiment compounds of formula I is A.11;
In another embodiment compounds of formula I is A.12;
In another embodiment compounds of formula I is A.13;
In another embodiment compounds of formula I is A.14;
In another embodiment compounds of formula I is A.15;
In another embodiment compounds of formula I is A.16;
In another embodiment compounds of formula I is A.17;
In another embodiment compounds of formula I is A.18;
In another embodiment compounds of formula I is A.19;
In another embodiment compounds of formula I is A.20;
In another embodiment compounds of formula I are selected from compounds of formula A.1, A.6, A.11 and A.16, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.1 to A.5, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.6 to A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.11 to A.15, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.16 to A.20, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.1 and A.6, Wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.2 and A.7, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.3 and A.8, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.4 and A.9, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I are selected from compounds of formula A.5 and A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is selected from A.1, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.2, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.3, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.4, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.5, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B2}, R^{B3}, and R^{B4} independently of each other are H, halogen, CN, C,-Ce-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.6, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.7, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.8, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1} and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.9, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.11, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C,-Ce-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.12, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.13, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.14, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.15, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl unsubstituted or substituted with halogen, —O—(C═O)—C₁-C₆-alkoxy, —O—(C═O)—C₁-C₆-alkyl or CN;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.16, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.17, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.18, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B1}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.19, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B3}, and R^{B4} independently of each other are H, halogen, CN, C₁—OC-alkyl, C₃—C-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment compounds of formula I is A.20, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.1 to I.8,

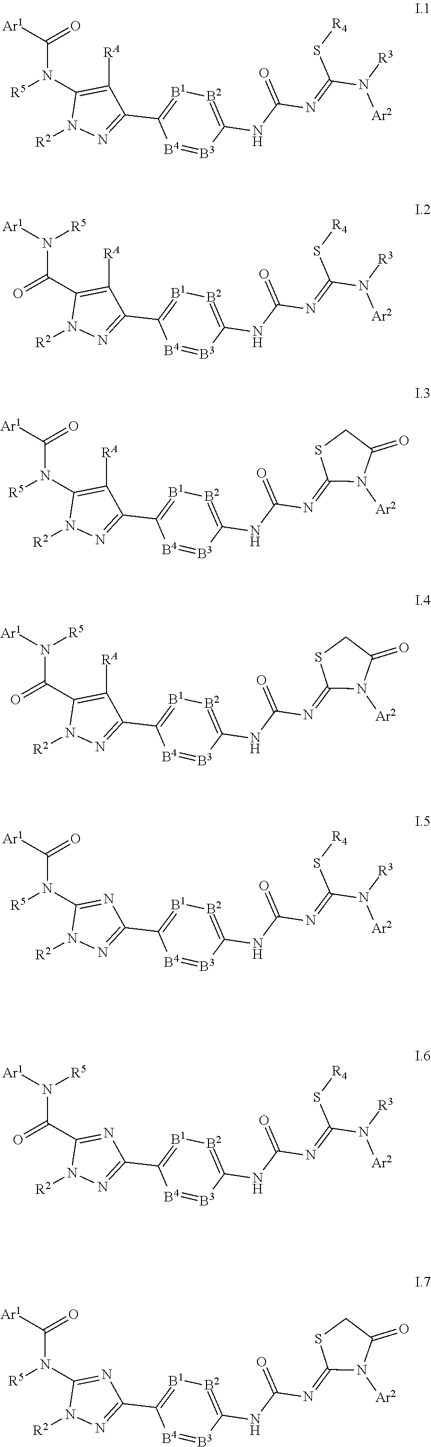

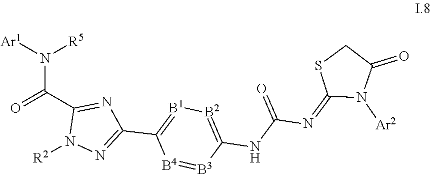

In another embodiment, the compound of formula I is selected from the compounds of formulae I.1 and 1.2;
In another embodiment, the compound of formula I is selected from the compounds of formulae I.1 and 1.3;
In another embodiment, the compound of formula I is selected from the compounds of formulae I.3 and I.4;
In another embodiment, the compound of formula I is selected from the compounds of formulae I.2 and I.4;
In another embodiment, the compound of formula I is selected from the compounds of formulae I.5 and I.6;
In another embodiment, the compound of formula I is selected from the compounds of formulae I.3 and I.7;
In another embodiment, the compound of formula I is selected from the compounds of formulae I.5 and I.7;
In another embodiment, the compound of formula I is selected from the compounds of formulae I.7 and I.8;
In another embodiment, the compound of formula I is selected from the compounds of formulae I.6 and 1.8;
In another embodiment, the compound of formula I is compound of formula I.1;
In another embodiment, the compound of formula I is compound of formula I.2;
In another embodiment, the compound of formula I is compound of formula I.3;
In another embodiment, the compound of formula I is compound of formula I.4;
In another embodiment, the compound of formula I is compound of formula I.5;
In another embodiment, the compound of formula I is compound of formula I.6;
In another embodiment, the compound of formula I is compound of formula I.7;
In another embodiment, the compound of formula I is compound of formula I.8;
In another embodiment, the compound of formula I is selected from the compounds of formulae I.1 to I.8, more preferably I.3 and I.7, wherein
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-CN;
R^{A} is H, halogen, CN, C₁-C₆-alkyl, or C₃-C₆-cycloalkyl;
R² is C₁-C₆-alkyl;
R³ is H or C₁-C₆-alkyl;
R⁴ is H or C₁-C₆-alkyl which is unsubstituted or substituted with —O—(C═O)—C₁-C₆-alkoxy;
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1}; is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen,
NR^{b}R^{c};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment, the compound of formula I is selected from the compounds of formulae I.1 to I.8, more preferably I.3 and I.7, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, halogen, CN, C₁-C₆-alkyl, or C₃-C₆-cycloalkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.1 to I.8, more preferably I.3 and I.7, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.3 and I.7, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4}; R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.1 and I.2, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.1 and 1.3, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.3 and I.4, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.2 and I.4, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.1, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.2, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compounds of formula I.3, wherein B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or —C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-CN;
R^{A} is H, halogen, CN, C₁-C₆-alkyl, or C₃—C-cycloalkyl;
R² is C₁-C₆-alkyl;
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen,
NR^{b}R^{c};
R^{a}, R^{b} and R^{e} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment, the compound of formula I is compound of formula I.3, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or C₁-C₆-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.4, wherein
R⁵ is H, C₁-C₆-alkyl, or C₁-C₆-alkyl-CN, preferably H, CH₃, C₂H₅, or —CH₂—CN, more preferably H or CH₃;
R^{A} is H, CN, or GC-alkyl, preferably CN or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.5 and I.6, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.5 and I.7, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.7 and I.8, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is selected from the compounds of formulae I.6 and 1.8, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.5, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.6, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl which is unsubstituted or substituted with —O—(C═O)—C₁-C₆-alkoxy;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compounds of formula I.7, wherein
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-CN;
R² is C₁-C₆-alkyl;
Ar¹ is phenyl, pyrimidinyl, pyridazinyl, thiophenyl, thiazolyl, or pyridyl, preferably phenyl, which moieties are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f},
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen,
NR^{b}R^{c};
R^{e}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another embodiment, the compound of formula I is compound of formula I.7, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
In another embodiment, the compound of formula I is compound of formula I.8, wherein
R⁵ is H, C₁-C₆-alkyl, or C₁-C₆-alkyl-CN, preferably H, CH₃, C₂H₅, or —CH₂—CN, more preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-30, preferably from Ar¹-1 to Ar¹-5;
Ar² is selected from Ar²-1 to Ar²-20, preferably from Ar²-1 to Ar²-6;
Particular compounds of formula I are the compounds of the formulae I.3, 1.4, 1.7, and 1.8 that are compiled in the following tables 1 to 720, wherein the combination of variables B¹, B², B³ and B⁴ for each compound of tables 1 to 720 corresponds to each line of Table B. Each of the groups mentioned for a substituent in the tables is furthermore per se, independently of the combination in which it is mentioned, a particularly preferred aspect of the substituent in question.
Table 1. Compounds of formula I.3 where R^{A} is CN, R² is H, R⁵ is H, Ar¹ is Ar¹-1, Ar² is Ar²-1.
Table 2. Compounds of formula I.3 where R^{A} is CN, R² is H, R⁵ is H, Ar¹ is Ar¹-1, Ar² is Ar²-2.
Table 3. Compounds of formula I.3 where R^{A} is CN, R² is H, R⁵ is H, Ar¹ is Ar¹-1, Ar² is Ar²-3.
Table 4. Compounds of formula I.3 where R^{A} is CN, R² is H, R⁵ is H, Ar¹ is Ar¹-1, Ar² is Ar²-4.
Table 5. Compounds of formula I.3 where R^{A} is CN, R² is H, R⁵ is H, Ar¹ is Ar¹-1, Ar² is Ar²-5.
Table 6. Compounds of formula I.3 where R^{A} is CN, R² is H, R⁵ is H, Ar¹ is Ar¹-1, Ar² is Ar²-6.
Table 7. Compounds of formula I.3 where R^{A} is CN, R² is H, R⁵ is H, Ar¹ is Ar¹-2, Ar² is Ar²-1.
Table 8. Compounds of formula I.3 where R^{A} is CN, R² is H, R⁵ is H, Ar¹ is Ar¹-2, Ar² is Ar²-2.
Table 9. Compounds of formula I.3 where R^{A} is CN, R² is H, R⁵ is H, Ar¹ is Ar¹-2, Ar² is Ar²-3.
Table 10. Compounds of formula I.3 where R^{A} is CN, R² is H, R⁵ is H, Ar¹ is Ar¹-2, Ar² is Ar²-4.
Table 11. Compounds of formula I.3 where R^{A} is CN, R² is H, R⁵ is H, Ar¹ is Ar¹-2, Ar² is Ar²-5.
Table 12. Compounds of formula I.3 where R^{A} is CN, R² is H, R⁵ is H, Ar¹ is Ar¹-2, Ar² is Ar²-6.
Table 13. Compounds of formula I.3 where R^{A} is CN, R² is H, R⁵ is H, Ar¹ is Ar¹-3, Ar² is Ar²-1.
Table 14. Compounds of formula I.3 where R^{A} is CN, R² is H, R⁵ is H, Ar¹ is Ar¹-3, Ar² is Ar²-2.
Table 15. Compounds of formula I.3 where R^{A} is CN, R² is H, R⁵ is H, Ar¹ is Ar¹-3, Ar² is Ar²-3.
Table 16. Compounds of formula I.3 where R^{A} is CN, R² is H, R⁵ is H, Ar¹ is Ar¹-3, Ar² is Ar²-4.
Table 17. Compounds of formula I.3 where R^{A} is CN, R² is H, R⁵ is H, Ar¹ is Ar¹-3, Ar² is Ar²-5.
Table 18. Compounds of formula I.3 where R^{A} is CN, R² is H, R⁵ is H, Ar¹ is Ar¹-3, Ar² is Ar²-6.
Table 19. Compounds of formula I.3 where R^{A} is CN, R² is H, R⁵ is H, Ar¹ is Ar¹-4, Ar² is Ar²-1.
Table 20. Compounds of formula I.3 where R^{A} is CN, R² is H, R⁵ is H, Ar¹ is Ar¹-4, Ar² is Ar²-2.
Table 21. Compounds of formula I.3 where R^{A} is CN, R² is H, R⁵ is H, Ar¹ is Ar¹-4, Ar² is Ar²-3.
Table 22. Compounds of formula I.3 where R^{A} is CN, R² is H, R⁵ is H, Ar¹ is Ar¹-4, Ar² is Ar²-4.
Table 23. Compounds of formula I.3 where R^{A} is CN, R² is H, R⁵ is H, Ar¹ is Ar¹-4, Ar² is Ar²-5.
Table 24. Compounds of formula I.3 where R^{A} is CN, R² is H, R⁵ is H, Ar¹ is Ar¹-4, Ar² is Ar²-6.
Table 25. Compounds of formula I.3 where R^{A} is CN, R² is H, R⁵ is H, Ar¹ is Ar¹-5, Ar² is Ar²-1.
Table 26. Compounds of formula I.3 where R^{A} is CN, R² is H, R⁵ is H, Ar¹ is Ar¹-5, Ar² is Ar²-2.
Table 27. Compounds of formula I.3 where R^{A} is CN, R² is H, R⁵ is H, Ar¹ is Ar¹-5, Ar² is Ar²-3.
Table 28. Compounds of formula I.3 where R^{A} is CN, R² is H, R⁵ is H, Ar¹ is Ar¹-5, Ar² is Ar²-4.
Table 29. Compounds of formula I.3 where R^{A} is CN, R² is H, R⁵ is H, Ar¹ is Ar¹-5, Ar² is Ar²-5.
Table 30. Compounds of formula I.3 where R^{A} is CN, R² is H, R⁵ is H, Ar¹ is Ar¹-5, Ar² is Ar²-6.
Table 31. Compounds of formula I.3 where R^{A} is CN, R² is H, R⁵ is CH₃, Ar¹ is Ar¹-1, Ar² is Ar²-1.
Table 32. Compounds of formula I.3 where R^{A} is CN, R² is H, R⁵ is CH₃, Ar¹ is Ar¹-1, Ar² is Ar²-2.
Table 33. Compounds of formula I.3 where R^{A} is CN, R² is H, R⁵ is CH₃, Ar¹ is Ar¹-1, Ar² is Ar²-3.
Table 34. Compounds of formula I.3 where R^{A} is CN, R² is H, R⁵ is CH₃, Ar¹ is Ar¹-1, Ar² is Ar²-4.
Table 35. Compounds of formula I.3 where R^{A} is CN, R² is H, R⁵ is CH₃, Ar¹ is Ar¹-1, Ar² is Ar²-5.
Table 36. Compounds of formula I.3 where R^{A} is CN, R² is H, R⁵ is CH₃, Ar¹ is Ar¹-1, Ar² is Ar²-6.
Table 37. Compounds of formula I.3 where R^{A} is CN, R² is H, R⁵ is CH₃, Ar¹ is Ar¹-2, Ar² is Ar²-1.
Table 38. Compounds of formula I.3 where R^{A} is CN, R² is H, R⁵ is CH₃, Ar¹ is Ar¹-2, Ar² is Ar²-2.
Table 39. Compounds of formula I.3 where R^{A} is CN, R² is H, R⁵ is CH₃, Ar¹ is Ar¹-2, Ar² is Ar²-3.
Table 40. Compounds of formula I.3 where R^{A} is CN, R² is H, R⁵ is CH₃, Ar¹ is Ar¹-2, Ar² is Ar²-4.
Table 41. Compounds of formula I.3 where R^{A} is CN, R² is H, R⁵ is CH₃, Ar¹ is Ar¹-2, Ar² is Ar²-5.
Table 42. Compounds of formula I.3 where R^{A} is CN, R² is H, R⁵ is CH₃, Ar¹ is Ar¹-2, Ar² is Ar²-6.
Table 43. Compounds of formula I.3 where R^{A} is CN, R² is H, R⁵ is CH₃, Ar¹ is Ar¹-3, Ar² is Ar²-1.
Table 44. Compounds of formula I.3 where R^{A} is CN, R² is H, R⁵ is CH₃, Ar¹ is Ar¹-3, Ar² is Ar²-2.
Table 45. Compounds of formula I.3 where R^{A} is CN, R² is H, R⁵ is CH₃, Ar¹ is Ar¹-3, Ar² is Ar²-3.
Table 46. Compounds of formula I.3 where R^{A} is CN, R² is H, R⁵ is CH₃, Ar¹ is Ar¹-3, Ar² is Ar²-4.
Table 47. Compounds of formula I.3 where R^{A} is CN, R² is H, R⁵ is CH₃, Ar¹ is Ar¹-3, Ar² is Ar²-5.
Table 48. Compounds of formula I.3 where R^{A} is CN, R² is H, R⁵ is CH₃, Ar¹ is Ar¹-3, Ar² is Ar²-6.
Table 49. Compounds of formula I.3 where R^{A} is CN, R² is H, R⁵ is CH₃, Ar¹ is Ar¹-4, Ar² is Ar²-1.
Table 50. Compounds of formula I.3 where R^{A} is CN, R² is H, R⁵ is CH₃, Ar¹ is Ar¹-4, Ar² is Ar²-2.
Table 51. Compounds of formula I.3 where R^{A} is CN, R² is H, R⁵ is CH₃, Ar¹ is Ar¹-4, Ar² is Ar²-3.
Table 52. Compounds of formula I.3 where R^{A} is CN, R² is H, R⁵ is CH₃, Ar¹ is Ar¹-4, Ar² is Ar²-4.
Table 53. Compounds of formula I.3 where R^{A} is CN, R² is H, R⁵ is CH₃, Ar¹ is Ar¹-4, Ar² is Ar²-5.
Table 54. Compounds of formula I.3 where R^{A} is CN, R² is H, R⁵ is CH₃, Ar¹ is Ar¹-4, Ar² is Ar²-6.
Table 55. Compounds of formula I.3 where R^{A} is CN, R² is H, R⁵ is CH₃, Ar¹ is Ar¹-5, Ar² is Ar²-1.
Table 56. Compounds of formula I.3 where R^{A} is CN, R² is H, R⁵ is CH₃, Ar¹ is Ar¹-5, Ar² is Ar²-2.
Table 57. Compounds of formula I.3 where R^{A} is CN, R² is H, R⁵ is CH₃, Ar¹ is Ar¹-5, Ar² is Ar²-3.
Table 58. Compounds of formula I.3 where R^{A} is CN, R² is H, R⁵ is CH₃, Ar¹ is Ar¹-5, Ar² is Ar²-4.
Table 59. Compounds of formula I.3 where R^{A} is CN, R² is H, R⁵ is CH₃, Ar¹ is Ar¹-5, Ar² is Ar²-5.
Table 60. Compounds of formula I.3 where R^{A} is CN, R² is H, R⁵ is CH₃, Ar¹ is Ar¹-5, Ar² is Ar²-6.
Table 61. Compounds of formula I.3 where R^{A} is CN, R² is CH₃, R⁵ is H, Ar¹ is Ar¹-1, Ar² is Ar²-1.
Table 62. Compounds of formula I.3 where R^{A} is CN, R² is CH₃, R⁵ is H, Ar¹ is Ar¹-1, Ar² is Ar²-2.
Table 63. Compounds of formula I.3 where R^{A} is CN, R² is CH₃, R⁵ is H, Ar¹ is Ar¹-1, Ar² is Ar²-3.
Table 64. Compounds of formula I.3 where R^{A} is CN, R² is CH₃, R⁵ is H, Ar¹ is Ar¹-1, Ar² is Ar²-4.
Table 65. Compounds of formula I.3 where R^{A} is CN, R² is CH₃, R⁵ is H, Ar¹ is Ar¹-1, Ar² is Ar²-5.
Table 66. Compounds of formula I.3 where R^{A} is CN, R² is CH₃, R⁵ is H, Ar¹ is Ar¹-1, Ar² is Ar²-6.
Table 67. Compounds of formula I.3 where R^{A} is CN, R² is CH₃, R⁵ is H, Ar¹ is Ar¹-2, Ar² is Ar²-1.
Table 68. Compounds of formula I.3 where R^{A} is CN, R² is CH₃, R⁵ is H, Ar¹ is Ar¹-2, Ar² is Ar²-2.
Table 69. Compounds of formula I.3 where R^{A} is CN, R² is CH₃, R⁵ is H, Ar¹ is Ar¹-2, Ar² is Ar²-3.
Table 70. Compounds of formula I.3 where R^{A} is CN, R² is CH₃, R⁵ is H, Ar¹ is Ar¹-2, Ar² is Ar²-4.
Table 71. Compounds of formula I.3 where R^{A} is CN, R² is CH₃, R⁵ is H, Ar¹ is Ar¹-2, Ar² is Ar²-5.
Table 72. Compounds of formula I.3 where R^{A} is CN, R² is CH₃, R⁵ is H, Ar¹ is Ar¹-2, Ar² is Ar²-6.
Table 73. Compounds of formula I.3 where R^{A} is CN, R² is CH₃, R⁵ is H, Ar¹ is Ar¹-3, Ar² is Ar²-1.
Table 74. Compounds of formula I.3 where R^{A} is CN, R² is CH₃, R⁵ is H, Ar¹ is Ar¹-3, Ar² is Ar²-2.
Table 75. Compounds of formula I.3 where R^{A} is CN, R² is CH₃, R⁵ is H, Ar¹ is Ar¹-3, Ar² is Ar²-3.
Table 76. Compounds of formula I.3 where R^{A} is CN, R² is CH₃, R⁵ is H, Ar¹ is Ar¹-3, Ar² is Ar²-4.
Table 77. Compounds of formula I.3 where R^{A} is CN, R² is CH₃, R⁵ is H, Ar¹ is Ar¹-3, Ar² is Ar²-5.
Table 78. Compounds of formula I.3 where R^{A} is CN, R² is CH₃, R⁵ is H, Ar¹ is Ar¹-3, Ar² is Ar²-6.
Table 79. Compounds of formula I.3 where R^{A} is CN, R² is CH₃, R⁵ is H, Ar¹ is Ar¹-4, Ar² is Ar²-1.
Table 80. Compounds of formula I.3 where R^{A} is CN, R² is CH₃, R⁵ is H, Ar¹ is Ar¹-4, Ar² is Ar²-2.
Table 81. Compounds of formula I.3 where R^{A} is CN, R² is CH₃, R⁵ is H, Ar¹ is Ar¹-4, Ar² is Ar²-3.
Table 82. Compounds of formula I.3 where R^{A} is CN, R² is CH₃, R⁵ is H, Ar¹ is Ar¹-4, Ar² is Ar²-4.
Table 83. Compounds of formula I.3 where R^{A} is CN, R² is CH₃, R⁵ is H, Ar¹ is Ar¹-4, Ar² is Ar²-5.
Table 84. Compounds of formula I.3 where R^{A} is CN, R² is CH₃, R⁵ is H, Ar¹ is Ar¹-4, Ar² is Ar²-6.
Table 85. Compounds of formula I.3 where R^{A} is CN, R² is CH₃, R⁵ is H, Ar¹ is Ar¹-5, Ar² is Ar²-1.
Table 86. Compounds of formula I.3 where R^{A} is CN, R² is CH₃, R⁵ is H, Ar¹ is Ar¹-5, Ar² is Ar²-2.
Table 87. Compounds of formula I.3 where R^{A} is CN, R² is CH₃, R⁵ is H, Ar¹ is Ar¹-5, Ar² is Ar²-3.
Table 88. Compounds of formula I.3 where R^{A} is CN, R² is CH₃, R⁵ is H, Ar¹ is Ar¹-5, Ar² is Ar²-4.
Table 89. Compounds of formula I.3 where R^{A} is CN, R² is CH₃, R⁵ is H, Ar¹ is Ar¹-5, Ar² is Ar²-5.
Table 90. Compounds of formula I.3 where R^{A} is CN, R² is CH₃, R⁵ is H, Ar¹ is Ar¹-5, Ar² is Ar²-6.
Table 91. Compounds of formula I.3 where R^{A} is CN, R² is CH₃, R⁵ is CH₃, Ar¹ is Ar¹-1, Ar² is Ar²-1.
Table 92. Compounds of formula I.3 where R^{A} is CN, R² is CH₃, R⁵ is CH₃, Ar¹ is Ar¹-1, Ar² is Ar²-2.
Table 93. Compounds of formula I.3 where R^{A} is CN, R² is CH₃, R⁵ is CH₃, Ar¹ is Ar¹-1, Ar² is Ar²-3.
Table 94. Compounds of formula I.3 where R^{A} is CN, R² is CH₃, R⁵ is CH₃, Ar¹ is Ar¹-1, Ar² is Ar²-4.
Table 95. Compounds of formula I.3 where R^{A} is CN, R² is CH₃, R⁵ is CH₃, Ar¹ is Ar¹-1, Ar² is Ar²-5.
Table 96. Compounds of formula I.3 where R^{A} is CN, R² is CH₃, R⁵ is CH₃, Ar¹ is Ar¹-1, Ar² is Ar²-6.
Table 97. Compounds of formula I.3 where R^{A} is CN, R² is CH₃, R⁵ is CH₃, Ar¹ is Ar¹-2, Ar² is Ar²-1.
Table 98. Compounds of formula I.3 where R^{A} is CN, R² is CH₃, R⁵ is CH₃, Ar¹ is Ar¹-2, Ar² is Ar²-2.
Table 99. Compounds of formula I.3 where R^{A} is CN, R² is CH₃, R⁵ is CH₃, Ar¹ is Ar¹-2, Ar² is Ar²-3.
Table 100. Compounds of formula I.3 where R^{A} is CN, R² is CH₃, R⁵ is CH₃, Ar¹ is Ar¹-2, Ar² is Ar²-4.
Table 101. Compounds of formula I.3 where R^{A} is CN, R² is CH₃, R⁵ is CH₃, Ar¹ is Ar¹-2, Ar² is Ar²-5.
Table 102. Compounds of formula I.3 where R^{A} is CN, R² is CH₃, R⁵ is CH₃, Ar¹ is Ar¹-2, Ar² is Ar²-6.
Table 103. Compounds of formula I.3 where R^{A} is CN, R² is CH₃, R⁵ is CH₃, Ar¹ is Ar¹-3, Ar² is Ar²-1.
Table 104. Compounds of formula I.3 where R^{A} is CN, R² is CH₃, R⁵ is CH₃, Ar¹ is Ar¹-3, Ar² is Ar²-2.
Table 105. Compounds of formula I.3 where R^{A} is CN, R² is CH₃, R⁵ is CH₃, Ar¹ is Ar¹-3, Ar² is Ar²-3.
Table 106. Compounds of formula I.3 where R^{A} is CN, R² is CH₃, R⁵ is CH₃, Ar¹ is Ar¹-3, Ar² is Ar²-4.
Table 107. Compounds of formula I.3 where R^{A} is CN, R² is CH₃, R⁵ is CH₃, Ar¹ is Ar¹-3, Ar² is Ar²-5.
Table 108. Compounds of formula I.3 where R^{A} is CN, R² is CH₃, R⁵ is CH₃, Ar¹ is Ar¹-3, Ar² is Ar²-6.
Table 109. Compounds of formula I.3 where R^{A} is CN, R² is CH₃, R^{S} is CH₃, Ar¹ is Ar¹-4, Ar² is Ar²-1.
Table 110. Compounds of formula I.3 where R^{A} is CN, R² is CH₃, R⁵ is CH₃, Ar¹ is Ar¹-4, Ar² is Ar²-2.
Table 111. Compounds of formula I.3 where R^{A} is CN, R² is CH₃, R⁵ is CH₃, Ar¹ is Ar¹-4, Ar² is Ar²-3.
Table 112. Compounds of formula I.3 where R^{A} is CN, R² is CH₃, R⁵ is CH₃, Ar¹ is Ar¹-4, Ar² is Ar²-4.
Table 113. Compounds of formula I.3 where R^{A} is CN, R² is CH₃, R⁵ is CH₃, Ar¹ is Ar¹-4, Ar² is Ar²-5.
Table 114. Compounds of formula I.3 where R^{A} is CN, R² is CH₃, R^{S} is CH₃, Ar¹ is Ar¹-4, Ar² is Ar²-6.
Table 115. Compounds of formula I.3 where R^{A} is CN, R² is CH₃, R^{S} is CH₃, Ar¹ is Ar¹-5, Ar² is Ar²-1.
Table 116. Compounds of formula I.3 where R^{A} is CN, R² is CH₃, R^{S} is CH₃, Ar¹ is Ar¹-5, Ar² is Ar²-2.
Table 117. Compounds of formula I.3 where R^{A} is CN, R² is CH₃, R^{S} is CH₃, Ar¹ is Ar¹-5, Ar² is Ar²-3.
Table 118. Compounds of formula I.3 where R^{A} is CN, R² is CH₃, R^{S} is CH₃, Ar¹ is Ar¹-5, Ar² is Ar²-4.
Table 119. Compounds of formula I.3 where R^{A} is CN, R² is CH₃, R^{S} is CH₃, Ar¹ is Ar¹-5, Ar² is Ar²-5.
Table 120. Compounds of formula I.3 where R^{A} is CN, R² is CH₃, R⁵ is CH₃, Ar¹ is Ar¹-5, Ar² is Ar²-6.
Table 121. Compounds of formula I.3 where R^{A} is CH₃, R² is H, R⁵ is H, Ar¹ is Ar¹-1, Ar² is Ar²-1.
Table 122. Compounds of formula I.3 where R^{A} is CH₃, R² is H, R⁵ is H, Ar¹ is Ar¹-1, Ar² is Ar²-2.
Table 123. Compounds of formula I.3 where R^{A} is CH₃, R² is H, R⁵ is H, Ar¹ is Ar¹-1, Ar² is Ar²-3.
Table 124. Compounds of formula I.3 where R^{A} is CH₃, R² is H, R⁵ is H, Ar¹ is Ar¹-1, Ar² is Ar²-4.
Table 125. Compounds of formula I.3 where R^{A} is CH₃, R² is H, R⁵ is H, Ar¹ is Ar¹-1, Ar² is Ar²-5.
Table 126. Compounds of formula I.3 where R^{A} is CH₃, R² is H, R⁵ is H, Ar¹ is Ar¹-1, Ar² is Ar²-6.
Table 127. Compounds of formula I.3 where R^{A} is CH₃, R² is H, R⁵ is H, Ar¹ is Ar¹-2, Ar² is Ar²-1.
Table 128. Compounds of formula I.3 where R^{A} is CH₃, R² is H, R⁵ is H, Ar¹ is Ar¹-2, Ar² is Ar²-2.
Table 129. Compounds of formula I.3 where R^{A} is CH₃, R² is H, R⁵ is H, Ar¹ is Ar¹-2, Ar² is Ar²-3.
Table 130. Compounds of formula I.3 where R^{A} is CH₃, R² is H, R⁵ is H, Ar¹ is Ar¹-2, Ar² is Ar²-4.
Table 131. Compounds of formula I.3 where R^{A} is CH₃, R² is H, R⁵ is H, Ar¹ is Ar¹-2, Ar² is Ar²-5.
Table 132. Compounds of formula I.3 where R^{A} is CH₃, R² is H, R⁵ is H, Ar¹ is Ar¹-2, Ar² is Ar²-6.
Table 133. Compounds of formula I.3 where R^{A} is CH₃, R² is H, R⁵ is H, Ar¹ is Ar¹-3, Ar² is Ar²-1.
Table 134. Compounds of formula I.3 where R^{A} is CH₃, R² is H, R⁵ is H, Ar¹ is Ar¹-3, Ar² is Ar²-2.
Table 135. Compounds of formula I.3 where R^{A} is CH₃, R² is H, R⁵ is H, Ar¹ is Ar¹-3, Ar² is Ar²-3.
Table 136. Compounds of formula I.3 where R^{A} is CH₃, R² is H, R⁵ is H, Ar¹ is Ar¹-3, Ar² is Ar²-4.
Table 137. Compounds of formula I.3 where R^{A} is CH₃, R² is H, R⁵ is H, Ar¹ is Ar¹-3, Ar² is Ar²-5.
Table 138. Compounds of formula I.3 where R^{A} is CH₃, R² is H, R⁵ is H, Ar¹ is Ar¹-3, Ar² is Ar²-6.
Table 139. Compounds of formula I.3 where R^{A} is CH₃, R² is H, R⁵ is H, Ar¹ is Ar¹-4, Ar² is Ar²-1.
Table 140. Compounds of formula I.3 where R^{A} is CH₃, R² is H, R⁵ is H, Ar¹ is Ar¹-4, Ar² is Ar²-2.
Table 141. Compounds of formula I.3 where R^{A} is CH₃, R² is H, R⁵ is H, Ar¹ is Ar¹-4, Ar² is Ar²-3.
Table 142. Compounds of formula I.3 where R^{A} is CH₃, R² is H, R⁵ is H, Ar¹ is Ar¹-4, Ar² is Ar²-4.
Table 143. Compounds of formula I.3 where R^{A} is CH₃, R² is H, R⁵ is H, Ar¹ is Ar¹-4, Ar² is Ar²-5.
Table 144. Compounds of formula I.3 where R^{A} is CH₃, R² is H, R⁵ is H, Ar¹ is Ar¹-4, Ar² is Ar²-6.
Table 145. Compounds of formula I.3 where R^{A} is CH₃, R² is H, R⁵ is H, Ar¹ is Ar¹-5, Ar² is Ar²-1.
Table 146. Compounds of formula I.3 where R^{A} is CH₃, R² is H, R⁵ is H, Ar¹ is Ar¹-5, Ar² is Ar²-2.
Table 147. Compounds of formula I.3 where R^{A} is CH₃, R² is H, R⁵ is H, Ar¹ is Ar¹-5, Ar² is Ar²-3.
Table 148. Compounds of formula I.3 where R^{A} is CH₃, R² is H, R⁵ is H, Ar¹ is Ar¹-5, Ar² is Ar²-4.
Table 149. Compounds of formula I.3 where R^{A} is CH₃, R² is H, R⁵ is H, Ar¹ is Ar¹-5, Ar² is Ar²-5.
Table 150. Compounds of formula I.3 where R^{A} is CH₃, R² is H, R⁵ is H, Ar¹ is Ar¹-5, Ar² is Ar²-6.
Table 151. Compounds of formula I.3 where R^{A} is CH₃, R² is H, R⁵ is CH₃, Ar¹ is Ar¹-1, Ar² is Ar²-1.
Table 152. Compounds of formula I.3 where R^{A} is CH₃, R² is H, R⁵ is CH₃, Ar¹ is Ar¹-1, Ar² is Ar²-2.
Table 153. Compounds of formula I.3 where R^{A} is CH₃, R² is H, R⁵ is CH₃, Ar¹ is Ar¹-1, Ar² is Ar²-3.
Table 154. Compounds of formula I.3 where R^{A} is CH₃, R² is H, R⁵ is CH₃, Ar¹ is Ar¹-1, Ar² is Ar²-4.
Table 155. Compounds of formula I.3 where R^{A} is CH₃, R² is H, R⁵ is CH₃, Ar¹ is Ar¹-1, Ar² is Ar²-5.
Table 156. Compounds of formula I.3 where R^{A} is CH₃, R² is H, R⁵ is CH₃, Ar¹ is Ar¹-1, Ar² is Ar²-6.
Table 157. Compounds of formula I.3 where R^{A} is CH₃, R² is H, R⁵ is CH₃, Ar¹ is Ar¹-2, Ar² is Ar²-1.
Table 158. Compounds of formula I.3 where R^{A} is CH₃, R² is H, R⁵ is CH₃, Ar¹ is Ar¹-2, Ar² is Ar²-2.
Table 159. Compounds of formula I.3 where R^{A} is CH₃, R² is H, R⁵ is CH₃, Ar¹ is Ar¹-2, Ar² is Ar²-3.
Table 160. Compounds of formula I.3 where R^{A} is CH₃, R² is H, R⁵ is CH₃, Ar¹ is Ar¹-2, Ar² is Ar²-4.
Table 161. Compounds of formula I.3 where R^{A} is CH₃, R² is H, R⁵ is CH₃, Ar¹ is Ar¹-2, Ar² is Ar²-5.
Table 162. Compounds of formula I.3 where R^{A} is CH₃, R² is H, R⁵ is CH₃, Ar¹ is Ar¹-2, Ar² is Ar²-6.
Table 163. Compounds of formula I.3 where R^{A} is CH₃, R² is H, R⁵ is CH₃, Ar¹ is Ar¹-3, Ar² is Ar²-1.
Table 164. Compounds of formula I.3 where R^{A} is CH₃, R² is H, R⁵ is CH₃, Ar¹ is Ar¹-3, Ar² is Ar²-2.
Table 165. Compounds of formula I.3 where R^{A} is CH₃, R² is H, R⁵ is CH₃, Ar¹ is Ar¹-3, Ar² is Ar²-3.
Table 166. Compounds of formula I.3 where R^{A} is CH₃, R² is H, R⁵ is CH₃, Ar¹ is Ar¹-3, Ar² is Ar²-4.
Table 167. Compounds of formula I.3 where R^{A} is CH₃, R² is H, R⁵ is CH₃, Ar¹ is Ar¹-3, Ar² is Ar²-5.
Table 168. Compounds of formula I.3 where R^{A} is CH₃, R² is H, R⁵ is CH₃, Ar¹ is Ar¹-3, Ar² is Ar²-6.
Table 169. Compounds of formula I.3 where R^{A} is CH₃, R² is H, R⁵ is CH₃, Ar¹ is Ar¹-4, Ar² is Ar²-1.
Table 170. Compounds of formula I.3 where R^{A} is CH₃, R² is H, R⁵ is CH₃, Ar¹ is Ar¹-4, Ar² is Ar²-2.
Table 171. Compounds of formula I.3 where R^{A} is CH₃, R² is H, R⁵ is CH₃, Ar¹ is Ar¹-4, Ar² is Ar²-3.
Table 172. Compounds of formula I.3 where R^{A} is CH₃, R² is H, R⁵ is CH₃, Ar¹ is Ar¹-4, Ar² is Ar²-4.
Table 173. Compounds of formula I.3 where R^{A} is CH₃, R² is H, R⁵ is CH₃, Ar¹ is Ar¹-4, Ar² is Ar²-5.
Table 174. Compounds of formula I.3 where R^{A} is CH₃, R² is H, R⁵ is CH₃, Ar¹ is Ar¹-4, Ar² is Ar²-6.
Table 175. Compounds of formula I.3 where R^{A} is CH₃, R² is H, R⁵ is CH₃, Ar¹ is Ar¹-5, Ar² is Ar²-1.
Table 176. Compounds of formula I.3 where R^{A} is CH₃, R² is H, R⁵ is CH₃, Ar¹ is Ar¹-5, Ar² is Ar²-2.
Table 177. Compounds of formula I.3 where R^{A} is CH₃, R² is H, R⁵ is CH₃, Ar¹ is Ar¹-5, Ar² is Ar²-3.
Table 178. Compounds of formula I.3 where R^{A} is CH₃, R² is H, R⁵ is CH₃, Ar¹ is Ar¹-5, Ar² is Ar²-4.
Table 179. Compounds of formula I.3 where R^{A} is CH₃, R² is H, R⁵ is CH₃, Ar¹ is Ar¹-5, Ar² is Ar²-5.
Table 180. Compounds of formula I.3 where R^{A} is CH₃, R² is H, R⁵ is CH₃, Ar¹ is Ar¹-5, Ar² is Ar²-6.
Table 181. Compounds of formula I.3 where R^{A} is CH₃, R² is CH₃, R⁵ is H, Ar¹ is Ar¹-1, Ar² is Ar²-1.
Table 182. Compounds of formula I.3 where R^{A} is CH₃, R² is CH₃, R⁵ is H, Ar¹ is Ar¹-1, Ar² is Ar²-2.
Table 183. Compounds of formula I.3 where R^{A} is CH₃, R² is CH₃, R⁵ is H, Ar¹ is Ar¹-1, Ar² is Ar²-3.
Table 184. Compounds of formula I.3 where R^{A} is CH₃, R² is CH₃, R⁵ is H, Ar¹ is Ar¹-1, Ar² is Ar²-4.
Table 185. Compounds of formula I.3 where R^{A} is CH₃, R² is CH₃, R⁵ is H, Ar¹ is Ar¹-1, Ar² is Ar²-5.
Table 186. Compounds of formula I.3 where R^{A} is CH₃, R² is CH₃, R⁵ is H, Ar¹ is Ar¹-1, Ar² is Ar²-6.
Table 187. Compounds of formula I.3 where R^{A} is CH₃, R² is CH₃, R⁵ is H, Ar¹ is Ar¹-2, Ar² is Ar²-1.
Table 188. Compounds of formula I.3 where R^{A} is CH₃, R² is CH₃, R⁵ is H, Ar¹ is Ar¹-2, Ar² is Ar²-2.
Table 189. Compounds of formula I.3 where R^{A} is CH₃, R² is CH₃, R⁵ is H, Ar¹ is Ar¹-2, Ar² is Ar²-3.
Table 190. Compounds of formula I.3 where R^{A} is CH₃, R² is CH₃, R⁵ is H, Ar¹ is Ar¹-2, Ar² is Ar²-4.
Table 191. Compounds of formula I.3 where R^{A} is CH₃, R² is CH₃, R⁵ is H, Ar¹ is Ar¹-2, Ar² is Ar²-5.
Table 192. Compounds of formula I.3 where R^{A} is CH₃, R² is CH₃, R⁵ is H, Ar¹ is Ar¹-2, Ar² is Ar²-6.
Table 193. Compounds of formula I.3 where R^{A} is CH₃, R² is CH₃, R⁵ is H, Ar¹ is Ar¹-3, Ar² is Ar²-1.
Table 194. Compounds of formula I.3 where R^{A} is CH₃, R² is CH₃, R⁵ is H, Ar¹ is Ar¹-3, Ar² is Ar²-2.
Table 195. Compounds of formula I.3 where R^{A} is CH₃, R² is CH₃, R⁵ is H, Ar¹ is Ar¹-3, Ar² is Ar²-3.
Table 196. Compounds of formula I.3 where R^{A} is CH₃, R² is CH₃, R⁵ is H, Ar¹ is Ar¹-3, Ar² is Ar²-4.
Table 197. Compounds of formula I.3 where R^{A} is CH₃, R² is CH₃, R⁵ is H, Ar¹ is Ar¹-3, Ar² is Ar²-5.
Table 198. Compounds of formula I.3 where R^{A} is CH₃, R² is CH₃, R⁵ is H, Ar¹ is Ar¹-3, Ar² is Ar²-6.
Table 199. Compounds of formula I.3 where R^{A} is CH₃, R² is CH₃, R⁵ is H, Ar¹ is Ar¹-4, Ar² is Ar²-1.
Table 200. Compounds of formula I.3 where R^{A} is CH₃, R² is CH₃, R⁵ is H, Ar¹ is Ar¹-4, Ar² is Ar²-2.
Table 201. Compounds of formula I.3 where R^{A} is CH₃, R² is CH₃, R⁵ is H, Ar¹ is Ar¹-4, Ar² is Ar²-3.
Table 202. Compounds of formula I.3 where R^{A} is CH₃, R² is CH₃, R⁵ is H, Ar¹ is Ar¹-4, Ar² is Ar²-4.
Table 203. Compounds of formula I.3 where R^{A} is CH₃, R² is CH₃, R⁵ is H, Ar¹ is Ar¹-4, Ar² is Ar²-5.
Table 204. Compounds of formula I.3 where R^{A} is CH₃, R² is CH₃, R⁵ is H, Ar¹ is Ar¹-4, Ar² is Ar²-6.
Table 205. Compounds of formula I.3 where R^{A} is CH₃, R² is CH₃, R⁵ is H, Ar¹ is Ar¹-5, Ar² is Ar²-1.
Table 206. Compounds of formula I.3 where R^{A} is CH₃, R² is CH₃, R⁵ is H, Ar¹ is Ar¹-5, Ar² is Ar²-2.
Table 207. Compounds of formula I.3 where R^{A} is CH₃, R² is CH₃, R⁵ is H, Ar¹ is Ar¹-5, Ar² is Ar²-3.
Table 208. Compounds of formula I.3 where R^{A} is CH₃, R² is CH₃, R⁵ is H, Ar¹ is Ar¹-5, Ar² is Ar²-4.
Table 209. Compounds of formula I.3 where R^{A} is CH₃, R² is CH₃, R⁵ is H, Ar¹ is Ar¹-5, Ar² is Ar²-5.
Table 210. Compounds of formula I.3 where R^{A} is CH₃, R² is CH₃, R⁵ is H, Ar¹ is Ar¹-5, Ar² is Ar²-6.
Table 211. Compounds of formula I.3 where R^{A} is CH₃, R² is CH₃, R⁵ is CH₃, Ar¹ is Ar¹-1, Ar² is Ar²-1.
Table 212. Compounds of formula I.3 where R^{A} is CH₃, R² is CH₃, R⁵ is CH₃, Ar¹ is Ar¹-1, Ar² is Ar²-2.
Table 213. Compounds of formula I.3 where R^{A} is CH₃, R² is CH₃, R⁵ is CH₃, Ar¹ is Ar¹-1, Ar² is Ar²-3.
Table 214. Compounds of formula I.3 where R^{A} is CH₃, R² is CH₃, R⁵ is CH₃, Ar¹ is Ar¹-1, Ar² is Ar²-4.
Table 215. Compounds of formula I.3 where R^{A} is CH₃, R² is CH₃, R⁵ is CH₃, Ar¹ is Ar¹-1, Ar² is Ar²-5.
Table 216. Compounds of formula I.3 where R^{A} is CH₃, R² is CH₃, R⁵ is CH₃, Ar¹ is Ar¹-1, Ar² is Ar²-6.
Table 217. Compounds of formula I.3 where R^{A} is CH₃, R² is CH₃, R⁵ is CH₃, Ar¹ is Ar¹-2, Ar² is Ar²-1.
Table 218. Compounds of formula I.3 where R^{A} is CH₃, R² is CH₃, R⁵ is CH₃, Ar¹ is Ar¹-2, Ar² is Ar²-2.
Table 219. Compounds of formula I.3 where R^{A} is CH₃, R² is CH₃, R^{S} is CH₃, Ar¹ is Ar¹-2, Ar² is Ar²-3.
Table 220. Compounds of formula I.3 where R^{A} is CH₃, R² is CH₃, R⁵ is CH₃, Ar¹ is Ar¹-2, Ar² is Ar²-4.
Table 221. Compounds of formula I.3 where R^{A} is CH₃, R² is CH₃, R⁵ is CH₃, Ar¹ is Ar¹-2, Ar² is Ar²-5.
Table 222. Compounds of formula I.3 where R^{A} is CH₃, R² is CH₃, R⁵ is CH₃, Ar¹ is Ar¹-2, Ar² is Ar²-6.
Table 223. Compounds of formula I.3 where R^{A} is CH₃, R² is CH₃, R⁵ is CH₃, Ar¹ is Ar¹-3, Ar² is Ar²-1.
Table 224. Compounds of formula I.3 where R^{A} is CH₃, R² is CH₃, R⁵ is CH₃, Ar¹ is Ar¹-3, Ar² is Ar²-2.
Table 225. Compounds of formula I.3 where R^{A} is CH₃, R² is CH₃, R⁵ is CH₃, Ar¹ is Ar¹-3, Ar² is Ar²-3.
Table 226. Compounds of formula I.3 where R^{A} is CH₃, R² is CH₃, R^{S} is CH₃, Ar¹ is Ar¹-3, Ar² is Ar²-4.
Table 227. Compounds of formula I.3 where R^{A} is CH₃, R² is CH₃, R^{S} is CH₃, Ar¹ is Ar¹-3, Ar² is Ar²-5.
Table 228. Compounds of formula I.3 where R^{A} is CH₃, R² is CH₃, R⁵ is CH₃, Ar¹ is Ar¹-3, Ar² is Ar²-6.
Table 229. Compounds of formula I.3 where R^{A} is CH₃, R² is CH₃, R⁵ is CH₃, Ar¹ is Ar¹-4, Ar² is Ar²-1.
Table 230. Compounds of formula I.3 where R^{A} is CH₃, R² is CH₃, R⁵ is CH₃, Ar¹ is Ar¹-4, Ar² is Ar²-2.
Table 231. Compounds of formula I.3 where R^{A} is CH₃, R² is CH₃, R⁵ is CH₃, Ar¹ is Ar¹-4, Ar² is Ar²-3.
Table 232. Compounds of formula I.3 where R^{A} is CH₃, R² is CH₃, R⁵ is CH₃, Ar¹ is Ar¹-4, Ar² is Ar²-4.
Table 233. Compounds of formula I.3 where R^{A} is CH₃, R² is CH₃, R⁵ is CH₃, Ar¹ is Ar¹-4, Ar² is Ar²-5.
Table 234. Compounds of formula I.3 where R^{A} is CH₃, R² is CH₃, R⁵ is CH₃, Ar¹ is Ar¹-4, Ar² is Ar²-6.
Table 235. Compounds of formula I.3 where R^{A} is CH₃, R² is CH₃, R⁵ is CH₃, Ar¹ is Ar¹-5, Ar² is Ar²-1.
Table 236. Compounds of formula I.3 where R^{A} is CH₃, R² is CH₃, R⁵ is CH₃, Ar¹ is Ar¹-5, Ar² is Ar²-2.
Table 237. Compounds of formula I.3 where R^{A} is CH₃, R² is CH₃, R⁵ is CH₃, Ar¹ is Ar¹-5, Ar² is Ar²-3.
Table 238. Compounds of formula I.3 where R^{A} is CH₃, R² is CH₃, R^{S} is CH₃, Ar¹ is Ar¹-5, Ar² is Ar²-4.
Table 239. Compounds of formula I.3 where R^{A} is CH₃, R² is CH₃, R⁵ is CH₃, Ar¹ is Ar¹-5, Ar² is Ar²-5.
Table 240. Compounds of formula I.3 where R^{A} is CH₃, R² is CH₃, R⁵ is CH₃, Ar¹ is Ar¹-5, Ar² is Ar²-6.
Table 241. Compounds of formula I.7 where R² is H, R⁵ is H, Ar¹ is Ar¹-1, Ar² is Ar²-1.
Table 242. Compounds of formula I.7 where R² is H, R⁵ is H, Ar¹ is Ar¹-1, Ar² is Ar²-2.
Table 243. Compounds of formula I.7 where R² is H, R⁵ is H, Ar¹ is Ar¹-1, Ar² is Ar²-3.
Table 244. Compounds of formula I.7 where R² is H, R⁵ is H, Ar¹ is Ar¹-1, Ar² is Ar²-4.
Table 245. Compounds of formula I.7 where R² is H, R⁵ is H, Ar¹ is Ar¹-1, Ar² is Ar²-5.
Table 246. Compounds of formula I.7 where R² is H, R⁵ is H, Ar¹ is Ar¹-1, Ar² is Ar²-6.
Table 247. Compounds of formula I.7 where R² is H, R⁵ is H, Ar¹ is Ar¹-2, Ar² is Ar²-1.
Table 248. Compounds of formula I.7 where R² is H, R⁵ is H, Ar¹ is Ar¹-2, Ar² is Ar²-2.
Table 249. Compounds of formula I.7 where R² is H, R⁵ is H, Ar¹ is Ar¹-2, Ar² is Ar²-3.
Table 250. Compounds of formula I.7 where R² is H, R⁵ is H, Ar¹ is Ar¹-2, Ar² is Ar²-4.
Table 251. Compounds of formula I.7 where R² is H, R⁵ is H, Ar¹ is Ar¹-2, Ar² is Ar²-5.
Table 252. Compounds of formula I.7 where R² is H, R⁵ is H, Ar¹ is Ar¹-2, Ar² is Ar²-6.
Table 253. Compounds of formula I.7 where R² is H, R⁵ is H, Ar¹ is Ar¹-3, Ar² is Ar²-1.
Table 254. Compounds of formula I.7 where R² is H, R⁵ is H, Ar¹ is Ar¹-3, Ar² is Ar²-2.
Table 255. Compounds of formula I.7 where R² is H, R⁵ is H, Ar¹ is Ar¹-3, Ar² is Ar²-3.
Table 256. Compounds of formula I.7 where R² is H, R⁵ is H, Ar¹ is Ar¹-3, Ar² is Ar²-4.
Table 257. Compounds of formula I.7 where R² is H, R⁵ is H, Ar¹ is Ar¹-3, Ar² is Ar²-5.
Table 258. Compounds of formula I.7 where R² is H, R⁵ is H, Ar¹ is Ar¹-3, Ar² is Ar²-6.
Table 259. Compounds of formula I.7 where R² is H, R⁵ is H, Ar¹ is Ar¹-4, Ar² is Ar²-1.
Table 260. Compounds of formula I.7 where R² is H, R⁵ is H, Ar¹ is Ar¹-4, Ar² is Ar²-2.
Table 261. Compounds of formula I.7 where R² is H, R⁵ is H, Ar¹ is Ar¹-4, Ar² is Ar²-3.
Table 262. Compounds of formula I.7 where R² is H, R⁵ is H, Ar¹ is Ar¹-4, Ar² is Ar²-4.
Table 263. Compounds of formula I.7 where R² is H, R⁵ is H, Ar¹ is Ar¹-4, Ar² is Ar²-5.
Table 264. Compounds of formula I.7 where R² is H, R⁵ is H, Ar¹ is Ar¹-4, Ar² is Ar²-6.
Table 265. Compounds of formula I.7 where R² is H, R⁵ is H, Ar¹ is Ar¹-5, Ar² is Ar²-1.
Table 266. Compounds of formula I.7 where R² is H, R⁵ is H, Ar¹ is Ar¹-5, Ar² is Ar²-2.
Table 267. Compounds of formula I.7 where R² is H, R⁵ is H, Ar¹ is Ar¹-5, Ar² is Ar²-3.
Table 268. Compounds of formula I.7 where R² is H, R⁵ is H, Ar¹ is Ar¹-5, Ar² is Ar²-4.
Table 269. Compounds of formula I.7 where R² is H, R⁵ is H, Ar¹ is Ar¹-5, Ar² is Ar²-5.
Table 270. Compounds of formula I.7 where R² is H, R⁵ is H, Ar¹ is Ar¹-5, Ar² is Ar²-6.
Table 271. Compounds of formula I.7 where R² is H, R⁵ is CH₃, Ar¹ is Ar¹-1, Ar² is Ar²-1.
Table 272. Compounds of formula I.7 where R² is H, R⁵ is CH₃, Ar¹ is Ar¹-1, Ar² is Ar²-2.
Table 273. Compounds of formula I.7 where R² is H, R⁵ is CH₃, Ar¹ is Ar¹-1, Ar² is Ar²-3.
Table 274. Compounds of formula I.7 where R² is H, R⁵ is CH₃, Ar¹ is Ar¹-1, Ar² is Ar²-4.
Table 275. Compounds of formula I.7 where R² is H, R⁵ is CH₃, Ar¹ is Ar¹-1, Ar² is Ar²-5.
Table 276. Compounds of formula I.7 where R² is H, R⁵ is CH₃, Ar¹ is Ar¹-1, Ar² is Ar²-6.
Table 277. Compounds of formula I.7 where R² is H, R⁵ is CH₃, Ar¹ is Ar¹-2, Ar² is Ar²-1.
Table 278. Compounds of formula I.7 where R² is H, R⁵ is CH₃, Ar¹ is Ar¹-2, Ar² is Ar²-2.
Table 279. Compounds of formula I.7 where R² is H, R⁵ is CH₃, Ar¹ is Ar¹-2, Ar² is Ar²-3.
Table 280. Compounds of formula I.7 where R² is H, R⁵ is CH₃, Ar¹ is Ar¹-2, Ar² is Ar²-4.
Table 281. Compounds of formula I.7 where R² is H, R⁵ is CH₃, Ar¹ is Ar¹-2, Ar² is Ar²-5.
Table 282. Compounds of formula I.7 where R² is H, R⁵ is CH₃, Ar¹ is Ar¹-2, Ar² is Ar²-6.
Table 283. Compounds of formula I.7 where R² is H, R⁵ is CH₃, Ar¹ is Ar¹-3, Ar² is Ar²-1.
Table 284. Compounds of formula I.7 where R² is H, R⁵ is CH₃, Ar¹ is Ar¹-3, Ar² is Ar²-2.
Table 285. Compounds of formula I.7 where R² is H, R⁵ is CH₃, Ar¹ is Ar¹-3, Ar² is Ar²-3.
Table 286. Compounds of formula I.7 where R² is H, R⁵ is CH₃, Ar¹ is Ar¹-3, Ar² is Ar²-4.
Table 287. Compounds of formula I.7 where R² is H, R⁵ is CH₃, Ar¹ is Ar¹-3, Ar² is Ar²-5.
Table 288. Compounds of formula I.7 where R² is H, R⁵ is CH₃, Ar¹ is Ar¹-3, Ar² is Ar²-6.
Table 289. Compounds of formula I.7 where R² is H, R⁵ is CH₃, Ar¹ is Ar¹-4, Ar² is Ar²-1.
Table 290. Compounds of formula I.7 where R² is H, R⁵ is CH₃, Ar¹ is Ar¹-4, Ar² is Ar²-2.
Table 291. Compounds of formula I.7 where R² is H, R⁵ is CH₃, Ar¹ is Ar¹-4, Ar² is Ar²-3.
Table 292. Compounds of formula I.7 where R² is H, R⁵ is CH₃, Ar¹ is Ar¹-4, Ar² is Ar²-4.
Table 293. Compounds of formula I.7 where R² is H, R⁵ is CH₃, Ar¹ is Ar¹-4, Ar² is Ar²-5.
Table 294. Compounds of formula I.7 where R² is H, R⁵ is CH₃, Ar¹ is Ar¹-4, Ar² is Ar²-6.
Table 295. Compounds of formula I.7 where R² is H, R⁵ is CH₃, Ar¹ is Ar¹-5, Ar² is Ar²-1.
Table 296. Compounds of formula I.7 where R² is H, R⁵ is CH₃, Ar¹ is Ar¹-5, Ar² is Ar²-2.
Table 297. Compounds of formula I.7 where R² is H, R⁵ is CH₃, Ar¹ is Ar¹-5, Ar² is Ar²-3.
Table 298. Compounds of formula I.7 where R² is H, R⁵ is CH₃, Ar¹ is Ar¹-5, Ar² is Ar²-4.
Table 299. Compounds of formula I.7 where R² is H, R⁵ is CH₃, Ar¹ is Ar¹-5, Ar² is Ar²-5.
Table 300. Compounds of formula I.7 where R² is H, R⁵ is CH₃, Ar¹ is Ar¹-5, Ar² is Ar²-6.
Table 301. Compounds of formula I.7 where R² is CH₃, R⁵ is H, Ar¹ is Ar¹-1, Ar² is Ar²-1.
Table 302. Compounds of formula I.7 where R² is CH₃, R⁵ is H, Ar¹ is Ar¹-1, Ar² is Ar²-2.
Table 303. Compounds of formula I.7 where R² is CH₃, R⁵ is H, Ar¹ is Ar¹-1, Ar² is Ar²-3.
Table 304. Compounds of formula I.7 where R² is CH₃, R⁵ is H, Ar¹ is Ar¹-1, Ar² is Ar²-4.
Table 305. Compounds of formula I.7 where R² is CH₃, R⁵ is H, Ar¹ is Ar¹-1, Ar² is Ar²-5.
Table 306. Compounds of formula I.7 where R² is CH₃, R⁵ is H, Ar¹ is Ar¹-1, Ar² is Ar²-6.
Table 307. Compounds of formula I.7 where R² is CH₃, R⁵ is H, Ar¹ is Ar¹-2, Ar² is Ar²-1.
Table 308. Compounds of formula I.7 where R² is CH₃, R⁵ is H, Ar¹ is Ar¹-2, Ar² is Ar²-2.
Table 309. Compounds of formula I.7 where R² is CH₃, R⁵ is H, Ar¹ is Ar¹-2, Ar² is Ar²-3.
Table 310. Compounds of formula I.7 where R² is CH₃, R⁵ is H, Ar¹ is Ar¹-2, Ar² is Ar²-4.
Table 311. Compounds of formula I.7 where R² is CH₃, R⁵ is H, Ar¹ is Ar¹-2, Ar² is Ar²-5.
Table 312. Compounds of formula I.7 where R² is CH₃, R⁵ is H, Ar¹ is Ar¹-2, Ar² is Ar²-6.
Table 313. Compounds of formula I.7 where R² is CH₃, R⁵ is H, Ar¹ is Ar¹-3, Ar² is Ar²-1.
Table 314. Compounds of formula I.7 where R² is CH₃, R⁵ is H, Ar¹ is Ar¹-3, Ar² is Ar²-2.
Table 315. Compounds of formula I.7 where R² is CH₃, R⁵ is H, Ar¹ is Ar¹-3, Ar² is Ar²-3.
Table 316. Compounds of formula I.7 where R² is CH₃, R⁵ is H, Ar¹ is Ar¹-3, Ar² is Ar²-4.
Table 317. Compounds of formula I.7 where R² is CH₃, R⁵ is H, Ar¹ is Ar¹-3, Ar² is Ar²-5.
Table 318. Compounds of formula I.7 where R² is CH₃, R⁵ is H, Ar¹ is Ar¹-3, Ar² is Ar²-6.
Table 319. Compounds of formula I.7 where R² is CH₃, R⁵ is H, Ar¹ is Ar¹-4, Ar² is Ar²-1.
Table 320. Compounds of formula I.7 where R² is CH₃, R⁵ is H, Ar¹ is Ar¹-4, Ar² is Ar²-2.
Table 321. Compounds of formula I.7 where R² is CH₃, R⁵ is H, Ar¹ is Ar¹-4, Ar² is Ar²-3.
Table 322. Compounds of formula I.7 where R² is CH₃, R⁵ is H, Ar¹ is Ar¹-4, Ar² is Ar²-4.
Table 323. Compounds of formula I.7 where R² is CH₃, R⁵ is H, Ar¹ is Ar¹-4, Ar² is Ar²-5.
Table 324. Compounds of formula I.7 where R² is CH₃, R⁵ is H, Ar¹ is Ar¹-4, Ar² is Ar²-6.
Table 325. Compounds of formula I.7 where R² is CH₃, R⁵ is H, Ar¹ is Ar¹-5, Ar² is Ar²-1.
Table 326. Compounds of formula I.7 where R² is CH₃, R⁵ is H, Ar¹ is Ar¹-5, Ar² is Ar²-2.
Table 327. Compounds of formula I.7 where R² is CH₃, R⁵ is H, Ar¹ is Ar¹-5, Ar² is Ar²-3.
Table 328. Compounds of formula I.7 where R² is CH₃, R⁵ is H, Ar¹ is Ar¹-5, Ar² is Ar²-4.
Table 329. Compounds of formula I.7 where R² is CH₃, R⁵ is H, Ar¹ is Ar¹-5, Ar² is Ar²-5.
Table 330. Compounds of formula I.7 where R² is CH₃, R⁵ is H, Ar¹ is Ar¹-5, Ar² is Ar²-6.
Table 331. Compounds of formula I.7 where R² is CH₃, R⁵ is CH₃, Ar¹ is Ar¹-1, Ar² is Ar²-1.
Table 332. Compounds of formula I.7 where R² is CH₃, R⁵ is CH₃, Ar¹ is Ar¹-1, Ar² is Ar²-2.
Table 333. Compounds of formula I.7 where R² is CH₃, R⁵ is CH₃, Ar¹ is Ar¹-1, Ar² is Ar²-3.
Table 334. Compounds of formula I.7 where R² is CH₃, R⁵ is CH₃, Ar¹ is Ar¹-1, Ar² is Ar²-4.
Table 335. Compounds of formula I.7 where R² is CH₃, R⁵ is CH₃, Ar¹ is Ar¹-1, Ar² is Ar²-5.
Table 336. Compounds of formula I.7 where R² is CH₃, R⁵ is CH₃, Ar¹ is Ar¹-1, Ar² is Ar²-6.
Table 337. Compounds of formula I.7 where R² is CH₃, R⁵ is CH₃, Ar¹ is Ar¹-2, Ar² is Ar²-1.
Table 338. Compounds of formula I.7 where R² is CH₃, R⁵ is CH₃, Ar¹ is Ar¹-2, Ar² is Ar²-2.
Table 339. Compounds of formula I.7 where R² is CH₃, R⁵ is CH₃, Ar¹ is Ar¹-2, Ar² is Ar²-3.
Table 340. Compounds of formula I.7 where R² is CH₃, R⁵ is CH₃, Ar¹ is Ar¹-2, Ar² is Ar²-4.
Table 341. Compounds of formula I.7 where R² is CH₃, R⁵ is CH₃, Ar¹ is Ar¹-2, Ar² is Ar²-5.
Table 342. Compounds of formula I.7 where R² is CH₃, R⁵ is CH₃, Ar¹ is Ar¹-2, Ar² is Ar²-6.
Table 343. Compounds of formula I.7 where R² is CH₃, R⁵ is CH₃, Ar¹ is Ar¹-3, Ar² is Ar²-1.
Table 344. Compounds of formula I.7 where R² is CH₃, R⁵ is CH₃, Ar¹ is Ar¹-3, Ar² is Ar²-2.
Table 345. Compounds of formula I.7 where R² is CH₃, R⁵ is CH₃, Ar¹ is Ar¹-3, Ar² is Ar²-3.
Table 346. Compounds of formula I.7 where R² is CH₃, R⁵ is CH₃, Ar¹ is Ar¹-3, Ar² is Ar²-4.
Table 347. Compounds of formula I.7 where R² is CH₃, R⁵ is CH₃, Ar¹ is Ar¹-3, Ar² is Ar²-5.
Table 348. Compounds of formula I.7 where R² is CH₃, R⁵ is CH₃, Ar¹ is Ar¹-3, Ar² is Ar²-6.
Table 349. Compounds of formula I.7 where R² is CH₃, R⁵ is CH₃, Ar¹ is Ar¹-4, Ar² is Ar²-1.
Table 350. Compounds of formula I.7 where R² is CH₃, R⁵ is CH₃, Ar¹ is Ar¹-4, Ar² is Ar²-2.
Table 351. Compounds of formula I.7 where R² is CH₃, R⁵ is CH₃, Ar^{C} is Ar¹-4, Ar² is Ar²-3.
Table 352. Compounds of formula I.7 where R² is CH₃, R⁵ is CH₃, Ar^{N} is Ar¹-4, Ar² is Ar²-4.
Table 353. Compounds of formula I.7 where R² is CH₃, R⁵ is CH₃, Ar¹ is Ar¹-4, Ar² is Ar²-5.
Table 354. Compounds of formula I.7 where R² is NH₃, R⁵ is CH₃, Ar¹ is Ar¹-4, Ar² is Ar²-6.
Table 355. Compounds of formula I.7 where R² is CH₃, R⁵ is CH₃, Ar¹ is Ar-5, Ar² is Ar²-1.
Table 356. Compounds of formula I.7 where R² is CH₃, R⁵ is CH₃, Ar¹ is Ar¹-5, Ar² is Ar²-2.
Table 357. Compounds of formula I.7 where R² is CH₃, R⁵ is CH₃, Ar¹ is Ar¹-5, Ar² is Ar²-3.
Table 358. Compounds of formula I.7 where R² is CH₃, R⁵ is CH₃, Ar¹ is Ar¹-5, Ar² is Ar²-4.
Table 359. Compounds of formula I.7 where R² is CH₃, R⁵ is CH₃, Ar¹ is Ar¹-5, Ar² is Ar²-5.
Table 360. Compounds of formula I.7 where R² is CH₃, R⁵ is CH₃, Ar¹ is Ar¹-5, Ar² is Ar²-6.

TABLE B

| Line | B¹ | B² | B³ | B⁴ |
|---|---|---|---|---|
| 1. | N | N | CH | CH |
| 2. | N | N | CH | C—Cl |
| 3. | N | N | CH | C—F |
| 4. | N | N | CH | C—Br |
| 5. | N | N | CH | C—CH₃ |
| 6. | N | N | C—Cl | CH |
| 7. | N | N | C—Cl | C—Cl |
| 8. | N | N | C—Cl | C—F |
| 9. | N | N | C—Cl | C—Br |
| 10. | N | N | C—Cl | C—CH₃ |
| 11. | N | N | C—F | CH |
| 12. | N | N | C—F | C—Cl |
| 13. | N | N | C—F | C—F |
| 14. | N | N | C—F | C—Br |
| 15. | N | N | C—F | C—CH₃ |
| 16. | N | N | C—Br | CH |
| 17. | N | N | C—Br | C—Cl |
| 18. | N | N | C—Br | C—F |
| 19. | N | N | C—Br | C—Br |
| 20. | N | N | C—Br | C—CH₃ |
| 21. | N | N | C—CH₃ | CH |
| 22. | N | N | C—CH₃ | C—Cl |
| 23. | N | N | C—CH₃ | C—F |
| 24 | N | N | C—CH₃ | C—Br |
| 25 | N | N | C—CH₃ | C—CH₃ |
| 26. | N | CH | N | CH |
| 27 | N | CH | N | C—Cl |
| 28 | N | CH | N | C—F |
| 29 | N | CH | N | C—Br |
| 30 | N | CH | N | C—CH₃ |
| 31. | N | CH | CH | CH |
| 32 | N | CH | CH | C—Cl |
| 33. | N | CH | CH | C—F |
| 34 | N | CH | CH | C—Br |
| 35. | N | CH | CH | C—CH₃ |
| 36 | N | CH | C—Cl | CH |
| 37. | N | CH | C—Cl | C—Cl |
| 38. | N | CH | C—Cl | C—F |
| 39. | N | CH | C—Cl | C—Br |
| 40. | N | CH | C—Cl | C—CH₃ |
| 41. | N | CH | C—F | CH |
| 42. | N | CH | C—F | C—Cl |
| 43. | N | CH | C—F | C—F |
| 44. | N | CH | C—F | C—Br |
| 45. | N | CH | C—F | C—CH₃ |
| 46. | N | CH | C—Br | CH |
| 47. | N | CH | C—Br | C—Cl |
| 48. | N | CH | C—Br | C—F |
| 49. | N | CH | C—Br | C—Br |
| 50. | N | CH | C—Br | C—CH₃ |
| 51. | N | CH | C—CH₃ | CH |
| 52. | N | CH | C—CH₃ | C—Cl |
| 53. | N | CH | C—CH₃ | C—F |
| 54. | N | CH | C—CH₃ | C—Br |
| 55. | N | CH | C—CH₃ | C—CH₃ |
| 56. | N | C—Cl | N | CH |
| 57. | N | C—Cl | N | C—Cl |
| 58. | N | C—Cl | N | C—F |
| 59. | N | C—Cl | N | C—Br |
| 60. | N | C—Cl | N | C—CH₃ |
| 61. | N | C—Cl | CH | CH |
| 62. | N | C—Cl | CH | C—Cl |
| 63. | N | C—Cl | CH | C—F |
| 64. | N | C—Cl | CH | C—Br |
| 65. | N | C—Cl | CH | C—CH₃ |
| 66. | N | C—Cl | C—Cl | CH |
| 67. | N | C—Cl | C—Cl | C—Cl |
| 68. | N | C—Cl | C—Cl | C—F |
| 69. | N | C—Cl | C—Cl | C—Br |
| 70. | N | C—Cl | C—Cl | C—CH₃ |
| 71. | N | C—Cl | C—F | CH |
| 72. | N | C—Cl | C—F | C—Cl |
| 73. | N | C—Cl | C—F | C—F |
| 74. | N | C—Cl | C—F | C—Br |
| 75. | N | C—Cl | C—F | C—CH₃ |
| 76. | N | C—Cl | C—Br | CH |
| 77. | N | C—Cl | C—Br | C—Cl |
| 78. | N | C—Cl | C—Br | C—F |
| 79. | N | C—Cl | C—Br | C—Br |
| 80. | N | C—Cl | C—Br | C—CH₃ |
| 81. | N | C—Cl | C—CH₃ | CH |
| 82. | N | C—Cl | C—CH₃ | C—Cl |
| 83. | N | C—Cl | C—CH₃ | C—F |
| 84. | N | C—Cl | C—CH₃ | C—Br |
| 85. | N | C—Cl | C—CH₃ | C—CH₃ |
| 86. | N | C—F | N | CH |
| 87. | N | C—F | N | C—Cl |
| 88. | N | C—F | N | C—F |
| 89. | N | C—F | N | C—Br |
| 90. | N | C—F | N | C—CH₃ |
| 91. | N | C—F | CH | CH |
| 92. | N | C—F | CH | C—Cl |
| 93. | N | C—F | CH | C—F |
| 94. | N | C—F | CH | C—Br |
| 95. | N | C—F | CH | C—CH₃ |
| 96. | N | C—F | C—Cl | CH |
| 97. | N | C—F | C—Cl | C—Cl |
| 98. | N | C—F | C—Cl | C—F |
| 99. | N | C—F | C—Cl | C—Br |
| 100. | N | C—F | C—Cl | C—CH₃ |
| 101. | N | C—F | C—F | CH |
| 102. | N | C—F | C—F | C—Cl |
| 103. | N | C—F | C—F | C—F |
| 104. | N | C—F | C—F | C—Br |
| 105. | N | C—F | C—F | C—CH₃ |
| 106. | N | C—F | C—Br | CH |
| 107. | N | C—F | C—Br | C—Cl |
| 108. | N | C—F | C—Br | C—F |
| 109. | N | C—F | C—Br | C—Br |
| 110. | N | C—F | C—Br | C—CH₃ |
| 111. | N | C—F | C—CH₃ | CH |
| 112. | N | C—F | C—CH₃ | C—Cl |
| 113. | N | C—F | C—CH₃ | C—F |
| 114. | N | C—F | C—CH₃ | C—Br |
| 115. | N | C—F | C—CH₃ | C—CH₃ |
| 116. | N | C—Br | N | CH |
| 117. | N | C—Br | N | C—Cl |
| 118. | N | C—Br | N | C—F |
| 119. | N | C—Br | N | C—Br |
| 120. | N | C—Br | N | C—CH₃ |
| 121. | N | C—Br | CH | CH |
| 122. | N | C—Br | CH | C—Cl |
| 123. | N | C—Br | CH | C—F |
| 124. | N | C—Br | CH | C—Br |
| 125. | N | C—Br | CH | C—CH₃ |
| 126. | N | C—Br | C—Cl | CH |
| 127. | N | C—Br | C—Cl | C—Cl |
| 128. | N | C—Br | C—Cl | C—F |
| 129. | N | C—Br | C—Cl | C—Br |
| 130. | N | C—Br | C—Cl | C—CH₃ |
| 131. | N | C—Br | C—F | CH |
| 132. | N | C—Br | C—F | C—Cl |
| 133. | N | C—Br | C—F | C—F |
| 134. | N | C—Br | C—F | C—Br |
| 135. | N | C—Br | C—F | C—CH₃ |
| 136. | N | C—Br | C—Br | CH |
| 137. | N | C—Br | C—Br | C—Cl |
| 138. | N | C—Br | C—Br | C—F |
| 139. | N | C—Br | C—Br | C—Br |
| 140. | N | C—Br | C—Br | C—CH₃ |
| 141. | N | C—Br | C—CH₃ | CH |
| 142. | N | C—Br | C—CH₃ | C—Cl |
| 143. | N | C—Br | C—CH₃ | C—F |
| 144. | N | C—Br | C—CH₃ | C—Br |
| 145. | N | C—Br | C—CH₃ | C—CH₃ |
| 146. | N | C—CH₃ | N | CH |
| 147. | N | C—CH₃ | N | C—Cl |
| 148. | N | C—CH₃ | N | C—F |
| 149. | N | C—CH₃ | N | C—Br |
| 150. | N | C—CH₃ | N | C—CH₃ |
| 151. | N | C—CH₃ | CH | CH |
| 152. | N | C—CH₃ | CH | C—Cl |
| 153. | N | C—CH₃ | CH | C—F |
| 154. | N | C—CH₃ | CH | C—Br |
| 155. | N | C—CH₃ | CH | C—CH₃ |
| 156. | N | C—CH₃ | C—Cl | CH |
| 157. | N | C—CH₃ | C—Cl | C—Cl |
| 158. | N | C—CH₃ | C—Cl | C—F |
| 159. | N | C—CH₃ | C—Cl | C—Br |
| 160. | N | C—CH₃ | C—Cl | C—CH₃ |
| 161. | N | C—CH₃ | C—F | CH |
| 162. | N | C—CH₃ | C—F | C—Cl |
| 163. | N | C—CH₃ | C—F | C—F |
| 164. | N | C—CH₃ | C—F | C—Br |
| 165. | N | C—CH₃ | C—F | C—CH₃ |
| 166. | N | C—CH₃ | C—Br | CH |
| 167. | N | C—CH₃ | C—Br | C—Cl |
| 168. | N | C—CH₃ | C—Br | C—F |
| 169. | N | C—CH₃ | C—Br | C—Br |
| 170. | N | C—CH₃ | C—Br | C—CH₃ |
| 171. | N | C—CH₃ | C—CH₃ | CH |
| 172. | N | C—CH₃ | C—CH₃ | C—Cl |
| 173. | N | C—CH₃ | C—CH₃ | C—F |
| 174. | N | C—CH₃ | C—CH₃ | C—Br |
| 175. | N | C—CH₃ | C—CH₃ | C—CH₃ |
| 176. | CH | N | N | CH |
| 177. | CH | N | N | C—Cl |
| 178. | CH | N | N | C—F |
| 179. | CH | N | N | C—Br |
| 180. | CH | N | N | C—CH₃ |
| 181. | CH | N | CH | CH |
| 182. | CH | N | CH | C—Cl |
| 183. | CH | N | CH | C—F |
| 184. | CH | N | CH | C—Br |
| 185. | CH | N | CH | C—CH₃ |
| 186 | CH | N | C—Cl | CH |
| 187. | CH | N | C—Cl | C—Cl |
| 188. | CH | N | C—Cl | C—F |
| 189. | CH | N | C—Cl | C—Br |
| 190. | CH | N | C—Cl | C—CH₃ |
| 191. | CH | N | C—F | CH |
| 192. | CH | N | C—F | C—Cl |
| 193. | CH | N | C—F | C—F |
| 194. | CH | N | C—F | C—Br |
| 195. | CH | N | C—F | C—CH₃ |
| 196. | CH | N | C—Br | CH |
| 197. | CH | N | C—Br | C—Cl |
| 198. | CH | N | C—Br | C—F |
| 199. | CH | N | C—Br | C—Br |
| 200. | CH | N | C—Br | C—CH₃ |
| 201. | CH | N | C—CH₃ | CH |
| 202. | CH | N | C—CH₃ | C—Cl |
| 203. | CH | N | C—CH₃ | C—F |
| 204. | CH | N | C—CH₃ | C—Br |
| 205. | CH | N | C—CH₃ | C—CH₃ |
| 206. | CH | CH | N | CH |
| 207. | CH | CH | N | C—Cl |
| 208. | CH | CH | N | C—F |
| 209. | CH | CH | N | C—Br |
| 210. | CH | CH | N | C—CH₃ |
| 211. | CH | CH | CH | CH |
| 212. | CH | CH | CH | C—Cl |
| 213. | CH | CH | CH | C—F |
| 214. | CH | CH | CH | C—Br |
| 215. | CH | CH | CH | C—CH₃ |
| 216. | CH | CH | C—Cl | CH |
| 217. | CH | CH | C—Cl | C—Cl |
| 218. | CH | CH | C—Cl | C—F |
| 219. | CH | CH | C—Cl | C—Br |
| 220. | CH | CH | C—Cl | C—CH₃ |
| 221. | CH | CH | C—F | CH |
| 222. | CH | CH | C—F | C—Cl |
| 223. | CH | CH | C—F | C—F |
| 224. | CH | CH | C—F | C—Br |
| 225. | CH | CH | C—F | C—CH₃ |
| 226. | CH | CH | C—Br | CH |
| 227. | CH | CH | C—Br | C—Cl |
| 228. | CH | CH | C—Br | C—F |
| 229. | CH | CH | C—Br | C—Br |
| 230. | CH | CH | C—Br | C—CH₃ |
| 231. | CH | CH | C—CH₃ | CH |
| 232. | CH | CH | C—CH₃ | C—Cl |
| 233. | CH | CH | C—CH₃ | C—F |
| 234. | CH | CH | C—CH₃ | C—Br |
| 235 | CH | CH | C—CH₃ | C—CH₃ |
| 236. | CH | C—Cl | N | CH |
| 237. | CH | C—Cl | N | C—Cl |
| 238. | CH | C—Cl | N | C—F |
| 239. | CH | C—Cl | N | C—Br |
| 240. | CH | C—Cl | N | C—CH₃ |
| 241. | CH | C—Cl | CH | CH |
| 242. | CH | C—Cl | CH | C—Cl |
| 243. | CH | C—Cl | CH | C—F |
| 244. | CH | C—Cl | CH | C—Br |
| 245. | CH | C—Cl | CH | C—CH₃ |
| 246. | CH | C—Cl | C—Cl | CH |
| 247. | CH | C—Cl | C—Cl | C—Cl |
| 248. | CH | C—Cl | C—Cl | C—F |
| 249. | CH | C—Cl | C—Cl | C—Br |
| 250. | CH | C—Cl | C—Cl | C—CH₃ |
| 251. | CH | C—Cl | C—F | CH |
| 252. | CH | C—Cl | C—F | C—Cl |
| 253. | CH | C—Cl | C—F | C—F |
| 254. | CH | C—Cl | C—F | C—Br |
| 255. | CH | C—Cl | C—F | C—CH₃ |
| 256. | CH | C—Cl | C—Br | CH |
| 257. | CH | C—Cl | C—Br | C—Cl |
| 258. | CH | C—Cl | C—Br | C—F |
| 259. | CH | C—Cl | C—Br | C—Br |
| 260. | CH | C—Cl | C—Br | C—CH₃ |
| 261. | CH | C—Cl | C—CH₃ | CH |
| 262. | CH | C—Cl | C—CH₃ | C—Cl |
| 263. | CH | C—Cl | C—CH₃ | C—F |
| 264. | CH | C—Cl | C—CH₃ | C—Br |
| 265. | CH | C—Cl | C—CH₃ | C—CH₃ |
| 266. | CH | C—F | N | CH |
| 267. | CH | C—F | N | C—Cl |
| 268. | CH | C—F | N | C—F |
| 269. | CH | C—F | N | C—Br |
| 270. | CH | C—F | N | C—CH₃ |
| 271. | CH | C—F | CH | CH |
| 272. | CH | C—F | CH | C—Cl |
| 273. | CH | C—F | CH | C—F |
| 274. | CH | C—F | CH | C—Br |
| 275. | CH | C—F | CH | C—CH₃ |
| 276. | CH | C—F | C—Cl | CH |
| 277. | CH | C—F | C—Cl | C—Cl |
| 278. | CH | C—F | C—Cl | C—F |
| 279. | CH | C—F | C—Cl | C—Br |
| 280. | CH | C—F | C—Cl | C—CH₃ |
| 281. | CH | C—F | C—F | CH |
| 282. | CH | C—F | C—F | C—Cl |
| 283. | CH | C—F | C—F | C—F |
| 284. | CH | C—F | C—F | C—Br |
| 285. | CH | C—F | C—F | C—CH₃ |
| 286. | CH | C—F | C—Br | CH |
| 287. | CH | C—F | C—Br | C—Cl |
| 288. | CH | C—F | C—Br | C—F |
| 289. | CH | C—F | C—Br | C—Br |
| 290. | CH | C—F | C—Br | C—CH₃ |
| 291. | CH | C—F | C—CH₃ | CH |
| 292. | CH | C—F | C—CH₃ | C—Cl |
| 293. | CH | C—F | C—CH₃ | C—F |
| 294. | CH | C—F | C—CH₃ | C—Br |
| 295. | CH | C—F | C—CH₃ | C—CH₃ |
| 296. | CH | C—Br | N | CH |
| 297. | CH | C—Br | N | C—Cl |
| 298. | CH | C—Br | N | C—F |
| 299. | CH | C—Br | N | C—Br |
| 300. | CH | C—Br | N | C—CH₃ |
| 301. | CH | C—Br | CH | CH |
| 302. | CH | C—Br | CH | C—Cl |
| 303. | CH | C—Br | CH | C—F |
| 304. | CH | C—Br | CH | C—Br |
| 305. | CH | C—Br | CH | C—CH₃ |
| 306. | CH | C—Br | C—Cl | CH |
| 307. | CH | C—Br | C—Cl | C—Cl |
| 308. | CH | C—Br | C—Cl | C—F |
| 309. | CH | C—Br | C—Cl | C—Br |
| 310. | CH | C—Br | C—Cl | C—CH₃ |
| 311. | CH | C—Br | C—F | CH |
| 312. | CH | C—Br | C—F | C—Cl |
| 313. | CH | C—Br | C—F | C—F |
| 314. | CH | C—Br | C—F | C—Br |
| 315. | CH | C—Br | C—F | C—CH₃ |
| 316. | CH | C—Br | C—Br | CH |
| 317. | CH | C—Br | C—Br | C—Cl |
| 318. | CH | C—Br | C—Br | C—F |
| 319. | CH | C—Br | C—Br | C—Br |
| 320. | CH | C—Br | C—Br | C—CH₃ |
| 321. | CH | C—Br | C—CH₃ | CH |
| 322. | CH | C—Br | C—CH₃ | C—Cl |
| 323. | CH | C—Br | C—CH₃ | C—F |
| 324. | CH | C—Br | C—CH₃ | C—Br |
| 325. | CH | C—Br | C—CH₃ | C—CH₃ |
| 326. | CH | C—CH₃ | N | CH |
| 327. | CH | C—CH₃ | N | C—Cl |
| 328. | CH | C—CH₃ | N | C—F |
| 329. | CH | C—CH₃ | N | C—Br |
| 330. | CH | C—CH₃ | N | C—CH₃ |
| 331. | CH | C—CH₃ | CH | CH |
| 332. | CH | C—CH₃ | CH | C—Cl |
| 333. | CH | C—CH₃ | CH | C—F |
| 334. | CH | C—CH₃ | CH | C—Br |
| 335. | CH | C—CH₃ | CH | C—CH₃ |
| 336. | CH | C—CH₃ | C—Cl | CH |
| 337. | CH | C—CH₃ | C—Cl | C—Cl |
| 338. | CH | C—CH₃ | C—Cl | C—F |
| 339. | CH | C—CH₃ | C—Cl | C—Br |
| 340. | CH | C—CH₃ | C—Cl | C—CH₃ |
| 341. | CH | C—CH₃ | C—F | CH |
| 342. | CH | C—CH₃ | C—F | C—Cl |
| 343. | CH | C—CH₃ | C—F | C—F |
| 344. | CH | C—CH₃ | C—F | C—Br |
| 345. | CH | C—CH₃ | C—F | C—CH₃ |
| 346. | CH | C—CH₃ | C—Br | CH |
| 347. | CH | C—CH₃ | C—Br | C—Cl |
| 348. | CH | C—CH₃ | C—Br | C—F |
| 349. | CH | C—CH₃ | C—Br | C—Br |
| 350. | CH | C—CH₃ | C—Br | C—CH₃ |
| 351. | CH | C—CH₃ | C—CH₃ | CH |
| 352. | CH | C—CH₃ | C—CH₃ | C—Cl |
| 353. | CH | C—CH₃ | C—CH₃ | C—F |
| 354. | CH | C—CH₃ | C—CH₃ | C—Br |
| 355. | CH | C—CH₃ | C—CH₃ | C—CH₃ |
| 356. | C—Cl | N | N | CH |
| 357. | C—Cl | N | N | C—Cl |
| 358. | C—Cl | N | N | C—F |
| 359. | C—Cl | N | N | C—Br |
| 360. | C—Cl | N | N | C—CH₃ |
| 361. | C—Cl | N | CH | CH |
| 362. | C—Cl | N | CH | C—Cl |
| 363. | C—Cl | N | CH | C—F |
| 364. | C—Cl | N | CH | C—Br |
| 365. | C—Cl | N | CH | C—CH₃ |
| 366. | C—Cl | N | C—Cl | CH |
| 367. | C—Cl | N | C—Cl | C—Cl |
| 368. | C—Cl | N | C—Cl | C—F |
| 369. | C—Cl | N | C—Cl | C—Br |
| 370. | C—Cl | N | C—Cl | C—CH₃ |
| 371. | C—Cl | N | C—F | CH |
| 372. | C—Cl | N | C—F | C—Cl |
| 373. | C—Cl | N | C—F | C—F |
| 374. | C—Cl | N | C—F | C—Br |
| 375. | C—Cl | N | C—F | C—CH₃ |
| 376. | C—Cl | N | C—Br | CH |
| 377. | C—Cl | N | C—Br | C—Cl |
| 378. | C—Cl | N | C—Br | C—F |
| 379. | C—Cl | N | C—Br | C—Br |
| 380. | C—Cl | N | C—Br | C—CH₃ |
| 381. | C—Cl | N | C—CH₃ | CH |
| 382. | C—Cl | N | C—CH₃ | C—Cl |
| 383. | C—Cl | N | C—CH₃ | C—F |
| 384. | C—Cl | N | C—CH₃ | C—Br |
| 385. | C—Cl | N | C—CH₃ | C—CH₃ |
| 386. | C—Cl | CH | N | CH |
| 387. | C—Cl | CH | N | C—Cl |
| 388. | C—Cl | CH | N | C—F |
| 389. | C—Cl | CH | N | C—Br |
| 390. | C—Cl | CH | N | C—CH₃ |
| 391. | C—Cl | CH | CH | CH |
| 392. | C—Cl | CH | CH | C—Cl |
| 393. | C—Cl | CH | CH | C—F |
| 394. | C—Cl | CH | CH | C—Br |
| 395. | C—Cl | CH | CH | C—CH₃ |
| 396. | C—Cl | CH | C—Cl | CH |
| 397. | C—Cl | CH | C—Cl | C—Cl |
| 398. | C—Cl | CH | C—Cl | C—F |
| 399. | C—Cl | CH | C—Cl | C—Br |
| 400. | C—Cl | CH | C—Cl | C—CH₃ |
| 401. | C—Cl | CH | C—F | CH |
| 402. | C—Cl | CH | C—F | C—Cl |
| 403. | C—Cl | CH | C—F | C—F |
| 404. | C—Cl | CH | C—F | C—Br |
| 405. | C—Cl | CH | C—F | C—CH₃ |
| 406. | C—Cl | CH | C—Br | CH |
| 407. | C—Cl | CH | C—Br | C—Cl |
| 408. | C—Cl | CH | C—Br | C—F |
| 409. | C—Cl | CH | C—Br | C—Br |
| 410. | C—Cl | CH | C—Br | C—CH₃ |
| 411. | C—Cl | CH | C—CH₃ | CH |
| 412. | C—Cl | CH | C—CH₃ | C—Cl |
| 413. | C—Cl | CH | C—CH₃ | C—F |
| 414. | C—Cl | CH | C—CH₃ | C—Br |
| 415. | C—Cl | CH | C—CH₃ | C—CH₃ |
| 416. | C—Cl | C—Cl | N | CH |
| 417. | C—Cl | C—Cl | N | C—Cl |
| 418. | C—Cl | C—Cl | N | C—F |
| 419. | C—Cl | C—Cl | N | C—Br |
| 420. | C—Cl | C—Cl | N | C—CH₃ |
| 421. | C—Cl | C—Cl | CH | CH |
| 422. | C—Cl | C—Cl | CH | C—Cl |
| 423. | C—Cl | C—Cl | CH | C—F |
| 424. | C—Cl | C—Cl | CH | C—Br |
| 425. | C—Cl | C—Cl | CH | C—CH₃ |
| 426. | C—Cl | C—Cl | C—Cl | CH |
| 427. | C—Cl | C—Cl | C—Cl | C—Cl |
| 428. | C—Cl | C—Cl | C—Cl | C—F |
| 429. | C—Cl | C—Cl | C—Cl | C—Br |
| 430. | C—Cl | C—Cl | C—Cl | C—CH₃ |
| 431. | C—Cl | C—Cl | C—F | CH |
| 432. | C—Cl | C—Cl | C—F | C—Cl |
| 433. | C—Cl | C—Cl | C—F | C—F |
| 434. | C—Cl | C—Cl | C—F | C—Br |
| 435. | C—Cl | C—Cl | C—F | C—CH₃ |
| 436. | C—Cl | C—Cl | C—Br | CH |
| 437. | C—Cl | C—Cl | C—Br | C—Cl |
| 438. | C—Cl | C—Cl | C—Br | C—F |
| 439. | C—Cl | C—Cl | C—Br | C—Br |
| 440. | C—Cl | C—Cl | C—Br | C—CH₃ |
| 441. | C—Cl | C—Cl | C—CH₃ | CH |
| 442. | C—Cl | C—Cl | C—CH₃ | C—Cl |
| 443. | C—Cl | C—Cl | C—CH₃ | C—F |
| 444 | C—Cl | C—Cl | C—CH₃ | C—Br |
| 445. | C—Cl | C—Cl | C—CH₃ | C—CH₃ |
| 446. | C—Cl | C—F | N | CH |
| 447. | C—Cl | C—F | N | C—Cl |
| 448 | C—Cl | C—F | N | C—F |
| 449. | C—Cl | C—F | N | C—Br |
| 450 | C—Cl | C—F | N | C—CH₃ |
| 451. | C—Cl | C—F | CH | CH |
| 452. | C—Cl | C—F | CH | C—Cl |
| 453. | C—Cl | C—F | CH | C—F |
| 454 | C—Cl | C—F | CH | C—Br |
| 455. | C—Cl | C—F | CH | C—CH₃ |
| 456. | C—Cl | C—F | C—Cl | CH |
| 457. | C—Cl | C—F | C—Cl | C—Cl |
| 458. | C—Cl | C—F | C—Cl | C—F |
| 459. | C—Cl | C—F | C—Cl | C—Br |
| 460. | C—Cl | C—F | C—Cl | C—CH₃ |
| 461. | C—Cl | C—F | C—F | CH |
| 462. | C—Cl | C—F | C—F | C—Cl |
| 463. | C—Cl | C—F | C—F | C—F |
| 464. | C—Cl | C—F | C—F | C—Br |
| 465. | C—Cl | C—F | C—F | C—CH₃ |
| 466. | C—Cl | C—F | C—Br | CH |
| 467. | C—Cl | C—F | C—Br | C—Cl |
| 468. | C—Cl | C—F | C—Br | C—F |
| 469 | C—Cl | C—F | C—Br | C—Br |
| 470. | C—Cl | C—F | C—Br | C—CH₃ |
| 471. | C—Cl | C—F | C—CH₃ | CH |
| 472. | C—Cl | C—F | C—CH₃ | C—Cl |
| 473. | C—Cl | C—F | C—CH₃ | C—F |
| 474. | C—Cl | C—F | C—CH₃ | C—Br |
| 475. | C—Cl | C—F | C—CH₃ | C—CH₃ |
| 476. | C—Cl | C—Br | N | CH |
| 477. | C—Cl | C—Br | N | C—Cl |
| 478. | C—Cl | C—Br | N | C—F |
| 479. | C—Cl | C—Br | N | C—Br |
| 480. | C—Cl | C—Br | N | C—CH₃ |
| 481. | C—Cl | C—Br | CH | CH |
| 482. | C—Cl | C—Br | CH | C—Cl |
| 483. | C—Cl | C—Br | CH | C—F |
| 484. | C—Cl | C—Br | CH | C—Br |
| 485. | C—Cl | C—Br | CH | C—CH₃ |
| 486. | C—Cl | C—Br | C—Cl | CH |
| 487. | C—Cl | C—Br | C—Cl | C—Cl |
| 488. | C—Cl | C—Br | C—Cl | C—F |
| 489. | C—Cl | C—Br | C—Cl | C—Br |
| 490. | C—Cl | C—Br | C—Cl | C—CH₃ |
| 491. | C—Cl | C—Br | C—F | CH |
| 492. | C—Cl | C—Br | C—F | C—Cl |
| 493. | C—Cl | C—Br | C—F | C—F |
| 494. | C—Cl | C—Br | C—F | C—Br |
| 495. | C—Cl | C—Br | C—F | C—CH₃ |
| 496. | C—Cl | C—Br | C—Br | CH |
| 497. | C—Cl | C—Br | C—Br | C—Cl |
| 498. | C—Cl | C—Br | C—Br | C—F |
| 499. | C—Cl | C—Br | C—Br | C—Br |
| 500. | C—Cl | C—Br | C—Br | C—CH₃ |
| 501. | C—Cl | C—Br | C—CH₃ | CH |
| 502. | C—Cl | C—Br | C—CH₃ | C—Cl |
| 503. | C—Cl | C—Br | C—CH₃ | C—F |
| 504. | C—Cl | C—Br | C—CH₃ | C—Br |
| 505. | C—Cl | C—Br | C—CH₃ | C—CH₃ |
| 506. | C—Cl | C—CH₃ | N | CH |
| 507. | C—Cl | C—CH₃ | N | C—Cl |
| 508. | C—Cl | C—CH₃ | N | C—F |
| 509. | C—Cl | C—CH₃ | N | C—Br |
| 510. | C—Cl | C—CH₃ | N | C—CH₃ |
| 511. | C—Cl | C—CH₃ | CH | CH |
| 512. | C—Cl | C—CH₃ | CH | C—Cl |
| 513. | C—Cl | C—CH₃ | CH | C—F |
| 514. | C—Cl | C—CH₃ | CH | C—Br |
| 515. | C—Cl | C—CH₃ | CH | C—CH₃ |
| 516. | C—Cl | C—CH₃ | C—Cl | CH |
| 517. | C—Cl | C—CH₃ | C—Cl | C—Cl |
| 518. | C—Cl | C—CH₃ | C—Cl | C—F |
| 519. | C—Cl | C—CH₃ | C—Cl | C—Br |
| 520. | C—Cl | C—CH₃ | C—Cl | C—CH₃ |
| 521. | C—Cl | C—CH₃ | C—F | CH |
| 522. | C—Cl | C—CH₃ | C—F | C—Cl |
| 523. | C—Cl | C—CH₃ | C—F | C—F |
| 524. | C—Cl | C—CH₃ | C—F | C—Br |
| 525. | C—Cl | C—CH₃ | C—F | C—CH₃ |
| 526. | C—Cl | C—CH₃ | C—Br | CH |
| 527. | C—Cl | C—CH₃ | C—Br | C—Cl |
| 528. | C—Cl | C—CH₃ | C—Br | C—F |
| 529. | C—Cl | C—CH₃ | C—Br | C—Br |
| 530. | C—Cl | C—CH₃ | C—Br | C—CH₃ |
| 531. | C—Cl | C—CH₃ | C—CH₃ | CH |
| 532. | C—Cl | C—CH₃ | C—CH₃ | C—Cl |
| 533. | C—Cl | C—CH₃ | C—CH₃ | C—F |
| 534. | C—Cl | C—CH₃ | C—CH₃ | C—Br |
| 535. | C—Cl | C—CH₃ | C—CH₃ | C—CH₃ |
| 536. | C—F | N | N | CH |
| 537. | C—F | N | N | C—Cl |
| 538. | C—F | N | N | C—F |
| 539. | C—F | N | N | C—Br |
| 540. | C—F | N | N | C—CH₃ |
| 541. | C—F | N | CH | CH |
| 542. | C—F | N | CH | C—Cl |
| 543. | C—F | N | CH | C—F |
| 544. | C—F | N | CH | C—Br |
| 545. | C—F | N | CH | C—CH₃ |
| 546. | C—F | N | C—Cl | CH |
| 547. | C—F | N | C—Cl | C—Cl |
| 548. | C—F | N | C—Cl | C—F |
| 549. | C—F | N | C—Cl | C—Br |
| 550. | C—F | N | C—Cl | C—CH₃ |
| 551. | C—F | N | C—F | CH |
| 552. | C—F | N | C—F | C—Cl |
| 553. | C—F | N | C—F | C—F |
| 554. | C—F | N | C—F | C—Br |
| 555. | C—F | N | C—F | C—CH₃ |
| 556. | C—F | N | C—Br | CH |
| 557. | C—F | N | C—Br | C—Cl |
| 558. | C—F | N | C—Br | C—F |
| 559. | C—F | N | C—Br | C—Br |
| 560. | C—F | N | C—Br | C—CH₃ |
| 561. | C—F | N | C—CH₃ | CH |
| 562. | C—F | N | C—CH₃ | C—Cl |
| 563. | C—F | N | C—CH₃ | C—F |
| 564. | C—F | N | C—CH₃ | C—Br |
| 565. | C—F | N | C—CH₃ | C—CH₃ |
| 566. | C—F | CH | N | CH |
| 567. | C—F | CH | N | C—Cl |
| 568. | C—F | CH | N | C—F |
| 569. | C—F | CH | N | C—Br |
| 570. | C—F | CH | N | C—CH₃ |
| 571. | C—F | CH | CH | CH |
| 572. | C—F | CH | CH | C—Cl |
| 573. | C—F | CH | CH | C—F |
| 574. | C—F | CH | CH | C—Br |
| 575. | C—F | CH | CH | C—CH₃ |
| 576. | C—F | CH | C—Cl | CH |
| 577. | C—F | CH | C—Cl | C—Cl |
| 578. | C—F | CH | C—Cl | C—F |
| 579. | C—F | CH | C—Cl | C—Br |
| 580. | C—F | CH | C—Cl | C—CH₃ |
| 581. | C—F | CH | C—F | CH |
| 582. | C—F | CH | C—F | C—Cl |
| 583. | C—F | CH | C—F | C—F |
| 584. | C—F | CH | C—F | C—Br |
| 585. | C—F | CH | C—F | C—CH₃ |
| 586. | C—F | CH | C—Br | CH |
| 587. | C—F | CH | C—Br | C—Cl |
| 588. | C—F | CH | C—Br | C—F |
| 589. | C—F | CH | C—Br | C—Br |
| 590. | C—F | CH | C—Br | C—CH₃ |
| 591. | C—F | CH | C—CH₃ | CH |
| 592. | C—F | CH | C—CH₃ | C—Cl |
| 593. | C—F | CH | C—CH₃ | C—F |
| 594. | C—F | CH | C—CH₃ | C—Br |
| 595. | C—F | CH | C—CH₃ | C—CH₃ |
| 596. | C—F | C—Cl | N | CH |
| 597. | C—F | C—Cl | N | C—Cl |
| 598. | C—F | C—Cl | N | C—F |
| 599. | C—F | C—Cl | N | C—Br |
| 600. | C—F | C—Cl | N | C—CH₃ |
| 601. | C—F | C—Cl | CH | CH |
| 602. | C—F | C—Cl | CH | C—Cl |
| 603. | C—F | C—Cl | CH | C—F |
| 604. | C—F | C—Cl | CH | C—Br |
| 605. | C—F | C—Cl | CH | C—CH₃ |
| 606. | C—F | C—Cl | C—Cl | CH |
| 607. | C—F | C—Cl | C—Cl | C—Cl |
| 608. | C—F | C—Cl | C—Cl | C—F |
| 609. | C—F | C—Cl | C—Cl | C—Br |
| 610. | C—F | C—Cl | C—Cl | C—CH₃ |
| 611. | C—F | C—Cl | C—F | CH |
| 612. | C—F | C—Cl | C—F | C—Cl |
| 613. | C—F | C—Cl | C—F | C—F |
| 614. | C—F | C—Cl | C—F | C—Br |
| 615. | C—F | C—Cl | C—F | C—CH₃ |
| 616. | C—F | C—Cl | C—Br | CH |
| 617. | C—F | C—Cl | C—Br | C—Cl |
| 618. | C—F | C—Cl | C—Br | C—F |
| 619. | C—F | C—Cl | C—Br | C—Br |
| 620. | C—F | C—Cl | C—Br | C—CH₃ |
| 621. | C—F | C—Cl | C—CH₃ | CH |
| 622. | C—F | C—Cl | C—CH₃ | C—Cl |
| 623. | C—F | C—Cl | C—CH₃ | C—F |
| 624. | C—F | C—Cl | C—CH₃ | C—Br |
| 625. | C—F | C—Cl | C—CH₃ | C—CH₃ |
| 626. | C—F | C—F | N | CH |
| 627. | C—F | C—F | N | C—Cl |
| 628. | C—F | C—F | N | C—F |
| 629. | C—F | C—F | N | C—Br |
| 630. | C—F | C—F | N | C—CH₃ |
| 631. | C—F | C—F | CH | CH |
| 632. | C—F | C—F | CH | C—Cl |
| 633. | C—F | C—F | CH | C—F |
| 634. | C—F | C—F | CH | C—Br |
| 635. | C—F | C—F | CH | C—CH₃ |
| 636. | C—F | C—F | C—Cl | CH |
| 637. | C—F | C—F | C—Cl | C—Cl |
| 638. | C—F | C—F | C—Cl | C—F |
| 639. | C—F | C—F | C—Cl | C—Br |
| 640. | C—F | C—F | C—Cl | C—CH₃ |
| 641. | C—F | C—F | C—F | CH |
| 642. | C—F | C—F | C—F | C—Cl |
| 643. | C—F | C—F | C—F | C—F |
| 644. | C—F | C—F | C—F | C—Br |
| 645. | C—F | C—F | C—F | C—CH₃ |
| 646. | C—F | C—F | C—Br | CH |
| 647. | C—F | C—F | C—Br | C—Cl |
| 648. | C—F | C—F | C—Br | C—F |
| 649. | C—F | C—F | C—Br | C—Br |
| 650. | C—F | C—F | C—Br | C—CH₃ |
| 651. | C—F | C—F | C—CH₃ | CH |
| 652. | C—F | C—F | C—CH₃ | C—Cl |
| 653. | C—F | C—F | C—CH₃ | C—F |
| 654. | C—F | C—F | C—CH₃ | C—Br |
| 655. | C—F | C—F | C—CH₃ | C—CH₃ |
| 656. | C—F | C—Br | N | CH |
| 657. | C—F | C—Br | N | C—Cl |
| 658. | C—F | C—Br | N | C—F |
| 659. | C—F | C—Br | N | C—Br |
| 660. | C—F | C—Br | N | C—CH₃ |
| 661. | C—F | C—Br | CH | CH |
| 662. | C—F | C—Br | CH | C—Cl |
| 663. | C—F | C—Br | CH | C—F |
| 664. | C—F | C—Br | CH | C—Br |
| 665. | C—F | C—Br | CH | C—CH₃ |
| 666. | C—F | C—Br | C—Cl | CH |
| 667. | C—F | C—Br | C—Cl | C—Cl |
| 668. | C—F | C—Br | C—Cl | C—F |
| 669. | C—F | C—Br | C—Cl | C—Br |
| 670. | C—F | C—Br | C—Cl | C—CH₃ |
| 671. | C—F | C—Br | C—F | CH |
| 672. | C—F | C—Br | C—F | C—Cl |
| 673. | C—F | C—Br | C—F | C—F |
| 674. | C—F | C—Br | C—F | C—Br |
| 675. | C—F | C—Br | C—F | C—CH₃ |
| 676. | C—F | C—Br | C—Br | CH |
| 677. | C—F | C—Br | C—Br | C—Cl |
| 678. | C—F | C—Br | C—Br | C—F |
| 679. | C—F | C—Br | C—Br | C—Br |
| 680. | C—F | C—Br | C—Br | C—CH₃ |
| 681. | C—F | C—Br | C—CH₃ | CH |
| 682. | C—F | C—Br | C—CH₃ | C—Cl |
| 683. | C—F | C—Br | C—CH₃ | C—F |
| 684. | C—F | C—Br | C—CH₃ | C—Br |
| 685. | C—F | C—Br | C—CH₃ | C—CH₃ |
| 686. | C—F | C—CH₃ | N | CH |
| 687. | C—F | C—CH₃ | N | C—Cl |
| 688. | C—F | C—CH₃ | N | C—F |
| 689. | C—F | C—CH₃ | N | C—Br |
| 690. | C—F | C—CH₃ | N | C—CH₃ |
| 691. | C—F | C—CH₃ | CH | CH |
| 692. | C—F | C—CH₃ | CH | C—Cl |
| 693. | C—F | C—CH₃ | CH | C—F |
| 694. | C—F | C—CH₃ | CH | C—Br |
| 695. | C—F | C—CH₃ | CH | C—CH₃ |
| 696. | C—F | C—CH₃ | C—Cl | CH |
| 697. | C—F | C—CH₃ | C—Cl | C—Cl |
| 698. | C—F | C—CH₃ | C—Cl | C—F |
| 699. | C—F | C—CH₃ | C—Cl | C—Br |
| 700. | C—F | C—CH₃ | C—Cl | C—CH₃ |
| 701. | C—F | C—CH₃ | C—F | CH |
| 702. | C—F | C—CH₃ | C—F | C—Cl |
| 703. | C—F | C—CH₃ | C—F | C—F |
| 704. | C—F | C—CH₃ | C—F | C—Br |
| 705. | C—F | C—CH₃ | C—F | C—CH₃ |
| 706. | C—F | C—CH₃ | C—Br | CH |
| 707. | C—F | C—CH₃ | C—Br | C—Cl |
| 708. | C—F | C—CH₃ | C—Br | C—F |
| 709. | C—F | C—CH₃ | C—Br | C—Br |
| 710. | C—F | C—CH₃ | C—Br | C—CH₃ |
| 711. | C—F | C—CH₃ | C—CH₃ | CH |
| 712. | C—F | C—CH₃ | C—CH₃ | C—Cl |
| 713. | C—F | C—CH₃ | C—CH₃ | C—F |
| 714. | C—F | C—CH₃ | C—CH₃ | C—Br |
| 715. | C—F | C—CH₃ | C—CH₃ | C—CH₃ |
| 716. | C—Br | N | N | CH |
| 717. | C—Br | N | N | C—Cl |
| 718. | C—Br | N | N | C—F |
| 719. | C—Br | N | N | C—Br |
| 720. | C—Br | N | N | C—CH₃ |
| 721. | C—Br | N | CH | CH |
| 722. | C—Br | N | CH | C—Cl |
| 723. | C—Br | N | CH | C—F |
| 724. | C—Br | N | CH | C—Br |
| 725. | C—Br | N | CH | C—CH₃ |
| 726. | C—Br | N | C—Cl | CH |
| 727. | C—Br | N | C—Cl | C—Cl |
| 728. | C—Br | N | C—Cl | C—F |
| 729. | C—Br | N | C—Cl | C—Br |
| 730. | C—Br | N | C—Cl | C—CH₃ |
| 731. | C—Br | N | C—F | CH |
| 732. | C—Br | N | C—F | C—Cl |
| 733. | C—Br | N | C—F | C—F |
| 734. | C—Br | N | C—F | C—Br |
| 735. | C—Br | N | C—F | C—CH₃ |
| 736. | C—Br | N | C—Br | CH |
| 737. | C—Br | N | C—Br | C—Cl |
| 738. | C—Br | N | C—Br | C—F |
| 739. | C—Br | N | C—Br | C—Br |
| 740. | C—Br | N | C—Br | C—CH₃ |
| 741. | C—Br | N | C—CH₃ | CH |
| 742. | C—Br | N | C—CH₃ | C—Cl |
| 743. | C—Br | N | C—CH₃ | C—F |
| 744. | C—Br | N | C—CH₃ | C—Br |
| 745. | C—Br | N | C—CH₃ | C—CH₃ |
| 746. | C—Br | CH | N | CH |
| 747. | C—Br | CH | N | C—Cl |
| 748. | C—Br | CH | N | C—F |
| 749. | C—Br | CH | N | C—Br |
| 750. | C—Br | CH | N | C—CH₃ |
| 751. | C—Br | CH | CH | CH |
| 752. | C—Br | CH | CH | C—Cl |
| 753. | C—Br | CH | CH | C—F |
| 754. | C—Br | CH | CH | C—Br |
| 755. | C—Br | CH | CH | C—CH₃ |
| 756. | C—Br | CH | C—Cl | CH |
| 757. | C—Br | CH | C—Cl | C—Cl |
| 758. | C—Br | CH | C—Cl | C—F |
| 759. | C—Br | CH | C—Cl | C—Br |
| 760. | C—Br | CH | C—Cl | C—CH₃ |
| 761. | C—Br | CH | C—F | CH |
| 762. | C—Br | CH | C—F | C—Cl |
| 763. | C—Br | CH | C—F | C—F |
| 764. | C—Br | CH | C—F | C—Br |
| 765. | C—Br | CH | C—F | C—CH₃ |
| 766. | C—Br | CH | C—Br | CH |
| 767. | C—Br | CH | C—Br | C—Cl |
| 768. | C—Br | CH | C—Br | C—F |
| 769. | C—Br | CH | C—Br | C—Br |
| 770. | C—Br | CH | C—Br | C—CH₃ |
| 771. | C—Br | CH | C—CH₃ | CH |
| 772. | C—Br | CH | C—CH₃ | C—Cl |
| 773. | C—Br | CH | C—CH₃ | C—F |
| 774. | C—Br | CH | C—CH₃ | C—Br |
| 775. | C—Br | CH | C—CH₃ | C—CH₃ |
| 776. | C—Br | C—Cl | N | CH |
| 777. | C—Br | C—Cl | N | C—Cl |
| 778. | C—Br | C—Cl | N | C—F |
| 779. | C—Br | C—Cl | N | C—Br |
| 780. | C—Br | C—Cl | N | C—CH₃ |
| 781. | C—Br | C—Cl | CH | CH |
| 782. | C—Br | C—Cl | CH | C—Cl |
| 783. | C—Br | C—Cl | CH | C—F |
| 784. | C—Br | C—Cl | CH | C—Br |
| 785. | C—Br | C—Cl | CH | C—CH₃ |
| 786. | C—Br | C—Cl | C—Cl | CH |
| 787. | C—Br | C—Cl | C—Cl | C—Cl |
| 788. | C—Br | C—Cl | C—Cl | C—F |
| 789. | C—Br | C—Cl | C—Cl | C—Br |
| 790. | C—Br | C—Cl | C—Cl | C—CH₃ |
| 791. | C—Br | C—Cl | C—F | CH |
| 792. | C—Br | C—Cl | C—F | C—Cl |
| 793. | C—Br | C—Cl | C—F | C—F |
| 794. | C—Br | C—Cl | C—F | C—Br |
| 795. | C—Br | C—Cl | C—F | C—CH₃ |
| 796. | C—Br | C—Cl | C—Br | CH |
| 797. | C—Br | C—Cl | C—Br | C—Cl |
| 798. | C—Br | C—Cl | C—Br | C—F |
| 799. | C—Br | C—Cl | C—Br | C—Br |
| 800. | C—Br | C—Cl | C—Br | C—CH₃ |
| 801. | C—Br | C—Cl | C—CH₃ | CH |
| 802. | C—Br | C—Cl | C—CH₃ | C—Cl |
| 803. | C—Br | C—Cl | C—CH₃ | C—F |
| 804. | C—Br | C—Cl | C—CH₃ | C—Br |
| 805. | C—Br | C—Cl | C—CH₃ | C—CH₃ |
| 806. | C—Br | C—F | N | CH |
| 807. | C—Br | C—F | N | C—Cl |
| 808. | C—Br | C—F | N | C—F |
| 809. | C—Br | C—F | N | C—Br |
| 810. | C—Br | C—F | N | C—CH₃ |
| 811. | C—Br | C—F | CH | CH |
| 812. | C—Br | C—F | CH | C—Cl |
| 813. | C—Br | C—F | CH | C—F |
| 814. | C—Br | C—F | CH | C—Br |
| 815. | C—Br | C—F | CH | C—CH₃ |
| 816. | C—Br | C—F | C—Cl | CH |
| 817. | C—Br | C—F | C—Cl | C—Cl |
| 818. | C—Br | C—F | C—Cl | C—F |
| 819. | C—Br | C—F | C—Cl | C—Br |
| 820. | C—Br | C—F | C—Cl | C—CH₃ |
| 821. | C—Br | C—F | C—F | CH |
| 822. | C—Br | C—F | C—F | C—Cl |
| 823. | C—Br | C—F | C—F | C—F |
| 824. | C—Br | C—F | C—F | C—Br |
| 825. | C—Br | C—F | C—F | C—CH₃ |
| 826. | C—Br | C—F | C—Br | CH |
| 827. | C—Br | C—F | C—Br | C—Cl |
| 828. | C—Br | C—F | C—Br | C—F |
| 829. | C—Br | C—F | C—Br | C—Br |
| 830. | C—Br | C—F | C—Br | C—CH₃ |
| 831. | C—Br | C—F | C—CH₃ | CH |
| 832. | C—Br | C—F | C—CH₃ | C—Cl |
| 833. | C—Br | C—F | C—CH₃ | C—F |
| 834. | C—Br | C—F | C—CH₃ | C—Br |
| 835. | C—Br | C—F | C—CH₃ | C—CH₃ |
| 836. | C—Br | C—Br | N | CP |
| 837. | C—Br | C—Br | N | C—Cl |
| 838. | C—Br | C—Br | N | C—F |
| 839. | C—Br | C—Br | N | C—Br |
| 840. | C—Br | C—Br | N | C—CH₃ |
| 841. | C—Br | C—Br | CH | CH |
| 842. | C—Br | C—Br | CH | C—Cl |
| 843. | C—Br | C—Br | CH | C—F |
| 844. | C—Br | C—Br | CH | C—Br |
| 845. | C—Br | C—Br | CH | C—CH₃ |
| 846. | C—Br | C—Br | C—Cl | CH |
| 847. | C—Br | C—Br | C—Cl | C—Cl |
| 848. | C—Br | C—Br | C—Cl | C—F |
| 849. | C—Br | C—Br | C—Cl | C—Br |
| 850. | C—Br | C—Br | C—Cl | C—CH₃ |
| 851. | C—Br | C—Br | C—F | CH |
| 852. | C—Br | C—Br | C—F | C—Cl |
| 853. | C—Br | C—Br | C—F | C—F |
| 854. | C—Br | C—Br | C—F | C—Br |
| 855. | C—Br | C—Br | C—F | C—CH₃ |
| 856. | C—Br | C—Br | C—Br | CH |
| 857. | C—Br | C—Br | C—Br | C—Cl |
| 858. | C—Br | C—Br | C—Br | C—F |
| 859. | C—Br | C—Br | C—Br | C—Br |
| 860. | C—Br | C—Br | C—Br | C—CH₃ |
| 861. | C—Br | C—Br | C—CH₃ | CH |
| 862. | C—Br | C—Br | C—CH₃ | C—Cl |
| 863. | C—Br | C—Br | C—CH₃ | C—F |
| 864. | C—Br | C—Br | C—CH₃ | C—Br |
| 865. | C—Br | C—Br | C—CH₃ | C—CH₃ |
| 866. | C—Br | C—CH₃ | N | CH |
| 867. | C—Br | C—CH₃ | N | C—Cl |
| 868. | C—Br | C—CH₃ | N | C—F |
| 869. | C—Br | C—CH₃ | N | C—Br |
| 870. | C—Br | C—CH₃ | N | C—CH₃ |
| 871. | C—Br | C—CH₃ | CH | CH |
| 872. | C—Br | C—CH₃ | CH | C—Cl |
| 873. | C—Br | C—CH₃ | CH | C—F |
| 874. | C—Br | C—CH₃ | CH | C—Br |
| 875. | C—Br | C—CH₃ | CH | C—CH₃ |
| 876. | C—Br | C—CH₃ | C—Cl | CH |
| 877. | C—Br | C—CH₃ | C—Cl | C—Cl |
| 878. | C—Br | C—CH₃ | C—Cl | C—F |
| 879. | C—Br | C—CH₃ | C—Cl | C—Br |
| 880. | C—Br | C—CH₃ | C—Cl | C—CH₃ |
| 881. | C—Br | C—CH₃ | C—F | CH |
| 882. | C—Br | C—CH₃ | C—F | C—Cl |
| 883. | C—Br | C—CH₃ | C—F | C—F |
| 884. | C—Br | C—CH₃ | C—F | C—Br |
| 885. | C—Br | C—CH₃ | C—F | C—CH₃ |
| 886. | C—Br | C—CH₃ | C—Br | CH |
| 887. | C—Br | C—CH₃ | C—Br | C—Cl |
| 888. | C—Br | C—CH₃ | C—Br | C—F |
| 889. | C—Br | C—CH₃ | C—Br | C—Br |
| 890. | C—Br | C—CH₃ | C—Br | C—CH₃ |
| 891. | C—Br | C—CH₃ | C—CH₃ | CH |
| 892. | C—Br | C—CH₃ | C—CH₃ | C—Cl |
| 893. | C—Br | C—CH₃ | C—CH₃ | C—F |
| 894. | C—Br | C—CH₃ | C—CH₃ | C—Br |
| 895. | C—Br | C—CH₃ | C—CH₃ | C—CH₃ |
| 896. | C—CH₃ | N | N | CH |
| 897. | C—CH₃ | N | N | C—Cl |
| 898. | C—CH₃ | N | N | C—F |
| 899. | C—CH₃ | N | N | C—Br |
| 900. | C—CH₃ | N | N | C—CH₃ |
| 901. | C—CH₃ | N | CH | CH |
| 902. | C—CH₃ | N | CH | C—Cl |
| 903. | C—CH₃ | N | CH | C—F |
| 904. | C—CH₃ | N | CH | C—Br |
| 905. | C—CH₃ | N | CH | C—CH₃ |
| 906. | C—CH₃ | N | C—Cl | CH |
| 907. | C—CH₃ | N | C—Cl | C—Cl |
| 908. | C—CH₃ | N | C—Cl | C—F |
| 909. | C—CH₃ | N | C—Cl | C—Br |
| 910. | C—CH₃ | N | C—Cl | C—CH₃ |
| 911. | C—CH₃ | N | C—F | CH |
| 912. | C—CH₃ | N | C—F | C—Cl |
| 913. | C—CH₃ | N | C—F | C—F |
| 914. | C—CH₃ | N | C—F | C—Br |
| 915. | C—CH₃ | N | C—F | C—CH₃ |
| 916. | C—CH₃ | N | C—Br | CH |
| 917. | C—CH₃ | N | C—Br | C—Cl |
| 918. | C—CH₃ | N | C—Br | C—F |
| 919. | C—CH₃ | N | C—Br | C—Br |
| 920. | C—CH₃ | N | C—Br | C—CH₃ |
| 921. | C—CH₃ | N | C—CH₃ | CH |
| 922. | C—CH₃ | N | C—CH₃ | C—Cl |
| 923. | C—CH₃ | N | C—CH₃ | C—F |
| 924. | C—CH₃ | N | C—CH₃ | C—Br |
| 925. | C—CH₃ | N | C—CH₃ | C—CH₃ |
| 926. | C—CH₃ | CH | N | CH |
| 927. | C—CH₃ | CH | N | C—Cl |
| 928. | C—CH₃ | CH | N | C—F |
| 929. | C—CH₃ | CH | N | C—Br |
| 930. | C—CH₃ | CH | N | C—CH₃ |
| 931. | C—CH₃ | CH | CH | CH |
| 932. | C—CH₃ | CH | CH | C—Cl |
| 933. | C—CH₃ | CH | CH | C—F |
| 934. | C—CH₃ | CH | CH | C—Br |
| 935. | C—CH₃ | CH | CH | C—CH₃ |
| 936. | C—CH₃ | CH | C—Cl | CH |
| 937. | C—CH₃ | CH | C—Cl | C—Cl |
| 938. | C—CH₃ | CH | C—Cl | C—F |
| 939. | C—CH₃ | CH | C—Cl | C—Br |
| 940. | C—CH₃ | CH | C—Cl | C—CH₃ |
| 941. | C—CH₃ | CH | C—F | CH |
| 942. | C—CH₃ | CH | C—F | C—Cl |
| 943. | C—CH₃ | CH | C—F | C—F |
| 944. | C—CH₃ | CH | C—F | C—Br |
| 945. | C—CH₃ | CH | C—F | C—CH₃ |
| 946. | C—CH₃ | CH | C—Br | CH |
| 947. | C—CH₃ | CH | C—Br | C—Cl |
| 948. | C—CH₃ | CH | C—Br | C—F |
| 949. | C—CH₃ | CH | C—Br | C—Br |
| 950. | C—CH₃ | CH | C—Br | C—CH₃ |
| 951. | C—CH₃ | CH | C—CH₃ | CH |
| 952. | C—CH₃ | CH | C—CH₃ | C—Cl |
| 953. | C—CH₃ | CH | C—CH₃ | C—F |
| 954. | C—CH₃ | CH | C—CH₃ | C—Br |
| 955. | C—CH₃ | CH | C—CH₃ | C—CH₃ |
| 956. | C—CH₃ | C—Cl | N | CH |
| 957. | C—CH₃ | C—Cl | N | C—Cl |
| 958. | C—CH₃ | C—Cl | N | C—F |
| 959. | C—CH₃ | C—Cl | N | C—Br |
| 960. | C—CH₃ | C—Cl | N | C—CH₃ |
| 961. | C—CH₃ | C—Cl | CH | CH |
| 962. | C—CH₃ | C—Cl | CH | C—Cl |
| 963. | C—CH₃ | C—Cl | CH | C—F |
| 964. | C—CH₃ | C—Cl | CH | C—Br |
| 965. | C—CH₃ | C—Cl | CH | C—CH₃ |
| 966. | C—CH₃ | C—Cl | C—Cl | CH |
| 967. | C—CH₃ | C—Cl | C—Cl | C—Cl |
| 968. | C—CH₃ | C—Cl | C—Cl | C—F |
| 969. | C—CH₃ | C—Cl | C—Cl | C—Br |
| 970. | C—CH₃ | C—Cl | C—Cl | C—CH₃ |
| 971. | C—CH₃ | C—Cl | C—F | CH |
| 972. | C—CH₃ | C—Cl | C—F | C—Cl |
| 973. | C—CH₃ | C—Cl | C—F | C—F |
| 974. | C—CH₃ | C—Cl | C—F | C—Br |
| 975. | C—CH₃ | C—Cl | C—F | C—CH₃ |
| 976. | C—CH₃ | C—Cl | C—Br | CH |
| 977. | C—CH₃ | C—Cl | C—Br | C—Cl |
| 978. | C—CH₃ | C—Cl | C—Br | C—F |
| 979. | C—CH₃ | C—Cl | C—Br | C—Br |
| 980. | C—CH₃ | C—Cl | C—Br | C—CH₃ |
| 981. | C—CH₃ | C—Cl | C—CH₃ | CH |
| 982. | C—CH₃ | C—Cl | C—CH₃ | C—Cl |
| 983. | C—CH₃ | C—Cl | C—CH₃ | C—F |
| 984. | C—CH₃ | C—Cl | C—CH₃ | C—Br |
| 985. | C—CH₃ | C—Cl | C—CH₃ | C—CH₃ |
| 986. | C—CH₃ | C—F | N | CH |
| 987. | C—CH₃ | C—F | N | C—Cl |
| 988. | C—CH₃ | C—F | N | C—F |
| 989. | C—CH₃ | C—F | N | C—Br |
| 990. | C—CH₃ | C—F | N | C—CH₃ |
| 991. | C—CH₃ | C—F | CH | CH |
| 992. | C—CH₃ | C—F | CH | C—Cl |
| 993. | C—CH₃ | C—F | CH | C—F |
| 994. | C—CH₃ | C—F | CH | C—Br |
| 995. | C—CH₃ | C—F | CH | C—CH₃ |
| 996. | C—CH₃ | C—F | C—Cl | CH |
| 997. | C—CH₃ | C—F | C—Cl | C—Cl |
| 998. | C—CH₃ | C—F | C—Cl | C—F |
| 999. | C—CH₃ | C—F | C—Cl | C—Br |
| 1000. | C—CH₃ | C—F | C—Cl | C—CH₃ |
| 1001. | C—CH₃ | C—F | C—F | CH |
| 1002. | C—CH₃ | C—F | C—F | C—Cl |
| 1003. | C—CH₃ | C—F | C—F | C—F |
| 1004. | C—CH₃ | C—F | C—F | C—Br |
| 1005. | C—CH₃ | C—F | C—F | C—CH₃ |
| 1006. | C—CH₃ | C—F | C—Br | CH |
| 1007. | C—CH₃ | C—F | C—Br | C—Cl |
| 1008. | C—CH₃ | C—F | C—Br | C—F |
| 1009. | C—CH₃ | C—F | C—Br | C—Br |
| 1010. | C—CH₃ | C—F | C—Br | C—CH₃ |
| 1011. | C—CH₃ | C—F | C—CH₃ | CH |
| 1012. | C—CH₃ | C—F | C—CH₃ | C—Cl |
| 1013. | C—CH₃ | C—F | C—CH₃ | C—F |
| 1014. | C—CH₃ | C—F | C—CH₃ | C—Br |
| 1015. | C—CH₃ | C—F | C—CH₃ | C—CH₃ |
| 1016. | C—CH₃ | C—Br | N | CH |
| 1017. | C—CH₃ | C—Br | N | C—Cl |
| 1018. | C—CH₃ | C—Br | N | C—F |
| 1055. | C—CH₃ | C—CH₃ | CH | C—CH₃ |
| 1056. | C—CH₃ | C—CH₃ | C—Cl | CH |
| 1057. | C—CH₃ | C—CH₃ | C—Cl | C—Cl |
| 1058. | C—CH₃ | C—CH₃ | C—Cl | C—F |
| 1059. | C—CH₃ | C—CH₃ | C—Cl | C—Br |
| 1060. | C—CH₃ | C—CH₃ | C—Cl | C—CH₃ |
| 1061. | C—CH₃ | C—CH₃ | C—F | CH |
| 1062. | C—CH₃ | C—CH₃ | C—F | C—Cl |
| 1063. | C—CH₃ | C—CH₃ | C—F | C—F |
| 1064. | C—CH₃ | C—CH₃ | C—F | C—Br |
| 1065. | C—CH₃ | C—CH₃ | C—F | C—CH₃ |
| 1066. | C—CH₃ | C—CH₃ | C—Br | CH |
| 1067. | C—CH₃ | C—CH₃ | C—Br | C—Cl |
| 1068. | C—CH₃ | C—CH₃ | C—Br | C—F |
| 1069. | C—CH₃ | C—CH₃ | C—Br | C—Br |
| 1070. | C—CH₃ | C—CH₃ | C—Br | C—CH₃ |
| 1071. | C—CH₃ | C—CH₃ | C—CH₃ | CH |
| 1072. | C—CH₃ | C—CH₃ | C—CH₃ | C—Cl |
| 1073. | C—CH₃ | C—CH₃ | C—CH₃ | C—F |
| 1074. | C—CH₃ | C—CH₃ | C—CH₃ | C—Br |
| 1075. | C—CH₃ | C—CH₃ | C—CH₃ | C—CH₃ |

Table 361 to Table 600: Includes all compounds disclosed in Table 1 to Table 240 respectively wherein compound of formula I.3 is replaced by compound of formula I.4;
Table 601 to Table 720: Includes all compounds disclosed in Table 241 to Table 360 respectively wherein compound of formula I.7 is replaced by compound of formula I.8;
The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emusifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agro-chemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. There-fore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aero-sol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

### Pests

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

### Animal Health

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I.

In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### EXAMPLES

With appropriate modification of the starting materials, the procedures as described in the preparation examples below were used to obtain further compounds of formula 1. The compounds obtained in this manner are listed in the table C that follows, together with physical data.

Compounds can be characterized e.g., by coupled High Performance Liquid Chromatography/mass spectrometry (HPLC/MS), by ¹H NMR and/or by their melting points.

Compounds can be characterized e.g., by coupled High Performance Liquid Chromatography/mass spectrometry (HPLC/MS), by ¹H NMR and/or by their melting points.

Analytical HPLC—Method 1: Agilent Eclipse Plus C18, 50×4.6 mm, ID 5 μm; Elution: A=10 mM Amm. Formate (0.1% Formic Acid), B=acetonitrile (0.1% Formic Acid), Flow=1.2 ml/min. at 30° C.; Gradient: 10% B to 100% B—3 min, hold for 1 min, 1 min—10% B. Run Time=5.01 min.

Analytical HPLC—Method 2: Kinetex XB C18 1,7μ 50×2.1 mm; A=water+0.1% TFA, B=acetonitrile, Flow=0.8 ml/min—1.0 ml/min in 1.5 min. at 60° C.; Gradient: 5% B to 100% B—1.5 min.

Analytical HPLC—Method 3: Diamonsil plus 5 μm, 30×3.0 mm; A=Water+0.05% TFA, B=acetonitrile, Flow=2 ml/min at 40° C.; Gradient: 5% B to 95% B—0.8 min, hold for 0.75 min, then 95% B to 5% B—0.25 min.

¹H NMR: The signals are characterized by chemical shift (ppm, d [delta]) vs. tetramethyl silane respectively, CDCl₃ for ¹³C NMR, by their multiplicity and by their integral (relative number of hydrogen atoms given). The following abbreviations are used to characterize the multiplicity of the signals: m=multiplet, q=quartet, t=triplet, d=doublet and s=singlet.

Abbreviations used are: d for day(s), h for hour(s), min for minute(s), RT/room temperature for 20-25° C., Rt for retention time; DMSO for dimethyl sulfoxide, OAc for acetate, EtOAc for ethyl acetate, IPA for isopropyl alcohol, MeOH for methanol, EtOH for ethanol, THF for tetrahydrofuran, DCM for dichloromethane, DMF for N,N-dimethylformamide and t-BuOH for tert-butanol.

### Example C-1

### Preparation of N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-4-(trifluoromethyl)benzamide (C-1)

### Step 1: Synthesis of 3-(4-bromophenyl)-2-methyl-3-oxo-propanenitrile

To a stirred solution of ethyl 4-bromobenzoate (30 g) in THF (600 mL) was added potassium tert-butoxide (29.39 g) at ambient temperature. After 10 min propionitrile (7.213 g) was added to a reaction mass at ambient temperature. The reaction mixture was stirred at ambient temperature for 1 h and after completion of the reaction, the mixture was diluted in water. The mixture was extracted with EtOAc and the organic extracts were dried over anhydrous sodium sulphate and evaporated in vacuo to obtain the title compound as a yellowish oil (24 g).

HPLC/MS (method 1): Rt: 1.821 min; m/z=236 (M−1)⁺.

### Step 2: Synthesis of 5-(4-bromophenyl)-2,4-dimethyl-pyrazol-3-amine

To a stirred solution of 3-(4-bromophenyl)-2-methyl-3-oxo-propanenitrile (25 g) in EtOH (250 mL) was added methylhydrazine (26.181 g, 85% solution) at ambient temperature. The reaction mixture was heated at 85° C. for 16 h and after completion of the reaction, EtOH was removed under reduced pressure, crude was dissolved in water. The mixture was extracted with EtOAc and the organic extracts dried over anhydrous sodium sulphate and evaporated in vacuo to obtain the title compound as a solid (23 g).

HPLC/MS (method 1): Rt: 1.656 min; m/z=266 (M+1)⁺.

### Step 3: Synthesis of N-[5-(4-bromophenyl)-2,4-dimethyl-pyrazol-3-yl]-4-(trifluoromethyl) benzamide

To a stirred solution of 5-(4-bromophenyl)-2,4-dimethyl-pyrazol-3-amine (43.5 g) in DCM (430 mL) were added 4-(trifluoromethyl)benzoic acid (31.075 g), N,N,N-diisopropyl ethylamine (118.29 g) and propylphosphonic anhydride (312.03 g, 50% solution in EtOAc) at ambient temperature. The reaction mixture was stirred at ambient temperature for 16 h. The reaction mixture was dissolved in water and the mixture was extracted with DCM. The organic extracts were washed with 1 N hydrochloric acid, dried over anhydrous sodium sulphate and evaporated in vacuo to obtain the title compound as a solid (44 g).

HPLC/MS (method 1): Rt: 2.066 min; m/z=438 (M+1)⁺.

### Step 4: Synthesis of tert-butyl N-[4-[1,4-dimethyl-5-[[4-(trifluoromethyl)benzoyl]amino]pyrazol-3-yl]phenyl]carbamate

To a stirred solution of N-[5-(4-bromophenyl)-2,4-dimethyl-pyrazol-3-yl]-4-(trifluoromethyl) benzamide (2.5 g) in 1,4-dioxane (30 mL) were added tert-butyl carbamate (0.802 g), Palladium(II) acetate (0.128 g), 2-dicyclohexylphosphino-2′,4′,6′-triisopropylbiphenyl (0.544 g) and cesium carbonate (3.717 g) at ambient temperature under innert atmosphere. The reaction mixture was heated at 100° C. for 16 h and after completion of the reaction, the mixture was dissolved in water. The mixture was extracted with EtOAc, the organic extracts were dried over anhydrous sodium sulphate and evaporated in vacuo. The residue obtained was subjected to silica gel flash column chromatography, eluting with a gradient of EtOAc and heptane to obtain the title compound as a solid (2.2 g).

HPLC/MS (method 1): Rt: 1.918 min; m/z=475 (M+1)⁺.

### Step 5: Synthesis of N-[5-(4-aminophenyl)-2,4-dimethyl-pyrazol-3-yl]-4-(trifluoromethyl) benzamide

To a stirred solution of tert-butyl N-[4-[1,4-dimethyl-5-[[4-(trifluoromethyl)benzoyl]amino]pyrazol-3-yl]phenyl]carbamate (3.4 g) in DCM (50 mL) was added trifluoro acetic acid (5 mL) at ambient temperature. The reaction mixture was heated at 50° C. for 16 h and after completion of the reaction, the mixture was neutralised with aqueous sodium bicarbonate solution. The mixture was extracted with EtOAc. The organic extracts were dried over anhydrous sodium sulphate and evaporated in vacuo. The residue obtained was subjected to silica gel flash column chromatography, eluting with a gradient of EtOAc and heptane to obtain the title compound as a solid (1.2 g).

HPLC/MS (method 1): Rt: 1.727 min; m/z=375 (M+1)⁺.

### Step 6: Synthesis of (4-nitrophenyl)N-[4-[1,4-dimethyl-5-[[4-(trifluoromethyl)benzoyl]amino]pyrazol-3-yl]phenyl]carbamate

To a stirred solution N-[5-(4-aminophenyl)-2,4-dimethyl-pyrazol-3-yl]-4-(trifluoromethyl) benzamide (0.2 g) in THF (4 mL) was added (4-nitrophenyl) carbonochloridate (0.108 g) at 0° C. under innert atmosphere. The reaction mixture was stirred at ambient temperature for 3 h and after completion of the reaction, the solvent from reaction mixture was evaporated in vacuo to obtain the title compound as a solid (0.2 g).

HPLC/MS (method 1): Rt: 1.885 min; m/z=540 (M+1)⁺.

### Step 7: Synthesis of N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-4-(trifluoromethyl)benzamide (C-1)

To a stirred solution of (4-nitrophenyl)N-[4-[1,4-dimethyl-5-[[4-(trifluoromethyl)benzoyl]amino]pyrazol-3-yl]phenyl]carbamate (0.2 g) in Acetonitrile (4 mL) were added 2-imino-3-(2-isopropyl-5-methyl-phenyl)thiazolidin-4-one (E1, 0.092 g), N,N,N-diisopropyl ethylamine (0.072 g) and potassium phosphate tribasic (0.118 g) at ambient temperature. The reaction mixture was stirred at ambient temperature for 19 h. After completion of the reaction, the mixture was diluted with water and extracted with EtOAc. The organic extracts were dried over anhydrous sodium sulphate and evaporated in vacuo. The residue obtained was subjected to preparative HPLC purification, eluting with a gradient of acetonitrile and water to obtain the title compound as a solid (0.17 g).

HPLC/MS (method 1): Rt: 1.960 min; m/z=649 (M+1)⁺.

¹H NMR (300 MHz, DMSO-d₆) δ 10.47 (s, 1H), 9.86 (s, 1H), 8.23 (d, J=8.1 Hz, 2H), 7.97 (d, J=8.2 Hz, 2H), 7.73 (d, J=8.6 Hz, 2H), 7.57 (d, J=8.6 Hz, 2H), 7.40 (d, J=8.0 Hz, 1H), 7.32-7.23 (m, 1H), 7.11-7.04 (m, 1H), 4.39-4.08 (m, 2H), 3.69 (s, 3H), 2.67 (h, J=6.8 Hz, 1H), 2.32 (s, 3H), 2.05 (s, 3H), 1.18 (d, J=6.8 Hz, 3H), 1.10 (d, J=6.8 Hz, 3H).

Synthesis of 2-imino-3-(2-isopropyl-5-methyl-phenyl)thiazolidin-4-one (E1)

### Step 1: Synthesis of 2-chloro-N-(2-isopropyl-5-methyl-phenyl)acetamide

To a stirred solution of 2-isopropyl-5-methyl-aniline (1.5 g) in THF (100 mL) was added triethyl amine (1.221 g) at ambient temperature and reaction mixture was cooled to 10° C. 2-chloroacetyl chloride was added to reaction mass drop wise over 30 min and reaction mixture was stirred at ambient temperature for 16 h. After completion of the reaction, the mixture was diluted in aqueous sodium bicarbonate solution and reaction mixture was extracted with EtOAc. The organic extracts were washed with brine solution, dried over anhydrous sodium sulphate and evaporated in vacuo to obtain the title compound as a yellowish oil (1.8 g). HPLC/MS (method 1): Rt: 1.813 min; m/z=223.70 (M−1)⁺.

### Step 2: Synthesis of 2-imino-3-(2-isopropyl-5-methyl-phenyl)thiazolidin-4-one (E1)

To a stirred solution of 2-chloro-N-(2-isopropyl-5-methyl-phenyl)acetamide (0.5 g) in acetone (10 mL) was added potassium thiocyanate (0.431 g) at ambient temperature and then reaction mixture was refluxed for 4 h. After completion of the reaction, the mixture was diluted in aqueous sodium bicarbonate solution and extracted with EtOAc. The organic extracts were washed with brine solution, dried over anhydrous sodium sulphate and evaporated in vacuo to obtain the title compound as a solid (0.42 g).

HPLC/MS (method 1): Rt: 1.675 min; m/z=249 (M+1)⁺.

### Example C-2

### Preparation of [(Z)-N′-[[4-[1,4-dimethyl-5-[[4-(trifluoromethyl)benzoyl]amino]pyrazol-3-yl]phenyl]carbamoyl]-N-(2-isopropyl-5-methyl-phenyl)carbamimidoyl]sulfanylmethyl acetate (C-2)

To a stirred solution of (4-nitrophenyl)N-[4-[1,4-dimethyl-5-[[4-(trifluoromethyl)benzoyl]amino]pyrazol-3-yl]phenyl]carbamate (0.45 g) in THF (9 mL) were added [N-(2-isopropyl-5-methyl-phenyl)carbamimidoyl]sulfanylmethyl acetate hydrobromide (E1′, 0.234 g) and N,N,N-diisopropyl ethylamine (0.194 g) at ambient temperature. The reaction mixture was stirred at ambient temperature for 24 h. After completion of the reaction, the mixture was diluted with water and extracted with EtOAc. The organic extracts were dried over anhydrous sodium sulphate and evaporated in vacuo. The residue obtained was subjected to preparative HPLC purification, eluting with a gradient of acetonitrile and water to obtain the title compound as a solid (0.18 g).

HPLC/MS (method 1): Rt: 2.205 min; m/z=681 (M+1)⁺.

¹H NMR (500 MHz, DMSO-d₆) δ 11.27 (s, 1H), 10.46 (s, 1H), 9.72 (s, 1H), 8.23 (dd, J=8.3, 4.3 Hz, 2H), 7.97 (dd, J=8.8, 2.6 Hz, 2H), 7.73 (d, J=8.3 Hz, 1H), 7.62 (d, J=8.2 Hz, 2H), 7.45 (s, 1H), 7.29 (d, J=8.0 Hz, 1H), 7.26-7.06 (m, 1H), 7.04 (d, J=1.8 Hz, 1H), 5.75 (d, J=9.0 Hz, 1H), 5.60 (s, 1H), 3.70 (d, J=4.6 Hz, 3H), 3.03 (p, J=6.8 Hz, 1H), 2.29 (d, J=6.3 Hz, 3H), 2.06 (d, J=11.8 Hz, 6H), 1.16 (dd, J=10.8, 6.8 Hz, 6H).

### Synthesis of [N-(2-isopropyl-5-methyl-phenyl)carbamimidoyl]sulfanylmethyl acetate hydrobromide (E1′)

### Step 1: N-[(2-isopropyl-5-methyl-phenyl)carbamothioyl]benzamide

To a stirred solution of 2-isopropyl-5-methyl-aniline (0.4 g) in acetone (5 mL) was added benzoyl isothiocyanate (0.481 g) at 0° C. under innert atmosphere. The reaction mixture was stirred at ambient temperature for 6 h and after completion of the reaction, the solvent from reaction mixture was evaporated in vacuo to obtain the title compound as a solid (0.8 g).

HPLC/MS (method 1): Rt: 2.153 min; m/z=313 (M+1)⁺.

### Step 2: [(1 E)-1-isobutyl-3-methyl-buta-1,3-dienyl]thiourea

A stirred solution of N-[(2-isopropyl-5-methyl-phenyl)carbamothioyl]benzamide (0.8 g) in 2 N sodium hydroxide (10 mL) was heated at 90° C. for 2 h. After completion of the reaction, the mixture was diluted with water and extracted with EtOAc. The organic extracts were dried over anhydrous sodium sulphate and evaporated in vacuo. The residue obtained was subjected to silica gel flash column chromatography, eluting with a gradient of EtOAc and heptane to obtain the title compound as a solid (0.32 g). HPLC/MS (method 1): Rt: 1.584 min: m/z=209 (M+1)⁺.

### Step 3: Synthesis of [N-(2-isopropyl-5-methyl-phenyl)carbamimidoyl]sulfanylmethyl acetate hydrobromide (E1′)

To a stirred solution of [(1E)-1-isobutyl-3-methyl-buta-1,3-dienyl]thiourea (0.1 g) in Acetone (2 mL) was added bromomethyl acetate (0.081 g) at ambient temperature. The reaction mixture was stirred at ambient temperature for 18 h and after completion of the reaction, the solvent from reaction mixture was evaporated in vacuo to obtain the title compound as a oil (0.1 g).

HPLC/MS (method 1): Rt: 1.936 min; m/z=281 (M+1)⁺.

### Example C-3

### Preparation of N-[5-[4-[(2-isopropyl-5-methyl-phenyl)carbamothioylcarbamoylamino]phenyl]-2,4-dimethyl-pyrazol-3-yl]-4-(trifluoromethyl)benzamide (C-3)

To a stirred solution of [(Z)-N′-[[4-[1,4-dimethyl-5-[[4-(trifluoromethyl)benzoyl]amino]pyrazol-3-yl]phenyl]carbamoyl]-N-(2-isopropyl-5-methyl-phenyl)carbamimidoyl]sulfanylmethylacetate (0.325 g) in MeOH (6 mL) was added 7 N Ammonia in MeOH (2 mL) at ambient temperature. The reaction mixture was stirred at ambient temperature for 18 h. After completion of the reaction, the solvent from reaction mixture was evaporated in vacuo. The residue obtained was subjected to silica gel flash column chromatography, eluting with a gradient of EtOAc and heptane to obtain the title compound as a solid (0.085 g).

HPLC/MS (method 1): Rt: 2.173 min; m/z=609 (M+1)⁺.

¹H NMR (500 MHz, DMSO-d₆) δ 11.68 (s, 1H), 10.48 (s, 1H), 10.16 (s, 1H), 9.41 (s, 1H), 8.23 (d, J=8.1 Hz, 2H), 7.97 (d, J=8.1 Hz, 2H), 7.71-7.65 (m, 2H), 7.55-7.50 (m, 2H), 7.28-7.21 (m, 2H), 7.15-7.09 (m, 1H), 3.71 (s, 3H), 3.01 (p, J=6.9 Hz, 1H), 2.29 (s, 3H), 2.07 (s, 3H), 1.18 (d, J=6.8 Hz, 6H).

### Example C-4

### Preparation of N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-N-methyl-4-(trifluoromethyl)benzamide (C-4)

### Step 1: N-[5-(4-bromophenyl)-2,4-dimethyl-pyrazol-3-yl]-N-methyl-4-(trifluormethyl)benzamide

N-[5-(4-bromophenyl)-2,4-dimethyl-pyrazol-3-yl]-4-(trifluormethyl)benzamide (0.5 g) was dissolved in DMF (12.5 mL) and cesium carbonate (0.74 g) and methyl iodide (0.2 g) were added subsequently. The reaction mixture was stirred for 3 h at 50° C. Once the starting material was consumed, the reaction mixture was allowed to cool to room temperature and EtOAc was added. The resulting precipitate was filtered, solvent evaporated and purified by column chromatography to yield N-[5-(4-bromophenyl)-2,4-dimethyl-pyrazol-3-yl]-N-methyl-4-(trifluormethyl)benzamide (0.52 g). ¹H NMR (500 MHz, Chloroform-d) δ 7.54-7.48 (m, 4H), 7.46-7.40 (m, 4H), 3.70 (s, 3H), 3.40 (s, 3H), 1.98 (s, 3H).

### Step 2: Tert-butyl-N-[4-[1,4-dimethyl-5-[methyl-[4-(trifluormethyl)benzoyl]amino]pyrazol-3-yl]phenyl]carbamate

N-[5-(4-bromophenyl)-2,4-dimethyl-pyrazol-3-yl]-N-methyl-4-(trifluormethyl)benzamide (0.52 g) was dissolved in 1,4-dioxane (5 mL) and tert-butyl carbamate (0.20 g), cesium carbonate (0.90 g), Palladium (II) acetate (0.04 g) and 2-dicyclohexylphosphino-2′,4′,6′-triisopropylbiphenyl (0.14 g) were added subsequently while purging under nitrogen. The resulting mixture was stirred overnight at 100° C. Once the starting material was consumed, the reaction mixture was allowed to cool to room temperature, quenched by addition of water (7 mL) and the organic phases were extracted twice with EtOAc. The combined organic phases were washed with saturated brine solution, dried over magnesium sulfate and evaporated to yield tert-butyl-N-[4-[1,4-dimethyl-5-[methyl-[4-(trifluormethyl)benzoyl]amino]pyrazol-3-yl]phenyl]carbamate (0.52 g).

¹H NMR (500 MHz, Chloroform-d) δ 8.05 (m, 2H), 7.82 (m, 2H), 7.57 (m, 2H), 7.46 (m, 2H), 3.75 (s, 3H), 2.12 (s, 3H), 1.68 (s, 3H), 1.59 (s, 9H).

### Step 3: N-[5-(4-aminophenyl)-2,4-dimethyl-pyrazol-3-yl]-N-methyl-4-(trifluoromethyl) benzamide

Tert-butyl-N-[4-[1,4-dimethyl-5-[methyl-[4-(trifluormethyl)benzoyl]amino]pyrazol-3-yl]phenyl]carbamate (0.52 g) was dissolved in DCM (50 mL) and Trifluoroacetic acid (0.25 mL) was added at room temperature. The reaction mixture was stirred for 5 days and additional trifluoroacetic acid (0.36 mL) was added until all the remaining starting material has been consumed. The reaction mixture was neutralized by addition of saturated sodium bicarbonate solution. The organic phases were dried over magnesium sulfate, evaporated and purified with column chromatography to yield N-[5-(4-aminophenyl)-2,4-dimethyl-pyrazol-3-yl]-N-methyl-4-(trifluoromethyl)benzamide (0.2 g). ¹H NMR (500 MHz, Chloroform-d) δ 7.54-7.48 (m, 4H), 7.46-7.40 (m, 4H), 3.72 (s, 3H), 3.42 (s, 3H), 1.99 (s, 3H).

### Step 4: N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-N-methyl-4-(trifluormethyl) benzamide (C-4)

N-[5-(4-aminophenyl)-2,4-dimethyl-pyrazol-3-yl]-N-methyl-4-(trifluoromethyl)benzamide (0.1 g) was dissolved in acetonitrile (7 mL) and cesium carbonate (0.07 g) and 4-nitrophenyl chloroformate (0.05 g) were added subsequently. The reaction mixture was stirred at room temperature for 5 h. Once TLC indicated consumption of starting material, 2-imino-3-(2-isopropyl-5-methyl-phenyl)thiazolidine-4-one (0.07 g) and cesium carbonate (0.08 g) were added. The reaction mixture was stirred overnight at room temperature, solvents were evaporated, and the residue purified by column chromatography to yield N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-N-methyl-4-(trifluormethyl)benzamide (0.04 g). ¹H NMR (400 MHz, Chloroform-d) δ 7.49-7.34 (m, 10H), 6.92-6.87 (m, 1H), 3.95 (d, J=3.1 Hz, 2H), 3.67 (s, 3H), 3.39 (s, 3H), 2.66 (m, 1H), 2.37 (s, 3H), 1.97 (s, 3H), 1.18 (m, 6H)

### Example C-5

### Preparation of N-[5-[4-[[(Z)-[3-(2-isopropyl-5-methyl-phenyl)-4-oxo-thiazolidin-2-ylidene]carbamoyl]amino]phenyl]-2-methyl-1,2,4-triazol-3-yl]-4-(trifluoromethoxy)benzamide (C-5)

### Step 1: Methyl 4-bromobenzenecarboximidate

To a stirred solution of 4-bromobenzonitrile (10 g) in methanol (100 mL) was added acetyl chloride (43.125 g) at 0° C. The reaction mixture was stirred at ambient temperature for 0.5 h and after completion of the reaction the mixture was evaporated. Solid precipitated was filtered and washed with methyl tert-butyl ether and dried in vacuo to obtain the title compound as off white solid (10 g). HPLC/MS (method 1): Rt: 0.86 min; m/z=214 (M)⁺.

### Step 2: methyl (1Z)-4-bromo-N-cyano-benzenecarboximidate

To a stirred solution of methyl 4-bromobenzenecarboximidate HCl salt (3 g) in water (50 mL) was added cyanamide (1.374 g) and di-sodium hydrogen phosphate (3.315 g) at ambient temperature. The reaction mixture was stirred at ambient temperature for 19 h and after completion of the reaction the mixture was diluted in water. The mixture was extracted with EtOAc and the organic extracts were dried over anhydrous sodium sulphate and evaporated in vacuo to obtain the title compound as a yellowish oil (3 g). ¹H NMR (300 MHz, Chloroform-d) δ 8.00 (d, J=8.7 Hz, 2H), 7.68 (d, J=8.7 Hz, 2H), 4.08 (s, 3H).

### Step 3: 5-(4-bromophenyl)-2-methyl-1,2,4-triazol-3-amine

To a stirred solution of methyl (1Z)-4-bromo-N-cyano-benzenecarboximidate (5 g) in MeOH (40 mL) and Acetic acid (10 mL) was added methylhydrazine (1.287 g, 85% solution) at ambient temperature. The reaction mixture was heated at 85° C. for 16 h and after completion of the reaction, the reaction mixture was cooled to ambient temperature, solid precipitated was filtered and washed with MeOH and dried in vacuo to obtain the title compound as off white solid (3 g).

HPLC/MS (method 1): Rt: 1.25 min; m/z=253 (M)⁺.

### Step 4: N-[5-(4-bromophenyl)-2-methyl-1,2,4-triazol-3-yl]-4-(trifluoromethoxy) benzamide

To a stirred solution of 5-(4-bromophenyl)-2-methyl-1,2,4-triazol-3-amine (1 g) in THF (10 mL) were added Triethylamine (0.8 g) and 4-(trifluoromethoxy)benzoyl chloride (0.97 g) at ambient temperature. The reaction mixture was stirred at ambient temperature for 18 h. The reaction mixture was dissolved in water and the mixture was extracted with DCM. The organic extracts were washed with aqueous sodium bicarbonate solution, dried over anhydrous sodium sulphate and evaporated in vacuo The residue obtained was subjected to silica gel flash column chromatography, eluting with a gradient of EtOAc and heptane to obtain the title compound as a solid (1 g). HPLC/MS (method 1): Rt: 2.13 min; m/z=441 (M)⁺.

### Step 5: Synthesis of tert-butyl N-[4-[1-methyl-5-[[4-(trifluoromethoxy)benzoyl]amino]-1,2,4-triazol-3-yl]phenyl]carbamate

To a stirred solution of N-[5-(4-bromophenyl)-2-methyl-1,2,4-triazol-3-yl]-4-(trifluoromethoxy) benzamide (6 g) in 1,4-dioxane (60 mL) were added tert-butyl carbamate (2.39 g), tris(dibenzylideneacetone)dipalladium(0) (1.245 g), 5-(di-tert-butylphosphino)-1′,3′,5′-triphenyl-1′H-[1,4′]bipyrazole (0.689 g) and potassium tert-butoxide (4.578 g) at ambient temperature under inert atmosphere. The reaction mixture was heated at 90° C. for 16 h and after completion of the reaction, the mixture was dissolved in water. The mixture was extracted with EtOAc, the organic extracts were dried over anhydrous sodium sulphate and evaporated in vacuo. The residue obtained was subjected to silica gel flash column chromatography, eluting with a gradient of EtOAc and heptane to obtain the title compound as a solid (5.8 g). HPLC/MS (method 1): Rt: 2.12 min; m/z=478 (M+1)⁺.

### Step 6: N-[5-(4-aminophenyl)-2-methyl-1,2,4-triazol-3-yl]-4-(trifluoromethoxy)benzamide

To a stirred solution of tert-butyl N-[4-[1-methyl-5-[[4-(trifluoromethoxy)benzoyl]amino]-1,2,4-triazol-3-yl]phenyl]carbamate (5.8 g) in DCM (80 mL) was added trifluoroacetic acid (5.61 mL) at ambient temperature. The reaction mixture was stirred at ambient temperature for 6 h and after completion of the reaction, the reaction mixture was quenched with water (100 mL) and neutralized with 10% sodium hydroxide solution. The mixture was extracted with EtOAc. The organic extracts dried over anhydrous sodium sulphate and evaporated in vacuo to obtain the title compound as a solid (4.1 g). HPLC/MS (method 1): Rt: 1.69 min; m/z=378 (M+1)⁺.

### Step 7: Synthesis of N-[5-[4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-4-(trifluoromethyl)benzamide (C-5)

To a stirred solution of N-[5-(4-aminophenyl)-2-methyl-1,2,4-triazol-3-yl]-4-(trifluoromethoxy) benzamide (0.2 g) in THE (4 mL) were added (4-nitrophenyl) carbonochloridate (0.112 g) and pyridine (0.168 g) at 0° C. The reaction mixture was stirred at ambient temperature for 3 h. After 3 h, 2-imino-3-(2-isopropyl-5-methyl-phenyl)thiazolidin-4-one (E1, 0.132 g) and pyridine (0.168 g) were added. The reaction mixture was stirred at 65° C. for 3 h. After completion of the reaction, the mixture was diluted with water and extracted with EtOAc. The organic extracts were dried over anhydrous sodium sulphate and evaporated in vacuo. The residue obtained was subjected to preparative HPLC purification, eluting with a gradient of acetonitrile and water to obtain the title compound as a solid (0.118 g).

HPLC/MS (method 1): Rt: 5.84 min; m/z=650 (M−1)⁺.

¹H NMR (500 MHz, DMSO-d₆) δ 11.28 (s, 1H), 9.95 (s, 1H), 8.20-8.15 (m, 2H), 7.86 (d, J=8.4 Hz, 2H), 7.76 (d, J=8.5 Hz, 2H), 7.58 (d, J=8.3 Hz, 2H), 7.40 (d, J=8.0 Hz, 1H), 7.28 (d, J=8.2 Hz, 1H), 7.07 (d, J=1.9 Hz, 1H), 4.21 (d, J=17.9 Hz, 1H), 4.10 (d, J=15 Hz, 1H), 3.74 (s, 3H), 2.67 (p, J=6.9 Hz, 1H), 2.32 (s, 3H), 1.17 (d, J=6.8 Hz, 3H), 1.10 (d, J=6.8 Hz, 3H).

### Example C-6

### Preparation of N-[5-[3-chloro-4-[[(Z)-[3-(2-isopropyl-5-methyl-phenyl)-4-oxo-thiazolidin-2-ylidene]carbamoyl]amino]phenyl]-2-methyl-1,2,4-triazol-3-yl]-4-(trifluoromethoxy)benzamide (C-6)

### Step 1: N-[5-(4-amino-3-chloro-phenyl)-2-methyl-1,2,4-triazol-3-yl]-4-(trifluoromethoxy)benz-amide

To a stirred solution of N-[5-(4-aminophenyl)-2-methyl-1,2,4-triazol-3-yl]-4-(trifluoromethoxy) benzamide (1.5 g) in acetonitrile (15 mL) was added N-chlorosuccinimide (0.531 g) at ambient temperature. The reaction mixture was stirred at 70° C. for 6 h and after completion of the reaction, the mixture was diluted in water. The mixture was extracted with EtOAc and the organic extracts were dried over anhydrous sodium sulphate and evaporated in vacuo. The residue obtained was subjected to silica gel flash column chromatography, eluting with a gradient of EtOAc and heptane to obtain the title compound as a yellow solid (1.25 g).

HPLC/MS (method 1): Rt: 2.02 min; m/z=412.2 (M+1)⁺.

### Step 2: N-[5-[3-chloro-4-[[(Z)-[3-(2-isopropyl-5-methyl-phenyl)-4-oxo-thiazolidin-2-ylidene]carbamoyl]amino]phenyl]-2-methyl-1,2,4-triazol-3-yl]-4-(trifluoromethoxy)benzamide (C-6)

To a stirred solution of N-[5-(4-amino-3-chloro-phenyl)-2-methyl-1,2,4-triazol-3-yl]-4-(trifluoromethoxy)benzamide (1.0 g) in THF (10 mL) was added (4-nitrophenyl) carbochloridate (0.587 g) at 0° C. under inert atmosphere. The reaction mixture was stirred at ambient temperature for 3 h. After this time acetonitrile (10 mL) was added. 2-imino-3-(2-isopropyl-5-methyl-phenyl)thiazolidin-4-one (E1, 0.905 g), N,N,N-diisopropyl ethylamine (0.628 g) and potassium phosphate tribasic (0.635 g) were added at 0° C. The reaction mixture was stirred at 27° C. for 16 h. After completion of the reaction, the mixture was diluted in water. The mixture was extracted with EtOAc and the organic extracts were dried over anhydrous sodium sulphate and evaporated in vacuo. The residue obtained was triturated in MeOH and filtered to obtain the title compound as a white solid (1.2 g). HPLC/MS (method 1): Rt: 2.34 min; m/z=686.4 (M+1)⁺. ¹H NMR (500 MHz, DMSO-d₆) δ 11.32 (s, 1H), 9.08 (s, 1H), 8.20-8.13 (d, 2H), 8.01-7.66 (m, 3H), 7.59 (d, J=8.3 Hz, 2H), 7.44-7.19 (m, 2H), 7.06 (s, 1H), 4.28-4.03 (m, 2H), 3.77 (s, 3H), 2.74-2.60 (m, 1H), 2.31 (s, 3H), 1.29-0.96 (m, 6H).

### Example C-7

### Preparation of N-[4-cyano-5-[4-[[(Z)-[3-(2-isopropyl-5-methyl-phenyl)-4-oxo-thiazolidin-2-ylidene]carbamoyl]amino]phenyl]-2-methyl-pyrazol-3-yl]-4-(trifluoromethoxy)benzamide (C-7)

### Step 1: Synthesis of 3,5-dibromo-1H-pyrazole-4-carbonitrile

To a stirred solution of 1H-pyrazole-4-carbonitrile (10 g) in EtOH (25 mL) and water (35 mL) were added sodium acetate (12.336 g) and bromine (6.86 g) at 10° C. The reaction mixture was stirred at ambient temperature for 2 h. The reaction mixture was dissolved in water and the mixture was extracted with DCM. The organic extracts dried over anhydrous sodium sulphate and evaporated in vacuo to obtain the title compound (4.9 g). HPLC/MS (method 1): Rt: 1.73 min; m/z=252 (M+2)⁺.

### Step 2: Synthesis of 3,5-dibromo-1-methyl-pyrazole-4-carbonitrile

To a stirred solution of 3,5-dibromo-1H-pyrazole-4-carbonitrile (4.9 g) in DMF (25 mL) were added cesium carbonate (9.545 g) and methyl iodide (4.158 g) at ambient temperature. The reaction mixture was stirred at ambient temperature for 17 h. The reaction mixture was dissolved in water, solid precipitated was filtered and washed with water and dried in vacuo to obtain the title compound as off white solid (50 g). HPLC/MS (method 1): Rt: 1.82 min; m/z=266 (M+1)⁺.

### Step 3: Synthesis of 5-amino-3-bromo-1-methyl-pyrazole-4-carbonitrile

To a stirred solution of 3,5-dibromo-1-methyl-pyrazole-4-carbonitrile (7 g) in N-methyl-2-pyrrolidone (14 mL) was added 2,4-dimethoxybenzylamine (8.83 mL) at ambient temperature. The reaction mixture was stirred at 140° C. for 2 h. After completion of reaction, 5N hydrochloric acid (50 mL) was added and reaction mixture was stirred at ambient temperature for 2 h. The reaction mixture was dissolved in water and the mixture was extracted with EtOAc. The organic extracts were dried over anhydrous sodium sulphate and evaporated in vacuo. The residue obtained was subjected to silica gel flash column chromatography, eluting with a gradient of EtOAc and heptane to obtain the title compound as a solid (5.8 g).

HPLC/MS (method 1): Rt: 1.01 min; m/z=203 (M+2)⁺.

### Step 4: Synthesis of N-(5-bromo-4-cyano-2-methyl-pyrazol-3-yl)-4-(trifluoromethoxy)benzamide

To a stirred solution of 5-amino-3-bromo-1-methyl-pyrazole-4-carbonitrile (7 g) in DCM (70 mL) were added pyridine (14 mL) and 4-(trifluoromethoxy)benzoyl chloride (6.7 mL) at 0° C. The reaction mixture was stirred at ambient temperature for 24 h. The reaction mixture was dissolved in water and the mixture was extracted with DCM. The organic extracts were washed with 2.5M, hydrochloric acid, dried over anhydrous sodium sulphate and evaporated in vacuo. The residue obtained was subjected to silica gel flash column chromatography, eluting with a gradient of EtOAc and heptane to obtain the title compound as a solid (2.5 g).

HPLC/MS (method 1): Rt: 2.02 min; m/z=389 (M)⁺.

### Step 5: Synthesis of N-[5-(4-aminophenyl)-4-cyano-2-methyl-pyrazol-3-yl]-4-(trifluoromethoxy) benzamide

To a stirred solution of N-(5-bromo-4-cyano-2-methyl-pyrazol-3-yl)-4-(trifluoromethoxy)benzamide (3.2 g) in 1,4-dioxane (30 mL) and water (3 mL) were added 4-(4,4,5,5-tetramethyl-1,3,2-dioxaborolan-2-yl)aniline (1.98 g), [1,1′-bis(diphenylphosphino)ferrocene]dichloropalladium(II) (0.602 g) and potassium carbonate (1.70 g) at ambient temperature under inert atmosphere. The reaction mixture was heated at 80° C. for 8 h and after completion of the reaction, the mixture was dissolved in water. The mixture was extracted with EtOAc, the organic extracts were dried over anhydrous sodium sulphate and evaporated in vacuo. The residue obtained was subjected to silica gel flash column chromatography, eluting with a gradient of EtOAc and heptane to obtain the title compound as a solid (2.05 g). HPLC/MS (method 1): Rt: 1.93 min; m/z=402 (M+1)⁺.

### Step 6: Synthesis of N-[4-cyano-5-[4-[[(Z)-[3-(2-isopropyl-5-methyl-phenyl)-4-oxo-thiazolidin-2-ylidene]carbamoyl]amino]phenyl]-2-methyl-pyrazol-3-yl]-4-(trifluoromethoxy)benzamide (C-7)

To a stirred solution of N-[5-(4-aminophenyl)-4-cyano-2-methyl-pyrazol-3-yl]-4-(trifluoro-methoxy) benzamide (0.2 g) in THF (4 mL) were added bis(2,5-dioxopyrrolidin-1-yl) carbonate (0.134 g) and pyridine (0.157 g) at 0° C. The reaction mixture was stirred at ambient temperature for 3 h. After 3h, 2-imino-3-(2-isopropyl-5-methyl-phenyl)thiazolidin-4-one (E1, 0.124 g) and pyridine (0.157 g) were added. The reaction mixture was stirred at 65° C. for 3 h. After completion of the reaction, the mixture was diluted with water and extracted with EtOAc. The organic extracts dried over anhydrous sodium sulphate and evaporated in vacuo. The residue obtained was subjected to silica gel flash column chromatography, eluting with a gradient of EtOAc and heptane to obtain the title compound as a solid (0.175 g). HPLC/MS (method 1): Rt: 2.523 min; m/z=675 (M−1)⁺. ¹H NMR (500 MHz, DMSO-d₆) δ 11.13 (s, 1H), 9.99 (s, 1H), 8.20-8.15 (m, 2H), 7.79 (q, J=8.5 Hz, 4H), 7.62 (d, J=8.3 Hz, 2H), 7.40 (d, J=8.3 Hz, 1H), 7.28 (d, J=8.0 Hz, 1H), 7.07 (s, 1H), 4.20 (s, 1H), 4.09 (d, J=18.0 Hz, 1H), 3.80 (d, J=2.1 Hz, 3H), 2.67 (p, J=7.3 Hz, 1H), 2.32 (s, 3H), 1.17 (d, J=6.9 Hz, 3H), 1.10 (d, J=6.9 Hz, 3H).

### Example C-8

### Preparation of N-[5-[3-chloro-4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-4-(trifluoromethoxy)benzamide (C-8)

### Step 1: Synthesis of N-[(1-amino-2-methyl-prop-1-enyl)-methyl-amino]formamide

To a stirred solution of ethyl 2-cyanopropanoate (4 g) in 1,4-dioxane (40 mL) were added methylhydrazine 80% Solution (1.993 g) at 0° C. The reaction mixture was heated at 110° C. for 17 h and after completion of the reaction, the mixture was evaporated. Solid precipitated was filtered and washed with heptane and dried to obtain the title compound as a solid (2.9 g).

HPLC/MS (method 1): Rt: 1.90 min; m/z=128.3 (M+1)⁺.

### Step 2: Synthesis of (5-amino-1,4-dimethyl-pyrazol-3-yl) trifluoromethanesulfonate

To a stirred solution of 3-amino-2,4-dimethyl-1H-pyrazol-5-one (1.6 g) in DMF (20 mL) were added 1,1,1-trifluoro-N-phenyl-N-(trifluoromethylsulfonyl)methanesulfonamide (5.39 g), N,N,N-diisopropyl ethylamine (4.87 g) at 0° C. The reaction mixture was stirred at ambient temperature for 17 h and after completion of the reaction, the mixture was dissolved in water. The mixture was extracted with EtOAc, the organic extracts were dried over anhydrous sodium sulphate and evaporated in vacuo. The residue obtained was subjected to silica gel flash column chromatography, eluting with a gradient of EtOAc and heptane to obtain the title compound as a solid (3.15 g).

HPLC/MS (method 1): Rt: 1.90 min; m/z=260 (M+1)⁺.

### Step 3: Synthesis of [1,4-dimethyl-5-[[4-(trifluoromethoxy)benzoyl]amino]pyrazol-3-yl]trifluoromethanesulfonate

To a stirred solution of (5-amino-1,4-dimethyl-pyrazol-3-yl) trifluoromethanesulfonate (3 g) in DCM (30 mL) were added pyridine (9.34 mL) and 4-(trifluoromethoxy)benzoyl chloride (2.85 mL) at 0° C. The reaction mixture was stirred at ambient temperature for 24 h. The reaction mixture was dissolved in water and extracted with DCM. The organic extracts were washed with 2.5M hydrochloric acid, dried over anhydrous sodium sulphate and evaporated in vacuo. The residue obtained was subjected to silica gel flash column chromatography, eluting with a gradient of EtOAc and heptane to obtain the title compound as a white solid (3.15 g).

HPLC/MS (method 1): Rt: 2.16 min; m/z=448 (M+1)⁺.

### Step 4: Synthesis of N-[5-(4-amino-3-chloro-phenyl)-2,4-dimethyl-pyrazol-3-yl]-4-(trifluoromethoxy)benzamide

To a stirred solution of [1,4-dimethyl-5-[[4-(trifluoromethoxy)benzoyl]amino]pyrazol-3-yl]trifluoromethanesulfonate (1 g) in 1,4-dioxane (10 mL) were added 2-chloro-4-(4,4,5,5-tetramethyl-1,3,2-dioxaborolan-2-yl)aniline (0.595 g), tetrakis(triphenylphosphine)palladium(0) (0.517 g) and potassium carbonate (0.618 g) at ambient temperature under inert atmosphere. The reaction mixture was heated at 120° C. for 18 h and after completion of the reaction, the mixture was dissolved in water. The mixture was extracted with EtOAc, the organic extracts were dried over anhydrous sodium sulphate and evaporated in vacuo. The residue obtained was subjected to silica gel flash column chromatography, eluting with a gradient of EtOAc and heptane to obtain the title compound as a solid (0.59 g).

HPLC/MS (method 1): Rt: 2.24 min; m/z=425 (M+1)⁺.

### Step 5: Synthesis of N-[5-[3-chloro-4-[[(Z)-[3-(2-isopropyl-5-methyl-phenyl)-4-oxo-thiazolidin-2-ylidene]carbamoyl]amino]phenyl]-2,4-dimethyl-pyrazol-3-yl]-4-(trifluoromethoxy)benzamide (C-8)

To a stirred solution of N-[5-(4-amino-3-chloro-phenyl)-2,4-dimethyl-pyrazol-3-yl]-4-(trifluoromethoxy)benzamide (0.6 g) in THF (12 mL) were added bis(2,5-dioxopyrrolidin-1-yl) carbonate (0.38 g) and pyridine (0.446 g) at 0° C. The reaction mixture was stirred at ambient temperature for 3 h. After 3 h, 2-imino-3-(2-isopropyl-5-methyl-phenyl)thiazolidin-4-one (E1, 0.351 g) and pyridine (0.446 g) were added. The reaction mixture was stirred at 65° C. for 3 h. After completion of the reaction, the mixture was diluted with water and extracted with EtOAc. The organic extracts were dried over anhydrous sodium sulphate and evaporated in vacuo. The residue obtained was subjected to silica gel flash column chromatography, eluting with a gradient of EtOAc and heptane to obtain the title compound as a solid (0.45 g).

HPLC/MS (method 1): Rt: 2.29 min; m/z=699 (M)⁺.

¹H NMR (500 MHz, DMSO-d₆) δ 10.41 (s, 1H), 9.10 (s, 1H), 8.31-8.11 (m, 2H), 7.87-7.53 (m, 5H), 7.42 (s, 1H), 7.30 (s, 1H), 7.10 (s, 1H), 4.24 (d, J=18.0 Hz, 1H), 4.11 (d, J=18.0 Hz, 1H), 3.72 (s, 3H), 2.70 (s, 1H), 2.34 (s, 3H), 2.09 (s, 3H), 1.22 (s, 3H), 1.12 (d, J=6.9 Hz, 3H).

### Example C-112

### Preparation of N-[5-[4-[[(Z)-[3-(2-isopropyl-5-methyl-phenyl)-4-oxo-thiazolidin-2-ylidene]carbamoyl]-methyl-amino]phenyl]-2,4-dimethyl-pyrazol-3-yl]-4-(trifluoromethoxy) benzamide

### Step 1: Synthesis of N-methyl-4-(4,4,5,5-tetramethyl-1,3,2-dioxaborolan-2-yl)aniline

To a stirred solution of 4-(4,4,5,5-tetramethyl-1,3,2-dioxaborolan-2-yl)aniline (1.0 g) in N,N-Dimethylformamide (10 mL) were added methyl iodide (0.777 g) and potassium carbonate (1.262 g) at ambient temperature under inert atmosphere. The reaction mass was stirred for 18 h at 50° C. and the progress of the reaction was monitored by TLC analysis. After completion of reaction, reaction mass was diluted with water (20 mL) and followed by extracted in EtOAc (25 mL×2). The combined organic extracts were dried over anhydrous sodium sulphate, filtered, and concentrated under reduced pressure. The crude product was purified by flash chromatography to afford title compound as a solid (1.0 g).

HPLC/MS (Method 1): Rt: 2.09 min; m/z=234.3 (M+1)+;

¹H NMR (500 MHz, DMSO-d6) δ 7.43-7.37 (m, 2H), 6.52-6.46 (m, 2H), 6.04 (q, J=4.9 Hz, 1H), 2.68 (d, J=5.0 Hz, 3H), 1.25 (s, 12H).

### Step 2: Synthesis of N-[2,4-dimethyl-5-[4-(methylamino)phenyl]pyrazol-3-yl]-4-(trifluoro-methoxy)benzamide

To a stirred solution of [1,4-dimethyl-5-[[4-(trifluoromethoxy)benzoyl]amino]pyrazol-3-yl]trifluoromethanesulfonate (1.2 g) in 1,4-Dioxane (12 mL) were added N-methyl-4-(4,4,5,5-tetramethyl-1,3,2-dioxaborolan-2-yl)aniline (0.688 g), Tetrakis (0.620 g) and potassium carbonate (0.742 g) at ambient temperature under inert atmosphere. The reaction mass was stirred for 18 h at 110° C. and the progress of the reaction was monitored by TLC analysis. After completion of reaction, reaction mass was diluted with Water (30 mL) and followed by extracted in EtOAc (40 mL×2). The combined organic extracts were dried over anhydrous sodium sulphate, filtered, and concentrated under reduced pressure. The crude product was purified by flash chromatography to afford title compound as a solid (1.01 g).

HPLC/MS (Method 1): Rt: 1.98 min; m/z=405.3 (M+1)+;

¹H NMR (300 MHz, DMSO-d₆) δ 10.29 (s, 1H), 8.15 (dd, J=8.7, 6.8 Hz, 2H), 7.67-7.54 (m, 4H), 7.41 (d, J=8.3 Hz, 2H), 6.59 (d, J=8.4 Hz, 1H), 3.63 (d, J=6.8 Hz, 3H), 2.71 (d, J=5.0 Hz, 3H), 2.00 (d, J=3.2 Hz, 3H).

### Step 3: Synthesis of N-[5-[4-[benzoylcarbamothioyl(methyl)amino]phenyl]-2,4-dimethyl-pyrazol-3-yl]-4-(trifluoromethoxy)benzamide

To a stirred solution of N-[2,4-dimethyl-5-[4-(methylamino)phenyl]pyrazol-3-yl]-4-(trifluoromethoxy)benzamide (1.01 g) in acetone (15 mL) was added benzoyl isothiocyanate (0.488 g) at ambient temperature under inert atmosphere. The reaction mass was stirred for 19 h at 50° C. and the progress of the reaction was monitored by TLC analysis. After completion of reaction, solvent was evaporated under reduced pressure. The crude product was purified by flash chromatography to afford title compound (1.1 g).

HPLC/MS (Method 1): Rt: 2.08 min: m/z=568.2 (M+1)+.

### Step 4: Synthesis of N-[5-[4-[carbamothioyl(methyl)amino]phenyl]-2,4-dimethyl-pyrazol-3-yl]-4-(trifluoromethoxy)benzamide

To a stirred solution of N-[5-[4-[benzoylcarbamothioyl(methyl)amino]phenyl]-2,4-dimethyl-pyrazol-3-yl]-4-(trifluoromethoxy)benzamide (1.1 g) in MeOH (11 mL) was added Aq. 2N NaOH (13 mL) at ambient temperature. The reaction mass was stirred for 22 h at 65° C. and the progress of the reaction was monitored by TLC analysis. After completion of reaction, reaction mass pH was adjusted to neutral using 2N HCl (5 mL) and followed by extracted in EtOAc (25 mL×2). The combined organic extracts were dried over anhydrous sodium sulphate, filtered, and concentrated under reduced pressure. The crude product was purified by flash chromatography to afford title compound as a solid (0.300 g).

HPLC/MS (Method 1): Rt: 1.97 min; m/z=464.2 (M+1)+;

¹H NMR (500 MHz, DMSO-d₆) δ 10.38 (s, 1H), 8.20-8.13 (m, 2H), 7.77-7.70 (m, 2H), 7.67-7.51 (m, 4H), 7.36-7.30 (m, 2H), 3.71 (s, 3H), 3.49 (s, 3H), 2.09 (s, 3H).

### Step 5: Synthesis of N-[5-[4-[(4,5-dioxothiazol-2-yl)-methyl-amino]phenyl]-2,4-dimethyl-pyrazol-3-yl]-4-(trifluoromethoxy)benzamide

To a stirred solution of N-[5-[4-[carbamothioyl(methyl)amino]phenyl]-2,4-dimethyl-pyrazol-3-yl]-4-(trifluoromethoxy)benzamide (0.1 g) in EtOAc (2 mL) were added oxalyl chloride (0.096 g) and Triethylamine (0.052 g) at ambient temperature under inert atmosphere. The reaction mass was stirred for 15 min at ambient temperature and the progress of the reaction was monitored by TLC analysis. After completion of the reaction, reaction mass was diluted with water (10 mL) and followed by extracted in DCM (10 mL×2). The combined organic extracts were dried over anhydrous sodium sulphate, filtered, and concentrated under reduced pressure to afford title compound as a solid (0.120 g).

HPLC/MS (Method 1): Rt: 2.02 min; m/z=517.2 (M)−.

### Step 6: Synthesis of N-[5-[4-[(2-isopropyl-5-methyl-phenyl)carbamothioylcarbamoyl-methyl-amino]phenyl]-2,4-dimethyl-pyrazol-3-yl]-4-(trifluoromethoxy)benzamide

To a stirred solution of N-[5-[4-[(4,5-dioxothiazol-2-yl)-methyl-amino]phenyl]-2,4-dimethyl-pyrazol-3-yl]-4-(trifluoromethoxy)benzamide (0.140 g) in toluene (2 mL) at ambient temperature and reaction mass was stirred for 25 min at 100° C. The reaction mass was cooled to ambient temperature and was added 2-isopropyl-5-methyl-aniline (0.044 g). The reaction mass was stirred for 2 h at ambient temperature and the progress of the reaction was monitored by TLC analysis. After completion of reaction, the solvent was concentrated under reduced pressure and the crude product was purified by flash chromatography to afford title compound as a solid (0.04 g).

HPLC/MS (Method 1): Rt: 2.39 min; m/z=639.4 (M+1)+.

### Step 7: Synthesis of N-[5-[4-[[(Z)-[3-(2-isopropyl-5-methyl-phenyl)-4-oxo-thiazolidin-2-ylidene]carbamoyl]-methyl-amino]phenyl]-2,4-dimethyl-pyrazol-3-yl]-4-(trifluoromethoxy)benzamide (C-112)

To a stirred solution of N-[5-[4-[(2-isopropyl-5-methyl-phenyl) carbamothioylcarbamoyl-methyl-amino]phenyl]-2,4-dimethyl-pyrazol-3-yl]-4-(trifluoromethoxy)benzamide (0.040 g) in EtOH (1 mL) were added sodium acetate (0.010 g) and methyl bromo acetate (0.014 g) at ambient temperature. Then reaction mass was stirred at 50° 0 for 3 h and monitored by TLC analysis. After completion of reaction, reaction mass was diluted with water (5 mL) and followed by extracted in EtOAc (5 mL×2). The combined organic extracts were dried over anhydrous sodium sulphate, filtered, and concentrated under reduced pressure. The crude product was purified by flash chromatography to afford title compound as a solid (0.015 g).

HPLC/MS (Method 1): Rt: 2.22 min; m/z=679.3 (M+1)+;

¹H NMR (500 MHz, DMSO-d₆) b 10.36 (s, 1H), 8.18 (d, J=8.3 Hz, 2H), 7.59 (d, J=8.4 Hz, 2H), 7.46-7.22 (m, 3H), 7.17-6.72 (m, 4H), 4.22 (d, J=18.0 Hz, 1H), 4.08 (d, J=18.0 Hz, 1H), 3.70 (s, 3H), 3.17 (d, J=71.8 Hz, 4H), 2.16 (s, 3H), 2.06 (s, 3H), 1.11-0.69 (m, 6H).

All other examples enlisted in table C are synthesized analogously to the methods mentioned in either general procedure or experimental procedure mentioned above.

TABLE C
(I)

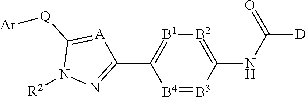

| No | Ar¹—Q | 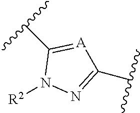 | 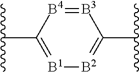 | 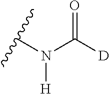 [SMILES: *NC([2H])=O] | LC MS/MZ | Rt min |
|---|---|---|---|---|---|---|
| C-1 | 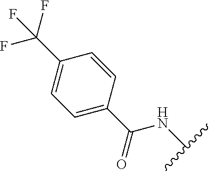 [SMILES: *NC(=O)c1ccc(C(F)(F)F)cc1] | 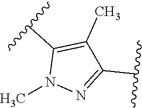 [SMILES: *c1nn(C)c(*)c1C] | 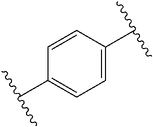 [SMILES: *c1ccc(*)cc1] | 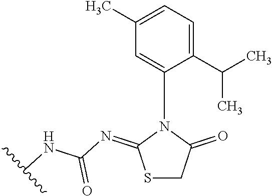 [SMILES: *NC(=O)/N=C1\SCC(=O)N1c1cc(C)ccc1C(C)C] | 649 (Method 1) | 1.96 |
| C-2 | 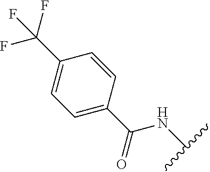 [SMILES: *NC(=O)c1ccc(C(F)(F)F)cc1] | 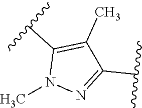 [SMILES: *c1nn(C)c(*)c1C] | 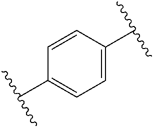 [SMILES: *c1ccc(*)cc1] | 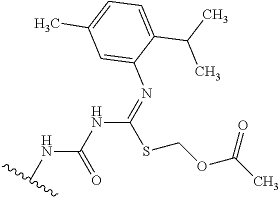 [SMILES: *NC(=O)N/C(=N\c1cc(C)ccc1C(C)C)SCOC(C)=O] | 681 (Method 1) | 2.20 |
| C-3 | 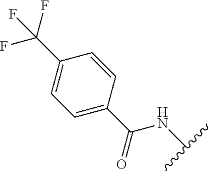 [SMILES: *NC(=O)c1ccc(C(F)(F)F)cc1] | 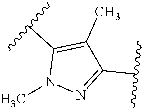 [SMILES: *c1nn(C)c(*)c1C] | 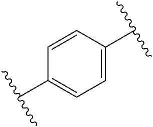 [SMILES: *c1ccc(*)cc1] | 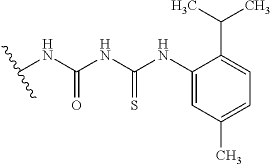 [SMILES: *NC(=O)NC(=S)Nc1cc(C)ccc1C(C)C] | 608 (Method 1) | 2.61 |
| C-4 | 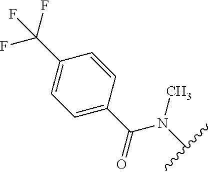 [SMILES: *N(C)C(=O)c1ccc(C(F)(F)F)cc1] | 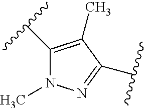 [SMILES: *c1nn(C)c(*)c1C] | 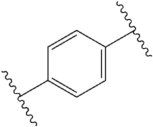 [SMILES: *c1ccc(*)cc1] | 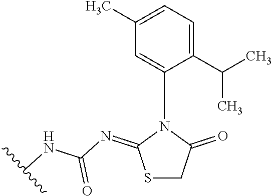 [SMILES: *NC(=O)/N=C1\SCC(=O)N1c1cc(C)ccc1C(C)C] | 663.2 (Method 2) | 1.33 |
| C-5 | 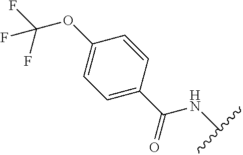 [SMILES: *NC(=O)c1ccc(OC(F)(F)F)cc1] | 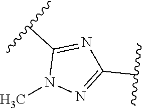 [SMILES: *c1nc(*)n(C)n1] | 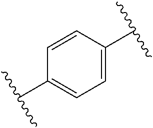 [SMILES: *c1ccc(*)cc1] | 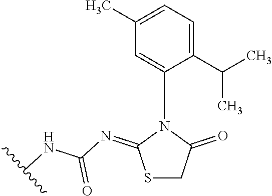 [SMILES: *NC(=O)/N=C1\SCC(=O)N1c1cc(C)ccc1C(C)C] | 650 (Method 1) | 2.56 |
| C-6 | 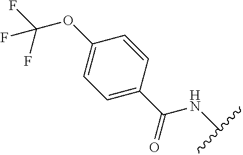 [SMILES: *NC(=O)c1ccc(OC(F)(F)F)cc1] | 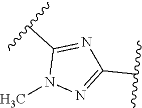 [SMILES: *c1nc(*)n(C)n1] | 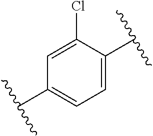 [SMILES: *c1ccc(*)c(Cl)c1] | 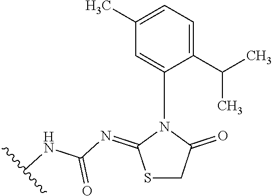 [SMILES: *NC(=O)/N=C1\SCC(=O)N1c1cc(C)ccc1C(C)C] | 686 (Method 1) | 2.25 |
| C-7 | 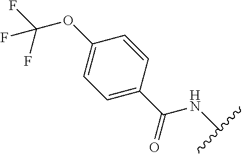 [SMILES: *NC(=O)c1ccc(OC(F)(F)F)cc1] | 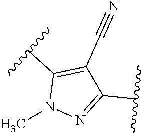 [SMILES: *c1nn(C)c(*)c1C#N] | 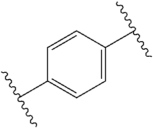 [SMILES: *c1ccc(*)cc1] | 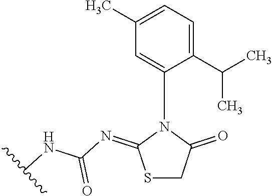 [SMILES: *NC(=O)/N=C1\SCC(=O)N1c1cc(C)ccc1C(C)C] | 675 (Method 1) | 2.55 |
| C-8 | 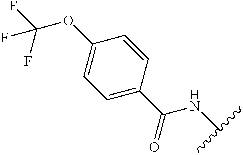 [SMILES: *NC(=O)c1ccc(OC(F)(F)F)cc1] | 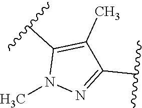 [SMILES: *c1nn(C)c(*)c1C] | 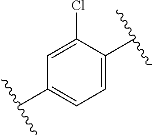 [SMILES: *c1ccc(*)c(Cl)c1] | 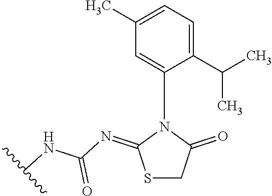 [SMILES: *NC(=O)/N=C1\SCC(=O)N1c1cc(C)ccc1C(C)C] | 699 (Method 1) | 2.27 |
| C-9 | 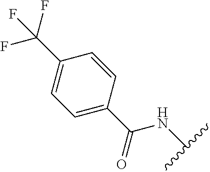 [SMILES: *NC(=O)c1ccc(C(F)(F)F)cc1] | 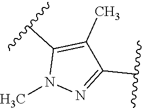 [SMILES: *c1nn(C)c(*)c1C] | 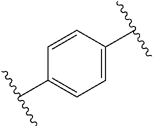 [SMILES: *c1ccc(*)cc1] | 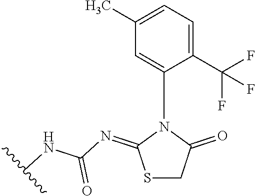 [SMILES: *NC(=O)/N=C1\SCC(=O)N1c1cc(C)ccc1C(F)(F)F] | 675 (Method 1) | 2.38 |
| C-10 | 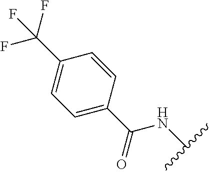 [SMILES: *NC(=O)c1ccc(C(F)(F)F)cc1] | 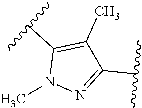 [SMILES: *c1nn(C)c(*)c1C] | 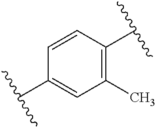 [SMILES: *c1ccc(*)c(C)c1] | 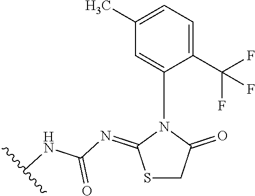 [SMILES: *NC(=O)/N=C1\SCC(=O)N1c1cc(C)ccc1C(F)(F)F] | 690 (Method 1) | 2.27 |
| C-11 | 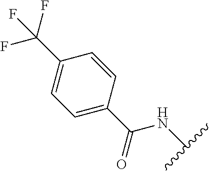 [SMILES: *NC(=O)c1ccc(C(F)(F)F)cc1] | 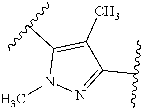 [SMILES: *c1nn(C)c(*)c1C] | 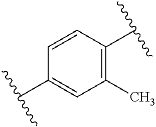 [SMILES: *c1ccc(*)c(C)c1] | 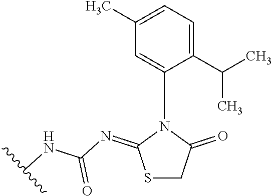 [SMILES: *NC(=O)/N=C1\SCC(=O)N1c1cc(C)ccc1C(C)C] | 663 (Method 1) | 2.34 |
| C-12 | 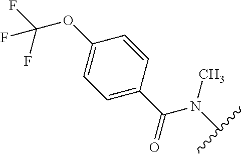 [SMILES: *N(C)C(=O)c1ccc(OC(F)(F)F)cc1] | 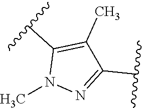 [SMILES: *c1nn(C)c(*)c1C] | 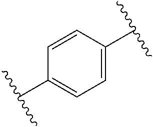 [SMILES: *c1ccc(*)cc1] | 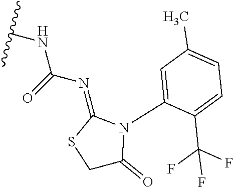 [SMILES: *NC(=O)/N=C1\SCC(=O)N1c1cc(C)ccc1C(F)(F)F] | 689 (Method 2) | 1.29 |
| C-13 | 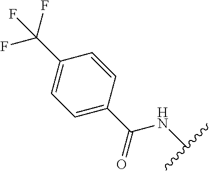 [SMILES: *NC(=O)c1ccc(C(F)(F)F)cc1] | 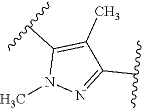 [SMILES: *c1nn(C)c(*)c1C] | 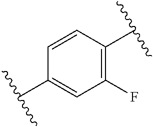 [SMILES: *c1ccc(*)c(F)c1] | 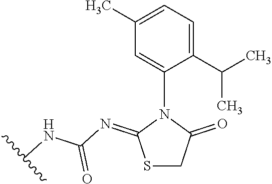 [SMILES: *NC(=O)/N=C1\SCC(=O)N1c1cc(C)ccc1C(C)C] | 667 (Method 1) | 2.41 |
| C-14 | 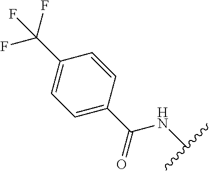 [SMILES: *NC(=O)c1ccc(C(F)(F)F)cc1] | 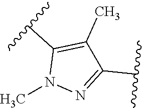 [SMILES: *c1nn(C)c(*)c1C] | 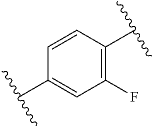 [SMILES: *c1ccc(*)c(F)c1] | 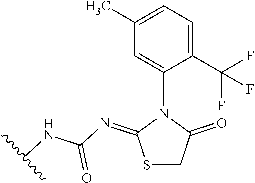 [SMILES: *NC(=O)/N=C1\SCC(=O)N1c1cc(C)ccc1C(F)(F)F] | 693 (Method 1) | 2.32 |
| C-15 | 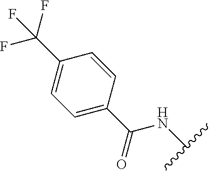 [SMILES: *NC(=O)c1ccc(C(F)(F)F)cc1] | 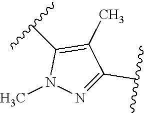 [SMILES: *c1nn(C)c(*)c1C] | 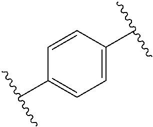 [SMILES: *c1ccc(*)cc1] | 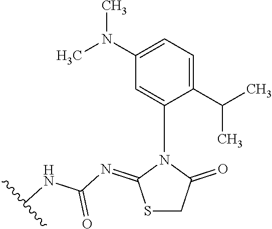 [SMILES: *NC(=O)/N=C1\SCC(=O)N1c1cc(N(C)C)ccc1C(C)C] | 678 (Method 1) | 2.58 |
| C-16 | 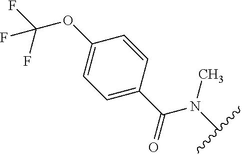 [SMILES: *N(C)C(=O)c1ccc(OC(F)(F)F)cc1] | 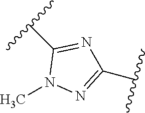 [SMILES: *c1nc(*)n(C)n1] | 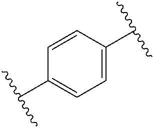 [SMILES: *c1ccc(*)cc1] | 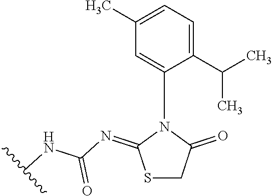 [SMILES: *NC(=O)/N=C1\SCC(=O)N1c1cc(C)ccc1C(C)C] | 666.8 (Method 1) | 2.48 |
| C-17 | 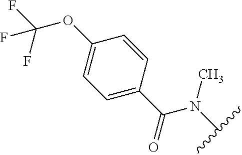 [SMILES: *N(C)C(=O)c1ccc(OC(F)(F)F)cc1] | 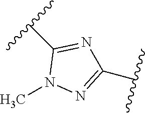 [SMILES: *c1nc(*)n(C)n1] | 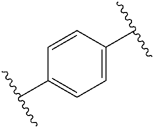 [SMILES: *c1ccc(*)cc1] | 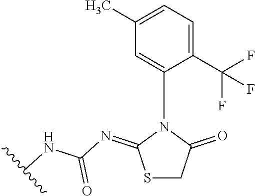 [SMILES: *NC(=O)/N=C1\SCC(=O)N1c1cc(C)ccc1C(F)(F)F] | 692.9 (Method 1) | 2.35 |
| C-18 | 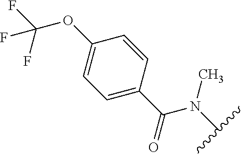 [SMILES: *N(C)C(=O)c1ccc(OC(F)(F)F)cc1] | 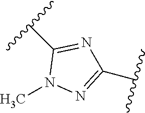 [SMILES: *c1nc(*)n(C)n1] | 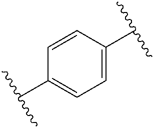 [SMILES: *c1ccc(*)cc1] | 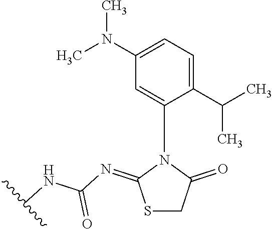 [SMILES: *NC(=O)/N=C1\SCC(=O)N1c1cc(N(C)C)ccc1C(C)C] | 695.7 (Method 1) | 1.78 |
| C-19 | 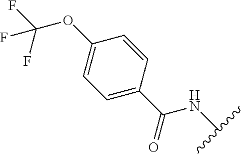 [SMILES: *NC(=O)c1ccc(OC(F)(F)F)cc1] | 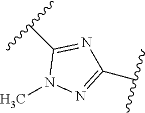 [SMILES: *c1nc(*)n(C)n1] | 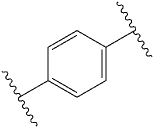 [SMILES: *c1ccc(*)cc1] | 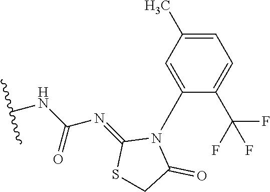 [SMILES: *NC(=O)/N=C1\SCC(=O)N1c1cc(C)ccc1C(F)(F)F] | 679 (Method 1) | 2.34 |
| C-20 | 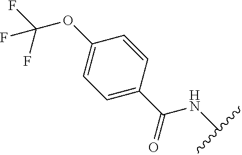 [SMILES: *NC(=O)c1ccc(OC(F)(F)F)cc1] | 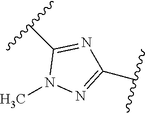 [SMILES: *c1nc(*)n(C)n1] | 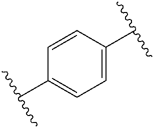 [SMILES: *c1ccc(*)cc1] | 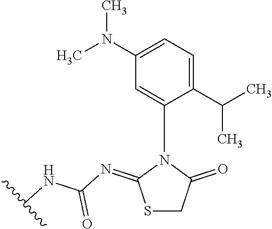 [SMILES: *NC(=O)/N=C1\SCC(=O)N1c1cc(N(C)C)ccc1C(C)C] | 679 (Method 1) | 2.70 |
| C-21 | 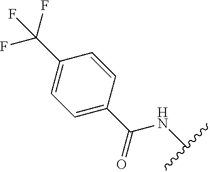 [SMILES: *NC(=O)c1ccc(C(F)(F)F)cc1] | 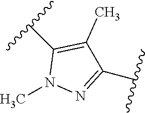 [SMILES: *c1nn(C)c(*)c1C] | 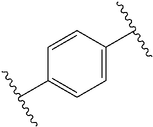 [SMILES: *c1ccc(*)cc1] | 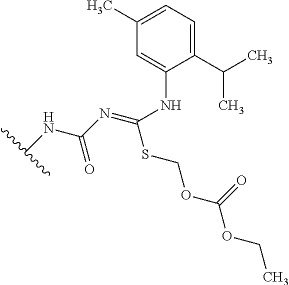 [SMILES: *NC(=O)/N=C(/Nc1cc(C)ccc1C(C)C)SCOC(=O)OCC] | 709 (Method 1) | 2.88 |
| C-22 | 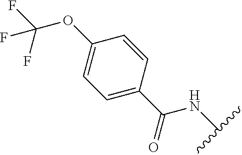 [SMILES: *NC(=O)c1ccc(OC(F)(F)F)cc1] | 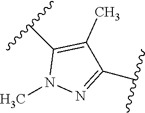 [SMILES: *c1nn(C)c(*)c1C] | 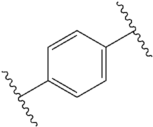 [SMILES: *c1ccc(*)cc1] | 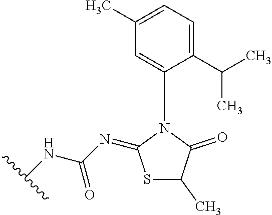 [SMILES: *NC(=O)/N=C1\SC(C)C(=O)N1c1cc(C)ccc1C(C)C] | 679.6 (Method 2) | 1.35 |
| C-23 | 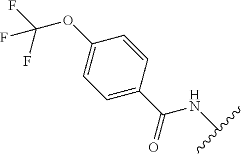 [SMILES: *NC(=O)c1ccc(OC(F)(F)F)cc1] | 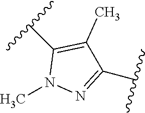 [SMILES: *c1nn(C)c(*)c1C] | 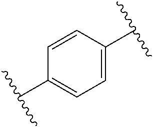 [SMILES: *c1ccc(*)cc1] | 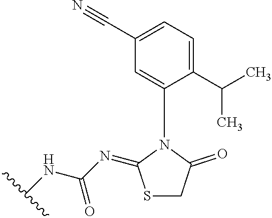 [SMILES: *NC(=O)/N=C1\SCC(=O)N1c1cc(C#N)ccc1C(C)C] | 676.6 (Method 2) | 1.25 |
| C-24 | 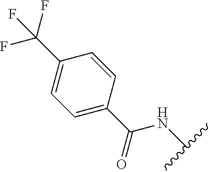 [SMILES: *NC(=O)c1ccc(C(F)(F)F)cc1] | 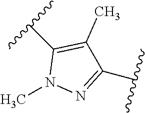 [SMILES: *c1nn(C)c(*)c1C] | 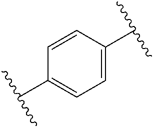 [SMILES: *c1ccc(*)cc1] | 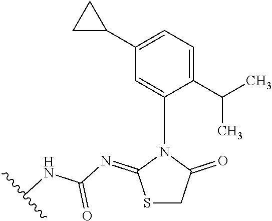 [SMILES: *NC(=O)/N=C1\SCC(=O)N1c1cc(C2CC2)ccc1C(C)C] | 675.7 (Method 1) | 2.16 |
| C-25 | 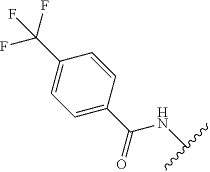 [SMILES: *NC(=O)c1ccc(C(F)(F)F)cc1] | 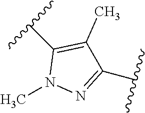 [SMILES: *c1nn(C)c(*)c1C] | 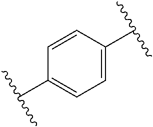 [SMILES: *c1ccc(*)cc1] | 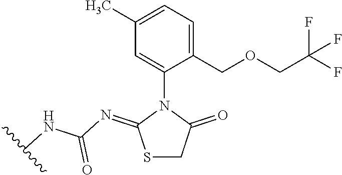 [SMILES: *NC(=O)/N=C1\SCC(=O)N1c1cc(C)ccc1COCC(F)(F)F] | 717 (Method 1) | 2.42 |
| C-26 | 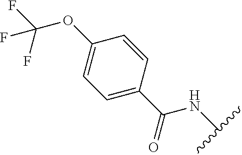 [SMILES: *NC(=O)c1ccc(OC(F)(F)F)cc1] | 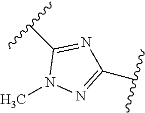 [SMILES: *c1nc(*)n(C)n1] | 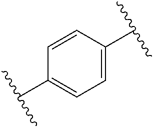 [SMILES: *c1ccc(*)cc1] | 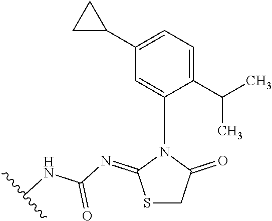 [SMILES: *NC(=O)/N=C1\SCC(=O)N1c1cc(C2CC2)ccc1C(C)C] | 678 (Method 1) | 2.58 |
| C-27 | 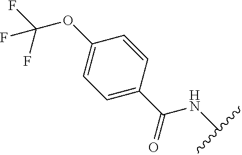 [SMILES: *NC(=O)c1ccc(OC(F)(F)F)cc1] | 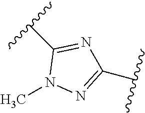 [SMILES: *c1nc(*)n(C)n1] | 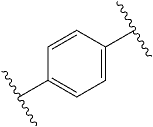 [SMILES: *c1ccc(*)cc1] | 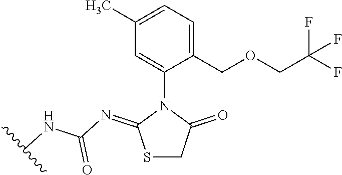 [SMILES: *NC(=O)/N=C1\SCC(=O)N1c1cc(C)ccc1COCC(F)(F)F] | 720 (Method 1) | 2.43 |
| C-28 | 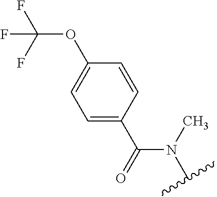 [SMILES: *N(C)C(=O)c1ccc(OC(F)(F)F)cc1] | 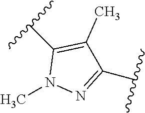 [SMILES: *c1nn(C)c(*)c1C] | 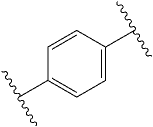 [SMILES: *c1ccc(*)cc1] | 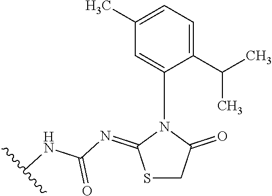 [SMILES: *NC(=O)/N=C1\SCC(=O)N1c1cc(C)ccc1C(C)C] | 679 (Method 1) | 1.35 |
| C-29 | 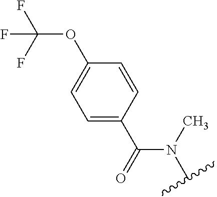 [SMILES: *N(C)C(=O)c1ccc(OC(F)(F)F)cc1] | 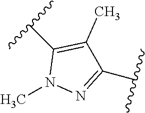 [SMILES: *c1nn(C)c(*)c1C] | 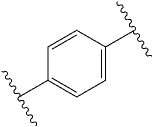 [SMILES: *c1ccc(*)cc1] | 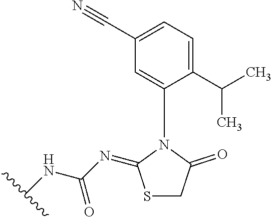 [SMILES: *NC(=O)/N=C1\SCC(=O)N1c1cc(C#N)ccc1C(C)C] | 690.3 (Method 2) | 1.29 |
| C-30 | 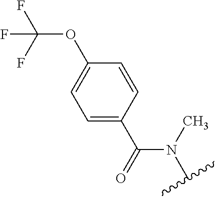 [SMILES: *N(C)C(=O)c1ccc(OC(F)(F)F)cc1] | 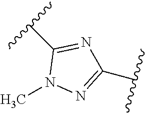 [SMILES: *c1nc(*)n(C)n1] | 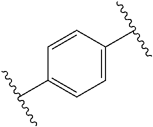 [SMILES: *c1ccc(*)cc1] | 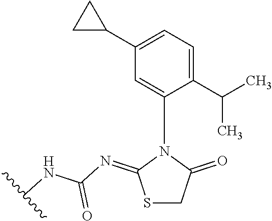 [SMILES: *NC(=O)/N=C1\SCC(=O)N1c1cc(C2CC2)ccc1C(C)C] | 693 (Method 1) | 2.57 |
| C-31 | 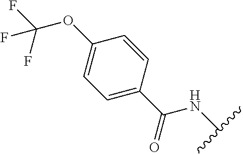 [SMILES: *NC(=O)c1ccc(OC(F)(F)F)cc1] | 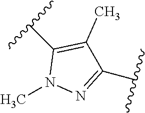 [SMILES: *c1nn(C)c(*)c1C] | 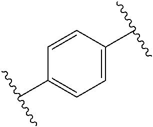 [SMILES: *c1ccc(*)cc1] | 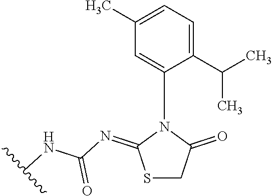 [SMILES: *NC(=O)/N=C1\SCC(=O)N1c1cc(C)ccc1C(C)C] | 663 (Method 1) | 2.52 |
| C-32 | 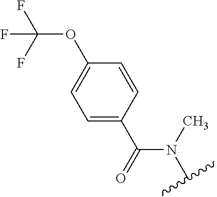 [SMILES: *N(C)C(=O)c1ccc(OC(F)(F)F)cc1] | 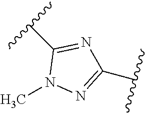 [SMILES: *c1nc(*)n(C)n1] | 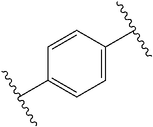 [SMILES: *c1ccc(*)cc1] | 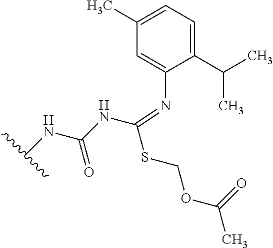 [SMILES: *NC(=O)N/C(=N\c1cc(C)ccc1C(C)C)SCOC(C)=O] | 699 (Method 1) | 2.77 |
| C-33 | 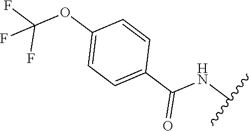 [SMILES: *NC(=O)c1ccc(OC(F)(F)F)cc1] | 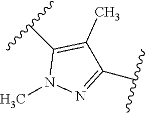 [SMILES: *c1nn(C)c(*)c1C] | 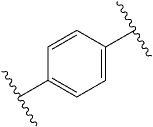 [SMILES: *c1ccc(*)cc1] | 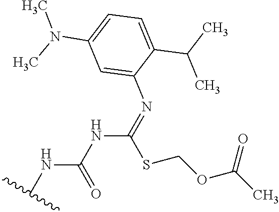 [SMILES: *NC(=O)N/C(=N\c1cc(N(C)C)ccc1C(C)C)SCOC(C)=O] | 724 (Method 1) | 2.76 |
| C-34 | 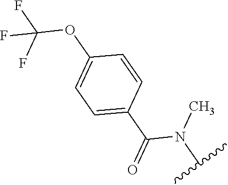 [SMILES: *N(C)C(=O)c1ccc(OC(F)(F)F)cc1] | 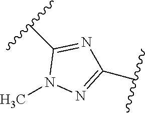 [SMILES: *c1nc(*)n(C)n1] | 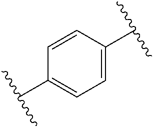 [SMILES: *c1ccc(*)cc1] | 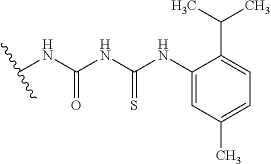 [SMILES: *NC(=O)NC(=S)Nc1cc(C)ccc1C(C)C] | 624 (Method 1) | 2.72 |
| C-35 | 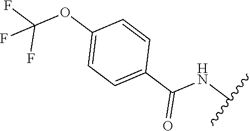 [SMILES: *NC(=O)c1ccc(OC(F)(F)F)cc1] | 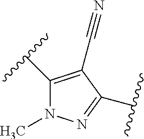 [SMILES: *c1nn(C)c(*)c1C#N] | 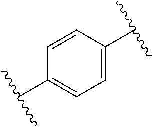 [SMILES: *c1ccc(*)cc1] | 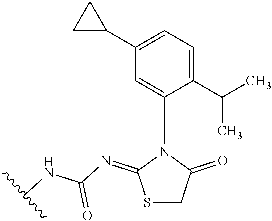 [SMILES: *NC(=O)/N=C1\SCC(=O)N1c1cc(C2CC2)ccc1C(C)C] | 702 (Method 1) | 2.59 |
| C-36 | 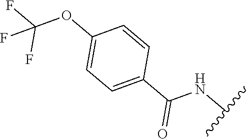 [SMILES: *NC(=O)c1ccc(OC(F)(F)F)cc1] | 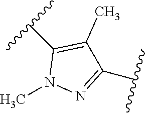 [SMILES: *c1nn(C)c(*)c1C] | 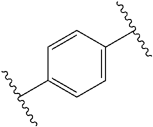 [SMILES: *c1ccc(*)cc1] | 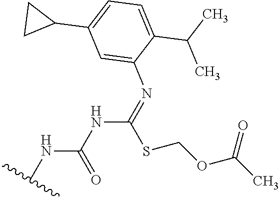 [SMILES: *NC(=O)N/C(=N\c1cc(C2CC2)ccc1C(C)C)SCOC(C)=O] | 721 (Method 1) | 2.80 |
| C-37 | 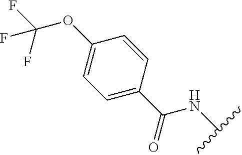 [SMILES: *NC(=O)c1ccc(OC(F)(F)F)cc1] | 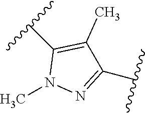 [SMILES: *c1nn(C)c(*)c1C] | 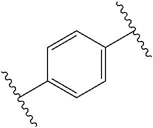 [SMILES: *c1ccc(*)cc1] | 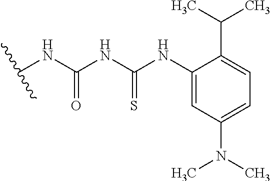 [SMILES: *NC(=O)NC(=S)Nc1cc(N(C)C)ccc1C(C)C] | 653 (Method 1) | 1.41 |
| C-38 | 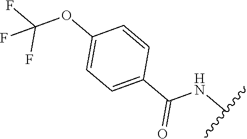 [SMILES: *NC(=O)c1ccc(OC(F)(F)F)cc1] | 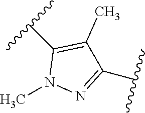 [SMILES: *c1nn(C)c(*)c1C] | 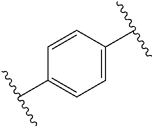 [SMILES: *c1ccc(*)cc1] | 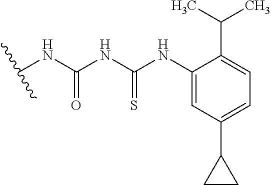 [SMILES: *NC(=O)NC(=S)Nc1cc(C2CC2)ccc1C(C)C] | 649 (Method 1) | 2.77 |
| C-39 | 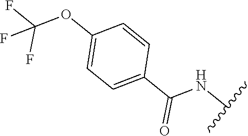 [SMILES: *NC(=O)c1ccc(OC(F)(F)F)cc1] | 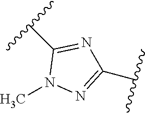 [SMILES: *c1nc(*)n(C)n1] | 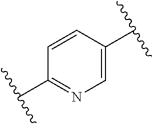 [SMILES: *c1ccc(*)nc1] | 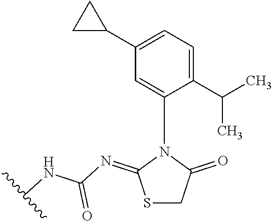 [SMILES: *NC(=O)/N=C1\SCC(=O)N1c1cc(C2CC2)ccc1C(C)C] | 677 (Method 1) | 2.45 |
| C-40 | 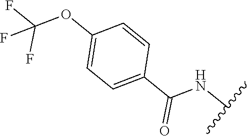 [SMILES: *NC(=O)c1ccc(OC(F)(F)F)cc1] | 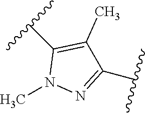 [SMILES: *c1nn(C)c(*)c1C] | 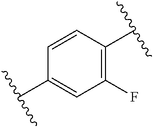 [SMILES: *c1ccc(*)c(F)c1] | 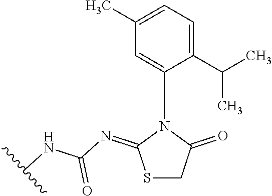 [SMILES: *NC(=O)/N=C1\SCC(=O)N1c1cc(C)ccc1C(C)C] | 681 (Method 1) | 2.54 |
| C-41 | 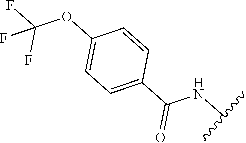 [SMILES: *NC(=O)c1ccc(OC(F)(F)F)cc1] | 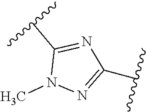 [SMILES: *c1nc(*)n(C)n1] | 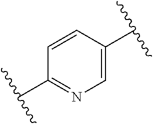 [SMILES: *c1ccc(*)nc1] | 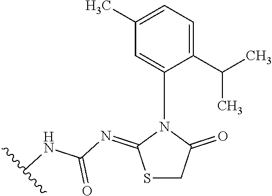 [SMILES: *NC(=O)/N=C1\SCC(=O)N1c1cc(C)ccc1C(C)C] | 651 (Method 1) | 0.83 |
| C-42 | 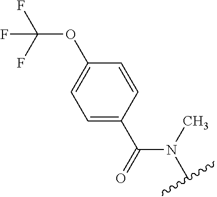 [SMILES: *N(C)C(=O)c1ccc(OC(F)(F)F)cc1] | 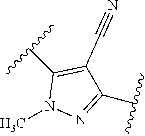 [SMILES: *c1nn(C)c(*)c1C#N] | 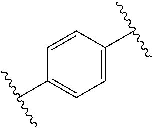 [SMILES: *c1ccc(*)cc1] | 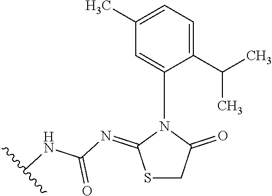 [SMILES: *NC(=O)/N=C1\SCC(=O)N1c1cc(C)ccc1C(C)C] | 690 (Method 1) | 2.21 |
| C-43 | 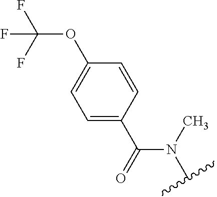 [SMILES: *N(C)C(=O)c1ccc(OC(F)(F)F)cc1] | 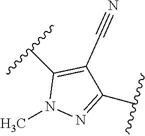 [SMILES: *c1nn(C)c(*)c1C#N] | 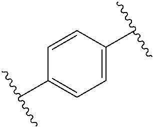 [SMILES: *c1ccc(*)cc1] | 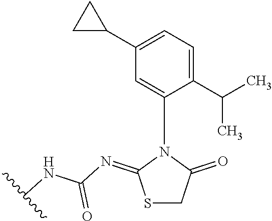 [SMILES: *NC(=O)/N=C1\SCC(=O)N1c1cc(C2CC2)ccc1C(C)C] | 716 (Method 1) | 2.23 |
| C-44 | 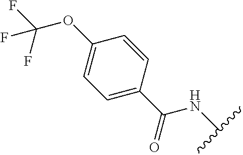 [SMILES: *NC(=O)c1ccc(OC(F)(F)F)cc1] | 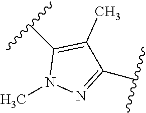 [SMILES: *c1nn(C)c(*)c1C] | 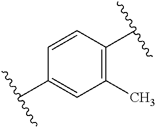 [SMILES: *c1ccc(*)c(C)c1] | 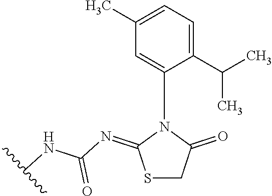 [SMILES: *NC(=O)/N=C1\SCC(=O)N1c1cc(C)ccc1C(C)C] | 679.53 (Method 1) | 2.18 |
| C-45 | 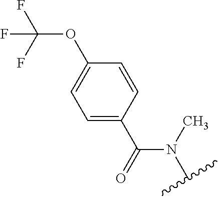 [SMILES: *N(C)C(=O)c1ccc(OC(F)(F)F)cc1] | 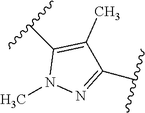 [SMILES: *c1nn(C)c(*)c1C] | 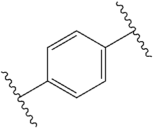 [SMILES: *c1ccc(*)cc1] | 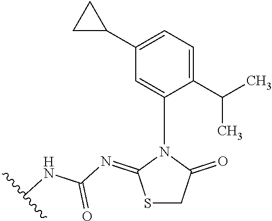 [SMILES: *NC(=O)/N=C1\SCC(=O)N1c1cc(C2CC2)ccc1C(C)C] | 705 (Method 1) | 2.61 |
| C-46 | 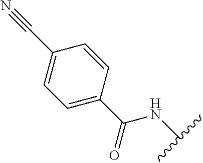 [SMILES: *NC(=O)c1ccc(C#N)cc1] | 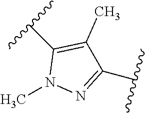 [SMILES: *c1nn(C)c(*)c1C] | 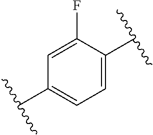 [SMILES: *c1ccc(*)c(F)c1] | 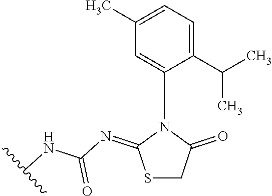 [SMILES: *NC(=O)/N=C1\SCC(=O)N1c1cc(C)ccc1C(C)C] | 624 (Method 1) | 2.07 |
| C-47 | 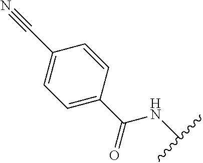 [SMILES: *NC(=O)c1ccc(C#N)cc1] | 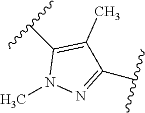 [SMILES: *c1nn(C)c(*)c1C] | 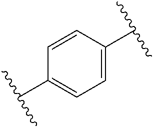 [SMILES: *c1ccc(*)cc1] | 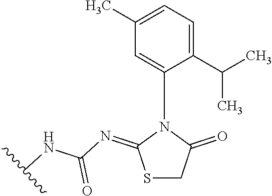 [SMILES: *NC(=O)/N=C1\SCC(=O)N1c1cc(C)ccc1C(C)C] | 606 (Method 1) | 2.27 |
| C-48 | 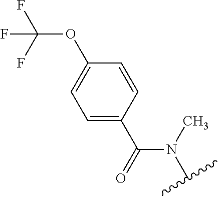 [SMILES: *N(C)C(=O)c1ccc(OC(F)(F)F)cc1] | 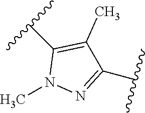 [SMILES: *c1nn(C)c(*)c1C] | 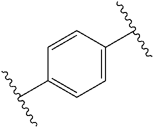 [SMILES: *c1ccc(*)cc1] | 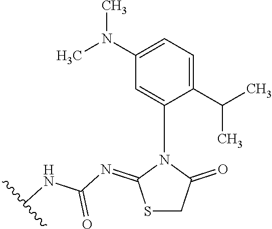 [SMILES: *NC(=O)/N=C1\SCC(=O)N1c1cc(N(C)C)ccc1C(C)C] | 709 (Method 1) | 2.53 |
| C-49 | 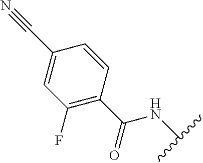 [SMILES: *NC(=O)c1ccc(C#N)cc1F] | 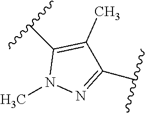 [SMILES: *c1nn(C)c(*)c1C] | 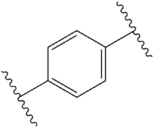 [SMILES: *c1ccc(*)cc1] | 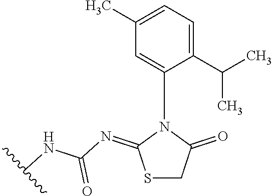 [SMILES: *NC(=O)/N=C1\SCC(=O)N1c1cc(C)ccc1C(C)C] | 624 (Method 1) | 2.28 |
| C-50 | 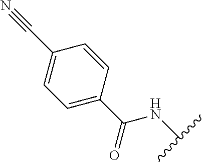 [SMILES: *NC(=O)c1ccc(C#N)cc1] | 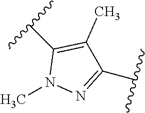 [SMILES: *c1nn(C)c(*)c1C] | 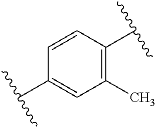 [SMILES: *c1ccc(*)c(C)c1] | 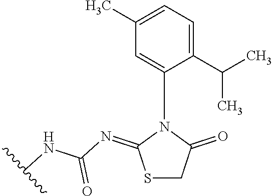 [SMILES: *NC(=O)/N=C1\SCC(=O)N1c1cc(C)ccc1C(C)C] | 620 (Method 1) | 2.26 |
| C-51 | 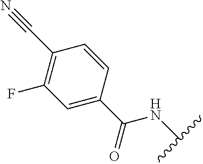 [SMILES: *NC(=O)c1ccc(C#N)c(F)c1] | 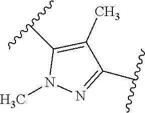 [SMILES: *c1nn(C)c(*)c1C] | 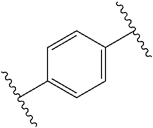 [SMILES: *c1ccc(*)cc1] | 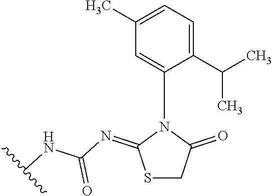 [SMILES: *NC(=O)/N=C1\SCC(=O)N1c1cc(C)ccc1C(C)C] | 622 (Method 1) | 2.32 |
| C-52 | 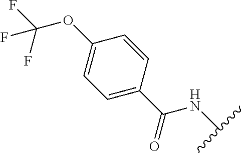 [SMILES: *NC(=O)c1ccc(OC(F)(F)F)cc1] | 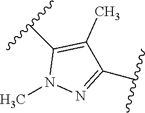 [SMILES: *c1nn(C)c(*)c1C] | 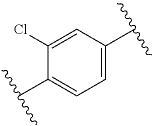 [SMILES: *c1ccc(*)c(Cl)c1] | 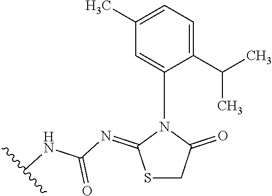 [SMILES: *NC(=O)/N=C1\SCC(=O)N1c1cc(C)ccc1C(C)C] | 696.9 (Method 1) | 2.59 |
| C-53 | 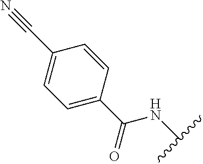 [SMILES: *NC(=O)c1ccc(C#N)cc1] | 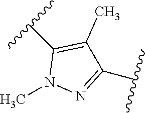 [SMILES: *c1nn(C)c(*)c1C] | 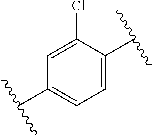 [SMILES: *c1ccc(*)c(Cl)c1] | 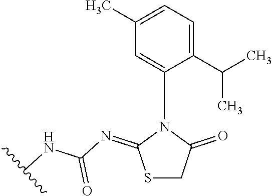 [SMILES: *NC(=O)/N=C1\SCC(=O)N1c1cc(C)ccc1C(C)C] | 640 (Method 1) | 2.13 |
| C-54 | 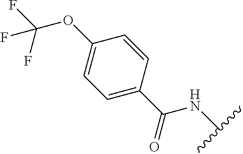 [SMILES: *NC(=O)c1ccc(OC(F)(F)F)cc1] | 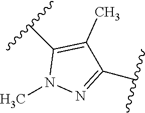 [SMILES: *c1nn(C)c(*)c1C] | 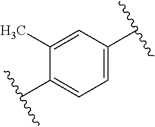 [SMILES: *c1ccc(*)c(C)c1] | 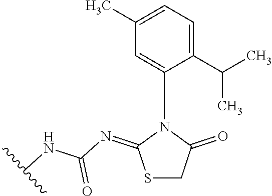 [SMILES: *NC(=O)/N=C1\SCC(=O)N1c1cc(C)ccc1C(C)C] | 679 (Method 1) | 2.2 |
| C-55 | 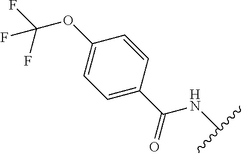 [SMILES: *NC(=O)c1ccc(OC(F)(F)F)cc1] | 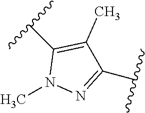 [SMILES: *c1nn(C)c(*)c1C] | 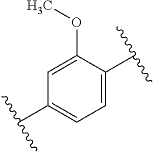 [SMILES: *c1ccc(*)c(OC)c1] | 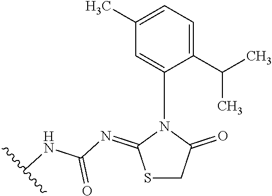 [SMILES: *NC(=O)/N=C1\SCC(=O)N1c1cc(C)ccc1C(C)C] | 695 (Method 1) | 2.22 |
| C-56 | 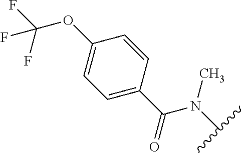 [SMILES: *N(C)C(=O)c1ccc(OC(F)(F)F)cc1] | 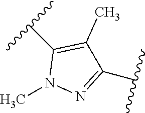 [SMILES: *c1nn(C)c(*)c1C] | 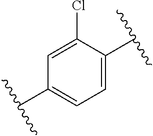 [SMILES: *c1ccc(*)c(Cl)c1] | 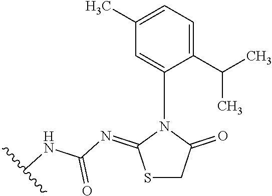 [SMILES: *NC(=O)/N=C1\SCC(=O)N1c1cc(C)ccc1C(C)C] | 713 (Method 1) | 2.31 |
| C-57 | 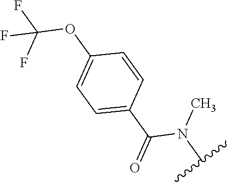 [SMILES: *N(C)C(=O)c1ccc(OC(F)(F)F)cc1] | 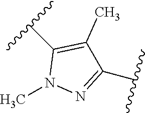 [SMILES: *c1nn(C)c(*)c1C] | 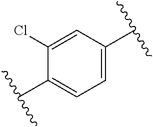 [SMILES: *c1ccc(*)c(Cl)c1] | 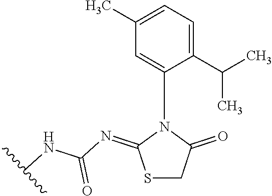 [SMILES: *NC(=O)/N=C1\SCC(=O)N1c1cc(C)ccc1C(C)C] | 713 (Method 1) | 2.24 |
| C-58 | 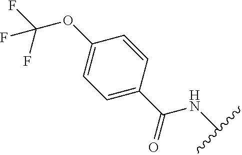 [SMILES: *NC(=O)c1ccc(OC(F)(F)F)cc1] | 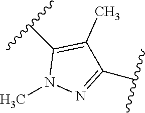 [SMILES: *c1nn(C)c(*)c1C] | 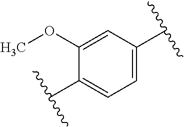 [SMILES: *c1ccc(*)c(OC)c1] | 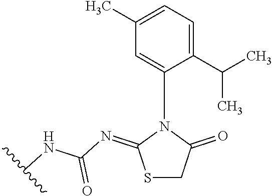 [SMILES: *NC(=O)/N=C1\SCC(=O)N1c1cc(C)ccc1C(C)C] | 695 (Method 1) | 2.18 |
| C-59 | 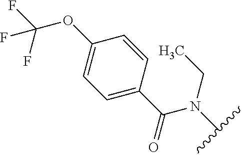 [SMILES: *N(CC)C(=O)c1ccc(OC(F)(F)F)cc1] | 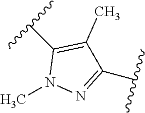 [SMILES: *c1nn(C)c(*)c1C] | 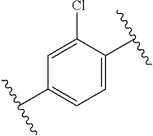 [SMILES: *c1ccc(*)c(Cl)c1] | 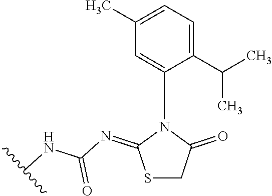 [SMILES: *NC(=O)/N=C1\SCC(=O)N1c1cc(C)ccc1C(C)C] | 727.3 (Method 1) | 2.36 |
| C-60 | 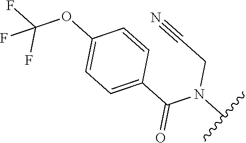 [SMILES: *N(CC#N)C(=O)c1ccc(OC(F)(F)F)cc1] | 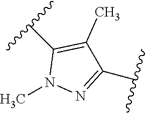 [SMILES: *c1nn(C)c(*)c1C] | 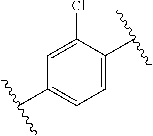 [SMILES: *c1ccc(*)c(Cl)c1] | 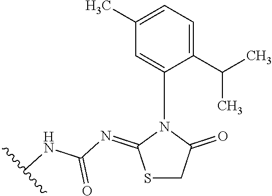 [SMILES: *NC(=O)/N=C1\SCC(=O)N1c1cc(C)ccc1C(C)C] | 738.3 (Method 1) | 2.28 |
| C-61 | 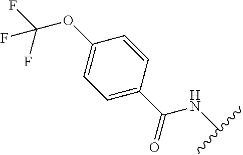 [SMILES: *NC(=O)c1ccc(OC(F)(F)F)cc1] | 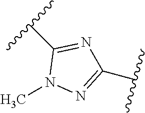 [SMILES: *c1nc(*)n(C)n1] | 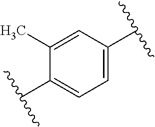 [SMILES: *c1ccc(*)c(C)c1] | 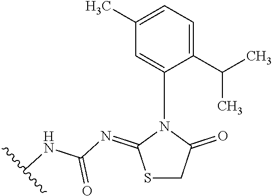 [SMILES: *NC(=O)/N=C1\SCC(=O)N1c1cc(C)ccc1C(C)C] | 666.3 (Method 1) | 2.17 |
| C-62 | 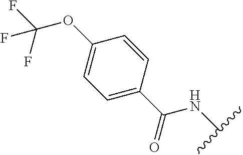 [SMILES: *NC(=O)c1ccc(OC(F)(F)F)cc1] | 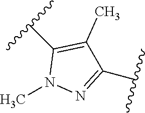 [SMILES: *c1nn(C)c(*)c1C] | 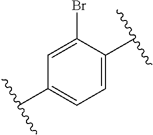 [SMILES: *c1ccc(*)c(Br)c1] | 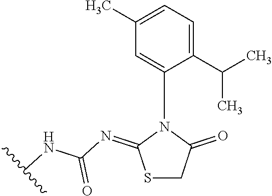 [SMILES: *NC(=O)/N=C1\SCC(=O)N1c1cc(C)ccc1C(C)C] | 745.2 (Method 1) | 2.29 |
| C-63 | 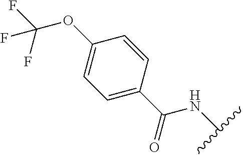 [SMILES: *NC(=O)c1ccc(OC(F)(F)F)cc1] | 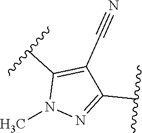 [SMILES: *c1nn(C)c(*)c1C#N] | 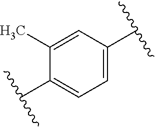 [SMILES: *c1ccc(*)c(C)c1] | 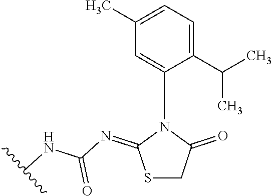 [SMILES: *NC(=O)/N=C1\SCC(=O)N1c1cc(C)ccc1C(C)C] | 690.2 (Method 1) | 2.17 |
| C-64 | 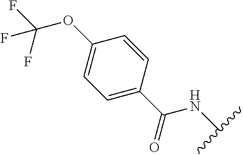 [SMILES: *NC(=O)c1ccc(OC(F)(F)F)cc1] | 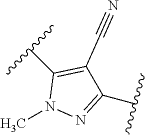 [SMILES: *c1nn(C)c(*)c1C#N] | 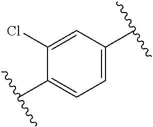 [SMILES: *c1ccc(*)c(Cl)c1] | 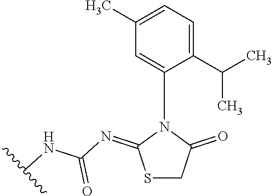 [SMILES: *NC(=O)/N=C1\SCC(=O)N1c1cc(C)ccc1C(C)C] | 710 (Method 1) | 2.21 |
| C-65 | 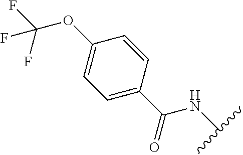 [SMILES: *NC(=O)c1ccc(OC(F)(F)F)cc1] | 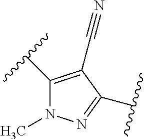 [SMILES: *c1nn(C)c(*)c1C#N] | 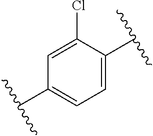 [SMILES: *c1ccc(*)c(Cl)c1] | 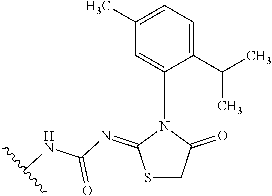 [SMILES: *NC(=O)/N=C1\SCC(=O)N1c1cc(C)ccc1C(C)C] | 710 (Method 1) | 2.3 |
| C-66 | 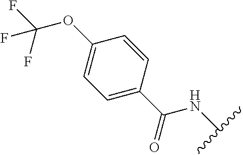 [SMILES: *NC(=O)c1ccc(OC(F)(F)F)cc1] | 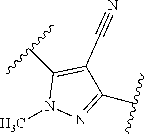 [SMILES: *c1nn(C)c(*)c1C#N] | 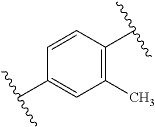 [SMILES: *c1ccc(*)c(C)c1] | 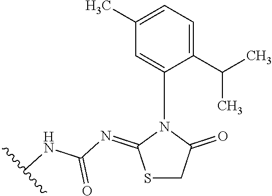 [SMILES: *NC(=O)/N=C1\SCC(=O)N1c1cc(C)ccc1C(C)C] | 690.3 (Method 1) | 2.21 |
| C-67 | 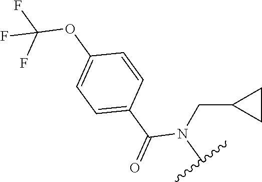 [SMILES: *N(CC1CC1)C(=O)c1ccc(OC(F)(F)F)cc1] | 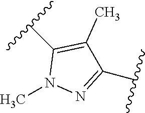 [SMILES: *c1nn(C)c(*)c1C] | 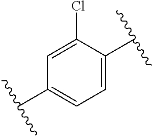 [SMILES: *c1ccc(*)c(Cl)c1] | 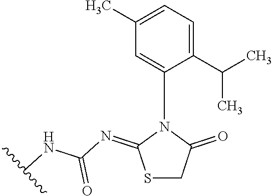 [SMILES: *NC(=O)/N=C1\SCC(=O)N1c1cc(C)ccc1C(C)C] | 753.4 (Method 1) | 2.4 |
| C-68 | 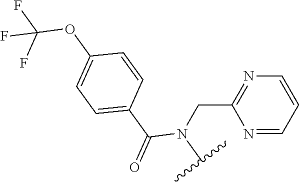 [SMILES: *N(Cc1ncccn1)C(=O)c1ccc(OC(F)(F)F)cc1] | 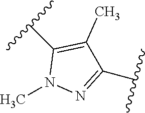 [SMILES: *c1nn(C)c(*)c1C] | 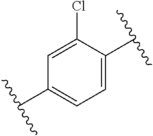 [SMILES: *c1ccc(*)c(Cl)c1] | 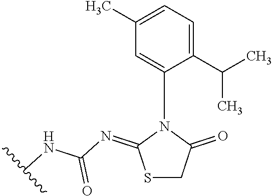 [SMILES: *NC(=O)/N=C1\SCC(=O)N1c1cc(C)ccc1C(C)C] | 791.4 (Method 1) | 2.29 |
| C-69 | 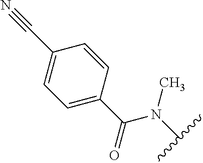 [SMILES: *N(C)C(=O)c1ccc(C#N)cc1] | 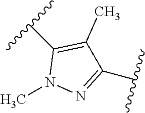 [SMILES: *c1nn(C)c(*)c1C] | 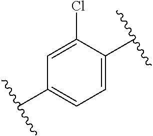 [SMILES: *c1ccc(*)c(Cl)c1] | 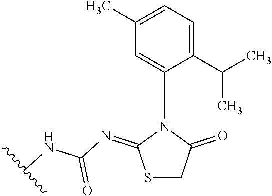 [SMILES: *NC(=O)/N=C1\SCC(=O)N1c1cc(C)ccc1C(C)C] | 654 (Method 1) | 2.26 |
| C-70 | 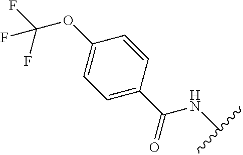 [SMILES: *NC(=O)c1ccc(OC(F)(F)F)cc1] | 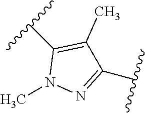 [SMILES: *c1nn(C)c(*)c1C] | 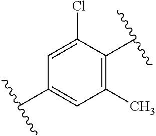 [SMILES: *c1cc(C)c(*)c(Cl)c1] | 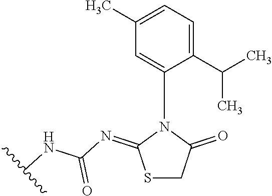 [SMILES: *NC(=O)/N=C1\SCC(=O)N1c1cc(C)ccc1C(C)C] | 713 (Method 1) | 2.21 |
| C-71 | 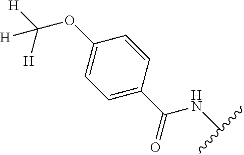 [SMILES: *NC(=O)c1ccc(OC)cc1] | 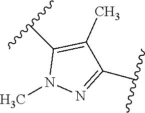 [SMILES: *c1nn(C)c(*)c1C] | 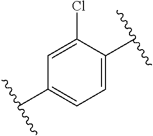 [SMILES: *c1ccc(*)c(Cl)c1] | 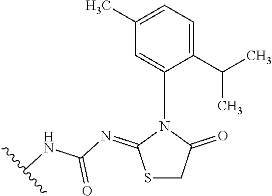 [SMILES: *NC(=O)/N=C1\SCC(=O)N1c1cc(C)ccc1C(C)C] | 645.5 (Method 1) | 2.22 |
| C-72 | 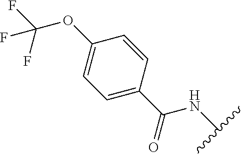 [SMILES: *NC(=O)c1ccc(OC(F)(F)F)cc1] | 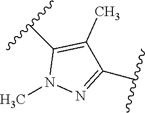 [SMILES: *c1nn(C)c(*)c1C] | 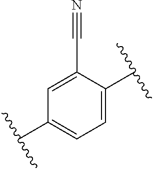 [SMILES: *c1ccc(*)c(C#N)c1] | 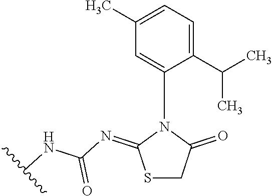 [SMILES: *NC(=O)/N=C1\SCC(=O)N1c1cc(C)ccc1C(C)C] | 690 (Method 1) | 2.19 |
| C-73 | 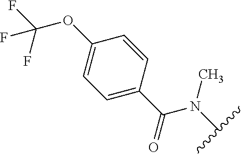 [SMILES: *N(C)C(=O)c1ccc(OC(F)(F)F)cc1] | 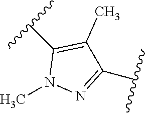 [SMILES: *c1nn(C)c(*)c1C] | 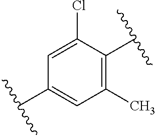 [SMILES: *c1cc(C)c(*)c(Cl)c1] | 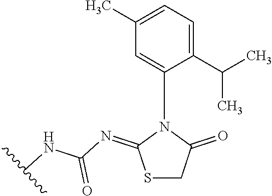 [SMILES: *NC(=O)/N=C1\SCC(=O)N1c1cc(C)ccc1C(C)C] | 727 (Method 1) | 2.22 |
| C-74 | 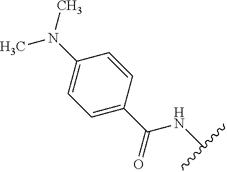 [SMILES: *NC(=O)c1ccc(N(C)C)cc1] | 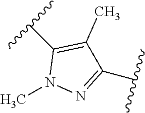 [SMILES: *c1nn(C)c(*)c1C] | 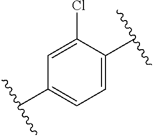 [SMILES: *c1ccc(*)c(Cl)c1] | 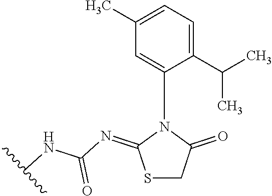 [SMILES: *NC(=O)/N=C1\SCC(=O)N1c1cc(C)ccc1C(C)C] | 658 (Method 1) | 2.16 |
| C-75 | 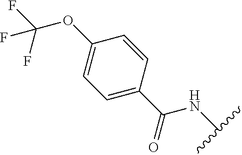 [SMILES: *NC(=O)c1ccc(OC(F)(F)F)cc1] | 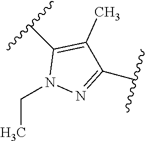 [SMILES: *c1nn(CC)c(*)c1C] | 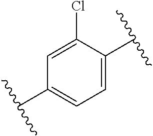 [SMILES: *c1ccc(*)c(Cl)c1] | 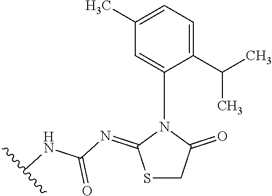 [SMILES: *NC(=O)/N=C1\SCC(=O)N1c1cc(C)ccc1C(C)C] | 713.4 (Method 1) | 2.4 |
| C-76 | 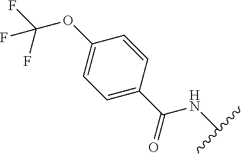 [SMILES: *NC(=O)c1ccc(OC(F)(F)F)cc1] | 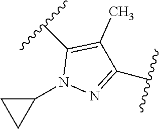 [SMILES: *c1nn(C2CC2)c(*)c1C] | 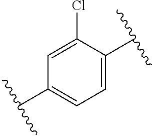 [SMILES: *c1ccc(*)c(Cl)c1] | 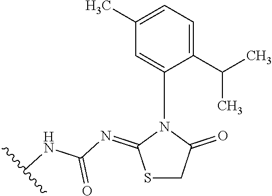 [SMILES: *NC(=O)/N=C1\SCC(=O)N1c1cc(C)ccc1C(C)C] | 725 (Method 1) | 2.34 |
| C-77 | 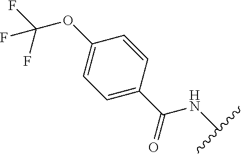 [SMILES: *NC(=O)c1ccc(OC(F)(F)F)cc1] | 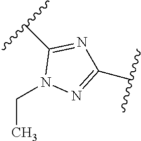 [SMILES: *c1nc(*)n(CC)n1] | 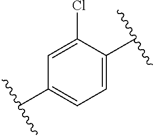 [SMILES: *c1ccc(*)c(Cl)c1] | 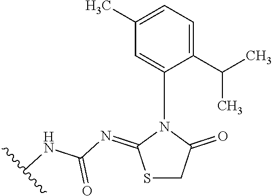 [SMILES: *NC(=O)/N=C1\SCC(=O)N1c1cc(C)ccc1C(C)C] | 700.4 (Method 1) | 2.41 |
| C-78 | 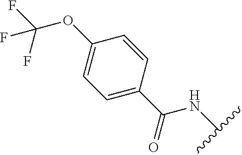 [SMILES: *NC(=O)c1ccc(OC(F)(F)F)cc1] | 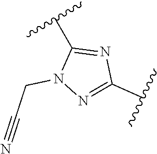 [SMILES: *c1nc(*)n(CC#N)n1] | 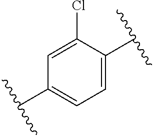 [SMILES: *c1ccc(*)c(Cl)c1] | 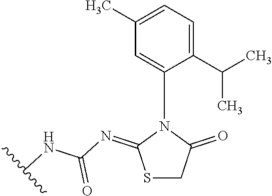 [SMILES: *NC(=O)/N=C1\SCC(=O)N1c1cc(C)ccc1C(C)C] | 711.4 (Method 1) | 2.33 |
| C-79 | 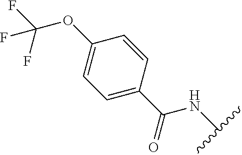 [SMILES: *NC(=O)c1ccc(OC(F)(F)F)cc1] | 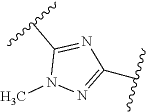 [SMILES: *c1nc(*)n(C)n1] | 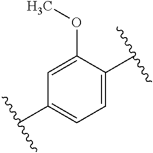 [SMILES: *c1ccc(*)c(OC)c1] | 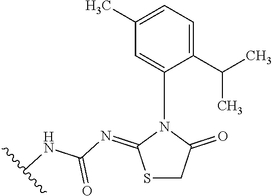 [SMILES: *NC(=O)/N=C1\SCC(=O)N1c1cc(C)ccc1C(C)C] | 682.2 (Method 1) | 2.18 |
| C-80 | 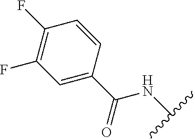 [SMILES: *NC(=O)c1ccc(F)c(F)c1] | 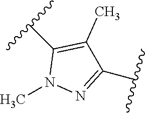 [SMILES: *c1nn(C)c(*)c1C] | 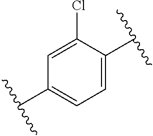 [SMILES: *c1ccc(*)c(Cl)c1] | 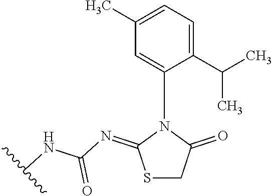 [SMILES: *NC(=O)/N=C1\SCC(=O)N1c1cc(C)ccc1C(C)C] | 651 (Method 1) | 2.2 |
| C-81 | 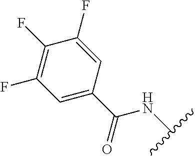 [SMILES: *NC(=O)c1cc(F)c(F)c(F)c1] | 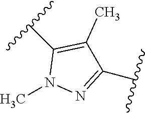 [SMILES: *c1nn(C)c(*)c1C] | 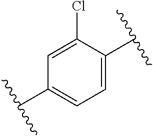 [SMILES: *c1ccc(*)c(Cl)c1] | 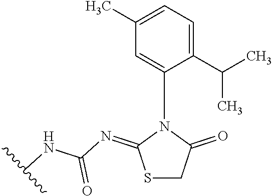 [SMILES: *NC(=O)/N=C1\SCC(=O)N1c1cc(C)ccc1C(C)C] | 669 (Method 1) | 2.26 |
| C-82 | 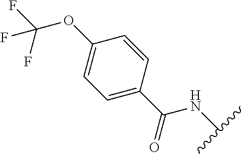 [SMILES: *NC(=O)c1ccc(OC(F)(F)F)cc1] | 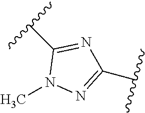 [SMILES: *c1nc(*)n(C)n1] | 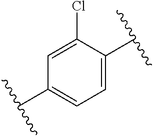 [SMILES: *c1ccc(*)c(Cl)c1] | 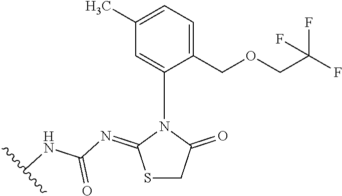 [SMILES: *NC(=O)/N=C1\SCC(=O)N1c1cc(C)ccc1COCC(F)(F)F] | 756.3 (Method 1) | 2.26 |
| C-83 | 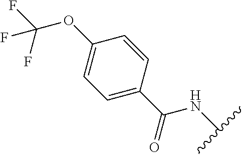 [SMILES: *NC(=O)c1ccc(OC(F)(F)F)cc1] | 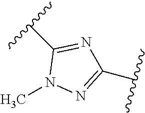 [SMILES: *c1nc(*)n(C)n1] | 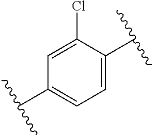 [SMILES: *c1ccc(*)c(Cl)c1] | 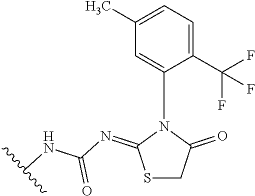 [SMILES: *NC(=O)/N=C1\SCC(=O)N1c1cc(C)ccc1C(F)(F)F] | 712.3 (Method 1) | 2.25 |
| C-84 | 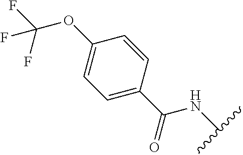 [SMILES: *NC(=O)c1ccc(OC(F)(F)F)cc1] | 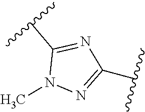 [SMILES: *c1nc(*)n(C)n1] | 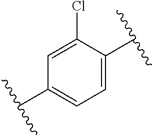 [SMILES: *c1ccc(*)c(Cl)c1] | 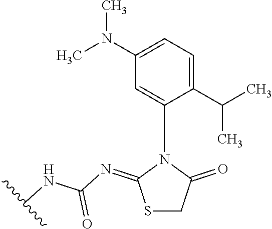 [SMILES: *NC(=O)/N=C1\SCC(=O)N1c1cc(N(C)C)ccc1C(C)C] | 715.4 (Method 1) | 2.33 |
| C-85 | 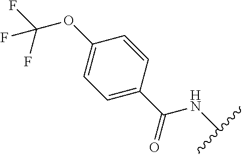 [SMILES: *NC(=O)c1ccc(OC(F)(F)F)cc1] | 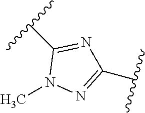 [SMILES: *c1nc(*)n(C)n1] | 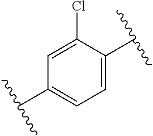 [SMILES: *c1ccc(*)c(Cl)c1] | 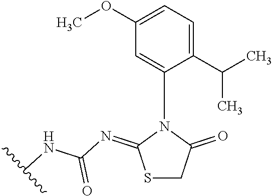 [SMILES: *NC(=O)/N=C1\SCC(=O)N1c1cc(OC)ccc1C(C)C] | 702.4 (Method 1) | 2.27 |
| C-86 | 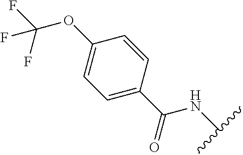 [SMILES: *NC(=O)c1ccc(OC(F)(F)F)cc1] | 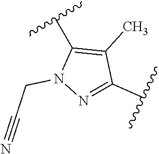 [SMILES: *c1nn(CC#N)c(*)c1C] | 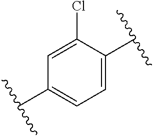 [SMILES: *c1ccc(*)c(Cl)c1] | 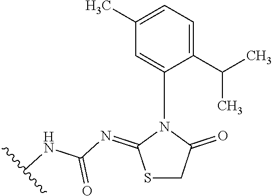 [SMILES: *NC(=O)/N=C1\SCC(=O)N1c1cc(C)ccc1C(C)C] | 724.4 (Method 1) | 2.33 |
| C-87 | 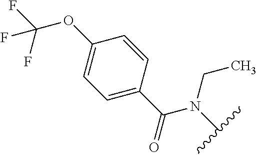 [SMILES: *N(CC)C(=O)c1ccc(OC(F)(F)F)cc1] | 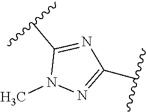 [SMILES: *c1nc(*)n(C)n1] | 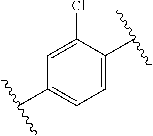 [SMILES: *c1ccc(*)c(Cl)c1] | 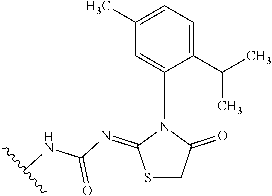 [SMILES: *NC(=O)/N=C1\SCC(=O)N1c1cc(C)ccc1C(C)C] | 714.4 (Method 1) | 2.45 |
| C-88 | 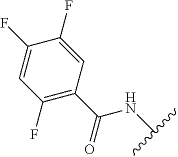 [SMILES: *NC(=O)c1cc(F)c(F)cc1F] | 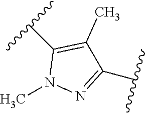 [SMILES: *c1nn(C)c(*)c1C] | 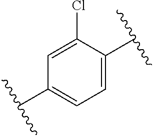 [SMILES: *c1ccc(*)c(Cl)c1] | 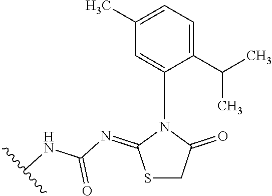 [SMILES: *NC(=O)/N=C1\SCC(=O)N1c1cc(C)ccc1C(C)C] | 669 (Method 1) | 2.23 |
| C-89 | 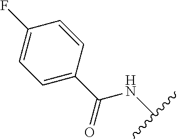 [SMILES: *NC(=O)c1ccc(F)cc1] | 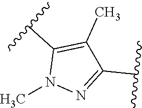 [SMILES: *c1nn(C)c(*)c1C] | 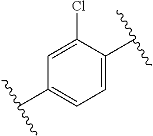 [SMILES: *c1ccc(*)c(Cl)c1] | 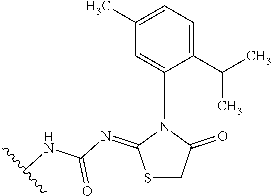 [SMILES: *NC(=O)/N=C1\SCC(=O)N1c1cc(C)ccc1C(C)C] | 633 (Method 1) | 2.18 |
| C-90 | 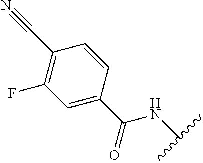 [SMILES: *NC(=O)c1ccc(C#N)c(F)c1] | 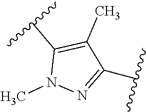 [SMILES: *c1nn(C)c(*)c1C] | 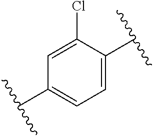 [SMILES: *c1ccc(*)c(Cl)c1] | 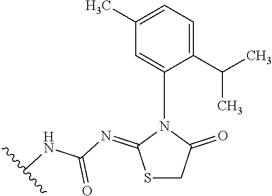 [SMILES: *NC(=O)/N=C1\SCC(=O)N1c1cc(C)ccc1C(C)C] | 658 (Method 1) | 2.17 |
| C-91 | 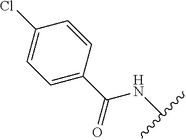 [SMILES: *NC(=O)c1ccc(Cl)cc1] | 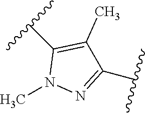 [SMILES: *c1nn(C)c(*)c1C] | 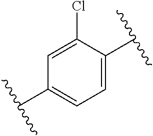 [SMILES: *c1ccc(*)c(Cl)c1] | 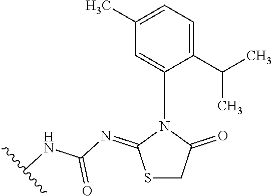 [SMILES: *NC(=O)/N=C1\SCC(=O)N1c1cc(C)ccc1C(C)C] | 649 (Method 1) | 2.23 |
| C-92 | 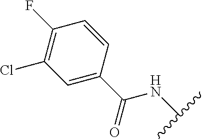 [SMILES: *NC(=O)c1ccc(F)c(Cl)c1] | 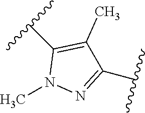 [SMILES: *c1nn(C)c(*)c1C] | 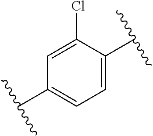 [SMILES: *c1ccc(*)c(Cl)c1] | 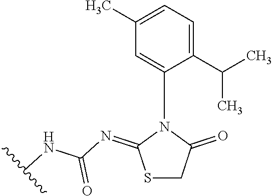 [SMILES: *NC(=O)/N=C1\SCC(=O)N1c1cc(C)ccc1C(C)C] | 667 (Method 1) | 2.26 |
| C-93 | 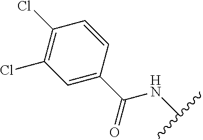 [SMILES: *NC(=O)c1ccc(Cl)c(Cl)c1] | 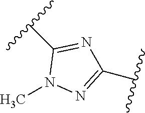 [SMILES: *c1nc(*)n(C)n1] | 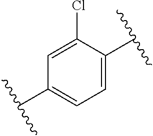 [SMILES: *c1ccc(*)c(Cl)c1] | 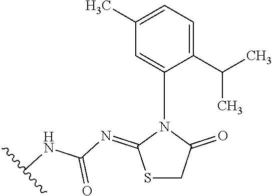 [SMILES: *NC(=O)/N=C1\SCC(=O)N1c1cc(C)ccc1C(C)C] | 672 (Method 1) | 1.40 |
| C-94 | 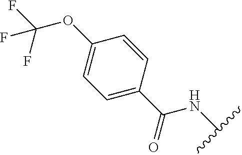 [SMILES: *NC(=O)c1ccc(OC(F)(F)F)cc1] | 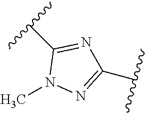 [SMILES: *c1nc(*)n(C)n1] | 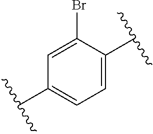 [SMILES: *c1ccc(*)c(Br)c1] | 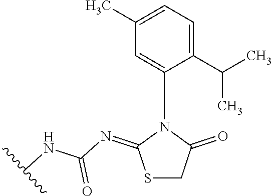 [SMILES: *NC(=O)/N=C1\SCC(=O)N1c1cc(C)ccc1C(C)C] | 732.2 (Method 1) | 2.26 |
| C-95 | 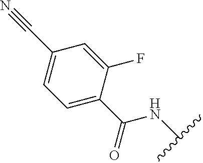 [SMILES: *NC(=O)c1ccc(C#N)cc1F] | 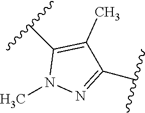 [SMILES: *c1nn(C)c(*)c1C] | 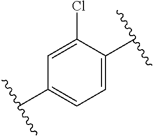 [SMILES: *c1ccc(*)c(Cl)c1] | 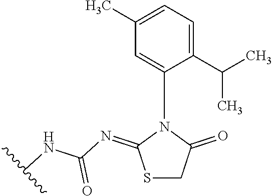 [SMILES: *NC(=O)/N=C1\SCC(=O)N1c1cc(C)ccc1C(C)C] | 658 (Method 1) | 2.13 |
| C-96 | 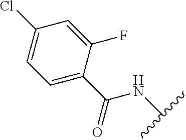 [SMILES: *NC(=O)c1ccc(Cl)cc1F] | 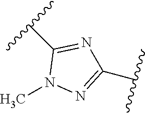 [SMILES: *c1nc(*)n(C)n1] | 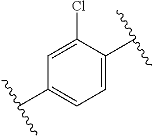 [SMILES: *c1ccc(*)c(Cl)c1] | 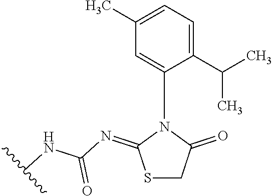 [SMILES: *NC(=O)/N=C1\SCC(=O)N1c1cc(C)ccc1C(C)C] | 654.2 (Method 2) | 1.34 |
| C-97 | 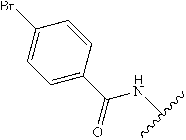 [SMILES: *NC(=O)c1ccc(Br)cc1] | 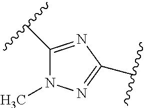 [SMILES: *c1nc(*)n(C)n1] | 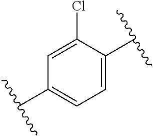 [SMILES: *c1ccc(*)c(Cl)c1] | 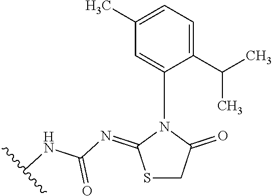 [SMILES: *NC(=O)/N=C1\SCC(=O)N1c1cc(C)ccc1C(C)C] | 682 (Method 2) | 1.35 |
| C-98 | 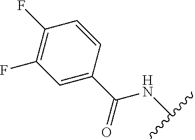 [SMILES: *NC(=O)c1ccc(F)c(F)c1] | 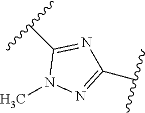 [SMILES: *c1nc(*)n(C)n1] | 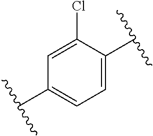 [SMILES: *c1ccc(*)c(Cl)c1] | 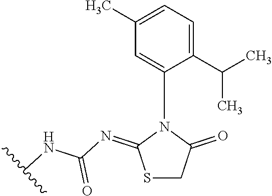 [SMILES: *NC(=O)/N=C1\SCC(=O)N1c1cc(C)ccc1C(C)C] | 638.1 (Method 2) | 1.32 |
| C-99 | 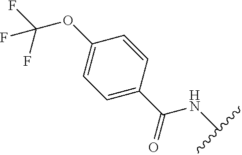 [SMILES: *NC(=O)c1ccc(OC(F)(F)F)cc1] | 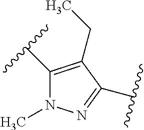 [SMILES: *c1nn(C)c(*)c1CC] | 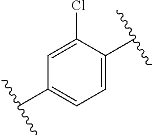 [SMILES: *c1ccc(*)c(Cl)c1] | 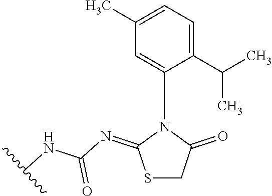 [SMILES: *NC(=O)/N=C1\SCC(=O)N1c1cc(C)ccc1C(C)C] | 713.4 (Method 1) | 2.39 |
| C-100 | 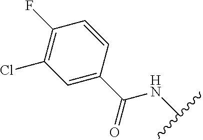 [SMILES: *NC(=O)c1ccc(F)c(Cl)c1] | 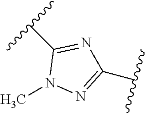 [SMILES: *c1nc(*)n(C)n1] | 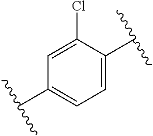 [SMILES: *c1ccc(*)c(Cl)c1] | 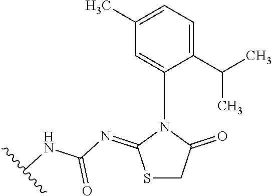 [SMILES: *NC(=O)/N=C1\SCC(=O)N1c1cc(C)ccc1C(C)C] | 654.1 (Method 2) | 1.36 |
| C-101 | 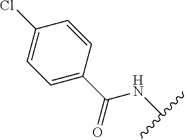 [SMILES: *NC(=O)c1ccc(Cl)cc1] | 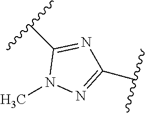 [SMILES: *c1nc(*)n(C)n1] | 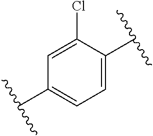 [SMILES: *c1ccc(*)c(Cl)c1] | 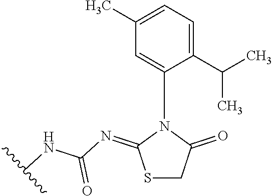 [SMILES: *NC(=O)/N=C1\SCC(=O)N1c1cc(C)ccc1C(C)C] | 636.2 (Method 2) | 1.34 |
| C-102 | 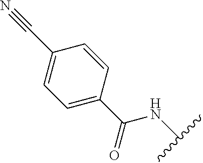 [SMILES: *NC(=O)c1ccc(C#N)cc1] | 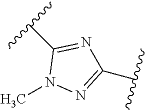 [SMILES: *c1nc(*)n(C)n1] | 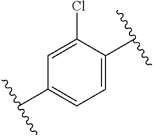 [SMILES: *c1ccc(*)c(Cl)c1] | 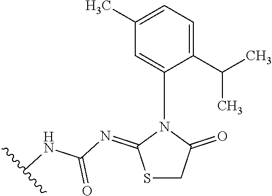 [SMILES: *NC(=O)/N=C1\SCC(=O)N1c1cc(C)ccc1C(C)C] | 627.1 (Method 2) | 1.24 |
| C-103 | 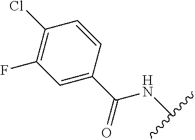 [SMILES: *NC(=O)c1ccc(Cl)c(F)c1] | 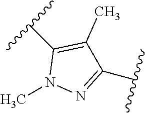 [SMILES: *c1nn(C)c(*)c1C] | 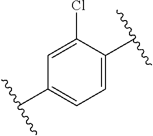 [SMILES: *c1ccc(*)c(Cl)c1] | 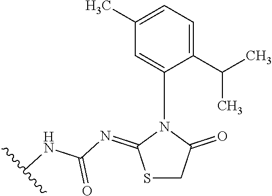 [SMILES: *NC(=O)/N=C1\SCC(=O)N1c1cc(C)ccc1C(C)C] | 667 (Method 1) | 2.25 |
| C-104 | 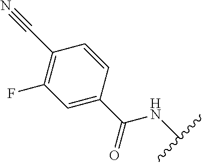 [SMILES: *NC(=O)c1ccc(C#N)c(F)c1] | 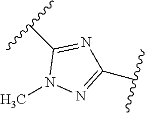 [SMILES: *c1nc(*)n(C)n1] | 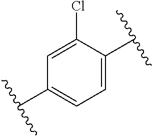 [SMILES: *c1ccc(*)c(Cl)c1] | 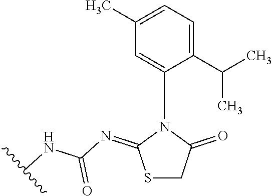 [SMILES: *NC(=O)/N=C1\SCC(=O)N1c1cc(C)ccc1C(C)C] | 645 (Method 2) | 1.3 |
| C-105 | 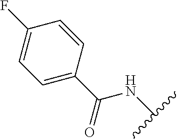 [SMILES: *NC(=O)c1ccc(F)cc1] | 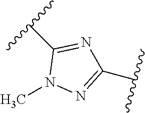 [SMILES: *c1nc(*)n(C)n1] | 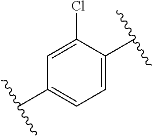 [SMILES: *c1ccc(*)c(Cl)c1] | 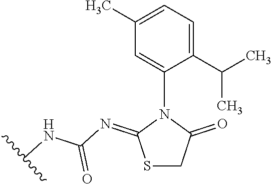 [SMILES: *NC(=O)/N=C1\SCC(=O)N1c1cc(C)ccc1C(C)C] | 620.1 (Method 2) | 1.29 |
| C-106 | 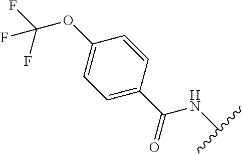 [SMILES: *NC(=O)c1ccc(OC(F)(F)F)cc1] | 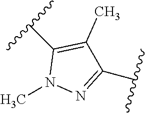 [SMILES: *c1nn(C)c(*)c1C] | 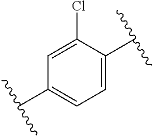 [SMILES: *c1ccc(*)c(Cl)c1] | 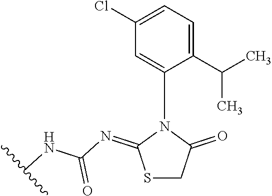 [SMILES: *NC(=O)/N=C1\SCC(=O)N1c1cc(Cl)ccc1C(C)C] | 719 (Method 2) | 1.38 |
| C-107 | 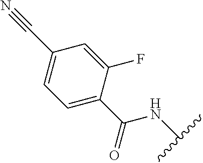 [SMILES: *NC(=O)c1ccc(C#N)cc1F] | 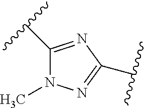 [SMILES: *c1nc(*)n(C)n1] | 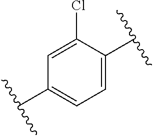 [SMILES: *c1ccc(*)c(Cl)c1] | 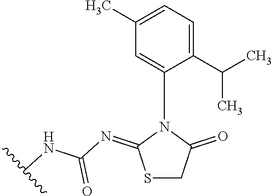 [SMILES: *NC(=O)/N=C1\SCC(=O)N1c1cc(C)ccc1C(C)C] | 645.1 (Method 2) | 1.24 |
| C-108 | 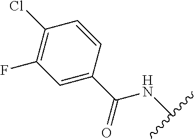 [SMILES: *NC(=O)c1ccc(Cl)c(F)c1] | 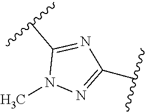 [SMILES: *c1nc(*)n(C)n1] | 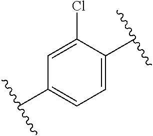 [SMILES: *c1ccc(*)c(Cl)c1] | 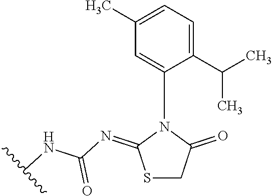 [SMILES: *NC(=O)/N=C1\SCC(=O)N1c1cc(C)ccc1C(C)C] | 654 (Method 2) | 1.37 |
| C-109 | 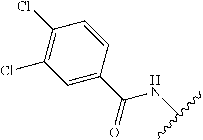 [SMILES: *NC(=O)c1ccc(Cl)c(Cl)c1] | 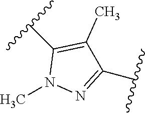 [SMILES: *c1nn(C)c(*)c1C] | 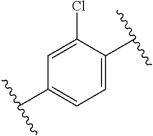 [SMILES: *c1ccc(*)c(Cl)c1] | 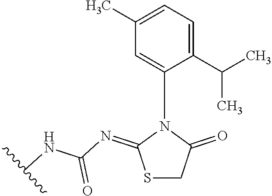 [SMILES: *NC(=O)/N=C1\SCC(=O)N1c1cc(C)ccc1C(C)C] | 685 (Method 1) | 2.32 |
| C-110 | 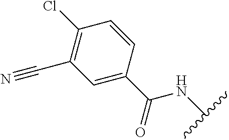 [SMILES: *NC(=O)c1ccc(Cl)c(C#N)c1] | 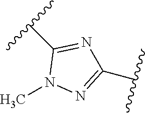 [SMILES: *c1nc(*)n(C)n1] | 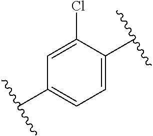 [SMILES: *c1ccc(*)c(Cl)c1] | 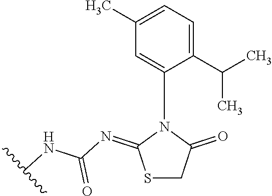 [SMILES: *NC(=O)/N=C1\SCC(=O)N1c1cc(C)ccc1C(C)C] | 661 (Method 2) | 1.32 |
| C-111 | 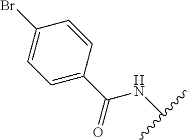 [SMILES: *NC(=O)c1ccc(Br)cc1] | 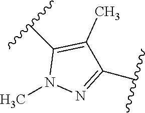 [SMILES: *c1nn(C)c(*)c1C] | 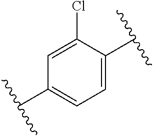 [SMILES: *c1ccc(*)c(Cl)c1] | 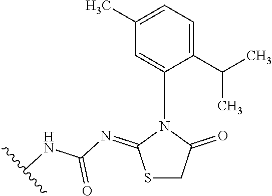 [SMILES: *NC(=O)/N=C1\SCC(=O)N1c1cc(C)ccc1C(C)C] | 695 (Method 1) | 2.3 |
| C-112 | 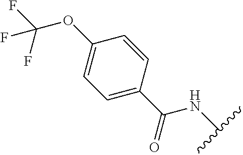 [SMILES: *NC(=O)c1ccc(OC(F)(F)F)cc1] | 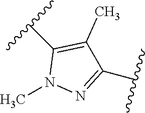 [SMILES: *c1nn(C)c(*)c1C] | 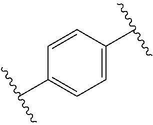 [SMILES: *c1ccc(*)cc1] | 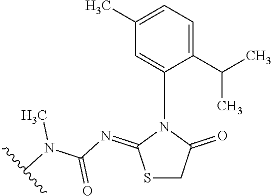 [SMILES: *N(C)C(=O)/N=C1\SCC(=O)N1c1cc(C)ccc1C(C)C] | 679.3 (Method 1) | 2.22 |
| C-113 | 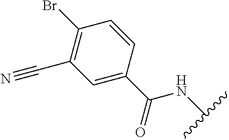 [SMILES: *NC(=O)c1ccc(Br)c(C#N)c1] | 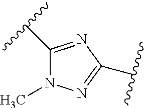 [SMILES: *c1nc(*)n(C)n1] | 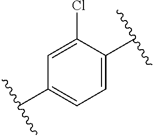 [SMILES: *c1ccc(*)c(Cl)c1] | 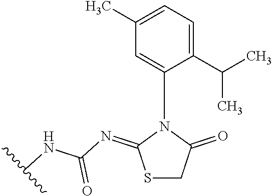 [SMILES: *NC(=O)/N=C1\SCC(=O)N1c1cc(C)ccc1C(C)C] | 707 (Method 2) | 1.35 |
| C-114 | 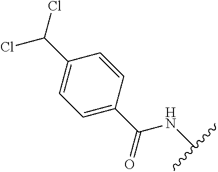 [SMILES: *NC(=O)c1ccc(C(Cl)Cl)cc1] | 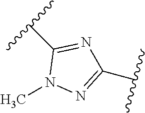 [SMILES: *c1nc(*)n(C)n1] | 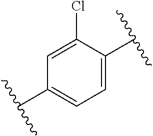 [SMILES: *c1ccc(*)c(Cl)c1] | 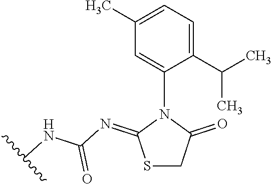 [SMILES: *NC(=O)/N=C1\SCC(=O)N1c1cc(C)ccc1C(C)C] | 685.8 (Method 2) | 1.36 |
| C-115 | 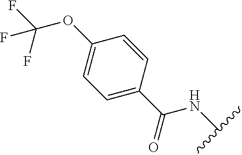 [SMILES: *NC(=O)c1ccc(OC(F)(F)F)cc1] | 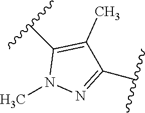 [SMILES: *c1nn(C)c(*)c1C] | 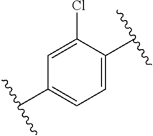 [SMILES: *c1ccc(*)c(Cl)c1] | 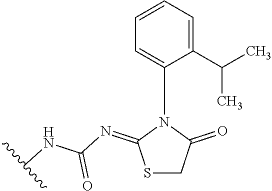 [SMILES: *NC(=O)/N=C1\SCC(=O)N1c1ccccc1C(C)C] | 685.1 (Method 2) | 1.35 |
| C-116 | 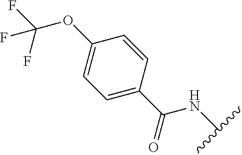 [SMILES: *NC(=O)c1ccc(OC(F)(F)F)cc1] | 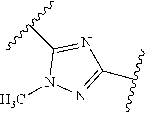 [SMILES: *c1nc(*)n(C)n1] | 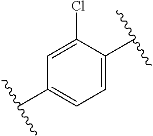 [SMILES: *c1ccc(*)c(Cl)c1] | 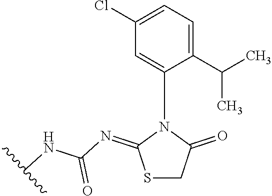 [SMILES: *NC(=O)/N=C1\SCC(=O)N1c1cc(Cl)ccc1C(C)C] | 706 (Method 2) | 1.38 |
| C-117 | 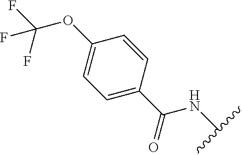 [SMILES: *NC(=O)c1ccc(OC(F)(F)F)cc1] | 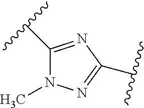 [SMILES: *c1nc(*)n(C)n1] | 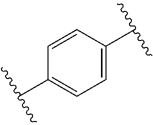 [SMILES: *c1ccc(*)cc1] | 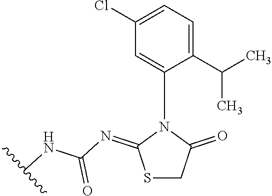 [SMILES: *NC(=O)/N=C1\SCC(=O)N1c1cc(Cl)ccc1C(C)C] | 672 (Method 2) | 1.30 |
| C-118 | 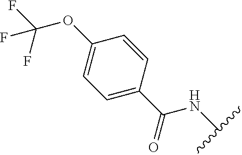 [SMILES: *NC(=O)c1ccc(OC(F)(F)F)cc1] | 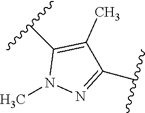 [SMILES: *c1nn(C)c(*)c1C] | 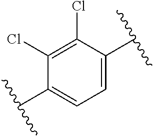 [SMILES: *c1ccc(*)c(Cl)c1Cl] | 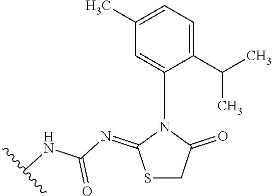 [SMILES: *NC(=O)/N=C1\SCC(=O)N1c1cc(C)ccc1C(C)C] | 733 (Method 1) | 2.33 |
| C-119 | 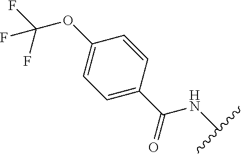 [SMILES: *NC(=O)c1ccc(OC(F)(F)F)cc1] | 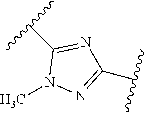 [SMILES: *c1nc(*)n(C)n1] | 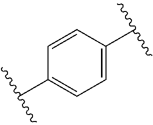 [SMILES: *c1ccc(*)cc1] | 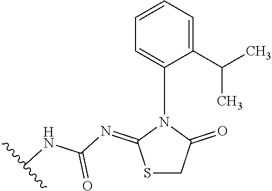 [SMILES: *NC(=O)/N=C1\SCC(=O)N1c1ccccc1C(C)C] | 638.1 (Method 2) | 1.26 |
| C-120 | 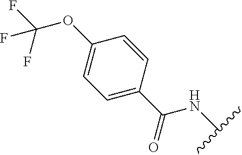 [SMILES: *NC(=O)c1ccc(OC(F)(F)F)cc1] | 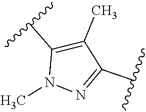 [SMILES: *c1nn(C)c(*)c1C] | 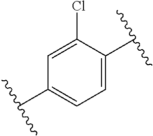 [SMILES: *c1ccc(*)c(Cl)c1] | 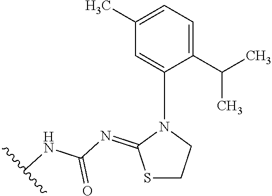 [SMILES: *NC(=O)/N=C1\SCCN1c1cc(C)ccc1C(C)C] | 685.2 (Method 1) | 2.38 |
| C-121 | 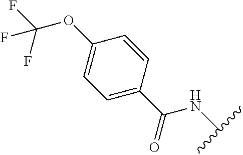 [SMILES: *NC(=O)c1ccc(OC(F)(F)F)cc1] | 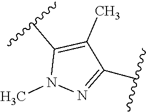 [SMILES: *c1nn(C)c(*)c1C] | 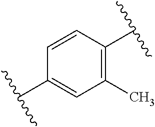 [SMILES: *c1ccc(*)c(C)c1] | 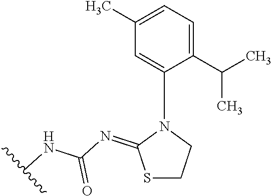 [SMILES: *NC(=O)/N=C1\SCCN1c1cc(C)ccc1C(C)C] | 665.3 (Method 1) | 2.26 |
| C-122 | 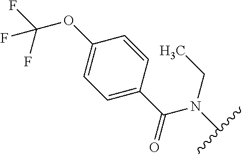 [SMILES: *N(CC)C(=O)c1ccc(OC(F)(F)F)cc1] | 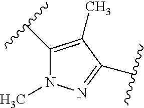 [SMILES: *c1nn(C)c(*)c1C] | 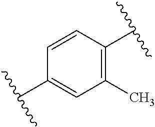 [SMILES: *c1ccc(*)c(C)c1] | 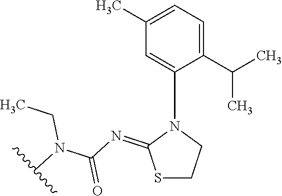 [SMILES: *N(CC)C(=O)/N=C1\SCCN1c1cc(C)ccc1C(C)C] | 721.4 (Method 1) | 2.47 |
| C-123 | 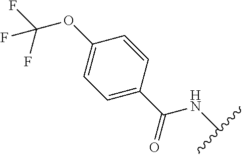 [SMILES: *NC(=O)c1ccc(OC(F)(F)F)cc1] | 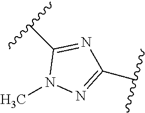 [SMILES: *c1nc(*)n(C)n1] | 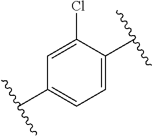 [SMILES: *c1ccc(*)c(Cl)c1] | 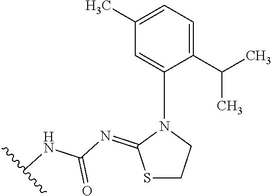 [SMILES: *NC(=O)/N=C1\SCCN1c1cc(C)ccc1C(C)C] | 672.3 (Method 1) | 2.36 |
| C-124 | 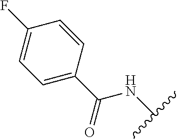 [SMILES: *NC(=O)c1ccc(F)cc1] | 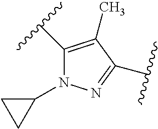 [SMILES: *c1nn(C2CC2)c(*)c1C] | 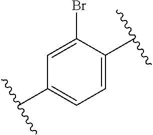 [SMILES: *c1ccc(*)c(Br)c1] | 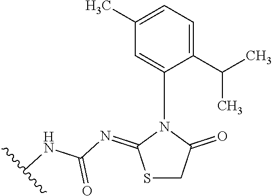 [SMILES: *NC(=O)/N=C1\SCC(=O)N1c1cc(C)ccc1C(C)C] | 702.9 (Method 3) | 1.29 |
| C-125 | 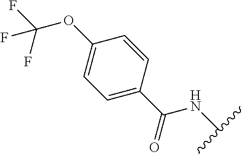 [SMILES: *NC(=O)c1ccc(OC(F)(F)F)cc1] | 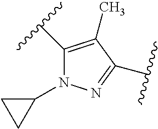 [SMILES: *c1nn(C2CC2)c(*)c1C] | 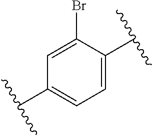 [SMILES: *c1ccc(*)c(Br)c1] | 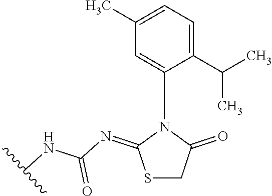 [SMILES: *NC(=O)/N=C1\SCC(=O)N1c1cc(C)ccc1C(C)C] | 771 (Method 1) | 2.36 |
| C-126 | 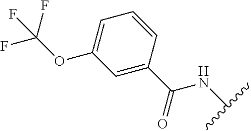 [SMILES: *NC(=O)c1cccc(OC(F)(F)F)c1] | 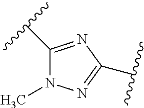 [SMILES: *c1nc(*)n(C)n1] | 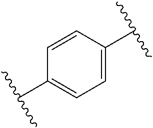 [SMILES: *c1ccc(*)cc1] | 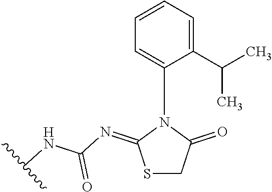 [SMILES: *NC(=O)/N=C1\SCC(=O)N1c1ccccc1C(C)C] | 638 (Method 2) | 1.28 |
| C-127 | 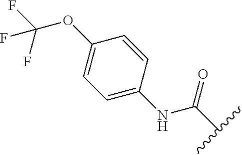 [SMILES: *C(=O)Nc1ccc(OC(F)(F)F)cc1] | 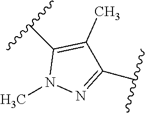 [SMILES: *c1nn(C)c(*)c1C] | 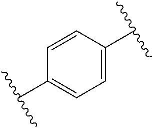 [SMILES: *c1ccc(*)cc1] | 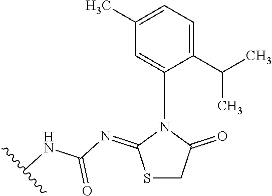 [SMILES: *NC(=O)/N=C1\SCC(=O)N1c1cc(C)ccc1C(C)C] | 665.3 (Method 1) | 2.29 |
| C-128 | 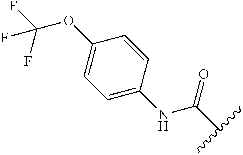 [SMILES: *C(=O)Nc1ccc(OC(F)(F)F)cc1] | 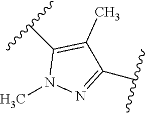 [SMILES: *c1nn(C)c(*)c1C] | 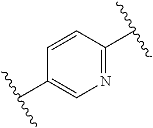 [SMILES: *c1ccc(*)nc1] | 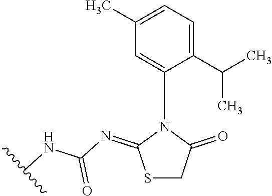 [SMILES: *NC(=O)/N=C1\SCC(=O)N1c1cc(C)ccc1C(C)C] | 666 (Method 1) | 2.14 |
| C-129 | 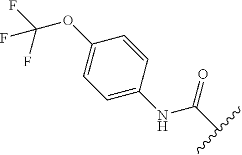 [SMILES: *C(=O)Nc1ccc(OC(F)(F)F)cc1] | 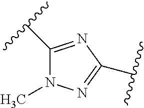 [SMILES: *c1nc(*)n(C)n1] | 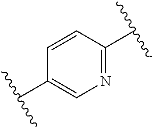 [SMILES: *c1ccc(*)nc1] | 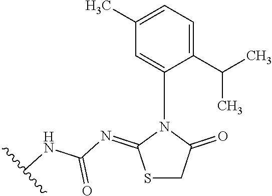 [SMILES: *NC(=O)/N=C1\SCC(=O)N1c1cc(C)ccc1C(C)C] | 653.3 (Method 1) | 2.11 |
| C-130 | 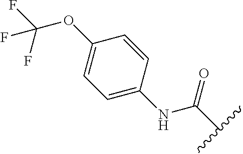 [SMILES: *C(=O)Nc1ccc(OC(F)(F)F)cc1] | 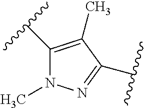 [SMILES: *c1nn(C)c(*)c1C] | 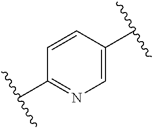 [SMILES: *c1ccc(*)nc1] | 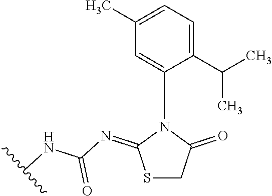 [SMILES: *NC(=O)/N=C1\SCC(=O)N1c1cc(C)ccc1C(C)C] | 666.3 (Method 1) | 2.11 |
| C-131 | 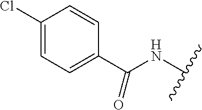 [SMILES: *NC(=O)c1ccc(Cl)cc1] | 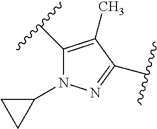 [SMILES: *c1nn(C2CC2)c(*)c1C] | 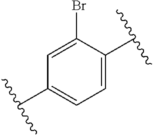 [SMILES: *c1ccc(*)c(Br)c1] | 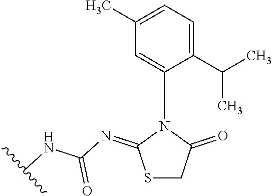 [SMILES: *NC(=O)/N=C1\SCC(=O)N1c1cc(C)ccc1C(C)C] | 721 (Method 1) | 2.32 |
| C-132 | 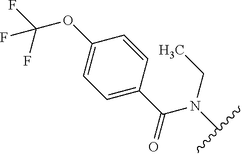 [SMILES: *N(CC)C(=O)c1ccc(OC(F)(F)F)cc1] | 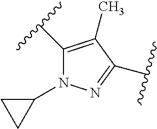 [SMILES: *c1nn(C2CC2)c(*)c1C] | 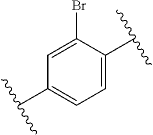 [SMILES: *c1ccc(*)c(Br)c1] | 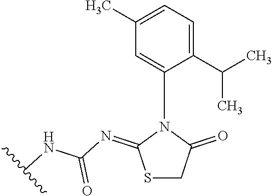 [SMILES: *NC(=O)/N=C1\SCC(=O)N1c1cc(C)ccc1C(C)C] | 799.1 (Method 3) | 1.28 |
| C-133 | 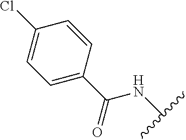 [SMILES: *NC(=O)c1ccc(Cl)cc1] | 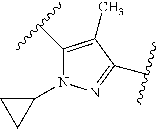 [SMILES: *c1nn(C2CC2)c(*)c1C] | 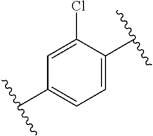 [SMILES: *c1ccc(*)c(Cl)c1] | 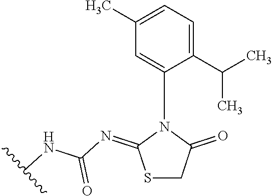 [SMILES: *NC(=O)/N=C1\SCC(=O)N1c1cc(C)ccc1C(C)C] | 675 (Method 1) | 2.29 |
| C-134 | 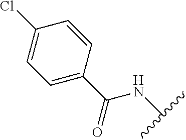 [SMILES: *NC(=O)c1ccc(Cl)cc1] | 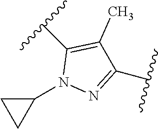 [SMILES: *c1nn(C2CC2)c(*)c1C] | 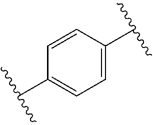 [SMILES: *c1ccc(*)cc1] | 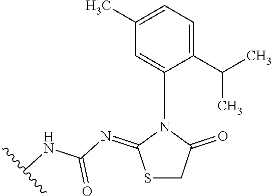 [SMILES: *NC(=O)/N=C1\SCC(=O)N1c1cc(C)ccc1C(C)C] | 641 (Method 1) | 2.18 |

### Biological Examples

### Example B.1: Action on Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds or mixtures were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

For experimental mixtures in these tests identical volumes of both mixing partners at the desired concentrations respectively, were mixed together.

After application, microtiter plates were incubated at 28 #1° C., 80 #5% RH for 2 days. Larval mortality was then visually assessed.

In this test, compounds C-1, C-2, C-3, C-4, C-6, C-7, C-8, C-9, C-10, C-11, C-12, C-13, C-14, C-15, C-16, C-17, C-18, C-21, C-22, C-23, C-24, C-25, C-27, C-28, C-29, C-30, C-31, C-32, C-33, C-34, C-35, C-36 C-37, C-38, C-40, C-42, C-43, C-44, C-45, C-46, C-47, C-48, C-49, C-50, C-51, C-52, C-53, C-54, C-55, C-56, C-57, C-58, C-59, C-60, C-61, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C-100, C-101, C-102, C-103, C-104, C-105, C-106, C-107, C-108, C-109, C-111, C-112, C-113, C-114, C-115, C-116, C-118, C-120, C-121, C-122, C-123, C-126, C-127, C-128, and C-130, resp., at 800 ppm showed at least 50% mortality in comparison with untreated controls.

### Example B.2: Action on Orchid Thrips (Dichromothrips corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic® HV at a rate of 0.01% v/v.

Thrips potency of each compound was evaluated by using a floral-immersion technique. All petals of individual, intact orchid flowers were dipped into treatment solution and allowed to dryin Petri dishes. Treated petals were placed into individual re-sealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-24, C-25, C-26, C-27, C-28, C-30, C-31, C-34, C-35, C-36, C-37, C-38, C-40, C-42, C-43, C-44, C-45, C-48, C-49, C-55, C-56, C-59, C-60, C-61, C-62, C-63, C-65, C-66, C-67, C-68, C-71, C-72, C-75, C-76, C-77, C-78, C-79, C-80, C-81, C-83, C-84, C-85, C-86, C-87, C-88, C-90, C-92, C-93, C-94, C-96, C-97, C-98, C-99, C-100, C-101, C-102, C-103, C-104, C-105, C-106, C-108, C-109, C-110, C-113, C-115, C-116, C-118, C-120, C-121, C-129, and C-130, resp., at 300 ppm showed at least 75% mortality in comparison with untreated controls.

### Example B.3: Action on Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% (v/v) water and 25% (v/v) DMSO. Different concentrations of formulated compounds were sprayed onto the insect diet at 5 pl, using a custom-built micro atomizer, at two replications.

After application, microtiter plates were incubated at about 25±1C and about 75±5% relative humidity for 5 days. Egg and larval mortality were then visually assessed.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-118, C-119, C-120, C-121, C-122, C-126, C-127, C-128, C-129, and C-130, resp., at 800 ppm showed at least 75% mortality in comparison with untreated controls.

### Example B.4: Action on Silverleaf Whitefly (Bemisia argentifolii) (Adults)

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24-hour photoperiod) to prevent trapping of heat inside the cup. Mortality was assessed 3 days after treatment, compared to untreated control plants.

In this test, compound C-6, C-32, C-59, C-60, C-61, C-65, and C-67, resp., at 300 ppm showed at least 75% mortality in comparison with untreated controls.

### Example B.5: Action on Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1C and about 80±5% relative humidity for 5 days. Egg and larval mortality were then visually assessed.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70 C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-117, C-118, C-120, C-121, C-122, C-123, C-126, C-127, C-128, C-129, and C-130, resp., at 800 ppm showed at least 75% mortality in comparison with untreated controls.

### Example B.6: Action on Diamond Back Moth (Plutella xylostella)

The active compound is dissolved at the desired concentration in a mixture of 1:1 (v/v) distilled water: acetone. Surfactant (Kinetic® HV) is added at a rate of 0.01% (v/v). The test solution is prepared at the day of use.

Leaves of cabbage were dipped in test solution and air-dried. Treated leaves were placed in petri dishes lined with moist filter paper and inoculated with ten 3rd instar larvae. Mortality was recorded 72 hours after treatment. Feeding damages were also recorded using a scale of 0-100%.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-30, C-31, C-34, C-35, C-36, C-39, C-40, C-41, C-42, C-43, C-44, C-45, C-46, C-47, C-48, C-49, C-50, C-51, C-52, C-53, C-54, C-55, C-56, and C-57, resp., at 300 ppm showed at least 75% mortality in comparison with untreated controls.

### Example B.7: Action on Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24-hour photoperiod) to prevent trapping of heat inside the bags. Mortality and reduced feeding were assessed 4 days after treatment, compared to untreated control plants.

In this test, compounds C-1, C-2, C-3, C-4, C-5, C-6, C-7, C-8, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-65, C-66, C-67, C-68, C-69, C-70, C-71, C-72, C-73, C-74, C-75, C-76, C-77, C-78, C-80, C-81, C-82, C-83, C-84, C-85, C-86, C-87, C-88, C-89, C-90, C-91, C-92, C-93, C-94, C-95, C-96, C-97, C-98, C-99, C-100, C-101, C-102, C-103, C-104, C-105, C-106, C-107, C-108, C-109, C-110, C-111, C-113, C-115, C-116, C-117, C-118, C-119, C-120, C-121, C-122, C-123, C-125, C-126, C-127, C-128, C-129, and C-130, resp., at 300 ppm showed at least 75% mortality in comparison with untreated controls.

### Example B.8: Action on Diamond Back Moth (Plutella Xylostella)

For evaluating control of diamond back moth (Plutella xylostella) the test unit consisted of 96-well-microtiter plates containing an insect diet and 15-25 P. xylostella eggs. The compounds or mixtures were formulated using a solution containing 75% water and 25% DMSO. Different concentrations of formulated compounds or mixtures were sprayed onto the insect diet at 5 μl, using a custom-built micro atomizer, at two replications. For experimental mixtures in these tests identical volumes of both mixing partners at the desired concentrations respectively, were mixed together. After application, microtiter plates were incubated at 28±1° C., 80±5% RH for 5 days. Egg and larval mortality was then visually assessed.

In this test, compounds C-58, C-59, C-60, C-61, C-62, C-63, C-64, C-65, C-66, C-67, C-68, C-69, C-70, C-71, C-72, C-73, C-74, C-75, C-76, C-77, C-78, C-70, C-80, C-81, C-82, C-83, C-84, C-85, C-86, C-87, C-88, C-90, C-91, C-92, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-117, C-118, C-119, C-122, C-123, C-126, C-127, C-128, C-129, and C-130, resp., at 800 ppm showed at least 75% mortality in comparison with untreated controls.

The beneficial activity of the compounds according to the invention over structurally close compounds known from prior art was demonstrated by the following comparative experiments:

| Structure/Example | Example | Test B.8 @ 25 ppm | |
|---|---|---|---|
| 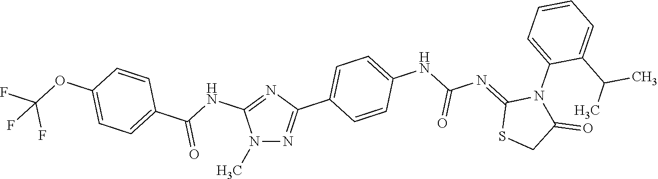 [SMILES: CC(C)c1ccccc1N1C(=O)CS/C1=N\C(=O)Nc1ccc(-c2nc(NC(=O)c3ccc(OC(F)(F)F)cc3)n(C)n2)cc1] | C-119 | 75 | |
| 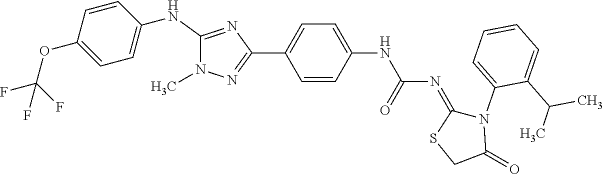 [SMILES: CC(C)c1ccccc1N1C(=O)CS/C1=N\C(=O)Nc1ccc(-c2nc(Nc3ccc(OC(F)(F)F)cc3)n(C)n2)cc1] | EP3974414 I-001 | 0 | |
| | | Test B.8 @ | Test B.8 @ |
| Structure/Example | Example | 25 ppm | 100 ppm |
| 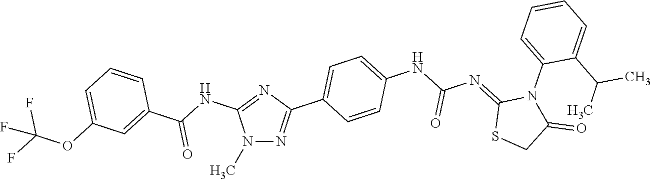 [SMILES: CC(C)c1ccccc1N1C(=O)CS/C1=N\C(=O)Nc1ccc(-c2nc(NC(=O)c3cccc(OC(F)(F)F)c3)n(C)n2)cc1] | C-126 | 75 | 100 |
| 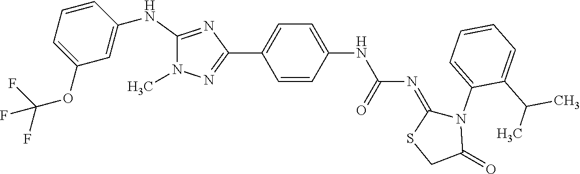 [SMILES: CC(C)c1ccccc1N1C(=O)CS/C1=N\C(=O)Nc1ccc(-c2nc(Nc3cccc(OC(F)(F)F)c3)n(C)n2)cc1] | EP3974414 I-006 | 38 | 0 |

1. A compound of formula I 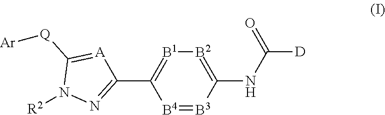
wherein
Q is —C(═O)—N(R⁵)— or —N(R⁵)—C(═O)—;
R⁵ is H, C₁-C₆-alkyl, C₃-C₆-cycloalkyl, C₁-C₆-alkyl-C₁-C₆-alkoxy, C₁-C₆-alkyl-C₃-C₆-cycloalkyl, phenyl, 5- or 6-membered hetaryl, —CH₂— phenyl, —CH₂-5- or -6-membered hetaryl, 1,3-dioxolan-2-ylmethyl, or halogen, wherein the alkyl, cycloalkyl, phenyl and hetaryl moieties are unsubstituted or substituted with halogen or CN;
A is N or CRA;
R^{A} is H, C₁-C₆-alkyl, C₃-C₆-cycloalkyl, C₁-C₆-alkoxy, halogen, CN, or
NR⁶R⁷ wherein the alkyl, alkoxy, and cycloalkyl moieties are unsubstituted or substituted with halogen or CN;
R² is H or C₁-C₆-alkyl, or C₃-C₆-cycloalkyl, wherein the alkyl and cycloalkyl moieties are unsubstituted or substituted with halogen, CN, or C₁-C₆-alkoxy;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA or DB, 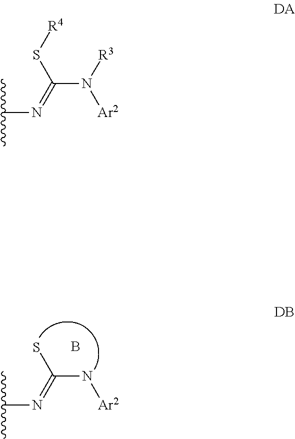
R³ is H, C₁-C₆-alkyl, or C₃-C₆-cycloalkyl, wherein the alkyl and cycloalkyl moieties are unsubstituted or substituted with halogen or CN;
R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkoxy, —O—(C═O)—C₁-C₆-alkyl; or CN; or
B is a 5- or 6-membered carbocyclic group, wherein 1 or 2 CH₂ moieties of the carbocyclic group may be replaced by a carbonyl group, O, or S, wherein the carbocyclic group is unsubstituted or substituted with R^{h};
Ar¹ is phenyl or 5- or 6-membered heteroaryl, which are unsubstituted or substituted with R^{Ar1}, wherein
R^{Ar1}; is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl, wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, S(═O)ₘR^{e}, —N═S(═O)—(C₁-C₆-alkyl)₂, or SO₂NR^{b}R^{c};
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl and heteroaryl moieties are unsubstituted or substituted with halogen, CN, C₁-C₆-alkyl, or C₁-C₆-alkoxy;
Ar² is phenyl or 5- or 6-membered heteroaryl, which are unsubstituted or substituted with R^{Ar2}, wherein
R^{Ar2} is halogen, CN, —SCN, SF₅, C₁-C₆-alkyl, C₁-C₆-alkoxy, C₂-C₆-alkenyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, cycloalkyl, and cycloalkoxy moieties are unsubstituted or substituted with halogen or CN, C(═O)—OR^{a}, NR^{b}R^{c}, C₁-C₆-alkylene-CN, C(═O)—NR^{b}R^{c};
R^{a}, R^{b}, and R^{c} are, identical or different, H, C₁-C₆-alkyl, C₂-C₆-alkenyl, C₃-C₆-cycloalkyl, C₃-C₆-cycloalkyl-C₁-C₄-alkyl, —C(═O)—C₁-C₆-alkyl, wherein the alkyl, alkenyl, and cycloalkyl moieties are unsubstituted or substituted with halogen;
R^{d} is H or C₁-C₆-alkyl;
R^{e} is C₁-C₆-alkyl or C₃-C₆-cycloalkyl, wherein the alkyl, cycloalkyl moieties are unsubstituted or substituted with halogen;
m is 0, 1, or 2;
R^{f} is halogen, OH, CN, SCN, SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, or C₃-C₆-cycloalkoxyx-C₁-C₄-alkyl, wherein the alkyl, alkoxy, alkenyl, alkynyl, cycloalkyl, and cycloalkoxy moieties are unsubstituted or substituted with halogen;
R^{h} is halogen, C₁-C₆-alkyl, or C₁-C₆-alkoxy;
and N-oxides, stereoisomers, tautomers, and agriculturally or veterinarily acceptable salts thereof.

2. The compound of formula I according to claim 1, wherein D is DB.

3. The compound of formula I according to claim 1, wherein D is a group selected from D5 to D7, which are unsubstituted or substituted with 1 or 2 substituents R^{h}

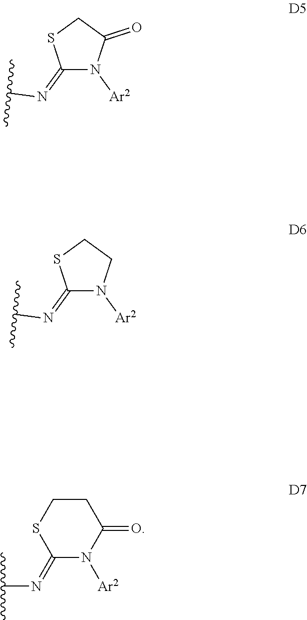

4. The compound of formula I according to claim 1 selected from the compounds of formula I.1 to I.8, wherein the variables are as defined in claim 1.

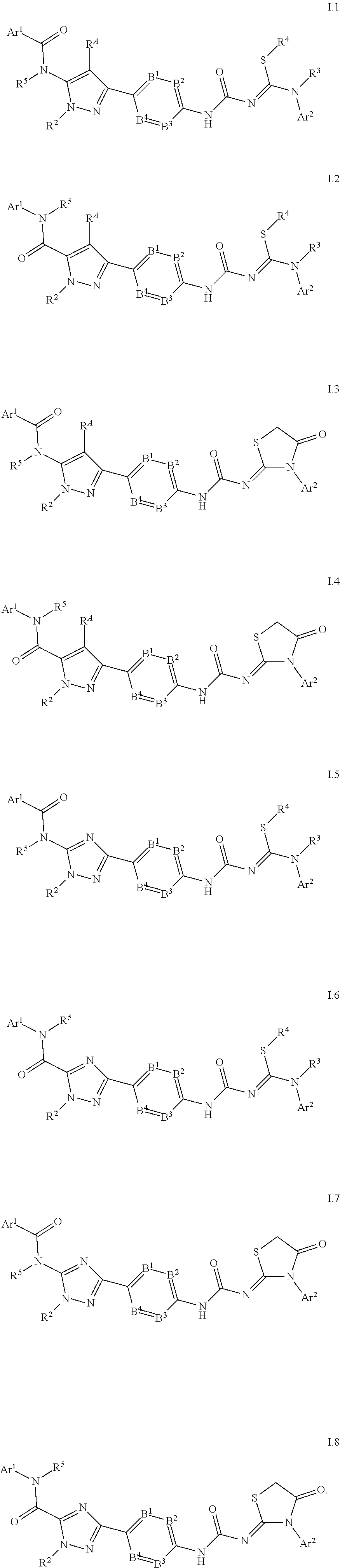

5. The compound of formula I according to claim 1, wherein
R⁵ is H, C₁-C₆-alkyl, or C₁-C₆-alkyl-CN;
R^{A} is H, CN, or C₁-C₆-alkyl;
R² is H or C₁-C₆-alkyl.

6. The compound of formula\ I according to claim 1, wherein B¹ is CR^{B1}, B² is CR^{B2}, and B³ is CR^{B3}.

7. The compound of formula I according to claim 1, wherein B¹ is N, B² is CR^{B2}, and B³ is CR^{B3}.

8. The compound of formula I according to claim 1, wherein R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen.

9. The compound of formula I according to claim 1, wherein
Ar¹ is phenyl which is unsubstituted or substituted with R^{Ar1};
R^{Ar1}; is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl, wherein the alkyl, alkoxy, alkenyl, alkynyl, cycloalkyl, C₃-C₆-heterocyclyl, and cycloalkoxy moieties are unsubstituted or substituted with R^{f}, C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b}, and R^{c}, identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

10. A composition comprising a compound of formula I according to claim 1, an N-oxide, or an agriculturally acceptable salt thereof, and a further active substance.

11. A method for combating or controlling an invertebrate pest comprising contacting said pest or its food supply, habitat or breeding grounds with a pesticidally effective amount of at least one compound according to claim 1.

12. A method for protecting growing plants from attack or infestation by an invertebrate pest comprising contacting a plant, or soil or water wherein the plant is growing, with a pesticidally effective amount of at least one compound according to claim 1.

13. A seed comprising a compound according to claim 1, or the enantiomers, diastereomers, or salts thereof, in an amount of from 0.1 g to 10 kg per 100 kg of seed.

14. (canceled)

15. A method for treating or protecting an animal from infestation or infection by an invertebrate pest comprising bringing the animal in contact with a pesticidally effective amount of at least one compound of the formula I according to claim 1, a stereoisomer thereof and/or at least one veterinarily acceptable salt thereof.
